(19) United States
(12) Patent Application Publication
(10) Pub. No.: US 2011/0086759 A1
(43) Pub. Date: Apr. 14, 2011

(54) CHEMICAL COMPOUNDS

(75) Inventors: Mary Bernadette Aspinall, Bracknell (GB); William Roderick Mound, Bracknell (GB); Jeffrey Steven Wailes, Bracknell (GB); William Guy Whittingham, Bracknell (GB); John Williams, Bracknell (GB); Caroline Louise Winn, Bracknell (GB); Worthington Paul Antony, Bracknell (GB)

(73) Assignee: SYNGENTA CROP PROTECTION, INC., Greensboro, NC (US)

(21) Appl. No.: 12/810,455

(22) PCT Filed: Dec. 18, 2008
(86) PCT No.: PCT/GB08/04184

§ 371 Date: Dec. 17, 2010

(30) Foreign Application Priority Data
Dec. 24, 2007 (GB) 0725218.2

(51) Int. Cl. A01N 25/32; C07D 239/02; C07D 401/12; C07D 403/12; A01N 43/54; A01N 43/60; A01P 3/00; A01P 5/00; A01P 7/00; A01P 13/00

(52) U.S. Cl. 504/103; 544/335; 544/333; 544/295; 504/239; 504/136

(57) ABSTRACT

The present invention relates to substituted pyrimidine derivatives of as well as N-oxides and agriculturally acceptable salts thereof, and their use to control undesired plant growth, in particular in crops of useful plants. The invention extends to herbicidal compositions comprising such compounds, N-oxides and/or salts as well as mixtures of the same with one or more further active ingredient (such as, for example, an herbicide, fungicide, insecticide and/or plant growth regulator) and/or a safener.

The present invention relates to substituted pyrimidine derivatives, as well as N-oxides thereof and agriculturally acceptable salts thereof, and their use to control undesired plant growth, in particular in crops of useful plants. The invention extends to herbicidal compositions comprising such compounds, N-oxides and/or salts as well as mixtures of the same with one or more further active ingredient (such as, for example, an herbicide, fungicide, insecticide and/or plant growth regulator) and/or a safener.

A large number of substituted pyrimidine derivatives are known in the art. For example, International Patent Publication No. WO 98/09960 describes 2,4-disubstituted pyrimidine derivatives for medicinal use, whilst Stansfield at al. (2004, Bioorg. Med. Chem. Lett. 14:5085-5088) describe 6-(4-methoxy-benzylamino)-5-nitro-2-thiophen-2yl-pyrimidine-4-carboxylic acid methyl ester as an intermediate in their exploration of 5,6-dihydroxypyrimidine-4-carboxylic acids as compounds for the treatment of hepatitis C. Reid & Gernfried (1988, Liebigs Annalen der Chemie 7:643-8) describe certain 4-pyrimidine carboxylic acids and derivatives thereof, wherein the pyrimidine ring carries inter alia a substituted amino group at the 6 position. These compounds are used as intermediates in the synthesis of diazepines and azaphenalenes. Britikova et al. (1966 Khimiya Geterotsiklicheskikh Soedinenii 5:783-90) describe the synthesis and properties of amino derivatives of 5-(hydroxymethyl)pyrimidine-4-carboxylic acid lactone, in studying derivatives of orotic acid and its analogs.

Substituted pyrimidine derivatives comprising an optionally substituted cyclopropyl or optionally substituted phenyl group at position 2 in combination with inter alia a nitro or optionally substituted amino group at the position 6 of the pyrimidine ring and their use as herbicides are disclosed in International Patent Publication No. WO 2005/063721. International Patent Publication No. WO 2007/082076 discloses a number of 2-(poly-substituted aryl)-6-amino-5-halo-4-pyrimidine carboxylic acids and their use as herbicides, whilst International Patent Publication No. WO 2007/092184 discloses certain substituted pyrimidine carboxylic acid derivatives as compounds capable of improving the harvestability of crops.

In part, due to the evolution of herbicide-resistant weed populations, and herbicide-resistant crops becoming volunteer weeds, there is a continuing need to control such undesired plant growth in particular in crops of useful plants. Other factors, for example, the demand for cheaper, more effective herbicides, and for herbicides with an improved environmental profile (e.g. safer, less toxic etc.) also drive the need to identify novel herbicidal compounds.

The present invention is based on the finding that substituted amino-pyrimidine derivatives, in particular where the amino group is substituted with an optionally substituted 3-8 membered saturated, unsaturated or aromatic ring, are particularly good herbicidal compounds.

Thus in a first aspect, the invention provides the use of a compound of formula (I)

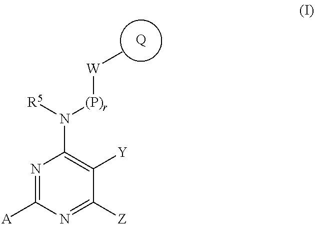

or salt or N-oxide thereof, wherein:A is halogen, optionally substituted alkyl, optionally substituted alkenyl, or an optionally substituted 3-8 membered ring optionally containing up to 4 heteroatoms; R⁵ is hydrogen, optionally substituted alkyl, optionally substituted cycloalkyl, formyl, optionally substituted alkylcarbonyl, optionally substituted cycloalkylcarbonyl, optionally substituted alkenylcarbonyl, optionally substituted alkynylcarbonyl, optionally substituted phenylcarbonyl, optionally substituted heteroarylcarbonyl, optionally substituted aminocarbonyl, optionally substituted alkoxycarbonyl, optionally substituted alkylsulphonyl, optionally substituted cycloalkylsulphonyl, optionally substituted alkenylsulphonyl, optionally substituted alkynylsulphonyl, optionally substituted phenylsulphonyl, optionally substituted amino, hydroxyl, optionally substituted alkoxy, optionally substituted cycloalkoxy, optionally substituted alkylcarbonyloxy, optionally substituted alkoxycarbonyloxy, optionally substituted aminocarbonyloxy, optionally substituted phenoxy, optionally substituted phenyl, optionally substituted heteroaryl, optionally substituted alkenyl, optionally substituted alkynyl, N═CR^{g}R^{h}, cyano, optionally substituted silyl, or optionally substituted phosphonyl; R^{g} is hydrogen, optionally substituted alkyl, optionally substituted cycloalkyl, optionally substituted phenyl, optionally substituted heteroaryl, optionally substituted amino, optionally substituted alkoxy, optionally substituted phenoxy, optionally substituted alkylthio, or optionally substituted phenylthio; R^{h} is optionally substituted alkyl, optionally substituted cycloalkyl, optionally substituted phenyl, optionally substituted heteroaryl, optionally substituted amino, optionally substituted alkoxy, or optionally substituted alkylthio; or R^{g} and R^{h} together with the C atom to which they are joined form an optionally substituted ring; P is CR¹⁰R¹¹; wherein R¹⁰ and R¹¹ are independently: hydrogen; cyano; nitro; alkyl; haloalkyl; hydroxyalkyl; alkoxyalkyl; carboxyalkyl; alkoxycarbonylalkyl; alkylcarbonyloxyalkyl; cycloalkyl; alkenyl; phenyl, optionally substituted by 1-3 groups R^{ea}; heteroaryl, optionally substituted by 1-3 groups R^{ea}; alkoxy; haloalkoxy; alkylthio; haloalkylthio; alkylsulphonylalkyl; alkylcarbonyl; alkoxycarbonyl; carboxy; aminocarbonyl; alkylaminocarbonyl; dialkylaminocarbonyl; or dialkylphosphonyl; each R^{ea} is independently: halogen, cyano, alkyl, haloalkyl, alkoxy, haloalkoxy, alkylthio, or haloalkylthio; W is a direct bond or an optionally substituted aliphatic chain, wherein said chain optionally comprises a heteroatom selected from O, N or S; r is an integer of 0 or 1 when W is a direct bond, or is the integer 1 when W is an optionally substituted aliphatic chain; Q is an optionally substituted 3-8 membered ring optionally containing up to 4 heteroatoms; Y is halogen, cyano, nitro, hydroxyl, optionally substituted alkyl, optionally substituted cycloalkyl, optionally substituted alkenyl, optionally substituted alkynyl, optionally substituted phenyl, optionally substituted heteroaryl, optionally substituted alkoxy, optionally substituted amino, optionally substituted alkylthio, optionally substituted alkylsulphinyl, optionally substituted alkylsulphonyl, optionally substituted dialkyl phosphonyl, optionally substituted trialkylsilyl; Z is (T)ₘ-(U)ₙ—V; wherein m is an integer of 0 to 1; n is an integer from 0 to 3, and n≧m; T is an oxygen or sulphur atom; U is CR^{w}R^{x}; each R^{w} is independently hydrogen, halogen, hydroxyl, optionally substituted alkyl, optionally substituted alkoxycarbonyl, or OR^{y} and each R^{y} is independently an optionally substituted alkyl or an optionally substituted alkylcarbonyl; each R^{x} is independently hydrogen, halogen, optionally substituted alkyl, OR^{Z} , and each R^{Z} is independently an optionally substituted alkyl group; or any geminal R^{w} and R^{x} together form a group selected from oxo, or ═NOR^{ca}, wherein R^{ca} is hydrogen or optionally substituted alkyl; or any geminal, vicinal or non-adjacent R^{w} and/or R^{x} together with the C atom(s) to which they are attached and any intervening atom form an optionally substituted 3-6 membered ring; or wherein when at least one R^{w} is OR^{y} and at least one R^{x} is OR^{Z}, said OR^{y} and OR^{Z} groups together with the C atom(s) to which they are attached and any intervening atom form an optionally substituted 5-6 membered heterocyclic ring; V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn}; R^{cb} is hydrogen, hydroxyl, optionally substituted alkoxy, optionally substituted cycloalkoxy, optionally substituted alkylthio, or optionally substituted amino; R^{cd} is optionally substituted alkoxy, optionally substituted cycloalkoxy, optionally substituted alkylthio, or optionally substituted amino; R^{ce} is hydrogen, optionally substituted alkyl, optionally substituted alkoxy, optionally substituted cycloalkoxy, or optionally substituted amino; R^{cf} is hydrogen, optionally substituted alkoxy, optionally substituted cycloalkoxy, optionally substituted alkylthio, or optionally substituted amino; R^{cg} and R^{ch} are each independently an optionally substituted alkoxy group; or R^{cg} and R^{ch}, together with the carbon to which they are joined, form a dioxolane or dioxane ring, which ring is optionally substituted; R^{ci} and R^{cj} are each independently an optionally substituted alkyl group; or R^{ci} and R^{cj}, together with the carbon and sulphur atoms to which they are joined, form an optionally substituted 5-6 membered ring; p and q are each independently an integer of 0,1, or 2; R^{ck}, R^{cl}, and R^{cm} are each independently an optionally substituted alkoxy group; or R^{ck} and R^{cl} and R^{cm} together with the carbon to which they are attached form an optionally substituted trioxabicyclo[2.2.2]octane ring system; and R^{cn} is hydrogen or an optionally substituted alkylcarbonyl group, as a herbicide.10 4

In a second aspect the invention provides novel compounds of formula (I), i.e. a compound of formula (I) as described hereinbefore, with the proviso that (i) Q is not a dioxolan-4-yl ring, or, (ii) said compound is not 6-methoxycarbonyl-4-[(4-methoxyphenyl)methylamino]-5-nitro-2-thiophen-2-yl-pyrimidine, 6-aminocarbonyl-4-[(2-aminophenyl)amino]-5-cyano-2-pyrrolidin-1-yl-pyrimidine, 6-dimethylaminocarbonyl-5-methyl-4-{[(N-methyl-N-phenyl-aminocarbonyl)-methyl]-amino}-2-pyridin-4-yl-pyrimidine, 5-methyl-4-{[N-methyl-N-phenyl-aminocarbonyl)-methyl]-amino}-2-pyrimidin-4-yl-pyrimidine-6-carboxylic acid, 6-ethoxycarbonyl-5-methyl-4-{[N-methyl-N-phenyl-aminocarbonyl)-methyl]-amino}-2-pyridin-4-yl-pyrimidine, 4-phenylamino-2-piperidin-1-yl-5-piperidin-1-ylmethyl-pyrimidine-6-carboxylic acid, or 4-[(2-aminophenyl)amino]-5-cyano-6-ethoxycarbonyl-2-pyrrolidin-1-yl-pyrimidine.

For the avoidance of doubt, the term “compound” as used herein includes all salts and N-oxides of said compound.

The compounds of formula (I) may exist in different geometric or optical isomers or different tautomeric forms. One or more centres of chirality may be present, in which case compounds of the formula (I) may be present as pure enantiomers, mixtures of enantiomers, pure diastereomers or mixtures of diastereomers. There may be double bonds present in the molecule, such as C═C or C═N bonds, in which case compounds of formula (I) may exist as single isomers or mixtures of isomers. Centres of tautomerisation may be present. This invention covers all such isomers and tautomers and mixtures thereof in all proportions as well as isotopic forms such as deuterated compounds.

Suitable acid addition salts include those with an inorganic acid such as hydrochloric, hydrobromic, sulfuric, nitric and phosphoric acids, or an organic carboxylic acid such as oxalic, tartaric, lactic, butyric, toluic, hexanoic and phthalic acids, or sulphonic acids such as methane, benzene and toluene sulphonic acids. Other examples of organic carboxylic acids include haloacids such as trifluoroacetic acid.

N-oxides are oxidised forms of tertiary amines or oxidised forms of nitrogen containing heteroaromatic compounds. They are described in many books for example in “Heterocyclic N-oxides” by Angelo Albini and Silvio Pietra, CRC Press, Boca Raton, Fla., 1991.

Each alkyl moiety either alone or as part of a larger group (such as alkoxyalkyl, alkylthio, alkylsulphinyl, alkylsulphonyl, alkylcarbonyl, haloalkylcarbonyl etc.) is a straight or branched chain and is, for example, methyl, ethyl, n-propyl, n-butyl, n-pentyl, n-hexyl, iso-propyl, n-butyl, sec-butyl, iso-butyl, tert-butyl or neo-pentyl. Unless otherwise stated herein, the alkyl groups are typically C₁ to C₁₀ alkyl groups, but are preferably C₁-C₈, even more preferably C₁-C₆ and most preferably C₁-C₄ alkyl groups.

Ring or chain forming alkylene, alkenylene and alkynyl groups can optionally be further substituted by one or more halogen, C₁-₃ alkyl and/or C₁₋₃ alkoxy groups.

When present, the optional substituents on an alkyl moiety (alone or as part of a larger group) include one or more of halogen, nitro, cyano, rhodano, isothiocyanato, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkylsilyl(C₁₋₆)alkoxy, C₁₋₆ alkoxycarbonyl(C₁₋₁₀)alkoxy, C₁₋₁₀ haloalkoxy, aryl(C₁₋₄)-alkoxy (where the aryl group is optionally substituted), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio (where the aryl group is optionally substituted), C₃₋₇ cycloalkylthio (where the cycloalkyl group is optionally substituted with C₁₋₆ alkyl or halogen), tri(C₁₋₄)alkylsilyl(C₁₋₆)alkylthio, arylthio (where the aryl group is optionally substituted), C₁₋₆ alkylsulfonyl, C₁₋₆ haloalkylsulfonyl, C₁₋₆ alkylsulfinyl, C₁₋₆ haloalkylsulfinyl, arylsulfonyl (where the aryl group may be optionally substituted), tri(C₁₋₄)alkylsilyl, aryldi(C₁₋₄)alkylsilyl, (C₁₋₄)alkyldiarylsilyl, triarylsilyl, aryl(C₁₋₄)alkylthio(C₁₋₄)alkyl, aryloxy(C₁₋₄)alkyl, formyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)aminocarbonyl, N—(C₁₋₃ alkyl)-N—(C₁₋₃ alkoxy)aminocarbonyl, C₁₋₆ alkylcarbonyloxy, arylcarbonyloxy (where the aryl group is optionally substituted), di(C₁₋₆)alkylaminocarbonyloxy, oximes and oxime-ethers such as ═NOalkyl, ═NOhaloalkyl and ═NOaryl (itself optionally substituted), aryl (itself optionally substituted), heteroaryl (itself optionally substituted), heterocyclyl (itself optionally substituted with C₁₋₆ alkyl or halogen), aryloxy (where the aryl group is optionally substituted), heteroaryloxy, (where the heteroaryl group is optionally substituted), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, C₂₋₆ alkenylcarbonyl, C₂₋₆ alkynylcarbonyl, C₃₋₆ alkenyloxycarbonyl, C₃₋₆ alkynyloxycarbonyl, aryloxycarbonyl (where the aryl group is optionally substituted) and arylcarbonyl (where the aryl group is optionally substituted).

Alkenyl and alkynyl moieties can be in the form of straight or branched chains, and the alkenyl moieties, where appropriate, can be of either the (E)- or (Z)-configuration. Examples are vinyl, allyl and propargyl. Alkenyl and alkynyl moieties can contain one or more double and/or triple bonds in any combination. It is understood, that the term allenyl falls within the scope of the terms alkeny and alkynyl.

When present, the optional substituents on alkenyl or alkynyl include those optional substituents given above for an alkyl moiety.

In the context of this specification acyl is optionally substituted C₁₋₆ alkylcarbonyl (for example acetyl), optionally substituted C₂₋₆ alkenylcarbonyl, optionally substituted C₃₋₆ cycloalkylcarbonyl (for example cyclopropylcarbonyl), optionally substituted C₂₋₆ alkynylcarbonyl, optionally substituted arylcarbonyl (for example benzoyl) or optionally substituted heteroarylcarbonyl.

Halogen is fluorine, chlorine, bromine or iodine.

Haloalkyl groups are alkyl groups which are substituted with one or more of the same or different halogen atoms and are, for example, CF₃, CF₂Cl, CF₂H, CCl₂H, FCH₂, ClCH₂, BrCH₂, CH₃CHF, (CH₃)₂CF, CF₃CH₂ or CHF₂CH₂.

In the context of the present specification ring systems may be saturated, unsaturated, or aromatic, and may also be fused, spiro or bridging ring systems. The terms “aryl”, “aromatic ring” and “aromatic ring system” as used herein refer to ring systems which may be mono-, bi- or tricyclic. Examples of such rings include phenyl, naphthalenyl, anthracenyl, indenyl or phenanthrenyl. A preferred aryl group is phenyl. In addition, the terms “heteroaryl”, “heteroaromatic ring” or “heteroaromatic ring system” refer to an aromatic ring system containing at least one heteroatom and consisting either of a single ring or of two or more fused rings. Preferably, single rings will contain up to three and bicyclic systems up to four heteroatoms which will preferably be chosen from nitrogen, oxygen and sulphur. Examples of such groups include furyl, thienyl, pyrrolyl, pyrazolyl, imidazolyl, 1,2,3-triazolyl, 1,2,4-triazolyl, oxazolyl, isoxazolyl, thiazolyl, isothiazolyl, 1,2,3-oxadiazolyl, 1,2,4-oxadiazolyl, 1,3,4-oxadiazolyl, 1,2,5-oxadiazolyl, 1,2,3-thiadiazolyl, 1,2,4-thiadiazolyl, 1,3,4-thiadiazolyl, 1,2,5-thiadiazolyl, pyridyl, pyrimidinyl, pyridazinyl, pyrazinyl, 1,2,3-triazinyl, 1,2,4-triazinyl, 1,3,5-triazinyl, benzofuryl, benzisofuryl, benzothienyl, benzisothienyl, indolyl, isoindolyl, indazolyl, benzothiazolyl, benzisothiazolyl, benzoxazolyl, benzisoxazolyl, benzimidazolyl, 2,1,3-benzoxadiazole, quinolinyl, isoquinolinyl, cinnolinyl, phthalazinyl, quinazolinyl, quinoxalinyl, naphthyridinyl, benzotriazinyl, purinyl, pteridinyl and indolizinyl. Preferred examples of heteroaromatic radicals include pyridyl, pyrimidyl, triazinyl, thienyl, furyl, oxazolyl, isoxazolyl, 2,1,3-benzoxadiazole and thiazolyl.

The terms heterocycle and heterocyclyl refer to a non-aromatic preferably monocyclic or bicyclic ring systems containing up to 10 atoms including one or more (preferably one or two) heteroatoms selected from O, S and N. Examples of such rings include 1,3-dioxolane, oxetane, tetrahydrofuran, morpholine, thiomorpholine and piperazine.

In the case of heteroaromatic or heterocyclic rings containing S as a heteroatom, the S atom may also be in the form of a mono- or di-oxide.

When present, the optional substituents on heterocyclyl include C₁₋₆ alkyl and C₁₋₆ haloalkyl, an oxo-group (allowing one of the carbon atoms in the ring to be in the form of a keto group), as well as those optional substituents given above for an alkyl moiety.

Cycloalkyl includes cyclopropyl, cyclobutyl, cyclopentyl and cyclohexyl. Cycloalkylalkyl is preferentially cyclopropylmethyl. Cycloalkenyl includes cyclopentenyl and cyclohexenyl.

When present, the optional substituents on cycloalkyl or cycloalkenyl include C₁₋₃ alkyl as well as those optional substituents given above for an alkyl moiety.

Carbocyclic rings include aryl, cycloalkyl and cycloalkenyl groups.

When present, the optional substituents on aryl or heteroaryl are selected independently, from halogen, nitro, cyano, rhodano, isothiocyanato, C₁₋₆ alkyl, C₁₋₆ haloalkyl, hydroxyalkyl, C₁₋₆ alkoxy-(C₁₋₆)alkyl, C₂₋₆ alkenyl, C₂₋₆ haloalkenyl, C₂₋₆ alkynyl, C₃₋₇ cycloalkyl (itself optionally substituted with C₁₋₆ alkyl or halogen), C₅₋₇ cycloalkenyl (itself optionally substituted with C₁₋₆ alkyl or halogen), hydroxy, C₁₋₁₀ alkoxy, C₁₋₁₀ alkoxy(C₁₋₁₀)alkoxy, tri(C₁₋₄)alkyl-silyl(C₁₋₆)alkoxy, C₁₋₆ alkoxycarbonyl(C₁₋₁₀)alkoxy, C₁₋₁₀ haloalkoxy, aryl(C₁₋₄)alkoxy (where the aryl group is optionally substituted with halogen or C₁₋₆ alkyl), C₃₋₇ cycloalkyloxy (where the cycloalkyl group is optionally substituted with C₁₋₆ alkyl or halogen), C₂₋₁₀ alkenyloxy, C₂₋₁₀ alkynyloxy, mercapto, C₁₋₁₀ alkylthio, C₁₋₁₀ haloalkylthio, aryl(C₁₋₄)alkylthio, C₃₋₇ cycloalkylthio (where the cycloalkyl group is optionally substituted with C₁₋₆ alkyl or halogen), tri(C₁₋₄-alkylsilyl(C₁₋₆)alkylthio, arylthio, C₁₋₆ alkylsulfonyl, C₁₋₆ haloalkylsulfonyl, C₁₋₆ alkylsulfinyl, C₁₋₆ haloalkylsulfinyl, arylsulfonyl, tri(C₁₋₄)alkylsilyl, aryldi(C₁₋₄)-alkylsilyl, (C₁₋₄)alkyldiarylsilyl, triarylsilyl, C₁₋₁₀ alkylcarbonyl, HO₂C, C₁₋₁₀ alkoxycarbonyl, aminocarbonyl, C₁₋₆ alkylaminocarbonyl, di(C₁₋₆ alkyl)-aminocarbonyl, N—(C₁₋₃ alkyl)-N—(C₁₋₃ alkoxy)aminocarbonyl, C₁₋₆ alkylcarbonyloxy, arylcarbonyloxy, di(C₁₋₆)alkylamino-carbonyloxy, aryl (itself optionally substituted with C₁₋₆ alkyl or halogen), heteroaryl (itself optionally substituted with C₁₋₆ alkyl or halogen), heterocyclyl (itself optionally substituted with C₁₋₆ alkyl or halogen), aryloxy (where the aryl group is optionally substituted with C₁₋₆ alkyl or halogen), heteroaryloxy (where the heteroaryl group is optionally substituted with C₁₋₆ alkyl or halogen), heterocyclyloxy (where the heterocyclyl group is optionally substituted with C₁₋₆ alkyl or halogen), amino, C₁₋₆ alkylamino, di(C₁₋₆)alkylamino, C₁₋₆ alkylcarbonylamino, N—(C₁₋₆)alkylcarbonyl-N—(C₁₋₆)alkylamino, arylcarbonyl, (where the aryl group is itself optionally substituted with halogen or C₁₋₆ alkyl) or two adjacent positions on an aryl or heteroaryl system may be cyclised to form a 5, 6 or 7 membered carbocyclic or heterocyclic ring, itself optionally substituted with halogen or C₁₋₆ alkyl. Further substituents for aryl or heteroaryl include aryl carbonyl amino (where the aryl group is substituted by C₁₋₆ alkyl or halogen), C₁₋₆alkoxycarbonylamino, C₁₋₆alkoxycarbonyl-N—(C₁₋₆)alkylamino, aryloxycarbonylamino (where the aryl group is substituted by C₁₋₆ alkyl or halogen), aryloxycarbonyl-N—(C₁₋₆)alkylamino (where the aryl group is substituted by C₁₋₆ alkyl or halogen), arylsulphonylamino (where the aryl group is substituted by C₁₋₆ alkyl or halogen), arylsulphonyl-N—(C₁₋₆)alkylamino (where the aryl group is substituted by C₁₋₆ alkyl or halogen), aryl-N—(C₁₋₆)alkylamino (where the aryl group is substituted by C₁₋₆ alkyl or halogen), arylamino (where the aryl group is substituted by C₁₋₆ alkyl or halogen), heteroaryl amino (where the heteroaryl group is substituted by C₁₋₆ alkyl or halogen), heterocyclylamino (where the heterocyclyl group is substituted by C₁₋₆ alkyl or halogen), aminocarbonylamino, C₁₋₆ alkylaminocarbonyl amino, di(C₁₋₆)alkylaminocarbonyl amino, arylaminocarbonyl amino (where the aryl group is substituted by C₁₋₆ alkyl or halogen), aryl-N—(C₁₋₆)alkylaminocarbonylamino (where the aryl group is substituted by C₁₋₆ alkyl or halogen), C₁₋₆ alkylaminocarbonyl-N—(C₁₋₆)alkyl amino, di(C₁₋₆)alkylaminocarbonyl-N—(C₁₋₆)alkyl amino, arylaminocarbonyl-N—(C₁₋₆)alkyl amino (where the aryl group is substituted by C₁₋₆ alkyl or halogen) and aryl-N—(C₁₋₆)alkylaminocarbonyl-N—(C₁₋₆)alkyl amino (where the aryl group is substituted by C₁₋₆ alkyl or halogen).

For substituted phenyl moieties, heterocyclyl and heteroaryl groups it is preferred that one or more substituents are independently selected from halogen, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₂₋₆ alkenyl, C₂₋₆ haloalkenyl, C₂₋₆ alkynyl, C₃₋₇ cycloalkyl, nitro, cyano, CO₂H, C₁₋₆ alkylcarbonyl, C₁₋₆ alkoxycarbonyl, aryl, heteroaryl, C₁₋₆ alkylamino, di(C₁₋₆ alkyl)amino, C₁₋₆ alkylaminocarbonyl, or di(C₁₋₆ alkyl)aminocarbonyl.

Haloalkenyl groups are alkenyl groups which are substituted with one or more of the same or different halogen atoms.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

In preferred embodiments of the invention, the preferred groups for A, R⁵, P, r, W, Q, Y, and Z, in any combination thereof, are as set out below.

According to the invention A is halogen, optionally substituted alkyl, optionally substituted alkenyl, or an optionally substituted 3-8 membered ring optionally containing up to 4 heteroatoms.

Preferably A is selected from: halogen; alkyl optionally substituted by one or more groups R^{a}; alkenyl optionally substituted by one or more groups R^{b}; phenyl optionally substituted by 1-4 groups R¹; or a 5 or 6 membered heteroaromatic ring optionally substituted by 1-4 groups R¹, said heteroaromatic ring containing 1-4 heteroatoms selected from O, N and S, provided said heteroaromatic ring contains only one O or one S atom; a 3-8 membered non-aromatic ring containing 0-3 heteroatoms chosen from oxygen, sulphur and nitrogen, said ring being optionally substituted by 1-8 groups R².

More preferably A is fluorine; chlorine; bromine; iodine; C₁₋₆ alkyl optionally substituted by 1-3 groups R^{a}; C₁₋₆ haloalkyl optionally substituted by 1-3 groups R^{a}; C₂₋₆ alkenyl optionally substituted by 1-3 groups R^{b}; C₃₋₈ cycloalkyl optionally substituted by 1-4 groups R²; a ring system selected from: phenyl, thiophene, furan, pyrrole, isoxazole, isothiazole, oxazole, thiazole, imidazole, pyrazole, 1,2,3-oxadiazole,1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,3,4-oxatriazole, 1,2,3,4-thiatriazole, tetrazole, pyridine, pyridazine, pyrimidine, pyrazine, 1,2,3-triazine, 1,2,4-triazine, 1,3,5-triazine, and 1,2,4,5-tetrazine, wherein said ring system is optionally substituted by 1-3 groups R¹; or a ring system selected from: aziridine, azetidine, pyrrolidine, piperidine, piperazine, azepane, oxetane, tetrahydrofuran, tetrahydropyran, morpholine, thiomorpholine, dihydrofuran, dihydropyran, dioxolane, 1,3-dioxane, 1,4-dioxane, 1,3-dioxepane, dithiolane, 1,3-dithiane, 1,4-dithiane, wherein said ring system is optionally substituted by 1-4 groups R².

Even more preferably A is F, Cl, Br, I, a ring system selected from: phenyl, thiophene, furan, pyrrole, isoxazole, isothiazole, oxazole, thiazole, imidazole, pyrazole, 1,2,4-oxadiazole, 1,3,4-oxadiazole, 1,2,4-thiadiazole, 1,3,4-thiadiazole, 1,2,3-triazole, 1,2,4-triazole, pyridine, pyridazine, pyrimidine, pyrazine, 1,2,4-triazine, and 1,3,5-triazine, wherein said ring system is optionally substituted by 1-3 groups R¹; or C₃₋₆ cycloalkyl, optionally substituted by 1-4 groups R²;

Yet more preferably A is F, Cl, Br, I, a phenyl ring optionally substituted by 1-3 groups R¹, or cyclopropyl optionally substituted by 1-2 groups R². Most preferably A is a phenyl ring optionally substituted by 1-3 groups R¹, or cyclopropyl.

Each R¹ is independently: halogen; cyano; nitro; azido; hydroxyl; alkyl optionally substituted by one or more groups R^{a}; alkenyl optionally substituted by one or more groups R^{b}; alkynyl optionally substituted by one or more R^{c}; cycloalkyl optionally substituted by one or more groups R^{d}; OR^{aa}; S(O)ₐR^{bb}; C(O)R^{cc}; NR^{dd}R^{ee}; SiR^{ff}R^{gg}R^{hh}; P(O)RⁱⁱR^{jj}; B(OR^{kk})(OR^{LL}); or 2 adjacent groups R¹ together with the atoms to which they are joined form a 5-7 membered ring, optionally containing 1 or 2 heteroatoms selected from O, N and S, said 5-7 membered ring being optionally substituted with 1-4 groups R³.

Preferably each R¹ is independently: halogen; cyano; nitro; hydroxyl; C₁₋₆ alkyl optionally substituted by 1-4 groups R^{a}; C₁₋₆ haloalkyl optionally substituted by 1-4 groups R^{a}; OR^{aa}; S(O)ₐR^{bb}; C(O)R^{cc}; or NR^{dd}R^{ee}; or 2 adjacent groups R¹ together with the atoms to which they are joined form a 5-or 6-membered ring optionally containing 1 or 2 heteroatoms selected from O, N and S, said ring being optionally substituted with 1-4 groups R³.

More preferably each R¹ is independently: halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio, amino, C₁₋₄ alkylamino, di(C₁₋₂)alkylamino; or 2 adjacent groups R¹ together with the atoms to which they are joined form a 6 membered aromatic ring optionally substituted with 1-2 groups selected from: halogen, cyano, C₁₋₄ alkyl, C₁₋₄ haloalkyl, C₁₋₄ alkoxy, C₁₋₄ haloalkoxy, C₁₋₄ alkylthio, and C₁₋₄ haloalkylthio.

Even more preferably each each R¹ is independently: halogen, cyano, C₁₋₂ alkyl, C₁₋₂ haloalkyl, C₁₋₂ alkoxy, C₁₋₂ haloalkoxy, or di(C₁₋₂)alkylamino.

Most preferably each R¹ is independently halogen, C₁₋₂alkoxy, or dimethylamino.

Each R² is independently: halogen; cyano; nitro; hydroxyl; alkyl optionally substituted by one or more R^{a}; alkenyl optionally substituted by one or more R^{b}, alkynyl optionally substituted by one or more R^{c}; cycloalkyl optionally substituted by one or more R^{d}; phenyl optionally substituted by one or more R^{e}; heteroaryl optionally substituted by one or more R^{f}; OR^{aa}; S(O)ₐR^{bb}; C(O)R^{cc}; NR^{dd}R^{ee}; P(O)RⁱⁱR^{jj}; or any two geminal groups R² together form a group selected from oxo, ═CR^{mm}Rⁿⁿ, ═NOR^{oo}, and ═NNR^{pp}R^{qq}; or two groups R² together with the atoms to which they are joined and any intervening atoms form a 3-7 membered ring system, wherein said ring system optionally contains 1-2 heteroatoms independently selected from O, S and N and is optionally substituted by 1-4 groups R⁴.

Preferably each R² is independently: halogen; cyano; nitro; hydroxyl; C₁₋₆ alkyl optionally substituted by 1-4 groups R^{a}; C₁₋₆ haloalkyl optionally substituted by 1-4 groups R^{a}; C₂₋₆ alkenyl optionally substituted by 1-3 groups R^{b}; OR^{aa}; S(O)ₐR^{bb}; C(O)R^{cc}; or NR^{dd}R^{ee}; or any two geminal groups R² together form a group selected from oxo, ═CR^{mm}Rⁿⁿ, ═NOR^{oo}, ═NNR^{pp}R^{qq}; or two groups R² together with the atoms to which they are joined and any intervening atoms form a 3-7 membered ring system, wherein said ring system optionally contains 1-2 heteroatoms independently selected from O, S and N and is optionally substituted by 1-4 groups R⁴.

More preferably each R² is independently: halogen, cyano, C₁₋₄ alkyl, C₁₋₄ haloalkyl, C₁₋₄ alkoxy, C₁₋₄ haloalkoxy, C₁₋₄ alkoxycarbonyl, or C₁₋₄ alkylaminocarbonyl; or any two geminal groups R² together form a group selected from oxo, ═CR^{mm}Rⁿⁿ; ═NOR^{oo}; or two groups R² together with the atoms to which they are joined and any intervening atoms form a 5-6 membered ring system, optionally substituted by 1-2 groups selected from: halogen, cyano, C₁₋₄ alkyl, C₁₋₄ haloalkyl, C₁₋₄ alkoxy, and C₁₋₄ haloalkoxy.

Most preferably each R² is independently: halogen, cyano, C₁₋₂ alkyl, C₁₋₂ haloalkyl, C₁₋₂ alkoxy, C₁₋₂ haloalkoxy, or C₁₋₃ alkoxycarbonyl;

Each R³ is independently: halogen; cyano; nitro; hydroxyl; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; SiR^{ff}R^{gg}R^{hh}; P(O)RⁱⁱR^{jj}; B(OR^{kk})(OR^{LL}); or any two geminal groups R³ together form a group selected from: oxo, ═CR^{mm}Rⁿⁿ, ═NOR^{oo}, and ═NNR^{pp}R^{qq}. Preferably each R³ is independently: halogen; cyano; nitro; hydroxyl; C₁₋₆ alkyl optionally substituted by 1-4 groups R^{a}; C₁₋₆ haloalkyl optionally substituted by 1-4 groups R^{a}; OR^{aa}; S(O)ₐR^{bb}; C(O)R^{cc}; or NR^{dd}R^{ee}; or any two geminal groups R³ together form a group selected from oxo, ═CR^{mm}Rⁿⁿ, ═NOR^{oo}, or ═NNR^{pp}R^{qq}.

Each R⁴ is independently: halogen; cyano; nitro; hydroxyl; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; P(O)RⁱⁱR^{jj}; B(OR^{kk})(OR^{LL}); or any two geminal groups R⁴ together form a group selected from: oxo, ═CR^{mm}Rⁿⁿ, ═NOR^{oo}, and ═NNR^{pp}R^{qq}.

Preferably each R⁴ is independently: halogen; cyano; nitro; hydroxyl; C₁₋₆ alkyl optionally substituted by 1-4 groups R^{a}; C₁₋₆ haloalkyl optionally substituted by 1-4 groups R^{a}; C₂₋₆ alkenyl optionally substituted by 1-3 groups R^{b}; OR^{aa}; S(O)ₐR^{bb}; C(O)R^{cc}; or NR^{dd}R^{ee}; or any two geminal groups R⁴ together form a group selected from oxo; ═CR^{mm}Rⁿⁿ; ═NOR^{oo}; ═NNR^{pp}R^{qq}.

Each R^{a} is independently: halogen; cyano; nitro; hydroxyl; cycloalkyl; OR^{aa}; S(O)ₐR^{bb}; C(O)R^{cc}; or NR^{dd}R^{ee}.

Preferably each R^{a} is independently: cyano, hydroxyl, C₃₋₆ cycloalkyl, OR^{aa}, S(O)ₐR^{bb}, C(O)R^{cc}, or NR^{dd}R^{ee}.

Each R^{b} is independently: halogen; cyano; nitro; hydroxyl; alkoxy; S(O)₂R^{bb}; C(O)R^{cc}; or P(O)RⁱⁱR^{jj}.

Preferably each R^{b} is independently: halogen, cyano, nitro, C₁₋₆ alkoxy, S(O)₂R^{bb}, or C(O)R^{cc}.

Each R^{c} is independently: halogen; cyano; alkoxy; S(O)₂R^{bb}; C(O)R^{cc}; or SiR^{ff}R^{gg}R^{hh}.

Each R^{d} is independently: halogen; cyano; nitro; hydroxyl; alkyl; alkenyl; alkynyl; cycloalkyl; alkoxy; S(O)ₐR^{bb}; or C(O)R^{cc}.

Each R^{e} is independently: halogen; cyano; nitro; hydroxyl; alkyl optionally substituted by one or more R^{a} wherein R^{a} is as defined above; OR^{aa}; S(O)ₐR^{bb}; C(O)R^{cc}; or NR^{dd}R^{ee}.

Each R^{f} is independently: halogen; cyano; nitro; hydroxyl; alkyl optionally substituted by one or more R^{a} wherein R^{a} is as defined above; OR^{aa}; S(O)ₐR^{bb}; C(O)R^{cc}; or NR^{dd}R^{ee}.

Each R^{aa} is independently: alkyl, haloalkyl, alkoxyalkyl, cycloalkylalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, dialkylaminocarbonyl, alkylimino, or dialkylimino.

Preferably each R^{aa} is independently: C₁₋₆ alkyl, C₁₋₆ haloalkyl, C₁-₄ alkoxy(C₁₋₄)alkyl, or C₁₋₆ alkylcarbonyl.

Each R^{bb} is independently: alkyl, haloalkyl, alkoxyalkyl, alkenyl, haloalkenyl, alkynyl, haloalkynyl, cycloalkyl, or alkylcarbonylamino;

Preferably each R^{bb} is independently: C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino;

ₐ is an integer selected from 0, 1, and 2.

R^{cc} is hydrogen, hydroxyl, alkyl, haloalkyl, alkoxyalkyl, alkenyl, haloalkenyl, alkynyl, haloalkynyl, cycloalkyl, alkoxy, haloalkoxy, alkoxyalkoxy, arylalkoxy, cycloalkoxy, amino, alkylamino, dialkylamino, or alkylsulphonylamino.

Preferably each R^{cc} is independently: hydrogen, hydroxyl, C₁₋₆ alkyl, C₁₋₆ haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino, or C₁₋₆ alkylsulphonylamino;

R^{dd} is hydrogen, alkyl, haloalkyl, alkoxyalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

R^{ee} is hydrogen, alkyl, haloalkyl, alkoxyalkyl, alkenyl, haloalkenyl, alkynyl, haloalkynyl, or cycloalkyl.

Or R^{dd} and R^{ee}, together with the N atom to which they are joined form a 4-6 membered ring, optionally containing one further heteroatom selected from O, N and S, said ring being optionally substituted by 1-4 groups selected from halogen, alkyl, alkoxy, alkylcarbonyl, alkoxycarbonyl, and oxo.

Preferably each R^{dd} is independently: hydrogen, C₁₋₆ alkyl, C₁₋₆ alkylcarbonyl, C₁₋₆ alkoxycarbonyl, C₁₋₆ alkylsulphonyl, C₁₋₆ haloalkylsulphonyl, aminocarbonyl, C₁₋₆ alkylaminocarbonyl, or di(C₁₋₄)alkylaminocarbonyl.

Preferably each R^{ee} is independently: hydrogen, C₁₋₆ alkyl, C₃₋₆ cycloalkyl; or R^{dd} and R^{ee}, together with the N atom to which they are joined form a 5- or 6-membered saturated ring optionally containing one further heteroatom selected from O, N and S, said ring being optionally substituted by 1 or 2 C₁₋₆ alkyl or oxo groups;

R^{ff}, R^{gg} and R^{hh} are each independently alkyl or haloalkyl. Rⁱⁱ is alkyl, haloalkyl, alkoxy, or haloalkoxy. R^{jj} is alkoxy or haloalkoxy. R^{kk} and R^{LL} are each independently hydrogen or alkyl; or R^{kk} and R^{LL} together with the O atoms and B atom to which they are joined form a 5- or 6-membered heterocyclic ring optionally substituted by 1-4 alkyl groups.

R^{mm} is hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

Preferably each R^{mm} is independently: hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl.

More preferably each R^{mm} is independently hydrogen, halogen, cyano, nitro, C₁₋₄ alkyl, or C₁₋₄ alkoxycarbonyl.

Rⁿⁿ is hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

Preferably each Rⁿⁿ is independently: hydrogen, halogen, cyano, nitro, C₁₋₆ alkyl, C₁₋₆ alkylcarbonyl, C₁₋₆ alkoxycarbonyl, C₁₋₆ alkylsulphonyl, or aminocarbonyl.

More preferably each Rⁿⁿ is independently: hydrogen, halogen, cyano, nitro, C₁₋₄ alkyl, or C₁₋₄ alkoxycarbonyl.

R^{oo} is hydrogen, alkyl, haloalkyl, alkoxyalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl.

Preferably each R^{oo} is independently hydrogen, C₁₋₆ alkyl, or C₃₋₆ cycloalkyl.

R^{pp} is hydrogen, alkyl, haloalkyl, alkoxyalkyl, or cycloalkyl.

R^{qq} is hydrogen, alkyl, haloalkyl, alkylcarbonyl, haloalkylcarbonyl, alkoxycarbonyl, aminocarbonyl, alkylaminocarbonyl, or dialkylaminocarbonyl, or R^{pp} and R^{qq} together with the nitrogen to which they are attached form a 5- or 6-membered optionally substituted ring, said ring optionally containing a further heteroatom selected from oxygen, sulphur and nitrogen, said substitution(s) being selected from: halogen and alkyl.

Preferably each R^{pp} is independently hydrogen, C₁₋₆ alkyl, or C₃₋₆ cycloalkyl. Preferably each R^{qq} is independently hydrogen or C₁₋₆ alkyl.

As defined herein R⁵ is hydrogen, optionally substituted alkyl, optionally substituted cycloalkyl, formyl, optionally substituted alkylcarbonyl, optionally substituted cycloalkylcarbonyl, optionally substituted alkenylcarbonyl, optionally substituted alkynylcarbonyl, optionally substituted phenylcarbonyl, optionally substituted heteroarylcarbonyl, optionally substituted aminocarbonyl, optionally substituted alkoxycarbonyl, optionally substituted alkylsulphonyl, optionally substituted cycloalkylsulphonyl, optionally substituted alkenylsulphonyl, optionally substituted alkynylsulphonyl, optionally substituted phenylsulphonyl, optionally substituted amino, hydroxyl, optionally substituted alkoxy, optionally substituted cycloalkoxy, optionally substituted alkylcarbonyloxy, optionally substituted alkoxycarbonyloxy, optionally substituted aminocarbonyloxy, optionally substituted phenoxy, optionally substituted phenyl, optionally substituted heteroaryl, optionally substituted alkenyl, optionally substituted alkynyl, N═CR^{g}R^{h}, cyano, optionally substituted silyl, or optionally substituted phosphonyl.

In certain embodiments R⁵ is selected from: hydrogen; alkyl optionally substituted by one or more R^{p}; cycloalkyl optionally substituted by one or more R^{q}; phenyl optionally substituted by one or more R^{r}; heteroaryl optionally substituted by one or more R^{s}; alkenyl optionally substituted by one or more R^{t}; alkynyl optionally substituted by one or more R^{u}; OR^{rr}; SO₂R^{ss}; C(O)R^{uu}; NR^{vv}R^{ww}; N═CR^{g}R^{h}; cyano; trialkylsilyl; and dialkyl phosphonyl.

Preferably R⁵ is: hydrogen; C₁₋₆ alkyl optionally substituted by 1-4 groups R^{p}; C₁₋₆ haloalkyl optionally substituted by 1-4 groups R^{p}; C₃₋₈ cycloalkyl optionally substituted by 1-4 groups R^{q}; phenyl optionally substituted by 1-3 groups R^{r}; heteroaryl optionally substituted by 1-3 groups R^{s}; C₃₋₆ alkenyl optionally substituted by 1-4 groups R^{t}; C₃₋₆ alkynyl optionally substituted by 1-4 groups R^{u}; SO₂R^{ss}; or C(O)R^{uu}.

More preferably R⁵ is hydrogen; C₁₋₄ alkyl optionally substituted by 1-2 groups R^{p}; C₁₋₄ haloalkyl optionally substituted by 1-2 groups R^{p}; C₃₋₄ alkenyl or C₃₋₄ alkynyl.

More preferably still, R⁵ is hydrogen, methyl, ethyl, n-propyl, iso-propyl, n-butyl, tert-butyl, trimethylsilylmethyl, prop-2-en-1-yl, prop-2-yn-1-yl, but-3-en-1-yl, phenylmethyl, CH2COOH, 2-hydroxyethyl, 1,1-dimethyl-2-hydroxyethyl, 2-hydroxyprop-1-yl, 2-methoxyethyl, 2-cyanoethyl, or 2-ethoxycarbonylethyl.

Most preferably R⁵ is hydrogen or C₁₋₂ alkyl.

R^{g} is hydrogen, optionally substituted alkyl, optionally substituted cycloalkyl, optionally substituted phenyl, optionally substituted heteroaryl, optionally substituted amino, optionally substituted alkoxy, optionally substituted phenoxy, optionally substituted alkylthio, or optionally substituted phenylthio.

R^{h} is optionally substituted alkyl, optionally substituted cycloalkyl, optionally substituted phenyl, optionally substituted heteroaryl, optionally substituted amino, optionally substituted alkoxy, or optionally substituted alkylthio; or R^{g} and R^{h} together with the C atom to which they are joined form an optionally substituted ring;

Preferably R^{g} is: hydrogen, alkyl optionally substituted by one or more R^{xx}, cycloalkyl optionally substituted by one or more R^{yy}, phenyl optionally substituted by one or more R^{zz}, heteroaryl optionally substituted by one or more R^{ab}, NR^{ac}R^{ad}, OR^{ae}, or SR^{af}.

Preferably R^{h} is: alkyl optionally substituted by one or more R^{xx}, cycloalkyl optionally substituted by one or more R^{yy}, phenyl optionally substituted by one or more R^{zz}, heteroaryl optionally substituted by one or more R^{ab}, NR^{ac}R^{ad}, OR^{ae}, or SR^{af}; or R^{g} and R^{h} together with the carbon atom to which they are joined form a ring that is optionally substituted by 1-4 groups R^{ag}.

Each R^{p} is independently: halogen; cyano; nitro; hydroxyl; cycloalkyl; phenyl optionally substituted by one or more R^{aj}; heteroaryl optionally substituted by one or more R^{ak}; alkoxy; alkoxyalkoxy; S(O)_{b}R^{am}; C(O)R^{an}; NR^{ao}R^{ap}; or trialkylsilyl.

Preferably each R^{p} is independently: cyano; hydroxyl; C₃₋₆ cycloalkyl; phenyl optionally substituted by 1-3 groups R^{aj}; heteroaryl optionally substituted by 1-3 groups R^{ak}; C₁₋₄ alkoxy; C₁₋₄ alkoxy(C₁₋₄)alkoxy; or tri(C₁₋₄)alkylsilyl.

More preferably each R^{p} is independently cyano, hydroxyl, phenyl, C₁₋₄ alkoxy, CO₂H, C₁₋₄alkoxycarbonyl or trimethylsilyl.

Each R^{q} is independently: halogen; cyano; nitro; hydroxyl; alkyl; haloalkyl; alkenyl; alkynyl; cycloalkyl; phenyl optionally substituted by one or more R^{aj}; heteroaryl optionally substituted by one or more R^{ak}; alkoxy; S(O)_{b}R^{am}; or C(O)R^{an};

Preferably each R^{q} is independently halogen, cyano, C₁₋₄ alkyl, C₁₋₄ haloalkyl, C₃₋₆ cycloalkyl, phenyl optionally substituted by 1-3 groups R^{aj}, or C(O)R^{an}. More preferably each R^{q} is independently halogen or C₁₋₄ alkyl.

Each R^{r} is independently halogen; cyano; nitro; hydroxyl; alkyl optionally substituted by one or more R^{aq}; alkenyl optionally substituted by one or more R^{ar}; alkynyl optionally substituted by one or more R^{as}; cycloalkyl optionally substituted by one or more R^{at}; OR^{al}; S(O)_{b}R^{am}; or C(O)R^{an}.

Preferably each R^{r} is independently halogen, cyano, nitro, C₁₋₄ alkyl, C₁₋₄ haloalkyl, C₂₋₈ alkoxyalkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl;

Each R^{s} is independently: halogen; cyano; nitro; hydroxyl; alkyl optionally substituted by one or more R^{aq}; alkenyl optionally substituted by one or more R^{ar}; alkynyl optionally substituted by one or more R^{as}; cycloalkyl optionally substituted by one or more R^{at}; OR^{at}; S(O)_{b}R^{am}; or C(O)R^{an}.

Preferably each R^{s} is independently halogen, cyano, C₁₋₄ alkyl, C₁₋₄ haloalkyl, C₂₋₈ alkoxyalkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl;

Each R^{t} is independently: halogen, cyano, cycloalkyl, phenyl, or alkoxycarbonyl.

Preferably each R^{t} is independently: halogen, cyano, or C₁₋₄ alkoxycarbonyl.

Each R^{u} is independently: halogen, cyano, cycloalkyl, phenyl, alkoxy, or alkoxycarbonyl.

Preferably each R^{u} is independently: cyano, or C₁₋₄ alkoxycarbonyl.

Each R^{rr} is independently: hydrogen; alkyl; haloalkyl; cycloalkyl; alkylcarbonyl; haloalkylcarbonyl; alkoxycarbonyl; aminocarbonyl; alkylaminocarbonyl; dialkylaminocarbonyl; or phenyl optionally substituted by one or more groups R^{zz}.

Each R^{ss} is independently: alkyl; haloalkyl; alkenyl; haloalkenyl; alkynyl; haloalkynyl; cycloalkyl; or phenyl optionally substituted by one or more groups R^{zz}.

Preferably each R^{ss} is independently C₁₋₄ alkyl, C₁₋₄ haloalkyl, or phenyl optionally substituted by 1-3 groups R^{zz}.

More preferably each R^{ss} is independently C₁₋₄ alkyl or phenyl optionally substituted by 1-3 groups R^{zz}.

Each R^{uu} is independently: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; NR^{ac}R^{ad}; or OR^{ae};

Preferably each R^{uu} is independently: hydrogen; C₁₋₄ alkyl optionally substituted by 1-4 groups R^{xx}; C₁₋₄ haloalkyl optionally substituted by 1-4 groups R^{xx}; C₃₋₆ cycloalkyl optionally substituted by 1-4 groups R^{yy}; phenyl optionally substituted by 1-3 groups R^{zz}; heteroaryl optionally substituted by 1-3 groups R^{ab}; NR^{ac}R^{ad}; or OR^{ae}.

More preferably each R^{uu} is independently C₁₋₄ alkyl, phenyl optionally substituted by 1-3 groups R^{zz}, C₁₋₄ alkoxy, or NR^{ac}R^{ad}.

Each R^{vv} is: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; SO₂R^{ss}; or C(O)R^{uu}.

Each R^{ww} is: hydrogen, alkyl optionally substituted by one or more R^{xx}; cycloalkyl optionally substituted by one or more R^{yy}; phenyl optionally substituted by one or more R^{zz}; or heteroaryl optionally substituted by one or more R^{ab}.

Alternatively R^{vv} and R^{ww} together with the nitrogen atom to which they are attached, form a 5-6 membered ring, which ring optionally contains an additional heteroatom selected from O, N and S, and is optionally substituted by 1-2 alkyl or alkylcarbonyl groups.

Each R^{xx} is independently: halogen; cyano; alkoxy; cycloalkyl; phenyl optionally substituted by one or more R^{zz}; heteroaryl optionally substituted by one or more R^{ab}; or alkoxycarbonyl.

Preferably each R^{xx} is independently: cyano, C₁₋₄ alkoxy, C₃₋₆ cycloalkyl, phenyl optionally substituted by 1-3 groups R^{zz}, heteroaryl optionally substituted by 1-3 groups R^{ab}, or C₁₋₄ alkoxycarbonyl.

Each R^{yy} is independently: halogen; cyano; alkyl; cycloalkyl; phenyl optionally substituted by one or more R^{zz}; heteroaryl optionally substituted by one or more R^{ab}; or alkoxycarbonyl.

Preferably each R^{yy} is independently: halogen, cyano, C₁₋₄ alkyl, or phenyl optionally substituted by 1-3 groups R^{zz}.

Each R^{zz} is independently: halogen; cyano; nitro; hydroxyl; alkyl optionally substituted by one or more R^{aq}; alkenyl optionally substituted by one or more R^{ar}; alkynyl optionally substituted by one or more R^{as}; OR^{al}; S(O)_{b}R^{am}; C(O)R^{an}.

Preferably each R^{zz} is independently: halogen, cyano, nitro, C₁₋₄ alkyl, C₁₋₄ haloalkyl, C₁₋₄ alkoxy(C₁₋₄)alkyl, C₁₋₄ alkoxy, C₁₋₄ haloalkoxy, C₁₋₄ alkylsulphonyl, C₁₋₄ haloalkylsulphonyl, or C(O)R^{an}.

More preferably each R^{zz} is independently halogen, C₁₋₄ alkyl, C₁₋₄ alkoxy, or C₁₋₄ alkylsulphonyl.

Each R^{ab} is independently: halogen; cyano; hydroxyl; alkyl optionally substituted by one or more R^{aq}; alkenyl optionally substituted by one or more R^{ar}; alkynyl optionally substituted by one or more R^{as}; OR^{al}; S(O)_{b}R^{am}; or C(O)R^{an}.

Preferably each R^{ab} is independently: halogen, cyano, C₁₋₄ alkyl, C₁₋₄ haloalkyl, C₁₋₄ alkoxy(C₁₋₄)alkyl, C₁₋₄ alkoxy, C₁₋₄ haloalkoxy, C₁₋₄ alkylsulphonyl, C₁₋₄ haloalkylsulphonyl, or C(O)R^{an}.

Each R^{ac} is independently: hydrogen; alkyl; haloalkyl; alkoxyalkyl; alkylcarbonyl; alkoxycarbonyl; or phenyl optionally substituted with one or more R^{zz}.

Each R^{ad} is independently: hydrogen; alkyl; haloalkyl; alkoxyalkyl; or phenyl optionally substituted with one or more R^{zz};

Alternatively R^{ac} and R^{ad} together with the N-atom to which they are joined form a 4-6 membered heterocyclic ring.

Preferably each R^{ac} is independently hydrogen, C₁₋₄ alkyl, C₁₋₄ haloalkyl, or C₁₋₄ alkoxy(C₁₋₄)alkyl.

Preferably each R^{ad} is independently hydrogen, C₁₋₄ alkyl, C₁₋₄ haloalkyl, or C₁₋₄ alkoxy(C₁₋₄)alkyl; or R^{ac} and R^{ad} together with the N atom to which they are joined form a 5- or 6-membered heterocyclic ring.

More preferably each R^{ac} and R^{ad} are independently hydrogen, or C₁₋₄ alkyl.

Each R^{ae} is independently: alkyl; phenylalkyl optionally substituted by one or more R^{zz}; or phenyl optionally substituted by one or more R^{zz}.

Preferably each R^{ae} is independently C₁₋₄ alkyl, phenyl C₁₋₄alkyl optionally substituted by 1-3 groups R^{zz}, or phenyl optionally substituted by 1-3 groups R^{zz}.

Each R^{af} is independently alkyl or phenyl and each R^{ag} is independently alkyl or haloalkyl.

Each R^{aj} is independently: halogen; cyano; nitro; alkyl; haloalkyl; alkoxyalkyl; alkenyl; alkynyl; alkoxy; haloalkoxy; alkylthio; haloalkylthio; alkylsulphonyl; haloalkylsulphonyl; alkoxycarbonyl; aminocarbonyl; alkylaminocarbonyl; dialkylaminocarbonyl; amino; alkylamino; dialkylamino; alkylcarbonylamino; alkylcarbonyl(alkyl)amino; or aminocarbonylamino.

Preferably each R^{aj} is independently halogen, cyano, nitro, C₁₋₄ alkyl, C₁₋₄ haloalkyl, C₁₋₄ alkoxyC₁₋₄ alkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl.

More preferably each R^{aj} is independently halogen, C₁₋₄ alkyl, C₁₋₄ haloalkyl, C₁₋₄ alkoxy, C₁₋₄ haloalkoxy, or C₁₋₄ alkylsulphonyl.

Each R^{ak} is independently: halogen; cyano; hydroxyl; alkyl; haloalkyl; alkoxyalkyl; alkenyl; alkynyl; alkoxy; haloalkoxy; alkylthio; haloalkylthio; alkylsulphonyl; haloalkylsulphonyl; alkoxycarbonyl; aminocarbonyl; alkylaminocarbonyl; dialkylaminocarbonyl; amino; alkylamino; dialkylamino; alkylcarbonylamino; alkylcarbonyl(alkyl)amino;or aminocarbonylamino;

Preferably each R^{ak} is independently halogen, cyano, C₁₋₄ alkyl, C₁₋₄ haloalkyl, C₁₋₄ alkoxy(C₁₋₄)alkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl.

More preferably each R^{ak} is independently halogen, C₁₋₄ alkyl, C₁₋₄ haloalkyl, C₁₋₄ alkoxy, C₁₋₄ haloalkoxy, or C₁₋₄ alkylsulphonyl.

_{b} is an integer selected from 0, 1, and 2.

Each R^{al} is independently: alkyl, haloalkyl, alkoxyalkyl, or alkylsulphonyl.

Each R^{am} is independently: alkyl, haloalkyl, alkenyl, alkynyl, phenyl, or alkylcarbonylamino.

Each R^{an} is independently: hydrogen, alkyl, haloalkyl, alkoxy, haloalkoxy, amino, alkylamino, dialkylamino, or alkylsulphonylamino.

Preferably each R^{an} is independently C₁₋₄ alkyl, C₁₋₄ haloalkyl, C₁₋₄ alkoxy, C₁₋₄ haloalkoxy, amino, C₁₋₄ alkylamino, or di(C₁₋₄)alkylamino.

Each R^{ao} is independently hydrogen, alkyl, alkoxyalkyl, phenylalkyl, formyl, alkylcarbonyl, alkoxycarbonyl, aminocarbonyl, alkylaminocarbonyl, or dialkylaminocarbonyl.

Each R^{ap} is independently: hydrogen, alkyl, alkoxyalkyl, or phenylalkyl.

Alternatively R^{ao} and R^{ap} together with the nitrogen atom to which they are attached form a 5-6 membered ring which optionally contains an additional heteroatom selected from O, N, and S, and is optionally substituted by 1-2 alkyl or alkylcarbonyl groups.

Each R^{aq} is independently: halogen, alkoxy, cyano, alkoxycarbonyl, or alkylsulphonyl.

Each R^{ar} is independently: halogen, cyano, nitro, alkoxy, alkylcarbonyl, alkoxycarbonyl, aminocarbonyl, alkylsulphonyl, or dialkyl phosphonyl.

Each R^{as} is independently: halogen, cyano, trialkylsilyl, alkylcarbonyl, alkoxycarbonyl, aminocarbonyl, or alkylsulphonyl.

Each R^{at} is independently: halogen, alkyl, or alkoxycarbonyl.

According to the invention P is defined herein as CR¹⁰R¹¹; wherein R¹⁰ and R¹¹ are independently: hydrogen; cyano; nitro; alkyl; haloalkyl; hydroxyalkyl; alkoxyalkyl; carboxyalkyl; alkoxycarbonylalkyl; alkylcarbonyloxyalkyl; cycloalkyl; alkenyl; phenyl, optionally substituted by 1-3 groups R^{ea}; heteroaryl, optionally substituted by 1-3 groups R^{ea}; alkoxy; haloalkoxy; alkylthio; haloalkylthio; alkylsulphonylalkyl; alkylcarbonyl; alkoxycarbonyl; carboxy; aminocarbonyl; alkylaminocarbonyl; dialkylaminocarbonyl; or dialkylphosphonyl; and wherein each R^{ea} is independently: halogen, cyano, alkyl, haloalkyl, alkoxy, haloalkoxy, alkylthio, or haloalkylthio.

Preferably R¹⁰ and R¹¹ are each independently: hydrogen; cyano; C₁₋₄ alkyl, C₁₋₄ haloalkyl, C₁₋₃ hydroxyalkyl, C₁₋₂ alkoxy(C₁₋₂)alkyl, carboxy(C₁₋₃)alkyl, C₁₋₃ alkoxy-carbonyl(C₁₋₃)alkyl, phenyl; C₁₋₄ alkoxycarbonyl, carboxy, aminocarbonyl, C₁₋₄ alkylaminocarbonyl, or di(C₁₋₃)alkylaminocarbonyl;

More preferably, R¹⁰ and R¹¹ are each independently: hydrogen; cyano; C₁₋₂ alkyl, C₁₋₂ haloalkyl, C₁₋₂ hydroxyalkyl, C₁₋₂ alkoxy(C₁₋₂)alkyl, C₁₋₃ alkoxycarbonyl, or carboxyl.

According to the invention W is defined as a direct bond, or an optionally substituted aliphatic chain, wherein said chain optionally contains a heteroatom selected from O, N or S.

Preferably W is a direct bond or an optionally substituted aliphatic chain selected from: a C₁₋₄ alkylene chain optionally substituted by 1-4 groups Rⁱ, a C₂₋₄ alkenylene chain optionally substituted by 1-3 groups R^{j}, and a C₂₋₄ alkynylene chain optionally substituted by 1-2 groups R^{k}; wherein one carbon atom in said optionally substituted aliphatic chain is optionally replaced by a heteroatom selected from O, S and N.

More preferably W is a direct bond, or a C₁₋₂ alkylene chain optionally substituted by 1-2 groups Rⁱ; wherein each Rⁱ is independently selected from: halogen, cyano, hydroxyl, C₁₋₄ alkyl, C₁₋₄ haloalkyl, C₁₋₃ hydroxyalkyl, C₁₋₂ alkoxy(C₁₋₂)alkyl, carboxy(C₁₋₃)alkyl, C₁₋₃ alkoxycarbonyl(C₁₋₃)alkyl, phenyl, C₁₋₄ alkoxycarbonyl, carboxy, aminocarbonyl, C₁₋₄ alkylaminocarbonyl, or di(C₁₋₃)alkylaminocarbonyl.

Most preferably W is a direct bond.

Each Rⁱ is independently: halogen; cyano; nitro; alkyl; haloalkyl; hydroxyalkyl; alkoxyalkyl; carboxyalkyl; alkoxycarbonylalkyl; alkylcarbonyloxyalkyl; cycloalkyl; alkenyl; phenyl optionally substituted by 1-3 groups R^{ea} (where R^{ea} is as defined hereinbefore); heteroaryl optionally substituted by 1-3 groups R^{ea} (where R^{ea} is as defined hereinbefore); hydroxyl; alkoxy; haloalkoxy; alkylthio; haloalkylthio; alkylcarbonyl; alkoxycarbonyl; carboxy; alkylcarbonyloxy; aminocarbonyl; alkylaminocarbonyl; dialkylaminocarbonyl; or dialkylphosphonyl; or any two geminal groups Rⁱ may together form an oxo group.

Preferably each Rⁱ is independently selected from: halogen, cyano, hydroxyl, C₁₋₄ alkyl, C₁₋₄ haloalkyl, C₁₋₃ hydroxyalkyl, C₁₋₂ alkoxy(C₁₋₂)alkyl, carboxy(C₁₋₃)alkyl, C₁₋₃ alkoxycarbonyl(C₁₋₃)alkyl, phenyl, C₁₋₄ alkoxycarbonyl, carboxy, aminocarbonyl, C₁₋₄ alkylaminocarbonyl, or di(C₁₋₃)alkylaminocarbonyl.

More preferably each Rⁱ is independently: cyano, C₁₋₂ alkyl, C₁₋₂ haloalkyl, C₁₋₂ hydroxyalkyl, C₁₋₂ alkoxy(C₁₋₂)alkyl, C₁₋₃ alkoxycarbonyl, or carboxyl.

Each R^{j} is independently: halogen; cyano; nitro; alkyl; haloalkyl; alkoxyalkyl; alkylcarbonyloxyalkyl; cycloalkyl; phenyl optionally substituted by 1-3 groups R^{ea} (where R^{ea} is as defined hereinbefore); heteroaryl optionally substituted by 1-3 groups R^{ea} (where R^{ea} is as defined hereinbefore); alkoxy; haloalkoxy; alkylthio; haloalkylthio; alkylcarbonyl; alkoxycarbonyl; aminocarbonyl, alkylaminocarbonyl; or dialkylaminocarbonyl.

Each R^{k} is independently: halogen; cyano; nitro; alkyl; haloalkyl; alkoxyalkyl; alkoxy; haloalkoxy; alkylthio; haloalkylthio; alkylcarbonyl; alkoxycarbonyl; aminocarbonyl; alkylaminocarbonyl; or dialkylaminocarbonyl.

According to the invention, r is defined herein as an integer of 0 or 1 when W is a direct bond, or is the integer 1 when W is an optionally substituted aliphatic chain.

According to the invention Q is an optionally substituted 3-8 membered ring optionally containing up to 4 heteroatoms.

Preferably Q is a phenyl ring optionally substituted by 1-4 groups R⁶; a 5- or 6-membered heteroaromatic ring containing 1-4 heteroatoms, wherein at least one of said heteroatoms is N and any further heteroatom is independently selected from N, S and O provided that there is no more than one O or S atom in said heteroaromatic ring, and wherein said heteroaromatic ring is optionally substituted by 1-4 groups R⁶; or, a 3-8 membered non-aromatic ring wherein said ring is optionally substituted by 1-8 groups R⁷ and wherein said ring contains 0-3 heteroatoms selected independently from O, S and N, provided that said ring is not a dioxolan-4-yl ring.

More preferably Q is a ring system selected from: phenyl, thiophene, furan, pyrrole, isoxazole, isothiazole, oxazole, thiazole, imidazole, pyrazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,3,4-oxatriazole, 1,2,3,4-thiatriazole, tetrazole, pyridine, pyridazine, pyrimidine, pyrazine, 1,2,3-triazine, 1,2,4-triazine, 1,3,5-triazine, 1,2,4,5-tetrazine, wherein said ring system is optionally substituted by 1-3 groups R⁶; an optionally substituted C₃₋₈ cycloalkyl or optionally substituted C₅₋₈cycloalkenyl, wherein said substituents are selected from 1-4 groups R⁷; or a ring system selected from: aziridine, azetidine, pyrrolidine, piperidine, piperazine, azepane, oxetane, tetrahydrofuran, tetrahydropyran, morpholine, thiomorpholine, dihydrofuran, dihydropyran, 2-linked dioxolane, 1,3-dioxane, 1,4-dioxane, 1,3-dioxepane, dithiolane, 1,3-dithiane, 1,4-dithiane, wherein said ring system is optionally substituted by 1-4 groups R⁷.

Even more preferably Q is a ring system selected from: phenyl, thiophene, furan, pyrrole, isoxazole, isothiazole, oxazole, thiazole, imidazole, pyrazole, 1,2,4-oxadiazole, 1,3,4-oxadiazole, 1,2,4-thiadiazole, 1,3,4-thiadiazole, 1,2,3-triazole, 1,2,4-triazole, pyridine, pyridazine, pyrimidine, pyrazine, 1,2,4-triazine, and 1,3,5-triazine, wherein said ring system is optionally substituted by 1-3 groups R⁶; or C₃₋₆ cycloalkyl optionally substituted by 1-4 groups R⁷; or a ring system selected from: oxetane, tetrahydrofuran, tetrahydropyran, dihydrofuran, dihydropyran, 2-linked dioxolane, 1,3-dioxane, dithiolane, and 1,3-dithiane, wherein said ring system is optionally substituted by 1-4 groups R⁷.

Most preferably Q is a ring selected from: phenyl, furan, thiophene, oxazole, and thiazole, wherein said ring is optionally substituted by 1-2 groups R⁶; or C₃₋₆ cycloalkyl optionally substituted by 1-2 groups R⁷.

Each R⁶ is independently: halogen; cyano; nitro; azido; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; phenyl, optionally substituted by 1-3 groups R^{dl}; heteroaryl optionally substituted by 1-3 groups R^{dl}; OR^{da}; S(O)_{d}R^{db;} C(O)R^{dc}; or NR^{dk}R^{de}; or 2 adjacent groups R⁶ together with the atoms to which they are joined to form a 5-7 membered ring, wherein said ring optionally contains 1 or 2 heteroatoms selected independently from O, S and N, and said ring is optionally substituted with 1-4 groups R⁸.

Preferably each R⁶ is independently: halogen; cyano; nitro; hydroxyl; C₁₋₆ alkyl optionally substituted by 1-4 groups R^{L}; C₁₋₆ haloalkyl optionally substituted by 1-4 groups R^{L}; OR^{da}; S(O)_{d}R^{db}; C(O)R^{dc}; NR^{dk}R^{de}; or 2 adjacent groups R⁶ together with the atoms to which they are joined form a 5- or 6-membered ring optionally containing 1 or 2 heteroatoms selected from O, S and N, and said ring is optionally substituted by 1-4 groups R⁸.

More preferably each R⁶ is independently: halogen, cyano, C₁₋₄ alkyl, C₁₋₄ haloalkyl, C₁₋₄ alkoxy, C₁₋₄ haloalkoxy, C₁₋₄ alkylthio, or C₁₋₄ haloalkylthio; or 2 adjacent groups R⁶ together with the atoms to which they are joined form a 6-membered aromatic ring optionally substituted with 1-2 groups selected independently from: halogen, cyano, C₁₋₄ alkyl, C₁₋₄ haloalkyl, C₁₋₄ alkoxy, C₁₋₄ haloalkoxy, C₁₋₄ alkylthio, and C₁₋₄ haloalkylthio.

Most preferably each R⁶ is independently: halogen, cyano, C₁₋₂ alkyl, C₁₋₂ haloalkyl, C₁₋₂ alkoxy, or C₁₋₂ haloalkoxy; or 2 adjacent groups R⁶ together with the atoms to which they are joined to form a 6 membered aromatic ring optionally substituted with 1-2 groups independently selected from: halogen, cyano, C₁₋₂ alkyl, C₁₋₂ haloalkyl, C₁₋₂ alkoxy, and C₁₋₂ haloalkoxy.

Each R^{dl} is independently: halogen, cyano, alkyl, haloalkyl, alkoxy, haloalkoxy, alkylthio, or haloalkylthio.

Each R⁷ is independently: halogen; cyano; nitro; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; phenyl optionally substituted by one or more R^{v}; heteroaryl optionally substituted by one or more R^{v}; OR^{da}; S(O)_{d}R^{db}; C(O)R^{dc}; or NR^{dk}R^{de}; or any two geminal groups R⁷ together form a group selected from: oxo, ═CR^{dm}R^{dn}, ═NOR^{do}, and ═NNR^{dp}R^{dq}; or two groups R⁷ together with the atom(s) to which they are joined and any intervening atoms form a 3-7 membered ring system, wherein said ring system optionally contains 1-2 heteroatoms independently selected from O, S and N and is optionally substituted by 1-4 groups R⁹.

Preferably each R⁷ is independently: halogen; cyano; nitro; hydroxyl; C₁₋₆ alkyl optionally substituted by 1-4 groups R^{L}; C₁₋₆ haloalkyl optionally substituted by 1-4 groups R^{L}; C₂₋₆ alkenyl optionally substituted by 1-3 groups, R^{m}; phenyl optionally substituted by 1-4 groups R^{v}; heteroaryl optionally substituted by 1-3 groups R^{v}; OR^{da}; S(O)_{d}R^{db}; C(O)R^{dc}; or NR^{dk}R^{de}; or any two geminal groups R⁷ together form a group selected from oxo, ═CR^{dm}R^{dn}, ═NOR^{do}, or ═NNR^{dp}R^{dq}; or two groups R⁷ together with the atom(s) to which they are joined and any intervening atoms form a 3-7 membered ring system, wherein said ring system optionally contains a heteroatom selected from O, N and S, and is optionally substituted by 1-4 groups R⁹.

More preferably each R⁷ is independently: halogen, cyano, C₁₋₄ alkyl, C₁₋₄ haloalkyl, C₁₋₄ alkoxy, C₁₋₄ haloalkoxy, C₁₋₄ alkoxycarbonyl, C₁₋₄ alkylaminocarbonyl, phenyl optionally substituted by 1-3 groups R^{v}, or heteroaryl optionally substituted by 1-3 groups R^{v}; or any two geminal groups R⁷ together form a group selected from oxo, ═CR^{dm}R^{dn}, ═NOR^{do}; or two groups R⁷ together with the atom(s) to which they are joined and any intervening atoms form a 5-6 membered ring system optionally substituted by 1 or 2 groups independently selected from: halogen, cyano, C₁₋₄ alkyl, C₁₋₄ haloalkyl, C₁₋₄ alkoxy, and C₁₋₄ haloalkoxy.

Most preferably each R⁷ is independently: halogen, cyano, C₁₋₂ alkyl, C₁₋₂ haloalkyl, C₁₋₂ alkoxy, C₁₋₂ haloalkoxy, or C₁₋₃ alkoxycarbonyl.

Each R^{L} is independently: halogen; cyano; nitro; hydroxyl; cycloalkyl; phenyl optionally substituted by 1-3 groups R^{dl} (wherein R^{dl} is as defined hereinbefore); heteroaryl optionally substituted by 1-3 groups R^{dl} (wherein R^{dl} is as defined hereinbefore); OR^{da}; S(O)_{d}R^{db}; C(O)R^{dc}; or NR^{dk}R^{de}.

Preferably each R^{L} is independently cyano, hydroxyl, C₃₋₆ cycloalkyl, OR^{da}, S(O)_{d}R^{db}, C(O)R^{dc}, or NR^{dk}R^{de}.

Each R^{m} is independently: halogen; cyano; nitro; hydroxyl; phenyl optionally substituted by 1-3 groups R^{dl} (wherein R^{dl} is as defined hereinbefore); heteroaryl optionally substituted by 1-3 groups R^{dl} (wherein R^{dl} is as defined hereinbefore); alkoxy; S(O)₂R^{db}; C(O)R^{dc}; or P(O)R^{di}R^{dj}.

Preferably each R^{m} is independently halogen, cyano, nitro, C₁₋₆ alkoxy, S(O)₂R^{db}, or C(O)R^{dc}.

Each Rⁿ is independently: halogen; cyano; alkoxy; S(O)₂R^{db}; C(O)R^{dc}; or SiR^{df}R^{dg}R^{dh}.

Each R^{o} is independently: halogen; cyano; nitro; hydroxyl; alkyl; alkenyl; alkynyl; cycloalkyl; alkoxy; S(O)_{d}R^{db}; or C(O)R^{dc}.

Each R^{v} is independently: halogen; cyano; nitro; hydroxyl; alkyl optionally substituted by one or more R^{L}; OR^{da}; S(O)_{d}R^{db}; C(O)R^{dc}; or NR^{dk}R^{de}.

Preferably each R^{v} is independently chosen from: halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio.

Each R^{da} is independently: alkyl; haloalkyl; alkoxyalkyl; cycloalkylalkyl; alkenyl; haloalkenyl; alkynyl; haloalkynyl; cycloalkyl; phenyl optionally substituted by 1-3 groups R^{dl} (wherein R^{dl} is as defined hereinbefore); heteroaryl optionally substituted by 1-3 groups R^{dl} (wherein R^{dl} is as defined hereinbefore); alkylcarbonyl; haloalkylcarbonyl; alkoxycarbonyl; haloalkoxycarbonyl; alkylsulphonyl; haloalkylsulphonyl; aminocarbonyl; alkylaminocarbonyl; dialkylaminocarbonyl; alkylimino; or dialkylimino.

Preferably each R^{da} is independently C₁₋₆ alkyl, C₁₋₆ haloalkyl, C₁₋₄ alkoxy(C₁₋₄)alkyl, or C₁₋₆ alkylcarbonyl

_{d} is an integer selected from 0, 1, and 2.

Each R^{db} is independently: alkyl; haloalkyl; alkoxyalkyl; alkenyl; haloalkenyl; alkynyl; haloalkynyl; cycloalkyl; or alkylcarbonylamino.

Preferably each R^{db} is independently C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino.

Each R^{dc} is independently: hydrogen; hydroxyl; alkyl; haloalkyl; alkoxyalkyl; alkenyl; haloalkenyl; alkynyl; haloalkynyl; cycloalkyl; alkoxy; haloalkoxy; alkoxyalkoxy; arylalkoxy; cycloalkoxy; amino; alkylamino; dialkylamino; or alkylsulphonylamino.

Preferably each R^{dc} is independently hydrogen, hydroxyl, C₁₋₆ alkyl, C₁₋₆ haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino, or C₁₋₆ alkylsulphonylamino.

Each R^{dk} is independently: hydrogen; alkyl; haloalkyl; alkoxyalkyl; alkenyl; haloalkenyl; alkynyl; haloalkynyl; cycloalkyl; alkylcarbonyl; haloalkylcarbonyl; alkoxycarbonyl; haloalkoxycarbonyl; alkylsulphonyl; haloalkylsulphonyl; aminocarbonyl; alkylaminocarbonyl; dialkylaminocarbonyl.

Preferably each R^{dk} is independently hydrogen, C₁₋₆ alkyl, C₁₋₆ alkylcarbonyl, C₁₋₆ alkoxycarbonyl, C₁₋₆ alkylsulphonyl, C₁₋₆ haloalkylsulphonyl, aminocarbonyl, C₁₋₆ alkylaminocarbonyl, or di(C₁₋₄)alkylaminocarbonyl.

Each R^{de} is independently: hydrogen; alkyl; haloalkyl; alkoxyalkyl; alkenyl; haloalkenyl; alkynyl; haloalkynyl; cycloalkyl; or R^{dk} and R^{de}, together with the N atom to which they are joined form a 4-6 membered saturated, unsaturated or aromatic ring, optionally containing one further heteroatom selected from O, N and S, said ring being optionally substituted by 1-4 groups selected from halogen, alkyl, alkoxy, alkylcarbonyl, alkoxycarbonyl, and oxo.

Preferably each R^{de} is independently hydrogen, C₁₋₆ alkyl, or C₃₋₆ cycloalkyl; or R^{dk} and R^{de}, together with the nitrogen atom to which they are joined form a 5- or 6-membered saturated ring optionally containing one further heteroatom selected from O, N, and S, said ring being optionally substituted by 1-2 C₁₋₆ alkyl or oxo groups.

Each R⁸ is independently: halogen; cyano; nitro; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; OR^{da}; S(O)_{d}R^{db}; C(O)R^{dc}; or NR^{dk}R^{de}; or any two geminal groups R⁸ together form a group selected from: oxo, ═CR^{dm}R^{dn}, ═NOR^{do}, and ═NNR^{dp}R^{dq}.

Preferably each R⁸ is independently: halogen, cyano, nitro, hydroxyl, C₁₋₆ alkyl optionally substituted by 1-4 groups R^{L}, C₁₋₆ haloalkyl optionally substituted by 1-4 groups R^{L}, OR^{da}, S(O)_{d}R^{db}, C(O)R^{dc}, or NR^{dk}R^{de}; or any two geminal groups R⁸ together form a group selected from oxo, ═CR^{dm}R^{dn}, ═NOR^{do}, and ═NNR^{dp}R^{dq}.

Each R^{dm} is independently: hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

Preferably each R^{dm} is independently hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl.

More preferably each R^{dm} is independently hydrogen, halogen, cyano, nitro, C₁₋₄ alkyl, or C₁₋₄ alkoxycarbonyl.

Each R^{dn} is independently: hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl.

Preferably each R^{dn} is independently hydrogen, halogen, cyano, nitro, C₁₋₆ alkyl, C₁₋₆ alkylcarbonyl, C₁₋₆ alkoxycarbonyl, C₁₋₆ alkylsulphonyl, or aminocarbonyl.

More preferably each R^{dn} is independently hydrogen, halogen, cyano, nitro, C₁₋₄ alkyl, or C₁₋₄ alkoxycarbonyl.

Each R^{do} is independently: hydrogen, alkyl, haloalkyl, alkoxyalkyl, cycloalkylalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl;

Preferably each R^{do} is independently hydrogen, C₁₋₆ alkyl, or C₃₋₆ cycloalkyl and more preferably each R^{do} is independently hydrogen, C₁₋₄ alkyl, or C₃₋₆ cycloalkyl.

Each R^{dp} is independently: hydrogen, alkyl, haloalkyl, alkoxyalkyl, or cycloalkyl. Preferably each R^{dp} is independently hydrogen, C₁₋₆ alkyl, or C₃₋₆ cycloalkyl.

Each R^{dq} is independently: hydrogen, alkyl, haloalkyl, alkylcarbonyl, haloalkylcarbonyl, alkoxycarbonyl, aminocarbonyl, alkylaminocarbonyl, or dialkylaminocarbonyl.

Preferably each R^{dq} is independently hydrogen or C₁₋₆ alkyl.

Alternatively R^{dp} and R^{dq} together with the nitrogen to which they are attached form a 5- or 6-membered ring, optionally containing an additional heteroatom selected from O, N and S, and being optionally substituted with one or more groups selected from halogen and alkyl.

Each R⁹ is independently: halogen; cyano; nitro; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; OR^{da}; S(O)_{d}R^{db}; C(O)R^{dc}; or NR^{dk}R^{de}; or any two geminal groups R⁹ together form a group selected from: oxo, ═CR^{dm}R^{dn}, ═NOR^{do}, and ═NNR^{dp}R^{dq}.

Preferably each R⁹ is independently: halogen; cyano; nitro; hydroxyl; C₁₋₆ alkyl optionally substituted by 1-4 groups R^{L}; C₁₋₆ haloalkyl optionally substituted by 1-4 groups R^{L}; C₂₋₆ alkenyl optionally substituted by 1-3 groups R^{m}; OR^{da}; S(O)_{d}R^{db}; C(O)R^{dc}; or NR^{dk}R^{de}; or any two geminal groups R⁹ together form a group selected from oxo, ═CR^{dm}R^{dn}, ═NOR^{do}, and ═NNR^{dp}R^{dq}.

Each R^{df}, R^{dg}, and R^{dh} is independently: alkyl or haloalkyl.

Each R^{di} is independently: alkyl, haloalkyl, alkoxy, or haloalkoxy.

Each R^{dj} is independently: alkoxy or haloalkoxy.

According to the invention Y is defined as halogen, cyano, nitro, hydroxyl, optionally substituted alkyl, optionally substituted cycloalkyl, optionally substituted alkenyl, optionally substituted alkynyl, optionally substituted phenyl, optionally substituted heteroaryl, optionally substituted alkoxy, optionally substituted amino, optionally substituted alkylthio, optionally substituted alkylsulphinyl, optionally substituted alkylsulphonyl, optionally substituted dialkyl phosphonyl, optionally substituted trialkylsilyl.

Preferably Y is halogen; cyano; nitro; hydroxyl; C₁₋₆ alkyl optionally substituted by 1-3 groups R^{ba}; C₁₋₆ haloalkyl optionally substituted by 1-3 groups R^{ba}; C₃₋₆ cycloalkyl optionally substituted by 1-3 groups R^{bc}; C₂₋₆ alkenyl optionally substituted by 1-3 groups R^{bd}; C₂₋₆ alkynyl optionally substituted by 1-3 groups R^{be}; phenyl optionally substituted by 1-4 groups R^{bf}; heteroaryl optionally substituted by 1-3 groups R^{bg}; OR^{bh}; NR^{bi}R^{bj}; S(O)_{c}R^{bk}; di(C₁₋₄)alkyl phosphonyl; or tri(C₁₋₄)alkylsilyl.

More preferably Y is halogen, C₁₋₃ alkyl, C₁₋₃ haloalkyl, C₁₋₂ alkoxy(C₁₋₂)alkyl, cyclopropyl optionally substituted by 1-2 groups R^{bc}, C₂₋₄ alkenyl, C₂₋₄ haloalkenyl, or C₂₋₄ alkynyl optionally substituted by 1-2 groups R^{be}.

Most preferably Y is halogen, C₁₋₂ alkyl, C₁₋₂ haloalkyl, C₁₋₂ alkoxy(C₁₋₂)alkyl, or C₂₋₄ alkenyl.

Each R^{ba} is independently: cyano; nitro; hydroxyl; C₁₋₄ alkoxy; C₁₋₄ alkylthio; C₁₋₄ alkylcarbonyl; or C₁₋₄ alkoxycarbonyl; or any 2 geminal groups R^{ba} together form a group selected from: oxo or ═NOR^{bl}.

Each R^{bc} is independently: halogen; cyano; C₁₋₄ alkyl; C₁₋₄ alkoxy; or C₁₋₄ alkoxycarbonyl. Preferably R^{bc} is independently halogen or C₁₋₂ alkyl.

Each R^{bd} is independently: halogen; cyano; C₁₋₄ alkylcarbonyl; or C₁₋₄ alkoxycarbonyl.

Each R^{be} is independently: halogen; cyano; hydroxyl; C₁₋₄ alkoxycarbonyl; or tri(C₁₋₄)alkylsilyl. Preferably each R^{be} is independently halogen or tri(C₁₋₃)alkylsilyl.

Each R^{bf} is independently: halogen; cyano; C₁₋₄ alkyl; C₁₋₄ haloalkyl; C₁₋₄ alkoxy(C₁₋₄)alkyl; C₂₋₄ alkenyl; C₁₋₄ alkoxy; C₁₋₄ haloalkoxy; C₁₋₄ alkylsulphonyl; or C₁₋₄ alkoxycarbonyl.

Each R^{bg} is independently: halogen; cyano; C₁₋₄ alkyl; C₁₋₄ haloalkyl; C₁₋₄ alkoxy(C₁₋₄)alkyl; C₁₋₄ alkoxy; C₁₋₄ haloalkoxy; C₁₋₄ alkylsulphonyl; or C₁₋₄ alkoxycarbonyl.

Each R^{bh} is independently: C₁₋₄ alkyl; C₁₋₄ haloalkyl; or C₁₋₄ alkylsulphonyl.

Each R^{bi} is independently: hydrogen; C₁₋₄ alkyl; C₁₋₄ alkylcarbonyl; C₁₋₄ alkoxycarbonyl; aminocarbonyl; C₁₋₄ alkylaminocarbonyl; or di(C₁₋₄)alkylaminocarbonyl.

Each R^{bj} is independently hydrogen or C₁₋₄ alkyl.

Each R^{bk} is independently C₁₋₄ alkyl or C₁₋₄ haloalkyl;

_{c} is an integer selected from 0, 1 and 2.

Each R^{bl} is independently hydrogen or C₁₋₄ alkyl.

According to the invention Z is defined as (T)ₘ-(U)ₙ—V. Preferably Z is (O)ₘ—(U)ₙ—V, more preferably Z is (O)ₘ—(CR^{w}R^{x})ₙ—C(O)R^{cb}, and most preferably Z is C(O)R^{cb}.

T is an oxygen or sulphur atom, preferably an oxygen atom.

ₘ is an integer of 0 to 1; ₙ is an integer from 0, 1, 2, and 3, and ₙ≧ₘ.

Preferably ₘ is an integer of 0 to 1; ₙ is an integer of 0 to 1 and ₙ≧ₘ.

U is CR^{w}R^{x}. Each R^{w} is independently hydrogen, halogen, hydroxyl, optionally substituted alkyl, optionally substituted alkoxycarbonyl, or OR^{y}.

Each R^{x} is independently hydrogen, halogen, optionally substituted alkyl, or OR^{Z}.

Alternatively, any geminal R^{w} and R^{x} together form a group selected from oxo, or ═NOR^{ca}; or any geminal, vicinal or non-adjacent R^{w} and/or R^{x} together with the C atom(s) to which they are attached and any intervening atom form an optionally substituted 3-6 membered ring.

R^{ca} is hydrogen or optionally substituted alkyl.

Preferably R^{w} is hydrogen, halogen, hydroxyl, C₁₋₄ alkyl, C₁₋₄ haloalkyl, C₁₋₃ alkoxy(C₁₋₃)alkyl, C₁₋₆ alkoxycarbonyl, C₁₋₄ alkoxy, C₁₋₄ alkylcarbonyloxy.

More preferably R^{w} is hydrogen or C₁₋₄ alkyl.

Preferably R^{x} is hydrogen, halogen, C₁₋₄ alkyl, C₁₋₄ haloalkyl, C₁₋₃ alkoxy(C₁₋₃)alkyl, C₁₋₄ alkoxy; or any geminal R^{w} and R^{x} together form a group selected from oxo, or ═NOR^{ca}, or any geminal, vicinal or non-adjacent R^{w} and/or R^{x} together with the C atom(s) to which they are joined and any intervening atom form a 3-6 membered ring. More preferably R^{x} is hydrogen.

Each R^{y} is independently an optionally substituted alkyl or an optionally substituted alkylcarbonyl.

Each R^{Z} is independently an optionally substituted alkyl group.

According to the invention V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn}, wherein R^{cb}, R^{cd}, R^{ce}, R^{cf}, R^{cg}, R^{ch}, R^{ci}, R^{cj}, R^{ck}, R^{cl}, R^{cm} and R^{cn} are as defined above.

Furthermore, when at least one R^{w} is OR^{y} and at least one R^{x} is OR^{Z}, said OR^{y} and OR^{Z} groups together with the C atom(s) to which they are attached and any intervening atom may form an optionally substituted 5-6 membered heterocyclic ring.

Preferably each R^{cb} is hydrogen, hydroxyl, C₁₋₁₀ alkylthio, amino, C₁₋₆ alkylamino, di(C₁₋₄)alkylamino, or OR^{co}. More preferably each R^{cb} is hydroxyl, C₁₋₁₀ alkylthio, or OR^{co}. Most preferably each R^{cb} is hydroxyl, C₁₋₁₀ alkoxy, or phenyl(C₁₋₂)alkoxy.

R^{cd} is C₁₋₂₀ alkoxy, C₁₋₁₀ alkylthio, amino, C₁₋₆ alkylamino, or di(C₁₋₄)alkylamino.

Each R^{ce} is hydrogen, C₁₋₆ alkyl, amino, C₁₋₆ alkylamino, di(C₁₋₄)alkylamino, C₁₋₆ alkoxy, or C₃₋₆ cycloalkoxy.

Each R^{cf} is hydrogen, C₁₋₆ alkoxy, C₁₋₆ alkylthio, amino, C₁₋₆ alkylamino, or di(C₁₋₄)alkylamino.

R^{cg} and R^{ch} are each independently C₁₋₄ alkoxy; or R^{cg} and R^{ch} together with the carbon to which they are joined form a dioxolane or dioxane ring, which is optionally substituted by 1-2 C₁₋₂ alkyl groups.

_{P} and _{q} are each independently an integer selected from 0, 1, and 2.

R^{ci} and R^{cj} are each independently C₁₋₄ alkyl; or R^{ci} and R^{cj} together with the carbon and sulphur atoms to which they are joined form a 5-6 membered ring, which is optionally substituted by 1-2 C₁₋₂ alkyl groups.

R^{ck}, R^{cl}, and R^{cm} are each independently an optionally substituted alkoxy group, preferably each are independently an unsubstituted C₁₋₄ alkoxy group. Alternatively R^{ck}, R^{ci}, and R^{cm} together with the carbon to which they are attached form an optionally substituted trioxabicyclo[2.2.2]octane ring system.

R^{cn} is hydrogen or C₁₋₄ alkylcarbonyl.

R^{co} is C₁₋₂₀ alkyl optionally substituted by 1-3 groups R^{cq}, C₁₋₂₀ haloalkyl optionally substituted by 1-3 groups R^{cq}, or C₃₋₆ cycloalkyl.

Each R^{cq} is independently: C₃₋₆ cycloalkyl, C₁₋₆ alkoxy, phenyl optionally substituted with 1-4 groups R^{cr}, or heteroaryl optionally substituted with 1-3 groups R^{cs};

Each R^{cr} and R^{cs} are independently: halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ alkoxycarbonyl, or amino.

In particularly preferred embodiments Z is selected from CO₂H, CO₂CH₃, CO₂CH₂CH₃, CO₂CH(CH₃)₂, CO₂CH₂CH₂CH₃, CO₂CH₂CH(CH₃)₂, or CO₂CH₂-phenyl.

The compounds described below are illustrative of novel compounds of the invention. Table 1 below provides 294 compounds designated compound numbers 1-1 to 1-294 respectively, of formula (I) wherein A is cyclopropyl, R⁵ is H, Y is Cl, and Z is COOH, and wherein the values of P, r, W and Q are as given in Table 1.

TABLE 1

| Cmpd. | | | | |
|---|---|---|---|---|
| No. | P | r | W | Q |
| 1-1 | — | 0 | direct bond | cyclopropyl |
| 1-2 | — | 0 | direct bond | 1-phenylcyclopropyl |
| 1-3 | — | 0 | direct bond | 1-(2-methoxyphenyl)-cyclopropyl |
| 1-4 | — | 0 | direct bond | 1-(3-trifluorophenyl)-cyclopropyl |
| 1-5 | — | 0 | direct bond | 1-(4-chlorophenyl)-cyclopropyl |
| 1-6 | — | 0 | direct bond | 1-(2,4-dichlorophenyl)-cyclopropyl |
| 1-7 | — | 0 | direct bond | 2-phenylcyclopropyl |
| 1-8 | — | 0 | direct bond | 2-(4-chlorophenyl)-2- |
| | | | | fluorocyclopropyl |
| 1-9 | — | 0 | direct bond | 2-methyl-2-phenylcyclopropyl |
| 1-10 | — | 0 | direct bond | 1-(pyrid-2-yl)-cyclopropyl |
| 1-11 | — | 0 | direct bond | 1-(pyrid-3-yl)-cyclopropyl |
| 1-12 | — | 0 | direct bond | 1-(pyrid-4-yl)-cyclopropyl |
| 1-13 | — | 0 | direct bond | 1-(6-methylpyrid-2-yl)-cyclopropyl |
| 1-14 | — | 0 | direct bond | 1-(6-chloropyrid-3-yl)-cyclopropyl |
| 1-15 | — | 0 | direct bond | 1-(6-methylpyrid-3-yl)-cyclopropyl |
| 1-16 | — | 0 | direct bond | 1-(4-chlorophenoxymethyl)- |
| | | | | cyclopropyl |
| 1-17 | — | 0 | direct bond | 1-(2-phenylethyl)-cyclopropyl |
| 1-18 | — | 0 | direct bond | 1-[2-(4-fluorophenyl)-ethyl]- |
| | | | | cyclopropyl |
| 1-19 | — | 0 | direct bond | 1-(2-phenylethenyl)-cyclopropyl |
| 1-20 | — | 0 | direct bond | 1-[2-(4-fluorophenyl)-ethenyl)]- |
| | | | | cyclopropyl |
| 1-21 | — | 0 | direct bond | 1-[2-(4-chlorophenyl)-ethenyl)]- |
| | | | | cyclopropyl |
| 1-22 | — | 0 | direct bond | 1-[2-(3,5-bis-trifluoromethylphenyl)- |
| | | | | ethenyl)]-cyclopropyl |
| 1-23 | — | 0 | direct bond | 1-(2-thiophen-2-ylethenyl)- |
| | | | | cyclopropyl |
| 1-24 | — | 0 | direct bond | 1-[2-(2-chlorophenyl)-ethenyl)]- |
| | | | | cyclopropyl |
| 1-25 | — | 0 | direct bond | 1-[2-(4-methylphenyl)-ethenyl)]- |
| | | | | cyclopropyl |
| 1-26 | — | 0 | direct bond | 1-[2-(2,6-difluorophenyl)-ethenyl)]- |
| | | | | cyclopropyl |
| 1-27 | — | 0 | direct bond | 1-[2-(2-fluorophenyl)-ethenyl)]- |
| | | | | cyclopropyl |
| 1-28 | — | 0 | direct bond | 1-[2-(2,4-dichlorophenyl)-ethenyl)]- |
| | | | | cyclopropyl |
| 1-29 | — | 0 | direct bond | 1-[2-(2,3-dichlorophenyl)-ethenyl)]- |
| | | | | cyclopropyl |
| 1-30 | — | 0 | direct bond | 1-[2-(3-chlorophenyl)-ethenyl)]- |
| | | | | cyclopropyl |
| 1-31 | — | 0 | direct bond | 1-[2-(2,4-difluorophenyl)-ethenyl)]- |
| | | | | cyclopropyl |
| 1-32 | — | 0 | direct bond | cyclobutyl |
| 1-33 | — | 0 | direct bond | cyclopentyl |
| 1-34 | — | 0 | direct bond | 2-hydroxycyclopentyl |
| 1-35 | — | 0 | direct bond | cyclohexyl |
| 1-36 | — | 0 | direct bond | 2-hydroxycyclohexyl |
| 1-37 | — | 0 | direct bond | 2-hydroxyindanyl |
| 1-38 | — | 0 | direct bond | 4-hydroxytetrahydrofuran-3-yl |
| 1-39 | — | 0 | direct bond | phenyl |
| 1-40 | — | 0 | direct bond | 3-trifluoromethylphenyl |
| 1-41 | — | 0 | direct bond | 4-methoxyphenyl |
| 1-42 | — | 0 | direct bond | 3,4-dichlorophenyl |
| 1-43 | CH₂ | 1 | direct bond | cyclopropyl |
| 1-44 | CH₂ | 1 | direct bond | 1-phenylcyclopropyl |
| 1-45 | CH₂ | 1 | direct bond | 2-phenylcyclopropyl |
| 1-46 | CH₂ | 1 | direct bond | 2,2-dichloro-3-methylcyclopropyl |
| 1-47 | CH₂ | 1 | direct bond | cyclobutyl |
| 1-48 | CH₂ | 1 | direct bond | cyclopentyl |
| 1-49 | CH₂ | 1 | direct bond | cyclohexyl |
| 1-50 | CH₂ | 1 | direct bond | 1-hydroxycyclohexyl |
| 1-51 | CH₂ | 1 | direct bond | 4-cyanocyclohexyl |
| 1-52 | CH₂ | 1 | direct bond | cyclooctyl |
| 1-53 | CH₂ | 1 | direct bond | tetrahydrofuran-2-yl |
| 1-54 | CH₂ | 1 | direct bond | 1,3-dioxolan-2-yl |
| 1-55 | CH₂ | 1 | direct bond | 2,5-dimethoxy-2,5-dihydrofuran-2-yl |
| 1-56 | CH₂ | 1 | direct bond | 3,4-dihydro-2H-pyran-2-yl |
| 1-57 | CH₂ | 1 | direct bond | 1-ethylpyrrolidin-2-yl |
| 1-58 | CH₂ | 1 | direct bond | 1-^{t}butoxycarbonylpiperidin-4-yl |
| 1-59 | CH₂ | 1 | direct bond | 1-hydroxy-6-methoxyindanyl |
| 1-60 | CH₂ | 1 | direct bond | phenyl |
| 1-61 | CH₂ | 1 | direct bond | 2-chlorophenyl |
| 1-62 | CH₂ | 1 | direct bond | 3-chlorophenyl |
| 1-63 | CH₂ | 1 | direct bond | 4-chlorophenyl |
| 1-64 | CH₂ | 1 | direct bond | 2-fluorophenyl |
| 1-65 | CH₂ | 1 | direct bond | 3-fluorophenyl |
| 1-66 | CH₂ | 1 | direct bond | 4-fluorophenyl |
| 1-67 | CH₂ | 1 | direct bond | 2-bromophenyl |
| 1-68 | CH₂ | 1 | direct bond | 3-bromophenyl |
| 1-69 | CH₂ | 1 | direct bond | 4-bromophenyl |
| 1-70 | CH₂ | 1 | direct bond | 3-iodophenyl |
| 1-71 | CH₂ | 1 | direct bond | 2-methylphenyl |
| 1-72 | CH₂ | 1 | direct bond | 3-methylphenyl |
| 1-73 | CH₂ | 1 | direct bond | 4-methylphenyl |
| 1-74 | CH₂ | 1 | direct bond | 4-isopropylphenyl |
| 1-75 | CH₂ | 1 | direct bond | 2-trifluoromethylphenyl |
| 1-76 | CH₂ | 1 | direct bond | 3-trifluoromethylphenyl |
| 1-77 | CH₂ | 1 | direct bond | 4-trifluoromethylphenyl |
| 1-78 | CH₂ | 1 | direct bond | 2-methoxyphenyl |
| 1-79 | CH₂ | 1 | direct bond | 3-methoxyphenyl |
| 1-80 | CH₂ | 1 | direct bond | 4-methoxyphenyl |
| 1-81 | CH₂ | 1 | direct bond | 4-difluoromethoxyphenyl |
| 1-82 | CH₂ | 1 | direct bond | 4-ⁿbutoxyphenyl |
| 1-83 | CH₂ | 1 | direct bond | 4-trifluoromethoxyphenyl |
| 1-84 | CH₂ | 1 | direct bond | 3-phenoxyphenyl |
| 1-85 | CH₂ | 1 | direct bond | 2-aminophenyl |
| 1-86 | CH₂ | 1 | direct bond | 4-aminophenyl |
| 1-87 | CH₂ | 1 | direct bond | 4-dimethylaminophenyl |
| 1-88 | CH₂ | 1 | direct bond | 2-morpholin-4-ylphenyl |
| 1-89 | CH₂ | 1 | direct bond | 4-(4-methylpiperazin-1- |
| | | | | ylmethyl)phenyl |
| 1-90 | CH₂ | 1 | direct bond | 2-nitrophenyl |
| 1-91 | CH₂ | 1 | direct bond | 3-nitrophenyl |
| 1-92 | CH₂ | 1 | direct bond | 4-nitrophenyl |
| 1-93 | CH₂ | 1 | direct bond | 4-aminosulphonylphenyl |
| 1-94 | CH₂ | 1 | direct bond | 4-methoxycarbonylphenyl |
| 1-95 | CH₂ | 1 | direct bond | 3-phenylphenyl |
| 1-96 | CH₂ | 1 | direct bond | 2,3-difluorophenyl |
| 1-97 | CH₂ | 1 | direct bond | 2,4-difluorophenyl |
| 1-98 | CH₂ | 1 | direct bond | 2,5-difluorophenyl |
| 1-99 | CH₂ | 1 | direct bond | 2,6-difluorophenyl |
| 1-100 | CH₂ | 1 | direct bond | 3,5-difluorophenyl |
| 1-101 | CH₂ | 1 | direct bond | 4-chloro-2-fluorophenyl |
| 1-102 | CH₂ | 1 | direct bond | 2-chloro-6-fluorophenyl |
| 1-103 | CH₂ | 1 | direct bond | 2,4-dichlorophenyl |
| 1-104 | CH₂ | 1 | direct bond | 2,6-dichlorophenyl |
| 1-105 | CH₂ | 1 | direct bond | 3,4-dichlorophenyl |
| 1-106 | CH₂ | 1 | direct bond | 2,4-dimethylphenyl |
| 1-107 | CH₂ | 1 | direct bond | 3-fluoro-5-trifluorophenyl |
| 1-108 | CH₂ | 1 | direct bond | 3,5-bis-trifluoromethylphenyl |
| 1-109 | CH₂ | 1 | direct bond | 2,3-dimethoxyphenyl |
| 1-110 | CH₂ | 1 | direct bond | 2,4-dimethoxyphenyl |
| 1-111 | CH₂ | 1 | direct bond | 2,6-dimethoxyphenyl |
| 1-112 | CH₂ | 1 | direct bond | 3,4-dimethoxyphenyl |
| 1-113 | CH₂ | 1 | direct bond | 3,5-dimethoxyphenyl |
| 1-114 | CH₂ | 1 | direct bond | 3,4-diethoxyphenyl |
| 1-115 | CH₂ | 1 | direct bond | benzo[1,3]dioxol-5-yl |
| 1-116 | CH₂ | 1 | direct bond | 4-chloro-3-methoxyphenyl |
| 1-117 | CH₂ | 1 | direct bond | 5-chloro-2-methoxyphenyl |
| 1-118 | CH₂ | 1 | direct bond | 2,3,6-trifluorophenyl |
| 1-119 | CH₂ | 1 | direct bond | 2,3,4-trifluorophenyl |
| 1-120 | CH₂ | 1 | direct bond | 2,4,5-trifluorophenyl |
| 1-121 | CH₂ | 1 | direct bond | 2,4-dichloro-6-methylphenyl |
| 1-122 | CH₂ | 1 | direct bond | 3,4,5-trimethoxyphenyl |
| 1-123 | CH₂ | 1 | direct bond | naphth-1-yl |
| 1-124 | CH₂ | 1 | direct bond | naphth-2-yl |
| 1-125 | CH₂ | 1 | direct bond | furan-2-yl |
| 1-126 | CH₂ | 1 | direct bond | 5-fluorofuran-2-yl |
| 1-127 | CH₂ | 1 | direct bond | 5-trifluoromethylfuran-2-yl |
| 1-128 | CH₂ | 1 | direct bond | 3-bromofuran-2-yl |
| 1-129 | CH₂ | 1 | direct bond | 5-bromofuran-2-yl |
| 1-130 | CH₂ | 1 | direct bond | 4-cyanofuran-2-yl |
| 1-131 | CH₂ | 1 | direct bond | 5-cyanofuran-2-yl |
| 1-132 | CH₂ | 1 | direct bond | 5-methoxycarbonylfuran-2-yl |
| 1-133 | CH₂ | 1 | direct bond | 5-nitrofuran-2-yl |
| 1-134 | CH₂ | 1 | direct bond | 5-ethylfuran-2-yl |
| 1-135 | CH₂ | 1 | direct bond | 5-methylfuran-2-yl |
| 1-136 | CH₂ | 1 | direct bond | 5-ethoxycarbonylfuran-2-yl |
| 1-137 | CH₂ | 1 | direct bond | 3-furanyl |
| 1-138 | CH₂ | 1 | direct bond | 2,5-dimethylfuran-3-yl |
| 1-139 | CH₂ | 1 | direct bond | 5-methyl-2-trifluoromethylfuran-3-yl |
| 1-140 | CH₂ | 1 | direct bond | thiophen-2-yl |
| 1-141 | CH₂ | 1 | direct bond | 5-chlorothiophen-2-yl |
| 1-142 | CH₂ | 1 | direct bond | 5-bromothiophen-2-yl |
| 1-143 | CH₂ | 1 | direct bond | 2-bromothiophen3-yl |
| 1-144 | CH₂ | 1 | direct bond | 1,5-dimethylpyrazol-3-yl |
| 1-145 | CH₂ | 1 | direct bond | imidazol-4-yl |
| 1-146 | CH₂ | 1 | direct bond | 2-chlorothiazol-5-yl |
| 1-147 | CH₂ | 1 | direct bond | 2-methylthiazol-5-yl |
| 1-148 | CH₂ | 1 | direct bond | oxazol-2-yl |
| 1-149 | CH₂ | 1 | direct bond | 4-methyl-1,2,5-oxadiazol-3-yl |
| 1-150 | CH₂ | 1 | direct bond | tetrazol-5-yl |
| 1-151 | CH₂ | 1 | direct bond | pyrid-2-yl |
| 1-152 | CH₂ | 1 | direct bond | 3-chloropyrid-2-yl |
| 1-153 | CH₂ | 1 | direct bond | 3-methylpyrid-2-yl |
| 1-154 | CH₂ | 1 | direct bond | 6-methylpyrid-2yl |
| 1-155 | CH₂ | 1 | direct bond | 3-chloro-5-trifluoromethylpyrid-2-yl |
| 1-156 | CH₂ | 1 | direct bond | pyrid-3-yl |
| 1-157 | CH₂ | 1 | direct bond | 6-chloropyrid-3-yl |
| 1-158 | CH₂ | 1 | direct bond | 6-methylpyrid-3-yl |
| 1-159 | CH₂ | 1 | direct bond | 6-trifluoromethylpyrid-3-yl |
| 1-160 | CH₂ | 1 | direct bond | pyrid-4-yl |
| 1-161 | CH₂ | 1 | direct bond | 2-methylpyrimidin-4-yl |
| 1-162 | CH₂ | 1 | direct bond | pyrazin-2-yl |
| 1-163 | CH₂ | 1 | direct bond | 5-methylpyrazin-2-yl |
| 1-164 | CH₂ | 1 | direct bond | 5-bromobenzofuran-2-yl |
| 1-165 | CH₂ | 1 | direct bond | indol-3-yl |
| 1-166 | CH₂ | 1 | direct bond | benzimidazol-2-yl |
| 1-167 | CH₂ | 1 | direct bond | 2-methyl-4,5,6,7-tetrahydro-2H- |
| | | | | indazol-5-yl |
| 1-168 | CH₂ | 1 | direct bond | benzothiazol-2-yl |
| 1-169 | CH₂ | 1 | direct bond | quinolin-2-yl |
| 1-170 | CH₂ | 1 | direct bond | 5,6,7,8-tetrahydroquinazolin-6-yl |
| 1-171 | CH(CH₃) | 1 | direct bond | cyclopropyl |
| 1-172 | CH(CH₃) | 1 | direct bond | 2,2-dichloro-3-methylcyclopropyl |
| 1-173 | CH(CH₃) | 1 | direct bond | cyclohexyl |
| 1-174 | CH(CH₃) | 1 | direct bond | phenyl |
| 1-175 | CH(CH₃) | 1 | direct bond | 4-fluorophenyl |
| 1-176 | CH(CH₃) | 1 | direct bond | 4-chlorophenyl |
| 1-177 | CH(CH₃) | 1 | direct bond | 4-bromophenyl |
| 1-178 | CH(CH₃) | 1 | direct bond | 4-pentylphenyl |
| 1-179 | CH(CH₃) | 1 | direct bond | 3-trifluoromethylphenyl |
| 1-180 | CH(CH₃) | 1 | direct bond | 3-methoxyphenyl |
| 1-181 | CH(CH₃) | 1 | direct bond | 4-methoxyphenyl |
| 1-182 | CH(CH₃) | 1 | direct bond | 4-nitrophenyl |
| 1-183 | CH(CH₃) | 1 | direct bond | 2,4-dimethylphenyl |
| 1-184 | CH(CH₃) | 1 | direct bond | naphth-1-yl |
| 1-185 | CH(CH₃) | 1 | direct bond | naphth-2-yl |
| 1-186 | CH(CH₃) | 1 | direct bond | furan-2-yl |
| 1-187 | CH(CH₃) | 1 | direct bond | 5-methylfuran-2-yl |
| 1-188 | CH(CH₃) | 1 | direct bond | pyrid-2-yl |
| 1-189 | C(CH₃)₂ | 1 | direct bond | phenyl |
| 1-190 | CH(CF₃) | 1 | direct bond | cyclopropyl |
| 1-191 | CH(CF₃) | 1 | direct bond | phenyl |
| 1-192 | CH(CF₃) | 1 | direct bond | furan-2-yl |
| 1-193 | CH(CF₃) | 1 | direct bond | 5-ethoxycarbonylfuran-2-yl |
| 1-194 | CH(CH₂CH₃) | 1 | direct bond | phenyl |
| 1-195 | CH(CH₂CH₃) | 1 | direct bond | 4-difluoromethoxyphenyl |
| 1-196 | CH(CH₂CH₃) | 1 | direct bond | 5-methylfuran-2-yl |
| 1-197 | CH(CH═CH₂) | 1 | direct bond | phenyl |
| 1-198 | CH(CH₂CH═CH₂) | 1 | direct bond | 4-chlorophenyl |
| 1-199 | CH(CH₂CH═CH₂) | 1 | direct bond | pyrid-2-yl |
| 1-200 | CH(cyclopropyl) | 1 | direct bond | 4-chlorophenyl |
| 1-201 | CH(CH₂OH) | 1 | direct bond | tetrahydrofuran-3-yl |
| 1-202 | CH(CH₂OH) | 1 | direct bond | tetrahydropyran-4-yl |
| 1-203 | CH(CH₂OH) | 1 | direct bond | phenyl |
| 1-204 | CH(CH₂OH) | 1 | direct bond | furan-2-yl |
| 1-205 | CH(CH₂SO₂Me) | 1 | direct bond | 4-methoxyphenyl |
| 1-206 | CH(CN) | 1 | direct bond | phenyl |
| 1-207 | CH(CN) | 1 | direct bond | furan-2-yl |
| 1-208 | CH(CN) | 1 | direct bond | 5-bromofuran-2-yl |
| 1-209 | CH(CN) | 1 | direct bond | 5-methylfuran-2-yl |
| 1-210 | CH(CO₂Me) | 1 | direct bond | phenyl |
| 1-211 | CH(CO₂Me) | 1 | direct bond | furan-2-yl |
| 1-212 | CH(CO₂Me) | 1 | direct bond | 5-methylfuran-2-yl |
| 1-213 | CH(CO₂Me) | 1 | direct bond | indol-3-yl |
| 1-214 | CH(CO₂Et) | 1 | direct bond | phenyl |
| 1-215 | CH₂ | 1 | CH₂ | cyclohexen-1-yl |
| 1-216 | CH₂ | 1 | CH₂ | 1,3-dioxolan-2-yl |
| 1-217 | CH₂ | 1 | CH₂ | pyrrolidin-1-yl |
| 1-218 | CH₂ | 1 | CH₂ | 1-methylpyrrolidin-2-yl |
| 1-219 | CH₂ | 1 | CH₂ | 2-oxoimidazolidin-1-yl |
| 1-220 | CH₂ | 1 | CH₂ | morpholin-4-yl |
| 1-221 | CH₂ | 1 | CH₂ | phenyl |
| 1-222 | CH₂ | 1 | CH₂ | 4-aminosulphonylphenyl |
| 1-223 | CH₂ | 1 | CH₂ | furan-2-yl |
| 1-224 | CH₂ | 1 | CH₂ | thiophen-2-yl |
| 1-225 | CH₂ | 1 | CH₂ | imidazol-4-yl |
| 1-226 | CH₂ | 1 | CH₂ | indol-3-yl |
| 1-227 | CH₂ | 1 | CH₂ | 5-methoxyindol-3-yl |
| 1-228 | CH₂ | 1 | CH₂ | pyrid-2-yl |
| 1-229 | CH₂ | 1 | CH₂ | 3-chloro-5-trifluoromethylpyrid-2-yl |
| 1-230 | CH(CH₃) | 1 | CH₂ | cyclopentyl |
| 1-231 | CH(CH₃) | 1 | CH₂ | indol-3-yl |
| 1-232 | CH₂ | 1 | CH(CH₃) | phenyl |
| 1-233 | CH₂ | 1 | CH(CH₃) | 4-chlorophenyl |
| 1-234 | CH₂ | 1 | CHF | 2-bromophenyl |
| 1-235 | CH₂ | 1 | CHF | 3-bromophenyl |
| 1-236 | CH₂ | 1 | CHF | 4-bromophenyl |
| 1-237 | CH₂ | 1 | CHF | 3-trifluoromethylphenyl |
| 1-238 | CH₂ | 1 | CHF | 2,4-difluorophenyl |
| 1-239 | CH₂ | 1 | CHF | 2,4-dichlorophenyl |
| 1-240 | CH₂ | 1 | CHF | 4-bromo-2-fluorophenyl |
| 1-241 | CH₂ | 1 | CF(CH₃) | 3-chlorophenyl |
| 1-242 | CH₂ | 1 | CF(CH₃) | 3-trifluoromethylphenyl |
| 1-243 | CH₂ | 1 | CF(CH₃) | 2,4-dichlorophenyl |
| 1-244 | CH₂ | 1 | CF(CH₂CH₂CH₃) | 2,4-dichlorophenyl |
| 1-245 | CH₂ | 1 | CH₂CH₂ | pyrrolidin-1-yl |
| 1-246 | CH₂ | 1 | CH₂CH₂ | 2-oxopyrrolidin-1-yl |
| 1-247 | CH₂ | 1 | CH₂CH₂ | 2-methylpiperidin-1-yl |
| 1-248 | CH₂ | 1 | CH₂CH₂ | morpholin-4-yl |
| 1-249 | CH₂ | 1 | CH₂CH₂ | phenyl |
| 1-250 | CH₂ | 1 | CH₂CH₂ | imidazol-1-yl |
| 1-251 | CH₂ | 1 | CH₂CH₂ | pyrid-3-yl |
| 1-252 | CH(CH₃) | 1 | CH₂CH₂ | phenyl |
| 1-253 | CH₂ | 1 | CH₂CH₂CH₂ | phenyl |
| 1-254 | CH₂ | 1 | CH═CH | phenyl |
| 1-255 | CH₂ | 1 | CH═CH | 2-methoxyphenyl |
| 1-256 | CH₂ | 1 | CH═CH | furan-2-yl |
| 1-257 | CH₂ | 1 | CH(OH) | phenyl |
| 1-258 | CH₂ | 1 | CH(OH) | 3-hydroxyphenyl |
| 1-259 | CH₂ | 1 | CH(OH) | 4-hydroxyphenyl |
| 1-260 | CH₂ | 1 | CH₂NH | phenyl |
| 1-261 | CH₂ | 1 | CH₂NH | naphth-1-yl |
| 1-262 | CH₂ | 1 | CH₂SCH₂ | phenyl |
| 1-263 | CH₂ | 1 | CH₂SCH₂ | 2,6-dichlorophenyl |
| 1-264 | CH₂ | 1 | CH(OH)CH₂O | 4-chlorophenyl |
| 1-265 | CH₂ | 1 | CO₂CH₂ | phenyl |
| 1-266 | CH₂ | 1 | CH₂CO₂CH₂ | phenyl |
| 1-267 | CH₂ | 1 | CHPh | phenyl |
| 1-268 | CH(CH₃) | 1 | CH₂O | phenyl |
| 1-269 | CH(CH₃) | 1 | CH₂O | 4-cyanophenyl |
| 1-270 | CH(CH₃) | 1 | CH(OH) | phenyl |
| 1-271 | CH(CH₃) | 1 | CH(OH) | 4-hydroxyphenyl |
| 1-272 | CH(CH₃) | 1 | CO₂CH₂ | phenyl |
| 1-273 | CH(CH₂OH) | 1 | CH₂ | phenyl |
| 1-274 | CH(CH₂OH) | 1 | CH₂ | 4-chlorophenyl |
| 1-275 | CH(CH₂OH) | 1 | CH(OH) | 4-nitrophenyl |
| 1-276 | CH(CH₂OH) | 1 | CH₂SCH₂ | phenyl |
| 1-277 | CHPh | 1 | direct bond | phenyl |
| 1-278 | CHPh | 1 | direct bond | 4-chlorophenyl |
| 1-279 | CHPh | 1 | CH₂ | phenyl |
| 1-280 | CHPh | 1 | CH(OH) | phenyl |
| 1-281 | CH(CO₂Me) | 1 | CH₂ | cyclopentyl |
| 1-282 | CH(CO₂Me) | 1 | CH₂ | cyclohexyl |
| 1-283 | CH(CO₂Me) | 1 | CH₂ | phenyl |
| 1-284 | CH(CO₂Me) | 1 | CH₂ | 4-fluorophenyl |
| 1-285 | CH(CO₂Me) | 1 | CH₂ | 4-chlorophenyl |
| 1-286 | CH(CO₂Me) | 1 | CH₂ | 3-trifluoromethylphenyl |
| 1-287 | CH(CO₂Me) | 1 | CH₂ | 4-hydroxyphenyl |
| 1-288 | CH(CO₂Me) | 1 | CH₂ | 4-^{t}butoxyphenyl |
| 1-289 | CH(CO₂Me) | 1 | CH₂ | imidazol-4-yl |
| 1-290 | CH(CO₂Me) | 1 | CH₂ | indol-3-yl |
| 1-291 | CH(CO₂Me) | 1 | CH₂ | quinolin-2-yl |
| 1-292 | CH(CO₂Et) | 1 | CH₂SCH₂ | phenyl |
| 1-293 | CH(CO₂^{t}Bu) | 1 | CH₂ | phenyl |
| 1-294 | CH(CH₂OH) | 1 | CH₂ | indol-3-yl |

294 compounds of formula (I), wherein A is cyclopropyl, Y is Cl, Z is CO₂H, R⁵ is CH₃, and the values of P, r, W and Q are as given in Table 1 for compounds 1-1 to 1-294, are designated as compound Nos. 2-1 to 2-294, respectively.

294 compounds of formula (I), wherein A is cyclopropyl, Y is Cl, Z is CO₂H, R⁵ is CH₂CH₃, and the values of P, r, W and Q are as given in Table 1 for compounds 1-1 to 1-294, are designated as compound Nos. 3-1 to 3-294, respectively.

294 compounds of formula (I), wherein A is cyclopropyl, Y is Cl, Z is CO₂CH₃, R⁵ is H, and the values of P, r, W and Q are as given in Table 1 for compounds 1-1 to 1-294, are designated as compound Nos. 4-1 to 4-294, respectively.

294 compounds of formula (I), wherein A is cyclopropyl, Y is Cl, Z is CO₂CH₃, R⁵ is CH₃, and the values of P, r, W and Q are as given in Table 1 for compounds 1-1 to 1-294, are designated as compound Nos. 5-1 to 5-294, respectively.

294 compounds of formula (I), wherein A is cyclopropyl, Y is Cl, Z is CO₂CH₃, R⁵ is CH₂CH₃, and the values of P, r, W and Q are as given in Table 1 for compounds 1-1 to 1-294, are designated as compound Nos. 6-1 to 6-294, respectively.

294 compounds of formula (I), wherein A is cyclopropyl, Y is Cl, Z is CO₂CH₂CH₃, R⁵ is H and the values of P, r, W and Q are as given in Table 1 for compounds 1-1 to 1-294, are designated as compound Nos. 7-1 to 7-294, respectively.

294 compounds of formula (I), wherein A is cyclopropyl, Y is Cl, Z is CO₂CH₂CH₃, R⁵ is CH₃ and the values of P, r, W and Q are as given in Table 1 for compounds 1-1 to 1-294, are designated as compound Nos. 8-1 to 8-294, respectively.

294 compounds of formula (I), wherein A is cyclopropyl, Y is Cl, Z is CO₂CH₂CH₃, R⁵ is CH₂CH₃ and the values of P, r, W and Q are as given in Table 1 for compounds 1-1 to 1-294, are designated as compound Nos. 9-1 to 9-294, respectively.

294 compounds of formula (I), wherein A is cyclopropyl, Y is Br, Z is CO₂H, R⁵ is H, and the values of P, r, W and Q are as given in Table 1 for compounds 1-1 to 1-294, are designated as compound Nos. 10-1 to 10-294, respectively.

294 compounds of formula (I), wherein A is cyclopropyl, Y is Br, Z is CO₂H, R⁵ is CH₃, and the values of P, r, W and Q are as given in Table 1 for compounds 1-1 to 1-294, are designated as compound Nos. 11-1 to 11-294, respectively.

294 compounds of formula (I), wherein A is cyclopropyl, Y is Br, Z is CO₂H, R⁵ is CH₂CH₃, and the values of P, r, W and Q are as given in Table 1 for compounds 1-1 to 1-294, are designated as compound Nos. 12-1 to 12-294, respectively.

294 compounds of formula (I), wherein A is cyclopropyl, Y is Br, Z is CO₂CH₃, R⁵ is H, and the values of P, r, W and Q are as given in Table 1 for compounds 1-1 to 1-294, are designated as compound Nos. 13-1 to 13-294, respectively.

294 compounds of formula (I), wherein A is cyclopropyl, Y is Br, Z is CO₂CH₃, R⁵ is CH₃, and the values of P, r, W and Q are as given in Table 1 for compounds 1-1 to 1-294, are designated as compound Nos. 14-1 to 14-294, respectively.

294 compounds of formula (I), wherein A is cyclopropyl, Y is Br, Z is CO₂CH₃, R⁵ is CH₂CH₃, and the values of P, r, W and Q are as given in Table 1 for compounds 1-1 to 1-294, are designated as compound Nos. 15-1 to 15-294, respectively.

294 compounds of formula (I), wherein A is cyclopropyl, Y is Br, Z is CO₂CH₂CH₃, R⁵ is H, and the values of P, r, W and Q are as given in Table 1 for compounds 1-1 to 1-294, are designated as compound Nos. 16-1 to 16-294, respectively.

294 compounds of formula (I), wherein A is cyclopropyl, Y is Br, Z is CO₂CH₂CH₃, R⁵ is CH₃, and the values of P, r, W and Q are as given in Table 1 for compounds 1-1 to 1-294, are designated as compound Nos. 17-1 to 17-294, respectively.

294 compounds of formula (I), wherein A is cyclopropyl, Y is Br, Z is CO₂CH₂CH₃, R⁵ is CH₂CH₃, and the values of P, r, W and Q are as given in Table 1 for compounds 1-1 to 1-294, are designated as compound Nos. 18-1 to 18-294, respectively.

294 compounds of formula (I), wherein A is cyclopropyl, Y is CH₃, Z is CO₂H, R⁵ is H, and the values of P, r, W and Q are as given in Table 1 for compounds 1-1 to 1-294, are designated as compound Nos. 19-1 to 19-294, respectively.

294 compounds of formula (I), wherein A is cyclopropyl, Y is CH₃, Z is CO₂H, R⁵ is CH₃, and the values of P, r, W and Q are as given in Table 1 for compounds 1-1 to 1-294, are designated as compound Nos. 20-1 to 20-294, respectively.

294 compounds of formula (I), wherein A is cyclopropyl, Y is CH₃, Z is CO₂H, R⁵ is CH₂CH₃, and the values of P, r, W and Q are as given in Table 1 for compounds 1-1 to 1-294, are designated as compound Nos. 21-1 to 21-294, respectively.

294 compounds of formula (I), wherein A is cyclopropyl, Y is CH₃, Z is CO₂CH₃, R⁵ is H, and the values of P, r, W and Q are as given in Table 1 for compounds 1-1 to 1-294, are designated as compound Nos. 22-1 to 22-294, respectively.

294 compounds of formula (I), wherein A is cyclopropyl, Y is CH₃, Z is CO₂CH₃, R⁵ is CH₃, and the values of P, r, W and Q are as given in Table 1 for compounds 1-1 to 1-294, are designated as compound Nos. 23-1 to 23-294, respectively.

294 compounds of formula (I), wherein A is cyclopropyl, Y is CH₃, Z is CO₂CH₃, R⁵ is CH₂CH₃, and the values of P, r, W and Q are as given in Table 1 for compounds 1-1 to 1-294, are designated as compound Nos. 24-1 to 24-294, respectively.

294 compounds of formula (I), wherein A is cyclopropyl, Y is CH₃, Z is CO₂CH₂CH₃, R⁵ is H and the values of P, r, W and Q are as given in Table 1 for compounds 1-1 to 1-294, are designated as compound Nos. 25-1 to 25-294, respectively.

294 compounds of formula (I), wherein A is cyclopropyl, Y is CH₃, Z is CO₂CH₂CH₃, R⁵ is CH₃ and the values of P, r, W and Q are as given in Table 1 for compounds 1-1 to 1-294, are designated as compound Nos. 26-1 to 26-294, respectively.

294 compounds of formula (I), wherein A is cyclopropyl, Y is CH₃, Z is CO₂CH₂CH₃, R⁵ is CH₂CH₃ and the values of P, r, W and Q are as given in Table 1 for compounds 1-1 to 1-294, are designated as compound Nos. 27-1 to 27-294, respectively.

294 compounds of formula (I), wherein A is cyclopropyl, Y is CH═CH₂, Z is CO₂H, R⁵ is H, and the values of P, r, W and Q are as given in Table 1 for compounds 1-1 to 1-294, are designated as compound Nos. 28-1 to 28-294, respectively.

294 compounds of formula (I), wherein A is cyclopropyl, Y is CH═CH₂, Z is CO₂H, R⁵ is CH₃, and the values of P, r, W and Q are as given in Table 1 for compounds 1-1 to 1-294, are designated as compound Nos. 29-1 to 29-294, respectively.

294 compounds of formula (I), wherein A is cyclopropyl, Y is CH═CH₂, Z is CO₂H, R⁵ is CH₂CH₃, and the values of P, r, W and Q are as given in Table 1 for compounds 1-1 to 1-294, are designated as compound Nos. 30-1 to 30-294, respectively.

294 compounds of formula (I), wherein A is cyclopropyl, Y is CH═CH₂, Z is CO₂CH₃, R⁵ is H, and the values of P, r, W and Q are as given in Table 1 for compounds 1-1 to 1-294, are designated as compound Nos. 31-1 to 31-294, respectively.

294 compounds of formula (I), wherein A is cyclopropyl, Y is CH═CH₂, Z is CO₂CH₃, R⁵ is CH₃, and the values of P, r, W and Q are as given in Table 1 for compounds 1-1 to 1-294, are designated as compound Nos. 32-1 to 32-294, respectively.

294 compounds of formula (I), wherein A is cyclopropyl, Y is CH═CH₂, Z is CO₂CH₃, R⁵ is CH₂CH₃, and the values of P, r, W and Q are as given in Table 1 for compounds 1-1 to 1-294, are designated as compound Nos. 33-1 to 33-294, respectively.

294 compounds of formula (I), wherein A is cyclopropyl, Y is CH═CH₂, Z is CO₂CH₂CH₃, R⁵ is H, and the values of P, r, W and Q are as given in Table 1 for compounds 1-1 to 1-294, are designated as compound Nos. 34-1 to 34-294, respectively.

294 compounds of formula (I), wherein A is cyclopropyl, Y is CH═CH₂, Z is CO₂CH₂CH₃, R⁵ is CH₃, and the values of P, r, W and Q are as given in Table 1 for compounds 1-1 to 1-294, are designated as compound Nos. 35-1 to 35-294, respectively.

294 compounds of formula (I), wherein A is cyclopropyl, Y is CH═CH₂, Z is CO₂CH₂CH₃, R⁵ is CH₂CH₃, and the values of P, r, W and Q are as given in Table 1 for compounds 1-1 to 1-294, are designated as compound Nos. 36-1 to 36-294, respectively.

294 compounds of formula (I), wherein A is 4-chlorophenyl, Y is Cl, Z is CO₂H, R⁵ is H, and the values of P, r, W and Q are as given in Table 1 for compounds 1-1 to 1-294, are designated as compound Nos. 37-1 to 37-294, respectively.

294 compounds of formula (I), wherein A is 4-chlorophenyl, Y is Cl, Z is CO₂H, R⁵ is CH₃, and the values of P, r, W and Q are as given in Table 1 for compounds 1-1 to 1-294, are designated as compound Nos. 38-1 to 38-294, respectively.

294 compounds of formula (I), wherein A is 4-chlorophenyl, Y is Cl, Z is CO₂H, R⁵ is CH₂CH₃, and the values of P, r, W and Q are as given in Table 1 for compounds 1-1 to 1-294, are designated as compound Nos. 39-1 to 39-294, respectively.

294 compounds of formula (I), wherein A is 4-chlorophenyl, Y is Cl, Z is CO₂CH₃, R⁵ is H, and the values of P, r, W and Q are as given in Table 1 for compounds 1-1 to 1-294, are designated as compound Nos. 40-1 to 40-294, respectively.

294 compounds of formula (I), wherein A is 4-chlorophenyl, Y is Cl, Z is CO₂CH₃, R⁵ is CH₃, and the values of P, r, W and Q are as given in Table 1 for compounds 1-1 to 1-294, are designated as compound Nos. 41-1 to 41-294, respectively.

294 compounds of formula (I), wherein A is 4-chlorophenyl, Y is Cl, Z is CO₂CH₃, R⁵ is CH₂CH₃, and the values of P, r, W and Q are as given in Table 1 for compounds 1-1 to 1-294, are designated as compound Nos. 42-1 to 42-294, respectively.

294 compounds of formula (I), wherein A is 4-chlorophenyl, Y is Cl, Z is CO₂CH₂CH₃, R⁵ is H and the values of P, r, W and Q are as given in Table 1 for compounds 1-1 to 1-294, are designated as compound Nos. 43-1 to 43-294, respectively.

294 compounds of formula (I), wherein A is 4-chlorophenyl, Y is Cl, Z is CO₂CH₂CH₃, R⁵ is CH₃ and the values of P, r, W and Q are as given in Table 1 for compounds 1-1 to 1-294, are designated as compound Nos. 44-1 to 44-294, respectively.

294 compounds of formula (I), wherein A is 4-chlorophenyl, Y is Cl, Z is CO₂CH₂CH₃, R⁵ is CH₂CH₃ and the values of P, r, W and Q are as given in Table 1 for compounds 1-1 to 1-294, are designated as compound Nos. 45-1 to 45-294, respectively.

294 compounds of formula (I), wherein A is 4-chlorophenyl, Y is Br, Z is CO₂H, R⁵ is H, and the values of P, r, W and Q are as given in Table 1 for compounds 1-1 to 1-294, are designated as compound Nos. 46-1 to 46-294, respectively.

294 compounds of formula (I), wherein A is 4-chlorophenyl, Y is Br, Z is CO₂H, R⁵ is CH₃, and the values of P, r, W and Q are as given in Table 1 for compounds 1-1 to 1-294, are designated as compound Nos. 47-1 to 47-294, respectively.

294 compounds of formula (I), wherein A is 4-chlorophenyl, Y is Br, Z is CO₂H, R⁵ is CH₂CH₃, and the values of P, r, W and Q are as given in Table 1 for compounds 1-1 to 1-294, are designated as compound Nos. 48-1 to 48-294, respectively.

294 compounds of formula (I), wherein A is 4-chlorophenyl, Y is Br, Z is CO₂CH₃, R⁵ is H, and the values of P, r, W and Q are as given in Table 1 for compounds 1-1 to 1-294, are designated as compound Nos. 49-1 to 49-294, respectively.

294 compounds of formula (I), wherein A is 4-chlorophenyl, Y is Br, Z is CO₂CH₃, R⁵ is CH₃, and the values of P, r, W and Q are as given in Table 1 for compounds 1-1 to 1-294, are designated as compound Nos. 50-1 to 50-294, respectively.

294 compounds of formula (I), wherein A is 4-chlorophenyl, Y is Br, Z is CO₂CH₃, R⁵ is CH₂CH₃, and the values of P, r, W and Q are as given in Table 1 for compounds 1-1 to 1-294, are designated as compound Nos. 51-1 to 51-294, respectively.

294 compounds of formula (I), wherein A is 4-chlorophenyl, Y is Br, Z is CO₂CH₂CH₃, R⁵ is H, and the values of P, r, W and Q are as given in Table 1 for compounds 1-1 to 1-294, are designated as compound Nos. 52-1 to 52-294, respectively.

294 compounds of formula (I), wherein A is 4-chlorophenyl, Y is Br, Z is CO₂CH₂CH₃, R⁵ is CH₃, and the values of P, r, W and Q are as given in Table 1 for compounds 1-1 to 1-294, are designated as compound Nos. 53-1 to 53-294, respectively.

294 compounds of formula (I), wherein A is 4-chlorophenyl, Y is Br, Z is CO₂CH₂CH₃, R⁵ is CH₂CH₃, and the values of P, r, W and Q are as given in Table 1 for compounds 1-1 to 1-294, are designated as compound Nos. 54-1 to 54-294, respectively.

294 compounds of formula (I), wherein A is 4-chlorophenyl, Y is CH₃, Z is CO₂H, R⁵ is H, and the values of P, r, W and Q are as given in Table 1 for compounds 1-1 to 1-294, are designated as compound Nos. 55-1 to 55-294, respectively.

294 compounds of formula (I), wherein A is 4-chlorophenyl, Y is CH₃, Z is CO₂H, R⁵ is CH₃, and the values of P, r, W and Q are as given in Table 1 for compounds 1-1 to 1-294, are designated as compound Nos. 56-1 to 56-294, respectively.

294 compounds of formula (I), wherein A is 4-chlorophenyl, Y is CH₃, Z is CO₂H, R⁵ is CH₂CH₃, and the values of P, r, W and Q are as given in Table 1 for compounds 1-1 to 1-294, are designated as compound Nos. 57-1 to 57-294, respectively.

294 compounds of formula (I), wherein A is 4-chlorophenyl, Y is CH₃, Z is CO₂CH₃, R⁵ is H, and the values of P, r, W and Q are as given in Table 1 for compounds 1-1 to 1-294, are designated as compound Nos. 58-1 to 58-294, respectively.

294 compounds of formula (I), wherein A is 4-chlorophenyl, Y is CH₃, Z is CO₂CH₃, R⁵ is CH₃, and the values of P, r, W and Q are as given in Table 1 for compounds 1-1 to 1-294, are designated as compound Nos. 59-1 to 59-294, respectively.

294 compounds of formula (I), wherein A is 4-chlorophenyl, Y is CH₃, Z is CO₂CH₃, R⁵ is CH₂CH₃, and the values of P, r, W and Q are as given in Table 1 for compounds 1-1 to 1-294, are designated as compound Nos. 60-1 to 60-294, respectively.

294 compounds of formula (I), wherein A is 4-chlorophenyl, Y is CH₃, Z is CO₂CH₂CH₃, R⁵ is H and the values of P, r, W and Q are as given in Table 1 for compounds 1-1 to 1-294, are designated as compound Nos. 61-1 to 61-294, respectively.

294 compounds of formula (I), wherein A is 4-chlorophenyl, Y is CH₃, Z is CO₂CH₂CH₃, R⁵ is CH₃ and the values of P, r, W and Q are as given in Table 1 for compounds 1-1 to 1-294, are designated as compound Nos. 62-1 to 62-294, respectively.

294 compounds of formula (I), wherein A is 4-chlorophenyl, Y is CH₃, Z is CO₂CH₂CH₃, R⁵ is CH₂CH₃ and the values of P, r, W and Q are as given in Table 1 for compounds 1-1 to 1-294, are designated as compound Nos. 63-1 to 63-294, respectively.

294 compounds of formula (I), wherein A is 4-chlorophenyl, Y is CH═CH₂, Z is CO₂H, R⁵ is H, and the values of P, r, W and Q are as given in Table 1 for compounds 1-1 to 1-294, are designated as compound Nos. 64-1 to 64-294, respectively.

294 compounds of formula (I), wherein A is 4-chlorophenyl, Y is CH═CH₂, Z is CO₂H, R⁵ is CH₃, and the values of P, r, W and Q are as given in Table 1 for compounds 1-1 to 1-294, are designated as compound Nos. 65-1 to 65-294, respectively.

294 compounds of formula (I), wherein A is 4-chlorophenyl, Y is CH═CH₂, Z is CO₂H, R⁵ is CH₂CH₃, and the values of P, r, W and Q are as given in Table 1 for compounds 1-1 to 1-294, are designated as compound Nos. 66-1 to 66-294, respectively.

294 compounds of formula (I), wherein A is 4-chlorophenyl, Y is CH═CH₂, Z is CO₂CH₃, R⁵ is H, and the values of P, r, W and Q are as given in Table 1 for compounds 1-1 to 1-294, are designated as compound Nos. 67-1 to 67-294, respectively.

294 compounds of formula (I), wherein A is 4-chlorophenyl, Y is CH═CH₂, Z is CO₂CH₃, R⁵ is CH₃, and the values of P, r, W and Q are as given in Table 1 for compounds 1-1 to 1-294, are designated as compound Nos. 68-1 to 68-294, respectively.

294 compounds of formula (I), wherein A is 4-chlorophenyl, Y is CH═CH₂, Z is CO₂CH₃, R⁵ is CH₂CH₃, and the values of P, r, W and Q are as given in Table 1 for compounds 1-1 to 1-294, are designated as compound Nos. 69-1 to 69-294, respectively.

294 compounds of formula (I), wherein A is 4-chlorophenyl, Y is CH═CH₂, Z is CO₂CH₂CH₃, R⁵ is H, and the values of P, r, W and Q are as given in Table 1 for compounds 1-1 to 1-294, are designated as compound Nos. 70-1 to 70-294, respectively.

294 compounds of formula (I), wherein A is 4-chlorophenyl, Y is CH═CH₂, Z is CO₂CH₂CH₃, R⁵ is CH₃, and the values of P, r, W and Q are as given in Table 1 for compounds 1-1 to 1-294, are designated as compound Nos. 71-1 to 71-294, respectively.

294 compounds of formula (I), wherein A is 4-chlorophenyl, Y is CH═CH₂, Z is CO₂CH₂CH₃, R⁵ is CH₂CH₃, and the values of P, r, W and Q are as given in Table 1 for compounds 1-1 to 1-294, are designated as compound Nos. 72-1 to 72-294, respectively.

294 compounds of formula (I), wherein A is 4-bromophenyl, Y is Cl, Z is CO₂H, R⁵ is H, and the values of P, r, W and Q are as given in Table 1 for compounds 1-1 to 1-294, are designated as compound Nos. 73-1 to 73-294, respectively.

294 compounds of formula (I), wherein A is 4-bromophenyl, Y is Cl, Z is CO₂H, R⁵ is CH₃, and the values of P, r, W and Q are as given in Table 1 for compounds 1-1 to 1-294, are designated as compound Nos. 74-1 to 74-294, respectively.

294 compounds of formula (I), wherein A is 4-bromophenyl, Y is Cl, Z is CO₂H, R⁵ is CH₂CH₃, and the values of P, r, W and Q are as given in Table 1 for compounds 1-1 to 1-294, are designated as compound Nos. 75-1 to 75-294, respectively.

294 compounds of formula (I), wherein A is 4-bromophenyl, Y is Cl, Z is CO₂CH₃, R⁵ is H, and the values of P, r, W and Q are as given in Table 1 for compounds 1-1 to 1-294, are designated as compound Nos. 76-1 to 76-294, respectively.

294 compounds of formula (I), wherein A is 4-bromophenyl, Y is Cl, Z is CO₂CH₃, R⁵ is CH₃, and the values of P, r, W and Q are as given in Table 1 for compounds 1-1 to 1-294, are designated as compound Nos. 77-1 to 77-294, respectively.

294 compounds of formula (I), wherein A is 4-bromophenyl, Y is Cl, Z is CO₂CH₃, R⁵ is CH₂CH₃, and the values of P, r, W and Q are as given in Table 1 for compounds 1-1 to 1-294, are designated as compound Nos. 78-1 to 78-294, respectively.

294 compounds of formula (I), wherein A is 4-bromophenyl, Y is Cl, Z is CO₂CH₂CH₃, R⁵ is H and the values of P, r, W and Q are as given in Table 1 for compounds 1-1 to 1-294, are designated as compound Nos. 79-1 to 79-294, respectively.

294 compounds of formula (I), wherein A is 4-bromophenyl, Y is Cl, Z is CO₂CH₂CH₃, R⁵ is CH₃ and the values of P, r, W and Q are as given in Table 1 for compounds 1-1 to 1-294, are designated as compound Nos. 80-1 to 80-294, respectively.

294 compounds of formula (I), wherein A is 4-bromophenyl, Y is Cl, Z is CO₂CH₂CH₃, R⁵ is CH₂CH₃ and the values of P, r, W and Q are as given in Table 1 for compounds 1-1 to 1-294, are designated as compound Nos. 81-1 to 81-294, respectively.

294 compounds of formula (I), wherein A is 4-bromophenyl, Y is Br, Z is CO₂H, R⁵ is H, and the values of P, r, W and Q are as given in Table 1 for compounds 1-1 to 1-294, are designated as compound Nos. 82-1 to 82-294, respectively.

294 compounds of formula (I), wherein A is 4-bromophenyl, Y is Br, Z is CO₂H, R⁵ is CH₃, and the values of P, r, W and Q are as given in Table 1 for compounds 1-1 to 1-294, are designated as compound Nos. 83-1 to 83-294, respectively.

294 compounds of formula (I), wherein A is 4-bromophenyl, Y is Br, Z is CO₂H, R⁵ is CH₂CH₃, and the values of P, r, W and Q are as given in Table 1 for compounds 1-1 to 1-294, are designated as compound Nos. 84-1 to 84-294, respectively.

294 compounds of formula (I), wherein A is 4-bromophenyl, Y is Br, Z is CO₂CH₃, R⁵ is H, and the values of P, r, W and Q are as given in Table 1 for compounds 1-1 to 1-294, are designated as compound Nos. 85-1 to 85-294, respectively.

294 compounds of formula (I), wherein A is 4-bromophenyl, Y is Br, Z is CO₂CH₃, R⁵ is CH₃, and the values of P, r, W and Q are as given in Table 1 for compounds 1-1 to 1-294, are designated as compound Nos. 86-1 to 86-294, respectively.

294 compounds of formula (I), wherein A is 4-bromophenyl, Y is Br, Z is CO₂CH₃, R⁵ is CH₂CH₃, and the values of P, r, W and Q are as given in Table 1 for compounds 1-1 to 1-294, are designated as compound Nos. 87-1 to 87-294, respectively.

294 compounds of formula (I), wherein A is 4-bromophenyl, Y is Br, Z is CO₂CH₂CH₃, R⁵ is H, and the values of P, r, W and Q are as given in Table 1 for compounds 1-1 to 1-294, are designated as compound Nos. 88-1 to 88-294, respectively.

294 compounds of formula (I), wherein A is 4-bromophenyl, Y is Br, Z is CO₂CH₂CH₃, R⁵ is CH₃, and the values of P, r, W and Q are as given in Table 1 for compounds 1-1 to 1-294, are designated as compound Nos. 89-1 to 89-294, respectively.

294 compounds of formula (I), wherein A is 4-bromophenyl, Y is Br, Z is CO₂CH₂CH₃, R⁵ is CH₂CH₃, and the values of P, r, W and Q are as given in Table 1 for compounds 1-1 to 1-294, are designated as compound Nos. 90-1 to 90-294, respectively.

294 compounds of formula (I), wherein A is 4-bromophenyl, Y is CH₃, Z is CO₂H, R⁵ is H, and the values of P, r, W and Q are as given in Table 1 for compounds 1-1 to 1-294, are designated as compound Nos. 91-1 to 91-294, respectively.

294 compounds of formula (I), wherein A is 4-bromophenyl, Y is CH₃, Z is CO₂H, R⁵ is CH₃, and the values of P, r, W and Q are as given in Table 1 for compounds 1-1 to 1-294, are designated as compound Nos. 92-1 to 92-294, respectively.

294 compounds of formula (I), wherein A is 4-bromophenyl, Y is CH₃, Z is CO₂H, R⁵ is CH₂CH₃, and the values of P, r, W and Q are as given in Table 1 for compounds 1-1 to 1-294, are designated as compound Nos. 93-1 to 93-294, respectively.

294 compounds of formula (I), wherein A is 4-bromophenyl, Y is CH₃, Z is CO₂CH₃, R⁵ is H, and the values of P, r, W and Q are as given in Table 1 for compounds 1-1 to 1-294, are designated as compound Nos. 94-1 to 94-294, respectively.

294 compounds of formula (I), wherein A is 4-bromophenyl, Y is CH₃, Z is CO₂CH₃, R⁵ is CH₃, and the values of P, r, W and Q are as given in Table 1 for compounds 1-1 to 1-294, are designated as compound Nos. 95-1 to 95-294, respectively.

294 compounds of formula (I), wherein A is 4-bromophenyl, Y is CH₃, Z is CO₂CH₃, R⁵ is CH₂CH₃, and the values of P, r, W and Q are as given in Table 1 for compounds 1-1 to 1-294, are designated as compound Nos. 96-1 to 96-294, respectively.

294 compounds of formula (I), wherein A is 4-bromophenyl, Y is CH₃, Z is CO₂CH₂CH₃, R⁵ is H and the values of P, r, W and Q are as given in Table 1 for compounds 1-1 to 1-294, are designated as compound Nos. 97-1 to 97-294, respectively.

294 compounds of formula (I), wherein A is 4-bromophenyl, Y is CH₃, Z is CO₂CH₂CH₃, R⁵ is CH₃ and the values of P, r, W and Q are as given in Table 1 for compounds 1-1 to 1-294, are designated as compound Nos. 98-1 to 98-294, respectively.

294 compounds of formula (I), wherein A is 4-bromophenyl, Y is CH₃, Z is CO₂CH₂CH₃, R⁵ is CH₂CH₃ and the values of P, r, W and Q are as given in Table 1 for compounds 1-1 to 1-294, are designated as compound Nos. 99-1 to 99-294, respectively.

294 compounds of formula (I), wherein A is 4-bromophenyl, Y is CH═CH₂, Z is CO₂H, R⁵ is H, and the values of P, r, W and Q are as given in Table 1 for compounds 1-1 to 1-294, are designated as compound Nos. 100-1 to 100-294, respectively.

294 compounds of formula (I), wherein A is 4-bromophenyl, Y is CH═CH₂, Z is CO₂H, R⁵ is CH₃, and the values of P, r, W and Q are as given in Table 1 for compounds 1-1 to 1-294, are designated as compound Nos. 101-1 to 101-294, respectively.

294 compounds of formula (I), wherein A is 4-bromophenyl, Y is CH═CH₂, Z is CO₂H, R⁵ is CH₂CH₃, and the values of P, r, W and Q are as given in Table 1 for compounds 1-1 to 1-294, are designated as compound Nos. 102-1 to 102-294, respectively.

294 compounds of formula (I), wherein A is 4-bromophenyl, Y is CH═CH₂, Z is CO₂CH₃, R⁵ is H, and the values of P, r, W and Q are as given in Table 1 for compounds 1-1 to 1-294, are designated as compound Nos. 103-1 to 103-294, respectively.

294 compounds of formula (I), wherein A is 4-bromophenyl, Y is CH═CH₂, Z is CO₂CH₃, R⁵ is CH₃, and the values of P, r, W and Q are as given in Table 1 for compounds 1-1 to 1-294, are designated as compound Nos. 104-1 to 104-294, respectively.

294 compounds of formula (I), wherein A is 4-bromophenyl, Y is CH═CH₂, Z is CO₂CH₃, R⁵ is CH₂CH₃, and the values of P, r, W and Q are as given in Table 1 for compounds 1-1 to 1-294, are designated as compound Nos. 105-1 to 105-294, respectively.

294 compounds of formula (I), wherein A is 4-bromophenyl, Y is CH═CH₂, Z is CO₂CH₂CH₃, R⁵ is H, and the values of P, r, W and Q are as given in Table 1 for compounds 1-1 to 1-294, are designated as compound Nos. 106-1 to 106-294, respectively.

294 compounds of formula (I), wherein A is 4-bromophenyl, Y is CH═CH₂, Z is CO₂CH₂CH₃, R⁵ is CH₃, and the values of P, r, W and Q are as given in Table 1 for compounds 1-1 to 1-294, are designated as compound Nos. 107-1 to 107-294, respectively.

294 compounds of formula (I), wherein A is 4-bromophenyl, Y is CH═CH₂, Z is CO₂CH₂CH₃, R⁵ is CH₂CH₃, and the values of P, r, W and Q are as given in Table 1 for compounds 1-1 to 1-294, are designated as compound Nos. 108-1 to 108-294, respectively.

294 compounds of formula (I), wherein A is 4-chloro-2-fluoro-3-methoxyphenyl, Y is Cl, Z is CO₂H, R⁵ is H, and the values of P, r, W and Q are as given in Table 1 for compounds 1-1 to 1-294, are designated as compound Nos. 109-1 to 109-294, respectively.

294 compounds of formula (I), wherein A is 4-chloro-2-fluoro-3-methoxyphenyl, Y is Cl, Z is CO₂H, R⁵ is CH₃, and the values of P, r, W and Q are as given in Table 1 for compounds 1-1 to 1-294, are designated as compound Nos. 110-1 to 110-294, respectively.

294 compounds of formula (I), wherein A is 4-chloro-2-fluoro-3-methoxyphenyl, Y is Cl, Z is CO₂H, R⁵ is CH₂CH₃, and the values of P, r, W and Q are as given in Table 1 for compounds 1-1 to 1-294, are designated as compound Nos. 111-1 to 111-294, respectively.

294 compounds of formula (I), wherein A is 4-chloro-2-fluoro-3-methoxyphenyl, Y is Cl, Z is CO₂CH₃, R⁵ is H, and the values of P, r, W and Q are as given in Table 1 for compounds 1-1 to 1-294, are designated as compound Nos. 112-1 to 112-294, respectively.

294 compounds of formula (I), wherein A is 4-chloro-2-fluoro-3-methoxyphenyl, Y is Cl, Z is CO₂CH₃, R⁵ is CH₃, and the values of P, r, W and Q are as given in Table 1 for compounds 1-1 to 1-294, are designated as compound Nos. 113-1 to 113-294, respectively.

294 compounds of formula (I), wherein A is 4-chloro-2-fluoro-3-methoxyphenyl, Y is Cl, Z is CO₂CH₃, R⁵ is CH₂CH₃, and the values of P, r, W and Q are as given in Table 1 for compounds 1-1 to 1-294, are designated as compound Nos. 114-1 to 114-294, respectively.

294 compounds of formula (I), wherein A is 4-chloro-2-fluoro-3-methoxyphenyl, Y is Cl, Z is CO₂CH₂CH₃, R⁵ is H and the values of P, r, W and Q are as given in Table 1 for compounds 1-1 to 1-294, are designated as compound Nos. 115-1 to 115-294, respectively.

294 compounds of formula (I), wherein A is 4-chloro-2-fluoro-3-methoxyphenyl, Y is Cl, Z is CO₂CH₂CH₃, R⁵ is CH₃ and the values of P, r, W and Q are as given in Table 1 for compounds 1-1 to 1-294, are designated as compound Nos. 116-1 to 116-294, respectively.

294 compounds of formula (I), wherein A is 4-chloro-2-fluoro-3-methoxyphenyl, Y is Cl, Z is CO₂CH₂CH₃, R⁵ is CH₂CH₃ and the values of P, r, W and Q are as given in Table 1 for compounds 1-1 to 1-294, are designated as compound Nos. 117-1 to 117-294, respectively.

294 compounds of formula (I), wherein A is 4-chloro-2-fluoro-3-methoxyphenyl, Y is Br, Z is CO₂H, R⁵ is H, and the values of P, r, W and Q are as given in Table 1 for compounds 1-1 to 1-294, are designated as compound Nos. 118-1 to 118-294, respectively.

294 compounds of formula (I), wherein A is 4-chloro-2-fluoro-3-methoxyphenyl, Y is Br, Z is CO₂H, R⁵ is CH₃, and the values of P, r, W and Q are as given in Table 1 for compounds 1-1 to 1-294, are designated as compound Nos. 119-1 to 119-294, respectively.

294 compounds of formula (I), wherein A is 4-chloro-2-fluoro-3-methoxyphenyl, Y is Br, Z is CO₂H, R⁵ is CH₂CH₃, and the values of P, r, W and Q are as given in Table 1 for compounds 1-1 to 1-294, are designated as compound Nos. 120-1 to 120-294, respectively.

294 compounds of formula (I), wherein A is 4-chloro-2-fluoro-3-methoxyphenyl, Y is Br, Z is CO₂CH₃, R⁵ is H, and the values of P, r, W and Q are as given in Table 1 for compounds 1-1 to 1-294, are designated as compound Nos. 121-1 to 121-294, respectively.

294 compounds of formula (I), wherein A is 4-chloro-2-fluoro-3-methoxyphenyl, Y is Br, Z is CO₂CH₃, R⁵ is CH₃, and the values of P, r, W and Q are as given in Table 1 for compounds 1-1 to 1-294, are designated as compound Nos. 122-1 to 122-294, respectively.

294 compounds of formula (I), wherein A is 4-chloro-2-fluoro-3-methoxyphenyl, Y is Br, Z is CO₂CH₃, R⁵ is CH₂CH₃, and the values of P, r, W and Q are as given in Table 1 for compounds 1-1 to 1-294, are designated as compound Nos. 123-1 to 123-294, respectively.

294 compounds of formula (I), wherein A is 4-chloro-2-fluoro-3-methoxyphenyl, Y is Br, Z is CO₂CH₂CH₃, R⁵ is H, and the values of P, r, W and Q are as given in Table 1 for compounds 1-1 to 1-294, are designated as compound Nos. 124-1 to 124-294, respectively.

294 compounds of formula (I), wherein A is 4-chloro-2-fluoro-3-methoxyphenyl, Y is Br, Z is CO₂CH₂CH₃, R⁵ is CH₃, and the values of P, r, W and Q are as given in Table 1 for compounds 1-1 to 1-294, are designated as compound Nos. 125-1 to 125-294, respectively.

294 compounds of formula (I), wherein A is 4-chloro-2-fluoro-3-methoxyphenyl, Y is Br, Z is CO₂CH₂CH₃, R⁵ is CH₂CH₃, and the values of P, r, W and Q are as given in Table 1 for compounds 1-1 to 1-294, are designated as compound Nos. 126-1 to 126-294, respectively.

294 compounds of formula (I), wherein A is 4-chloro-2-fluoro-3-methoxyphenyl, Y is CH₃, Z is CO₂H, R⁵ is H, and the values of P, r, W and Q are as given in Table 1 for compounds 1-1 to 1-294, are designated as compound Nos. 127-1 to 127-294, respectively.

294 compounds of formula (I), wherein A is 4-chloro-2-fluoro-3-methoxyphenyl, Y is CH₃, Z is CO₂H, R⁵ is CH₃, and the values of P, r, W and Q are as given in Table 1 for compounds 1-1 to 1-294, are designated as compound Nos. 128-1 to 128-294, respectively.

294 compounds of formula (I), wherein A is 4-chloro-2-fluoro-3-methoxyphenyl, Y is CH₃, Z is CO₂H, R⁵ is CH₂CH₃, and the values of P, r, W and Q are as given in Table 1 for compounds 1-1 to 1-294, are designated as compound Nos. 129-1 to 129-294, respectively.

294 compounds of formula (I), wherein A is 4-chloro-2-fluoro-3-methoxyphenyl, Y is CH₃, Z is CO₂CH₃, R⁵ is H, and the values of P, r, W and Q are as given in Table 1 for compounds 1-1 to 1-294, are designated as compound Nos. 130-1 to 130-294, respectively.

294 compounds of formula (I), wherein A is 4-chloro-2-fluoro-3-methoxyphenyl, Y is CH₃, Z is CO₂CH₃, R⁵ is CH₃, and the values of P, r, W and Q are as given in Table 1 for compounds 1-1 to 1-294, are designated as compound Nos. 131-1 to 131-294, respectively.

294 compounds of formula (I), wherein A is 4-chloro-2-fluoro-3-methoxyphenyl, Y is CH₃, Z is CO₂CH₃, R⁵ is CH₂CH₃, and the values of P, r, W and Q are as given in Table 1 for compounds 1-1 to 1-294, are designated as compound Nos. 132-1 to 132-294, respectively.

294 compounds of formula (I), wherein A is 4-chloro-2-fluoro-3-methoxyphenyl, Y is CH₃, Z is CO₂CH₂CH₃, R⁵ is H and the values of P, r, W and Q are as given in Table 1 for compounds 1-1 to 1-294, are designated as compound Nos. 133-1 to 133-294, respectively.

294 compounds of formula (I), wherein A is 4-chloro-2-fluoro-3-methoxyphenyl, Y is CH₃, Z is CO₂CH₂CH₃, R⁵ is CH₃ and the values of P, r, W and Q are as given in Table 1 for compounds 1-1 to 1-294, are designated as compound Nos. 134-1 to 134-294, respectively.

294 compounds of formula (I), wherein A is 4-chloro-2-fluoro-3-methoxyphenyl, Y is CH₃, Z is CO₂CH₂CH₃, R⁵ is CH₂CH₃ and the values of P, r, W and Q are as given in Table 1 for compounds 1-1 to 1-294, are designated as compound Nos. 135-1 to 135-294, respectively.

294 compounds of formula (I), wherein A is 4-chloro-2-fluoro-3-methoxyphenyl, Y is CH═CH₂, Z is CO₂H, R⁵ is H, and the values of P, r, W and Q are as given in Table 1 for compounds 1-1 to 1-294, are designated as compound Nos. 136-1 to 136-294, respectively.

294 compounds of formula (I), wherein A is 4-chloro-2-fluoro-3-methoxyphenyl, Y is CH═CH₂, Z is CO₂H, R⁵ is CH₃, and the values of P, r, W and Q are as given in Table 1 for compounds 1-1 to 1-294, are designated as compound Nos. 137-1 to 138-294, respectively.

294 compounds of formula (I), wherein A is 4-chloro-2-fluoro-3-methoxyphenyl, Y is CH═CH₂, Z is CO₂H, R⁵ is CH₂CH₃, and the values of P, r, W and Q are as given in Table 1 for compounds 1-1 to 1-294, are designated as compound Nos. 138-1 to 138-294, respectively.

294 compounds of formula (I), wherein A is 4-chloro-2-fluoro-3-methoxyphenyl, Y is CH═CH₂, Z is CO₂CH₃, R⁵ is H, and the values of P, r, W and Q are as given in Table 1 for compounds 1-1 to 1-294, are designated as compound Nos. 139-1 to 139-294, respectively.

294 compounds of formula (I), wherein A is 4-chloro-2-fluoro-3-methoxyphenyl, Y is CH═CH₂, Z is CO₂CH₃, R⁵ is CH₃, and the values of P, r, W and Q are as given in Table 1 for compounds 1-1 to 1-294, are designated as compound Nos. 140-1 to 140-294, respectively.

294 compounds of formula (I), wherein A is 4-chloro-2-fluoro-3-methoxyphenyl, Y is CH═CH₂, Z is CO₂CH₃, R⁵ is CH₂CH₃, and the values of P, r, W and Q are as given in Table 1 for compounds 1-1 to 1-294, are designated as compound Nos. 141-1 to 141-294, respectively.

294 compounds of formula (I), wherein A is 4-chloro-2-fluoro-3-methoxyphenyl, Y is CH═CH₂, Z is CO₂CH₂CH₃, R⁵ is H, and the values of P, r, W and Q are as given in Table 1 for compounds 1-1 to 1-294, are designated as compound Nos. 142-1 to 142-294, respectively.

294 compounds of formula (I), wherein A is 4-chloro-2-fluoro-3-methoxyphenyl, Y is CH═CH₂, Z is CO₂CH₂CH₃, R⁵ is CH₃, and the values of P, r, W and Q are as given in Table 1 for compounds 1-1 to 1-294, are designated as compound Nos. 143-1 to 143-294, respectively.

294 compounds of formula (I), wherein A is 4-chloro-2-fluoro-3-methoxyphenyl, Y is CH═CH₂, Z is CO₂CH₂CH₃, R⁵ is CH₂CH₃, and the values of P, r, W and Q are as given in Table 1 for compounds 1-1 to 1-294, are designated as compound Nos. 144-1 to 144-294, respectively.

294 compounds of formula (I), wherein A is cyclopropyl, Y is Cl, Z is CO₂CH₃, R⁵ is ⁿpropyl and the values of P, r, W and Q are as given in Table 1 for compounds 1-1 to 1-294, are designated as compound Nos. 145-1 to 145-294, respectively.

294 compounds of formula (I), wherein A is cyclopropyl, Y is Cl, Z is CO₂CH₃, R⁵ is isopropyl and the values of P, r, W and Q are as given in Table 1 for compounds 1-1 to 1-294, are designated as compound Nos. 146-1 to 146-294, respectively.

294 compounds of formula (I), wherein A is cyclopropyl, Y is Cl, Z is CO₂CH₃, R⁵ is ⁿbutyl and the values of P, r, W and Q are as given in Table 1 for compounds 1-1 to 1-294, are designated as compound Nos. 147-1 to 147-294, respectively.

294 compounds of formula (I), wherein A is cyclopropyl, Y is Cl, Z is CO₂CH₃, R⁵ is ^{s}butyl and the values of P, r, W and Q are as given in Table 1 for compounds 1-1 to 1-294, are designated as compound Nos. 148-1 to 148-294, respectively.

294 compounds of formula (I), wherein A is cyclopropyl, Y is Cl, Z is CO₂CH₃, R⁵ is ^{t}butyl and the values of P, r, W and Q are as given in Table 1 for compounds 1-1 to 1-294, are designated as compound Nos. 149-1 to 149-294, respectively.

294 compounds of formula (I), wherein A is cyclopropyl, Y is Cl, Z is CO₂CH₃, R⁵ is trimethylsilylmethyl and the values of P, r, W and Q are as given in Table 1 for compounds 1-1 to 1-294, are designated as compound Nos. 150-1 to 150-294, respectively.

294 compounds of formula (I), wherein A is cyclopropyl, Y is Cl, Z is CO₂CH₃, R⁵ is prop-2-en-1-yl and the values of P, r, W and Q are as given in Table 1 for compounds 1-1 to 1-294, are designated as compound Nos. 151-1 to 151-294, respectively.

294 compounds of formula (I), wherein A is cyclopropyl, Y is Cl, Z is CO₂CH₃, R⁵ is 3-methylbut-2-en-1-yl and the values of P, r, W and Q are as given in Table 1 for compounds 1-1 to 1-294, are designated as compound Nos. 152-1 to 152-294, respectively.

294 compounds of formula (I), wherein A is cyclopropyl, Y is Cl, Z is CO₂CH₃, R⁵ is prop-2-yn-1-yl and the values of P, r, W and Q are as given in Table 1 for compounds 1-1 to 1-294, are designated as compound Nos. 152-1 to 152-294, respectively.

294 compounds of formula (I), wherein A is cyclopropyl, Y is Cl, Z is CO₂CH₃, R⁵ is but-3-en-1-yl and the values of P, r, W and Q are as given in Table 1 for compounds 1-1 to 1-294, are designated as compound Nos. 154-1 to 154-294, respectively.

294 compounds of formula (I), wherein A is cyclopropyl, Y is Cl, Z is CO₂CH₃, R⁵ is phenylmethyl and the values of P, r, W and Q are as given in Table 1 for compounds 1-1 to 1-294, are designated as compound Nos. 155-1 to 155-294, respectively.

294 compounds of formula (I), wherein A is cyclopropyl, Y is Cl, Z is CO₂CH₃, R⁵ is CH₂CO₂H and the values of P, r, W and Q are as given in Table 1 for compounds 1-1 to 1-294, are designated as compound Nos. 156-1 to 156-294, respectively.

294 compounds of formula (I), wherein A is cyclopropyl, Y is Cl, Z is CO₂CH₃, R⁵ is 2-hydroxyethyl and the values of P, r, W and Q are as given in Table 1 for compounds 1-1 to 1-294, are designated as compound Nos. 157-1 to 157-294, respectively.

294 compounds of formula (I), wherein A is cyclopropyl, Y is Cl, Z is CO₂CH₃, R⁵ is 1,1-dimethyl-2-hydroxyethyl and the values of P, r, W and Q are as given in Table 1 for compounds 1-1 to 1-294, are designated as compound Nos. 158-1 to 158-294, respectively.

294 compounds of formula (I), wherein A is cyclopropyl, Y is Cl, Z is CO₂CH₃, R⁵ is 2-hydroxyprop-1-yl and the values of P, r, W and Q are as given in Table 1 for compounds 1-1 to 1-294, are designated as compound Nos. 159-1 to 159-294, respectively.

294 compounds of formula (I), wherein A is cyclopropyl, Y is Cl, Z is CO₂CH₃, R⁵ is 2-methoxyethyl and the values of P, r, W and Q are as given in Table 1 for compounds 1-1 to 1-294, are designated as compound Nos. 160-1 to 160-294, respectively.

294 compounds of formula (I), wherein A is cyclopropyl, Y is Cl, Z is CO₂CH₃, R⁵ is 2-cyanoethyl and the values of P, r, W and Q are as given in Table 1 for compounds 1-1 to 1-294, are designated as compound Nos. 161-1 to 161-294, respectively.

294 compounds of formula (I), wherein A is cyclopropyl, Y is Cl, Z is CO₂CH₃, R⁵ is 2-ethoxycarbonylethyl and the values of P, r, W and Q are as given in Table 1 for compounds 1-1 to 1-294, are designated as compound Nos. 162-1 to 162-294, respectively.

294 compounds of formula (I), wherein A is 4-chloro-2-fluoro-3-methoxyphenyl, Y is Cl, Z is CO₂CH₃, R⁵ is ⁿpropyl and the values of P, r, W and Q are as given in Table 1 for compounds 1-1 to 1-294, are designated as compound Nos. 163-1 to 163-294, respectively.

294 compounds of formula (I), wherein A is 4-chloro-2-fluoro-3-methoxyphenyl, Y is Cl, Z is CO₂CH₃, R⁵ is isopropyl and the values of P, r, W and Q are as given in Table 1 for compounds 1-1 to 1-294, are designated as compound Nos. 164-1 to 164-294, respectively.

294 compounds of formula (I), wherein A is 4-chloro-2-fluoro-3-methoxyphenyl, Y is Cl, Z is CO₂CH₃, R⁵ is ⁿbutyl and the values of P, r, W and Q are as given in Table 1 for compounds 1-1 to 1-294, are designated as compound Nos. 165-1 to 165-294, respectively.

294 compounds of formula (I), wherein A is 4-chloro-2-fluoro-3-methoxyphenyl, Y is Cl, Z is CO₂CH₃, R⁵ is ^{t}butyl and the values of P, r, W and Q are as given in Table 1 for compounds 1-1 to 1-294, are designated as compound Nos. 166-1 to 166-294, respectively.

294 compounds of formula (I), wherein A is 4-chloro-2-fluoro-3-methoxyphenyl, Y is Cl, Z is CO₂CH₃, R⁵ is trimethylsilylmethyl and the values of P, r, W and Q are as given in Table 1 for compounds 1-1 to 1-294, are designated as compound Nos. 167-1 to 167-294, respectively.

294 compounds of formula (I), wherein A is 4-chloro-2-fluoro-3-methoxyphenyl, Y is Cl, Z is CO₂CH₃, R⁵ is prop-2-en-1-yl and the values of P, r, W and Q are as given in Table 1 for compounds 1-1 to 1-294, are designated as compound Nos. 168-1 to 168-294, respectively.

294 compounds of formula (I), wherein A is 4-chloro-2-fluoro-3-methoxyphenyl, Y is Cl, Z is CO₂CH₃, R⁵ is prop-2-yn-1-yl and the values of P, r, W and Q are as given in Table 1 for compounds 1-1 to 1-294, are designated as compound Nos. 169-1 to 169-294, respectively.

294 compounds of formula (I), wherein A is 4-chloro-2-fluoro-3-methoxyphenyl, Y is Cl, Z is CO₂CH₃, R⁵ is but-3-en-1-yl and the values of P, r, W and Q are as given in Table 1 for compounds 1-1 to 1-294, are designated as compound Nos. 170-1 to 170-294, respectively.

294 compounds of formula (I), wherein A is 4-chloro-2-fluoro-3-methoxyphenyl, Y is Cl, Z is CO₂CH₃, R⁵ is phenylmethyl and the values of P, r, W and Q are as given in Table 1 for compounds 1-1 to 1-294, are designated as compound Nos. 171-1 to 171-294, respectively.

294 compounds of formula (I), wherein A is 4-chloro-2-fluoro-3-methoxyphenyl, Y is Cl, Z is CO₂CH₃, R⁵ is CH₂CO₂H and the values of P, r, W and Q are as given in Table 1 for compounds 1-1 to 1-294, are designated as compound Nos. 172-1 to 172-294, respectively.

294 compounds of formula (I), wherein A is 4-chloro-2-fluoro-3-methoxyphenyl, Y is Cl, Z is CO₂CH₃, R⁵ is 2-hydroxyethyl and the values of P, r, W and Q are as given in Table 1 for compounds 1-1 to 1-294, are designated as compound Nos. 173-1 to 173-294, respectively.

294 compounds of formula (I), wherein A is 4-chloro-2-fluoro-3-methoxyphenyl, Y is Cl, Z is CO₂CH₃, R⁵ is 1,1-dimethyl-2-hydroxyethyl and the values of P, r, W and Q are as given in Table 1 for compounds 1-1 to 1-294, are designated as compound Nos. 174-1 to 174-294, respectively.

294 compounds of formula (I), wherein A is 4-chloro-2-fluoro-3-methoxyphenyl, Y is Cl, Z is CO₂CH₃, R⁵ is 2-hydroxyprop-1-yl and the values of P, r, W and Q are as given in Table 1 for compounds 1-1 to 1-294, are designated as compound Nos. 175-1 to 175-294, respectively.

294 compounds of formula (I), wherein A is 4-chloro-2-fluoro-3-methoxyphenyl, Y is Cl, Z is CO₂CH₃, R⁵ is 2-methoxyethyl and the values of P, r, W and Q are as given in Table 1 for compounds 1-1 to 1-294, are designated as compound Nos. 176-1 to 176-294, respectively.

294 compounds of formula (I), wherein A is 4-chloro-2-fluoro-3-methoxyphenyl, Y is Cl, Z is CO₂CH₃, R⁵ is 2-cyanoethyl and the values of P, r, W and Q are as given in Table 1 for compounds 1-1 to 1-294, are designated as compound Nos. 177-1 to 177-294, respectively.

294 compounds of formula (I), wherein A is 4-chloro-2-fluoro-3-methoxyphenyl, Y is Cl, Z is CO₂CH₃, R⁵ is 2-ethoxycarbonylethyl and the values of P, r, W and Q are as given in Table 1 for compounds 1-1 to 1-294, are designated as compound Nos. 178-1 to 178-294, respectively.

294 compounds of formula (I), wherein A is cyclopropyl, Y is ethyl, Z is CO₂CH₃, R⁵ is H and the values of P, r, W and Q are as given in Table 1 for compounds 1-1 to 1-294, are designated as compound Nos. 179-1 to 179-294, respectively.

294 compounds of formula (I), wherein A is cyclopropyl, Y is ⁿpropyl, Z is CO₂CH₃, R⁵ is H and the values of P, r, W and Q are as given in Table 1 for compounds 1-1 to 1-294, are designated as compound Nos. 180-1 to 180-294, respectively.

294 compounds of formula (I), wherein A is cyclopropyl, Y is isopropyl, Z is CO₂CH₃, R⁵ is H and the values of P, r, W and Q are as given in Table 1 for compounds 1-1 to 1-294, are designated as compound Nos. 181-1 to 181-294, respectively.

294 compounds of formula (I), wherein A is cyclopropyl, Y is prop-1-enyl, Z is CO₂CH₃, R⁵ is H and the values of P, r, W and Q are as given in Table 1 for compounds 1-1 to 1-294, are designated as compound Nos. 182-1 to 182-294, respectively.

294 compounds of formula (I), wherein A is cyclopropyl, Y is prop-2-en-1-yl, Z is CO₂CH₃, R⁵ is H and the values of P, r, W and Q are as given in Table 1 for compounds 1-1 to 1-294, are designated as compound Nos. 183-1 to 183-294, respectively.

294 compounds of formula (I), wherein A is cyclopropyl, Y is 1-methylethenyl, Z is CO₂CH₃, R⁵ is H and the values of P, r, W and Q are as given in Table 1 for compounds 1-1 to 1-294, are designated as compound Nos. 184-1 to 184-294, respectively.

294 compounds of formula (I), wherein A is cyclopropyl, Y is ethynyl, Z is CO₂CH₃, R⁵ is H and the values of P, r, W and Q are as given in Table 1 for compounds 1-1 to 1-294, are designated as compound Nos. 185-1 to 185-294, respectively.

294 compounds of formula (I), wherein A is cyclopropyl, Y is 2-trimethylsilylethynyl, Z is CO₂CH₃, R⁵ is H and the values of P, r, W and Q are as given in Table 1 for compounds 1-1 to 1-294, are designated as compound Nos. 186-1 to 186-294, respectively.

294 compounds of formula (I), wherein A is cyclopropyl, Y is 4-ethenylphenyl, Z is CO₂CH₃, R⁵ is H and the values of P, r, W and Q are as given in Table 1 for compounds 1-1 to 1-294, are designated as compound Nos. 187-1 to 187-294, respectively.

294 compounds of formula (I), wherein A is 4-chloro-2-fluoro-3-methoxyphenyl, Y is ethyl, Z is CO₂CH₃, R⁵ is H and the values of P, r, W and Q are as given in Table 1 for compounds 1-1 to 1-294, are designated as compound Nos. 188-1 to 188-294, respectively.

294 compounds of formula (I), wherein A is 4-chloro-2-fluoro-3-methoxyphenyl, Y is ⁿpropyl, Z is CO₂CH₃, R⁵ is H and the values of P, r, W and Q are as given in Table 1 for compounds 1-1 to 1-294, are designated as compound Nos. 189-1 to 189-294, respectively.

294 compounds of formula (I), wherein A is 4-chloro-2-fluoro-3-methoxyphenyl, Y is isopropyl, Z is CO₂CH₃, R⁵ is H and the values of P, r, W and Q are as given in Table 1 for compounds 1-1 to 1-294, are designated as compound Nos. 190-1 to 190-294, respectively.

294 compounds of formula (I), wherein A is 4-chloro-2-fluoro-3-methoxyphenyl, Y is prop-1-enyl, Z is CO₂CH₃, R⁵ is H and the values of P, r, W and Q are as given in Table 1 for compounds 1-1 to 1-294, are designated as compound Nos. 191-1 to 191-294, respectively.

294 compounds of formula (I), wherein A is 4-chloro-2-fluoro-3-methoxyphenyl, Y is prop-2-en-1-yl, Z is CO₂CH₃, R⁵ is H and the values of P, r, W and Q are as given in Table 1 for compounds 1-1 to 1-294, are designated as compound Nos. 192-1 to 192-294, respectively.

294 compounds of formula (I), wherein A is 4-chloro-2-fluoro-3-methoxyphenyl, Y is 1-methylethenyl, Z is CO₂CH₃, R⁵ is H and the values of P, r, W and Q are as given in Table 1 for compounds 1-1 to 1-294, are designated as compound Nos. 193-1 to 193-294, respectively.

294 compounds of formula (I), wherein A is 4-chloro-2-fluoro-3-methoxyphenyl, Y is ethynyl, Z is CO₂CH₃, R⁵ is H and the values of P, r, W and Q are as given in Table 1 for compounds 1-1 to 1-294, are designated as compound Nos. 194-1 to 194-294, respectively.

294 compounds of formula (I), wherein A is 4-chloro-2-fluoro-3-methoxyphenyl, Y is 2-trimethylsilylethynyl, Z is CO₂CH₃, R⁵ is H and the values of P, r, W and Q are as given in Table 1 for compounds 1-1 to 1-294, are designated as compound Nos. 195-1 to 195-294, respectively.

294 compounds of formula (I), wherein A is 4-chloro-2-fluoro-3-methoxyphenyl, Y is 4-ethenylphenyl, Z is CO₂CH₃, R⁵ is H and the values of P, r, W and Q are as given in Table 1 for compounds 1-1 to 1-294, are designated as compound Nos. 196-1 to 196-294, respectively.

294 compounds of formula (I), wherein A is cyclopropyl, Y is Cl, Z is CO₂CH(CH₃)₂, R⁵ is H and the values of P, r, W and Q are as given in Table 1 for compounds 1-1 to 1-294, are designated as compound Nos. 197-1 to 197-294, respectively.

294 compounds of formula (I), wherein A is cyclopropyl, Y is Cl, Z is CO₂CH₂CH₂CH₃, R⁵ is H and the values of P, r, W and Q are as given in Table 1 for compounds 1-1 to 1-294, are designated as compound Nos. 198-1 to 198-294, respectively.

294 compounds of formula (I), wherein A is cyclopropyl, Y is Cl, Z is CO₂CH₂CH(CH₃)₂, R⁵ is H and the values of P, r, W and Q are as given in Table 1 for compounds 1-1 to 1-294, are designated as compound Nos. 199-1 to 199-294, respectively.

294 compounds of formula (I), wherein A is cyclopropyl, Y is Cl, Z is CO₂CH₂Ph, R⁵ is H and the values of P, r, W and Q are as given in Table 1 for compounds 1-1 to 1-294, are designated as compound Nos. 200-1 to 200-294, respectively.

294 compounds of formula (I), wherein A is cyclopropyl, Y is Cl, Z is CONH₂, R⁵ is H and the values of P, r, W and Q are as given in Table 1 for compounds 1-1 to 1-294, are designated as compound Nos. 201-1 to 201-294, respectively.

294 compounds of formula (I), wherein A is 4-chloro-2-fluoro-3-methoxyphenyl, Y is Cl, Z is CO₂CH(CH₃)₂, R⁵ is H and the values of P, r, W and Q are as given in Table 1 for compounds 1-1 to 1-294, are designated as compound Nos. 202-1 to 202-294, respectively.

294 compounds of formula (I), wherein A is 4-chloro-2-fluoro-3-methoxyphenyl, Y is Cl, Z is CO₂CH₂CH₂CH₃, R⁵ is H and the values of P, r, W and Q are as given in Table 1 for compounds 1-1 to 1-294, are designated as compound Nos. 203-1 to 203-294, respectively.

294 compounds of formula (I), wherein A is 4-chloro-2-fluoro-3-methoxyphenyl, Y is Cl, Z is CO₂CH₂CH(CH₃)₂, R⁵ is H and the values of P, r, W and Q are as given in Table 1 for compounds 1-1 to 1-294, are designated as compound Nos. 204-1 to 204-294, respectively.

294 compounds of formula (I), wherein A is 4-chloro-2-fluoro-3-methoxyphenyl, Y is Cl, Z is CO₂CH₂Ph, R⁵ is H and the values of P, r, W and Q are as given in Table 1 for compounds 1-1 to 1-294, are designated as compound Nos. 205-1 to 205-294, respectively.

294 compounds of formula (I), wherein A is 4-chloro-2-fluoro-3-methoxyphenyl, Y is Cl, Z is CON H₂, R⁵ is H and the values of P, r, W and Q are as given in Table 1 for compounds 1-1 to 1-294, are designated as compound Nos. 206-1 to 206-294, respectively.

Table 2 below provides 178 compounds designated compound numbers 207-1 to 207-178 respectively, of general formula (I) wherein A is Cl, W is a direct bond and Y, Z, R⁵, P, r and Q have the values as listed.

TABLE 2

| Compound | | | | | | |
|---|---|---|---|---|---|---|
| Number | Y | Z | R⁵ | P | r | Q |
| 207-1 | Cl | CO₂H | H | — | 0 | cyclopropyl |
| 207-2 | Cl | CO₂H | H | CH₂ | 1 | cyclopropyl |
| 207-3 | Cl | CO₂H | H | CH(CH₃) | 1 | cyclopropyl |
| 207-4 | Cl | CO₂H | H | — | 0 | cyclobutyl |
| 207-5 | Cl | CO₂H | H | CH(CH₂OH) | 1 | tetrahydrofuran-3-yl |
| 207-6 | Cl | CO₂H | H | CH₂ | 1 | phenyl |
| 207-7 | Cl | CO₂H | H | CH(CH₃) | 1 | phenyl |
| 207-8 | Cl | CO₂H | H | CH(CH═CH₂) | 1 | phenyl |
| 207-9 | Cl | CO₂H | H | CH₂ | 1 | 2-fluorophenyl |
| 207-10 | Cl | CO₂H | H | CH₂ | 1 | 4-fluorophenyl |
| 207-11 | Cl | CO₂H | H | CH₂ | 1 | 2-chlorophenyl |
| 207-12 | Cl | CO₂H | H | CH₂ | 1 | 4-chlorophenyl |
| 207-13 | Cl | CO₂H | H | CH₂ | 1 | 3-bromophenyl |
| 207-14 | Cl | CO₂H | H | CH₂ | 1 | 4-dimethylaminophenyl |
| 207-15 | Cl | CO₂H | H | CH₂ | 1 | 2,4-difluorophenyl |
| 207-16 | Cl | CO₂H | H | CH₂ | 1 | 2,6-dichlorophenyl |
| 207-17 | Cl | CO₂H | H | CH₂ | 1 | 2,4-dimethoxyphenyl |
| 207-18 | Cl | CO₂H | H | CH₂ | 1 | 2,3,6-trifluorophenyl |
| 207-19 | Cl | CO₂H | H | CH₂ | 1 | furan-2-yl |
| 207-20 | Cl | CO₂H | H | CH(CH₂OH) | 1 | furan-2-yl |
| 207-21 | Cl | CO₂H | H | CH₂ | 1 | 5-methylfuran-2-yl |
| 207-22 | Cl | CO₂H | H | CH₂ | 1 | 5-bromofuran-2-yl |
| 207-23 | Cl | CO₂H | H | CH₂ | 1 | thiophen-2-yl |
| 207-24 | Cl | CO₂H | H | CH₂ | 1 | 3-methylpyrid-2-yl |
| 207-25 | Cl | CO₂H | H | CH₂ | 1 | pyrid-4-yl |
| 207-26 | Cl | CO₂H | Me | CH₂ | 1 | 2-chlorophenyl |
| 207-27 | Cl | CO₂H | Me | CH₂ | 1 | furan-2-yl |
| 207-28 | Cl | CO₂H | Et | CH₂ | 1 | phenyl |
| 207-29 | Cl | CO₂H | Et | CH₂ | 1 | 2-fluorophenyl |
| 207-30 | Cl | CO₂Me | H | — | 0 | cyclopropyl |
| 207-31 | Cl | CO₂Me | H | CH₂ | 1 | cyclopropyl |
| 207-32 | Cl | CO₂Me | H | CH(CH₃) | 1 | cyclopropyl |
| 207-33 | Cl | CO₂Me | H | — | 0 | cyclobutyl |
| 207-34 | Cl | CO₂Me | H | CH(CH₂OH) | 1 | tetrahydrofuran-3-yl |
| 207-35 | Cl | CO₂Me | H | CH₂ | 1 | phenyl |
| 207-36 | Cl | CO₂Me | H | CH(CH₃) | 1 | phenyl |
| 207-37 | Cl | CO₂Me | H | CH(CH═CH₂) | 1 | phenyl |
| 207-38 | Cl | CO₂Me | H | CH₂ | 1 | 2-fluorophenyl |
| 207-39 | Cl | CO₂Me | H | CH₂ | 1 | 4-fluorophenyl |
| 207-40 | Cl | CO₂Me | H | CH₂ | 1 | 2-chlorophenyl |
| 207-41 | Cl | CO₂Me | H | CH₂ | 1 | 4-chlorophenyl |
| 207-42 | Cl | CO₂Me | H | CH₂ | 1 | 3-bromophenyl |
| 207-43 | Cl | CO₂Me | H | CH₂ | 1 | 4-dimethylaminophenyl |
| 207-44 | Cl | CO₂Me | H | CH₂ | 1 | 2,4-difluorophenyl |
| 207-45 | Cl | CO₂Me | H | CH₂ | 1 | 2,6-dichlorophenyl |
| 207-46 | Cl | CO₂Me | H | CH₂ | 1 | 2,4-dimethoxyphenyl |
| 207-47 | Cl | CO₂Me | H | CH₂ | 1 | 2,3,6-trifluorophenyl |
| 207-48 | Cl | CO₂Me | H | CH₂ | 1 | furan-2-yl |
| 207-49 | Cl | CO₂Me | H | CH(CH₂OH) | 1 | furan-2-yl |
| 207-50 | Cl | CO₂Me | H | CH₂ | 1 | 5-methylfuran-2-yl |
| 207-51 | Cl | CO₂Me | H | CH₂ | 1 | 5-bromofuran-2-yl |
| 207-52 | Cl | CO₂Me | H | CH₂ | 1 | thiophen-2-yl |
| 207-53 | Cl | CO₂Me | H | CH₂ | 1 | 3-methylpyrid-2-yl |
| 207-54 | Cl | CO₂Me | H | CH₂ | 1 | pyrid-4-yl |
| 207-55 | Cl | CO₂Me | Me | CH₂ | 1 | 2-chlorophenyl |
| 207-56 | Cl | CO₂Me | Me | CH₂ | 1 | furan-2-yl |
| 207-57 | Cl | CO₂Me | Et | CH₂ | 1 | phenyl |
| 207-58 | Cl | CO₂Me | Et | CH₂ | 1 | 2-fluorophenyl |
| 207-59 | Cl | CO₂Et | H | — | 0 | cyclopropyl |
| 207-60 | Cl | CO₂Et | H | CH₂ | 1 | cyclopropyl |
| 207-61 | Cl | CO₂Et | H | CH(CH₃) | 1 | cyclopropyl |
| 207-62 | Cl | CO₂Et | H | — | 0 | cyclobutyl |
| 207-63 | Cl | CO₂Et | H | CH(CH₂OH) | 1 | tetrahydrofuran-3-yl |
| 207-64 | Cl | CO₂Et | H | CH₂ | 1 | phenyl |
| 207-65 | Cl | CO₂Et | H | CH(CH₃) | 1 | phenyl |
| 207-66 | Cl | CO₂Et | H | CH(CH═CH₂) | 1 | phenyl |
| 207-67 | Cl | CO₂Et | H | CH₂ | 1 | 2-fluorophenyl |
| 207-68 | Cl | CO₂Et | H | CH₂ | 1 | 4-fluorophenyl |
| 207-69 | Cl | CO₂Et | H | CH₂ | 1 | 2-chlorophenyl |
| 207-70 | Cl | CO₂Et | H | CH₂ | 1 | 4-chlorophenyl |
| 207-71 | Cl | CO₂Et | H | CH₂ | 1 | 3-bromophenyl |
| 207-72 | Cl | CO₂Et | H | CH₂ | 1 | 4-dimethylaminophenyl |
| 207-73 | Cl | CO₂Et | H | CH₂ | 1 | 2,4-difluorophenyl |
| 207-74 | Cl | CO₂Et | H | CH₂ | 1 | 2,6-dichlorophenyl |
| 207-75 | Cl | CO₂Et | H | CH₂ | 1 | 2,4-dimethoxyphenyl |
| 207-76 | Cl | CO₂Et | H | CH₂ | 1 | 2,3,6-trifluorophenyl |
| 207-77 | Cl | CO₂Et | H | CH₂ | 1 | furan-2-yl |
| 207-78 | Cl | CO₂Et | H | CH(CH₂OH) | 1 | furan-2-yl |
| 207-79 | Cl | CO₂Et | H | CH₂ | 1 | 5-methylfuran-2-yl |
| 207-80 | Cl | CO₂Et | H | CH₂ | 1 | 5-bromofuran-2-yl |
| 207-81 | Cl | CO₂Et | H | CH₂ | 1 | thiophen-2-yl |
| 207-82 | Cl | CO₂Et | H | CH₂ | 1 | 3-methylpyrid-2-yl |
| 207-83 | Cl | CO₂Et | H | CH₂ | 1 | pyrid-4-yl |
| 207-84 | Cl | CO₂Et | Me | CH₂ | 1 | 2-chlorophenyl |
| 207-85 | Cl | CO₂Et | Me | CH₂ | 1 | furan-2-yl |
| 207-86 | Cl | CO₂Et | Et | CH₂ | 1 | phenyl |
| 207-87 | Cl | CO₂Et | Et | CH₂ | 1 | 2-fluorophenyl |
| 207-88 | Cl | CONHCH₂phenyl | H | CH₂ | 1 | phenyl |
| 207-89 | Cl | CONHCH₂(4- | H | CH₂ | 1 | 4-chlorophenyl |
| | | chlorophenyl) | | | | |
| 207-90 | Cl | CONHCH₂furan- | H | CH₂ | 1 | furan-2-yl |
| | | 2-yl | | | | |
| 207-91 | Cl | CONHCH₂thiophen- | H | CH₂ | 1 | thiophen-2-yl |
| | | 2-yl | | | | |
| 207-92 | Br | CO₂H | H | — | 0 | cyclopropyl |
| 207-93 | Br | CO₂H | H | CH₂ | 1 | cyclopropyl |
| 207-94 | Br | CO₂H | H | CH(CH₃) | 1 | cyclopropyl |
| 207-95 | Br | CO₂H | H | — | 0 | cyclobutyl |
| 207-96 | Br | CO₂H | H | CH(CH₂OH) | 1 | tetrahydrofuran-3-yl |
| 207-97 | Br | CO₂H | H | CH₂ | 1 | phenyl |
| 207-98 | Br | CO₂H | H | CH(CH₃) | 1 | phenyl |
| 207-99 | Br | CO₂H | H | CH(CH═CH₂) | 1 | phenyl |
| 207-100 | Br | CO₂H | H | CH₂ | 1 | 2-fluorophenyl |
| 207-101 | Br | CO₂H | H | CH₂ | 1 | 4-fluorophenyl |
| 207-102 | Br | CO₂H | H | CH₂ | 1 | 2-chlorophenyl |
| 207-103 | Br | CO₂H | H | CH₂ | 1 | 4-chlorophenyl |
| 207-104 | Br | CO₂H | H | CH₂ | 1 | 3-bromophenyl |
| 207-105 | Br | CO₂H | H | CH₂ | 1 | 4-dimethylaminophenyl |
| 207-106 | Br | CO₂H | H | CH₂ | 1 | 2,4-difluorophenyl |
| 207-107 | Br | CO₂H | H | CH₂ | 1 | 2,6-dichlorophenyl |
| 207-108 | Br | CO₂H | H | CH₂ | 1 | 2,4-dimethoxyphenyl |
| 207-109 | Br | CO₂H | H | CH₂ | 1 | 2,3,6-trifluorophenyl |
| 207-110 | Br | CO₂H | H | CH₂ | 1 | furan-2-yl |
| 207-111 | Br | CO₂H | H | CH(CH₂OH) | 1 | furan-2-yl |
| 207-112 | Br | CO₂H | H | CH₂ | 1 | 5-methylfuran-2-yl |
| 207-113 | Br | CO₂H | H | CH₂ | 1 | 5-bromofuran-2-yl |
| 207-114 | Br | CO₂H | H | CH₂ | 1 | thiophen-2-yl |
| 207-115 | Br | CO₂H | H | CH₂ | 1 | 3-methylpyrid-2-yl |
| 207-116 | Br | CO₂H | H | CH₂ | 1 | pyrid-4-yl |
| 207-117 | Br | CO₂H | Me | CH₂ | 1 | 2-chlorophenyl |
| 207-118 | Br | CO₂H | Me | CH₂ | 1 | furan-2-yl |
| 207-119 | Br | CO₂H | Et | CH₂ | 1 | phenyl |
| 207-120 | Br | CO₂H | Et | CH₂ | 1 | 2-fluorophenyl |
| 207-121 | Br | CO₂Me | H | — | 0 | cyclopropyl |
| 207-122 | Br | CO₂Me | H | CH₂ | 1 | cyclopropyl |
| 207-123 | Br | CO₂Me | H | CH(CH₃) | 1 | cyclopropyl |
| 207-124 | Br | CO₂Me | H | — | 0 | cyclobutyl |
| 207-125 | Br | CO₂Me | H | CH(CH₂OH) | 1 | tetrahydrofuran-3-yl |
| 207-126 | Br | CO₂Me | H | CH₂ | 1 | phenyl |
| 207-127 | Br | CO₂Me | H | CH(CH₃) | 1 | phenyl |
| 207-128 | Br | CO₂Me | H | CH(CH═CH₂) | 1 | phenyl |
| 207-129 | Br | CO₂Me | H | CH₂ | 1 | 2-fluorophenyl |
| 207-130 | Br | CO₂Me | H | CH₂ | 1 | 4-fluorophenyl |
| 207-131 | Br | CO₂Me | H | CH₂ | 1 | 2-chlorophenyl |
| 207-132 | Br | CO₂Me | H | CH₂ | 1 | 4-chlorophenyl |
| 207-133 | Br | CO₂Me | H | CH₂ | 1 | 3-bromophenyl |
| 207-134 | Br | CO₂Me | H | CH₂ | 1 | 4-dimethylaminophenyl |
| 207-135 | Br | CO₂Me | H | CH₂ | 1 | 2,4-difluorophenyl |
| 207-136 | Br | CO₂Me | H | CH₂ | 1 | 2,6-dichlorophenyl |
| 207-137 | Br | CO₂Me | H | CH₂ | 1 | 2,4-dimethoxyphenyl |
| 207-138 | Br | CO₂Me | H | CH₂ | 1 | 2,3,6-trifluorophenyl |
| 207-139 | Br | CO₂Me | H | CH₂ | 1 | furan-2-yl |
| 207-140 | Br | CO₂Me | H | CH(CH₂OH) | 1 | furan-2-yl |
| 207-141 | Br | CO₂Me | H | CH₂ | 1 | 5-methylfuran-2-yl |
| 207-142 | Br | CO₂Me | H | CH₂ | 1 | 5-bromofuran-2-yl |
| 207-143 | Br | CO₂Me | H | CH₂ | 1 | thiophen-2-yl |
| 207-144 | Br | CO₂Me | H | CH₂ | 1 | 3-methylpyrid-2-yl |
| 207-145 | Br | CO₂Me | H | CH₂ | 1 | pyrid-4-yl |
| 207-146 | Br | CO₂Me | Me | CH₂ | 1 | 2-chlorophenyl |
| 207-147 | Br | CO₂Me | Me | CH₂ | 1 | furan-2-yl |
| 207-148 | Br | CO₂Me | Et | CH₂ | 1 | phenyl |
| 207-149 | Br | CO₂Me | Et | CH₂ | 1 | 2-fluorophenyl |
| 207-150 | Br | CO₂Et | H | — | 0 | cyclopropyl |
| 207-151 | Br | CO₂Et | H | CH₂ | 1 | cyclopropyl |
| 207-152 | Br | CO₂Et | H | CH(CH₃) | 1 | cyclopropyl |
| 207-153 | Br | CO₂Et | H | — | 0 | cyclobutyl |
| 207-154 | Br | CO₂Et | H | CH(CH₂OH) | 1 | tetrahydrofuran-3-yl |
| 207-155 | Br | CO₂Et | H | CH₂ | 1 | phenyl |
| 207-156 | Br | CO₂Et | H | CH(CH₃) | 1 | phenyl |
| 207-157 | Br | CO₂Et | H | CH(CH═CH₂) | 1 | phenyl |
| 207-158 | Br | CO₂Et | H | CH₂ | 1 | 2-fluorophenyl |
| 207-159 | Br | CO₂Et | H | CH₂ | 1 | 4-fluorophenyl |
| 207-160 | Br | CO₂Et | H | CH₂ | 1 | 2-chlorophenyl |
| 207-161 | Br | CO₂Et | H | CH₂ | 1 | 4-chlorophenyl |
| 207-162 | Br | CO₂Et | H | CH₂ | 1 | 3-bromophenyl |
| 207-163 | Br | CO₂Et | H | CH₂ | 1 | 4-dimethylaminophenyl |
| 207-164 | Br | CO₂Et | H | CH₂ | 1 | 2,4-difluorophenyl |
| 207-165 | Br | CO₂Et | H | CH₂ | 1 | 2,6-dichlorophenyl |
| 207-166 | Br | CO₂Et | H | CH₂ | 1 | 2,4-dimethoxyphenyl |
| 207-167 | Br | CO₂Et | H | CH₂ | 1 | 2,3,6-trifluorophenyl |
| 207-168 | Br | CO₂Et | H | CH₂ | 1 | furan-2-yl |
| 207-169 | Br | CO₂Et | H | CH(CH₂OH) | 1 | furan-2-yl |
| 207-170 | Br | CO₂Et | H | CH₂ | 1 | 5-methylfuran-2-yl |
| 207-171 | Br | CO₂Et | H | CH₂ | 1 | 5-bromofuran-2-yl |
| 207-172 | Br | CO₂Et | H | CH₂ | 1 | thiophen-2-yl |
| 207-173 | Br | CO₂Et | H | CH₂ | 1 | 3-methylpyrid-2-yl |
| 207-174 | Br | CO₂Et | H | CH₂ | 1 | pyrid-4-yl |
| 207-175 | Br | CO₂Et | Me | CH₂ | 1 | 2-chlorophenyl |
| 207-176 | Br | CO₂Et | Me | CH₂ | 1 | furan-2-yl |
| 207-177 | Br | CO₂Et | Et | CH₂ | 1 | phenyl |
| 207-178 | Br | CO₂Et | Et | CH₂ | 1 | 2-fluorophenyl |

178 compounds of formula (I) wherein A is isopropyl, W is a direct bond and Y, Z, R⁵, P, r and Q are as given in Table 2 for compounds 207-1 to 207-178, are designated as compound Nos. 208-1 to 208-178, respectively.

178 compounds of formula (I) wherein A is 2-methylcyclopropyl, W is a direct bond and Y, Z, R⁵, P, r and Q are as given in Table 2 for compounds 207-1 to 207-178, are designated as compound Nos. 209-1 to 209-178, respectively.

178 compounds of formula (I) wherein A is 4-iodophenyl, W is a direct bond and Y, Z, R⁵, P, r and Q are as given in Table 2 for compounds 207-1 to 207-178, are designated as compound Nos. 210-1 to 210-178, respectively.

178 compounds of formula (I) wherein A is 2,4-dichlorophenyl, W is a direct bond and Y, Z, R⁵, P, r and Q are as given in Table 2 for compounds 207-1 to 207-178, are designated as compound Nos. 211-1 to 211-178, respectively.

178 compounds of formula (I) wherein A is 3,4-dichlorophenyl, W is a direct bond and Y, Z, R⁵, P, r and Q are as given in Table 2 for compounds 207-1 to 207-178, are designated as compound Nos. 212-1 to 212-178, respectively.

178 compounds of formula (I) wherein A is 4-chloro-2-methylphenyl, W is a direct bond and Y, Z, R⁵, P, r and Q are as given in Table 2 for compounds 207-1 to 207-178, are designated as compound Nos. 213-1 to 213-178, respectively.

178 compounds of formula (I) wherein A is 4-chloro-2-methoxyphenyl, W is a direct bond and Y, Z, R⁵, P, r and Q are as given in Table 2 for compounds 207-1 to 207-178, are designated as compound Nos. 214-1 to 214-178, respectively.

178 compounds of formula (I) wherein A is 4-chloro-2,6-difluorophenyl, W is a direct bond and Y, Z, R⁵, P, r and Q are as given in Table 2 for compounds 207-1 to 207-178, are designated as compound Nos. 215-1 to 215-178, respectively.

178 compounds of formula (I) wherein A is 4-chloro-3-methylphenyl, W is a direct bond and Y, Z, R⁵, P, r and Q are as given in Table 2 for compounds 207-1 to 207-178, are designated as compound Nos. 216-1 to 216-178, respectively.

178 compounds of formula (I) wherein A is 4-chloro-3-trifluoromethylphenyl, W is a direct bond and Y, Z, R⁵, P, r and Q are as given in Table 2 for compounds 207-1 to 207-178, are designated as compound Nos. 217-1 to 217-178, respectively.

178 compounds of formula (I) wherein A is 4-chloro-2-fluorophenyl, W is a direct bond and Y, Z, R⁵, P, r and Q are as given in Table 2 for compounds 207-1 to 207-178, are designated as compound Nos. 218-1 to 218-178, respectively.

178 compounds of formula (I) wherein A is 4-chloro-3-nitrophenyl, W is a direct bond and Y, Z, R⁵, P, r and Q are as given in Table 2 for compounds 207-1 to 207-178, are designated as compound Nos. 219-1 to 219-178, respectively.

178 compounds of formula (I) wherein A is 4-chloro-2-trifluoromethylphenyl, W is a direct bond and Y, Z, R⁵, P, r and Q are as given in Table 2 for compounds 207-1 to 207-178, are designated as compound Nos. 220-1 to 220-178, respectively.

178 compounds of formula (I) wherein A is 4-chloro-3-cyanophenyl, W is a direct bond and Y, Z, R⁵, P, r and Q are as given in Table 2 for compounds 207-1 to 207-178, are designated as compound Nos. 221-1 to 221-178, respectively.

178 compounds of formula (I) wherein A is 4-chloro-3-fluorophenyl, W is a direct bond and Y, Z, R⁵, P, r and Q are as given in Table 2 for compounds 207-1 to 207-178, are designated as compound Nos. 222-1 to 222-178, respectively.

178 compounds of formula (I) wherein A is 2,4,6-trichlorophenyl, W is a direct bond and Y, Z, R⁵, P, r and Q are as given in Table 2 for compounds 207-1 to 207-178, are designated as compound Nos. 223-1 to 223-178, respectively.

178 compounds of formula (I) wherein A is 2,4-dichloro-3-fluorophenyl, W is a direct bond and Y, Z, R⁵, P, r and Q are as given in Table 2 for compounds 207-1 to 207-178, are designated as compound Nos. 224-1 to 224-178, respectively.

178 compounds of formula (I) wherein A is 4-chloro-2,3-difluorophenyl, W is a direct bond and Y, Z, R⁵, P, r and Q are as given in Table 2 for compounds 207-1 to 207-178, are designated as compound Nos. 225-1 to 225-178, respectively.

178 compounds of formula (I) wherein A is 3,4-dichloro-2-fluorophenyl, W is a direct bond and Y, Z, R⁵, P, r and Q are as given in Table 2 for compounds 207-1 to 207-178, are designated as compound Nos. 226-1 to 226-178, respectively.

178 compounds of formula (I) wherein A is 2,4-dichloro-6-fluorophenyl, W is a direct bond and Y, Z, R⁵, P, r and Q are as given in Table 2 for compounds 207-1 to 207-178, are designated as compound Nos. 227-1 to 227-178, respectively.

178 compounds of formula (I) wherein A is 2,4,5-trichlorophenyl, W is a direct bond and Y, Z, R⁵, P, r and Q are as given in Table 2 for compounds 207-1 to 207-178, are designated as compound Nos. 228-1 to 228-178, respectively.

178 compounds of formula (I) wherein A is 4-chloro-3-methoxyphenyl, W is a direct bond and Y, Z, R⁵, P, r and Q are as given in Table 2 for compounds 207-1 to 207-178, are designated as compound Nos. 229-1 to 229-178, respectively.

178 compounds of formula (I) wherein A is 4-chloro-2-fluoro-3-trifluoromethylphenyl, W is a direct bond and Y, Z, R⁵, P, r and Q are as given in Table 2 for compounds 207-1 to 207-178, are designated as compound Nos. 230-1 to 230-178, respectively.

178 compounds of formula (I) wherein A is 2,3,4-trichlorophenyl, W is a direct bond and Y, Z, R⁵, P, r and Q are as given in Table 2 for compounds 207-1 to 207-178, are designated as compound Nos. 231-1 to 231-178, respectively.

178 compounds of formula (I) wherein A is 4-chloro-3,5-difluorophenyl, W is a direct bond and Y, Z, R⁵, P, r and Q are as given in Table 2 for compounds 207-1 to 207-178, are designated as compound Nos. 232-1 to 232-178, respectively.

178 compounds of formula (I) wherein A is 4-trifluoromethylphenyl, W is a direct bond and Y, Z, R⁵, P, r and Q are as given in Table 2 for compounds 207-1 to 207-178, are designated as compound Nos. 233-1 to 233-178, respectively.

178 compounds of formula (I) wherein A is 2,4-bis-trifluoromethylphenyl, W is a direct bond and Y, Z, R⁵, P, r and Q are as given in Table 2 for compounds 207-1 to 207-178, are designated as compound Nos. 234-1 to 234-178, respectively.

178 compounds of formula (I) wherein A is 3-chloro-4-trifluoromethylphenyl, W is a direct bond and Y, Z, R⁵, P, r and Q are as given in Table 2 for compounds 207-1 to 207-178, are designated as compound Nos. 235-1 to 235-178, respectively.

178 compounds of formula (I) wherein A is 2-chloro-4-trifluoromethylphenyl, W is a direct bond and Y, Z, R⁵, P, r and Q are as given in Table 2 for compounds 207-1 to 207-178, are designated as compound Nos. 236-1 to 236-178, respectively.

178 compounds of formula (I) wherein A is 4-methylphenyl, W is a direct bond and Y, Z, R⁵, P, r and Q are as given in Table 2 for compounds 207-1 to 207-178, are designated as compound Nos. 237-1 to 237-178, respectively.

178 compounds of formula (I) wherein A is 2-chloro-4-methylphenyl, W is a direct bond and Y, Z, R⁵, P, r and Q are as given in Table 2 for compounds 207-1 to 207-178, are designated as compound Nos. 238-1 to 238-178, respectively.

178 compounds of formula (I) wherein A is 3-chloro-4-methylphenyl, W is a direct bond and Y, Z, R⁵, P, r and Q are as given in Table 2 for compounds 207-1 to 207-178, are designated as compound Nos. 239-1 to 239-178, respectively.

178 compounds of formula (I) wherein A is 3-fluoro-4-methylphenyl, W is a direct bond and Y, Z, R⁵, P, r and Q are as given in Table 2 for compounds 207-1 to 207-178, are designated as compound Nos. 240-1 to 240-178, respectively.

178 compounds of formula (I) wherein A is 2,4,5-trimethylphenyl, W is a direct bond and Y, Z, R⁵, P, r and Q are as given in Table 2 for compounds 207-1 to 207-178, are designated as compound Nos. 241-1 to 241-178, respectively.

178 compounds of formula (I) wherein A is 4-fluorophenyl, W is a direct bond and Y, Z, R⁵, P, r and Q are as given in Table 2 for compounds 207-1 to 207-178, are designated as compound Nos. 242-1 to 242-178, respectively.

178 compounds of formula (I) wherein A is 4-fluoro-3-methoxyphenyl, W is a direct bond and Y, Z, R⁵, P, r and Q are as given in Table 2 for compounds 207-1 to 207-178, are designated as compound Nos. 243-1 to 243-178, respectively.

178 compounds of formula (I) wherein A is 4-fluoro-3-trifluoromethylphenyl, W is a direct bond and Y, Z, R⁵, P, r and Q are as given in Table 2 for compounds 207-1 to 207-178, are designated as compound Nos. 244-1 to 244-178, respectively.

178 compounds of formula (I) wherein A is 3-chloro-4-fluorophenyl, W is a direct bond and Y, Z, R⁵, P, r and Q are as given in Table 2 for compounds 207-1 to 207-178, are designated as compound Nos. 245-1 to 245-178, respectively.

178 compounds of formula (I) wherein A is 4-fluoro-3-methylphenyl, W is a direct bond and Y, Z, R⁵, P, r and Q are as given in Table 2 for compounds 207-1 to 207-178, are designated as compound Nos. 246-1 to 246-178, respectively.

178 compounds of formula (I) wherein A is 2,3,4-trifluorophenyl, W is a direct bond and Y, Z, R⁵, P, r and Q are as given in Table 2 for compounds 207-1 to 207-178, are designated as compound Nos. 247-1 to 247-178, respectively.

178 compounds of formula (I) wherein A is 4-trifluoromethoxyphenyl, W is a direct bond and Y, Z, R⁵, P, r and Q are as given in Table 2 for compounds 207-1 to 207-178, are designated as compound Nos. 248-1 to 248-178, respectively.

178 compounds of formula (I) wherein A is 2-fluoro-3-methoxyphenyl, W is a direct bond and Y, Z, R⁵, P, r and Q are as given in Table 2 for compounds 207-1 to 207-178, are designated as compound Nos. 249-1 to 249-178, respectively.

178 compounds of formula (I) wherein A is 4-methoxyphenyl, W is a direct bond and Y, Z, R⁵, P, r and Q are as given in Table 2 for compounds 207-1 to 207-178, are designated as compound Nos. 250-1 to 250-178, respectively.

178 compounds of formula (I) wherein A is 4-methoxy-2-methylphenyl, W is a direct bond and Y, Z, R⁵, P, r and Q are as given in Table 2 for compounds 207-1 to 207-178, are designated as compound Nos. 251-1 to 251-178, respectively.

178 compounds of formula (I) wherein A is 2,4-dimethoxyphenyl, W is a direct bond and Y, Z, R⁵, P, r and Q are as given in Table 2 for compounds 207-1 to 207-178, are designated as compound Nos. 252-1 to 252-178, respectively.

178 compounds of formula (I) wherein A is 3-fluoro-4-methoxyphenyl, W is a direct bond and Y, Z, R⁵, P, r and Q are as given in Table 2 for compounds 207-1 to 207-178, are designated as compound Nos. 253-1 to 253-178, respectively.

178 compounds of formula (I) wherein A is 3-chloro-4-methoxyphenyl, W is a direct bond and Y, Z, R⁵, P, r and Q are as given in Table 2 for compounds 207-1 to 207-178, are designated as compound Nos. 254-1 to 254-178, respectively.

178 compounds of formula (I) wherein A is 3,4-dimethylphenyl, W is a direct bond and Y, Z, R⁵, P, r and Q are as given in Table 2 for compounds 207-1 to 207-178, are designated as compound Nos. 255-1 to 255-178, respectively.

178 compounds of formula (I) wherein A is 4-methyl-3-nitrophenyl, W is a direct bond and Y, Z, R⁵, P, r and Q are as given in Table 2 for compounds 207-1 to 207-178, are designated as compound Nos. 256-1 to 256-178, respectively.

178 compounds of formula (I) wherein A is 2-fluoro-4-methylphenyl, W is a direct bond and Y, Z, R⁵, P, r and Q are as given in Table 2 for compounds 207-1 to 207-178, are designated as compound Nos. 257-1 to 257-178, respectively.

178 compounds of formula (I) wherein A is 2-fluoro-4-methoxyphenyl, W is a direct bond and Y, Z, R⁵, P, r and Q are as given in Table 2 for compounds 207-1 to 207-178, are designated as compound Nos. 258-1 to 258-178, respectively.

178 compounds of formula (I) wherein A is 2,3,4-trimethoxyphenyl, W is a direct bond and Y, Z, R⁵, P, r and Q are as given in Table 2 for compounds 207-1 to 207-178, are designated as compound Nos. 259-1 to 259-178, respectively.

178 compounds of formula (I) wherein A is 3,4-dimethoxyphenyl, W is a direct bond and Y, Z, R⁵, P, r and Q are as given in Table 2 for compounds 207-1 to 207-178, are designated as compound Nos. 260-1 to 260-178, respectively.

178 compounds of formula (I) wherein A is 4-chloro-3-dimethylamino-2-fluorophenyl, W is a direct bond and Y, Z, R⁵, P, r and Q are as given in Table 2 for compounds 207-1 to 207-178, are designated as compound Nos. 261-1 to 261-178, respectively.

178 compounds of formula (I) wherein A is 5-chlorothiophen-2-yl, W is a direct bond and Y, Z, R⁵, P, r and Q are as given in Table 2 for compounds 207-1 to 207-178, are designated as compound Nos. 262-1 to 262-178, respectively.

178 compounds of formula (I) wherein A is 6-chloropyrid-3-yl, W is a direct bond and Y, Z, R⁵, P, r and Q are as given in Table 2 for compounds 207-1 to 207-178, are designated as compound Nos. 263-1 to 263-178, respectively.

178 compounds of formula (I) wherein A is 4-methylpiperidin-1-yl, W is a direct bond and Y, Z, R⁵, P, r and Q are as given in Table 2 for compounds 207-1 to 207-178, are designated as compound Nos. 264-1 to 264-178, respectively.

Table 3 below provides 18 compounds designated compound numbers 265-1 to 265-16 respectively, of general formula (I) wherein Y is Cl, R⁵ is H, P is CH₂, r is 1, W is a direct bond and A, Z and Q have the values as listed.

TABLE 3

| Compound | | | |
|---|---|---|---|
| Number | A | Z | Q |
| 265-1 | cyclopropyl | CONHCH₂phenyl | phenyl |
| 265-2 | cyclopropyl | CONHCH₂(4-chlorophenyl) | 4-chlorophenyl |
| 265-3 | cyclopropyl | CONHCH₂furan-2-yl | furan-2-yl |
| 265-4 | cyclopropyl | CONHCH₂thiophen-2-yl | thiophen-2-yl |
| 265-5 | 4-chlorophenyl | CONHCH₂phenyl | phenyl |
| 265-6 | 4-chlorophenyl | CONHCH₂(4-chlorophenyl) | 4-chlorophenyl |
| 265-7 | 4-chlorophenyl | CONHCH₂furan-2-yl | furan-2-yl |
| 265-8 | 4-chlorophenyl | CONHCH₂thiophen-2-yl | thiophen-2-yl |
| 265-9 | 4-bromophenyl | CONHCH₂phenyl | phenyl |
| 265-10 | 4-bromophenyl | CONHCH₂(4-chlorophenyl) | 4-chlorophenyl |
| 265-11 | 4-bromophenyl | CONHCH₂furan-2-yl | furan-2-yl |
| 265-12 | 4-bromophenyl | CONHCH₂thiophen-2-yl | thiophen-2-yl |
| 265-13 | 4-chloro-2-fluoro-3- | CONHCH₂phenyl | phenyl |
| | methoxyphenyl | | |
| 265-14 | 4-chloro-2-fluoro-3- | CONHCH₂(4-chlorophenyl) | 4-chlorophenyl |
| | methoxyphenyl | | |
| 265-15 | 4-chloro-2-fluoro-3- | CONHCH₂furan-2-yl | furan-2-yl |
| | methoxyphenyl | | |
| 265-16 | 4-chloro-2-fluoro-3- | CONHCH₂thiophen-2-yl | thiophen-2-yl |
| | methoxyphenyl | | |
| 265-17 | cyclopropyl | OCH₂CO₂H | cyclopropyl |
| 265-18 | cyclopropyl | OCH₂CO₂CH₃ | cyclopropyl |
| 265-19 | cyclopropyl | OCH₂CO₂C(CH₃)₃ | cyclopropyl |
| 265-20 | cyclopropyl | OCH₂CONHCH₂cyclopropyl | cyclopropyl |

20 compounds of formula (I) wherein Y is methyl, R⁵ is H, P is CH₂, r is 1, W is a direct bond and A, Z and Q are as given in Table 3 for compounds 265-1 to 265-20, are designated as compound Nos. 266-1 to 266-20 respectively.

General methods for the production of compounds of formula (I) are described below. Unless otherwise stated in the text, the substitutents A, R⁵, P, W, Q, Y and Z are as defined hereinbefore. The abbreviation LG as used herein refers to any suitable leaving group, and includes halogen, sulphonate, and sulphone groups. The groups R as used herein are, independently of each other, alkyl or substituted alkyl groups. The groups R′ may, independently of each other, take a range of values depending on the particular structure of the molecule in which they are present; the skilled man will recognise what values are applicable in each case, particularly in view of the definition of compounds of formula (I) as described hereinbefore.

Compounds of formula (I) may be prepared from compounds of formula (A) as shown in reaction scheme 1.

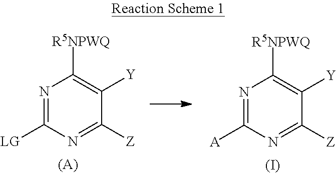

For example, a compound of formula (I), in which A is a ring linked to the pyrimidine ring through a nitrogen atom, may be prepared by reaction of a compound of formula (A) with A-H (e.g. piperidine, pyrrole), optionally in the presence of a suitable base (e.g. an amine base, such as triethylamine), in a suitable solvent (for example an alcohol, such as methanol)—see reaction scheme 2 below. The reaction may be performed at ambient temperature or preferably, at an elevated temperature. This transformation may also be performed in the presence of a suitable metal (e.g. palladium) catalyst, optionally complexed by any suitable ligands (e.g. phosphine ligands, such as Josiphos).

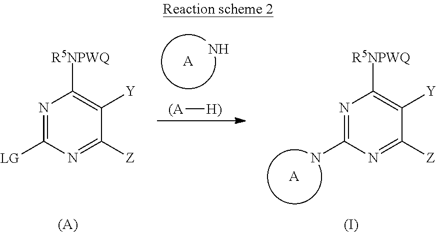

In a second example (see reaction scheme 3) a compound of formula (I), in which A is a group attached through a carbon atom, may be prepared by reacting a suitable metal or metalloid derivative A-M (e.g. a boronic acid or ester, a trialkyltin derivative, a zinc derivative or a Grignard reagent) with a compound of formula A in the presence of a suitable base (e.g. an inorganic base, such as potassium phosphate or caesium fluoride, or an organic base, such as triethylamine), a metal source (e.g. a palladium source such as Pd₂(dba)₃) and, optionally, a ligand for the metal (e.g. a phosphine ligand, such as X-Phos) in a suitable solvent (for example a single solvent, such as acetonitrile, or a mixed solvent system, such as a mixture of dimethoxyethane and water). The metal catalyst and ligands may also be added as a single, pre-formed, complex (e.g. a palladium/phosphine complex, such as palladium tetrakistriphenylphosphine, bis(triphenylphosphine)palladium dichloride or [1,1′-bis(diphenylphosphino)ferrocene]palladium dichloride).

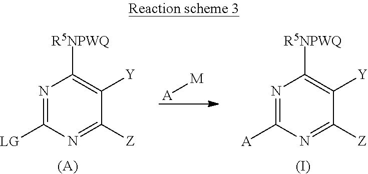

As an additional example, a compound of formula (I) in which A is an alkenyl group may be prepared using a Heck reaction in which the group A component containing the double bond may be reacted with a compound of formula (A) in the presence of a suitable metal catalyst (for example a palladium derivative, such as palladium acetate), optionally with a suitable ligand for the metal, and a suitable base (for example an inorganic base, such as potassium phosphate) in a suitable solvent (for example N-methylpyrrolidone).

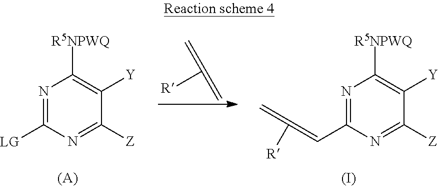

Alternatively, compounds of formula (I) may be prepared from compounds of formula (B), wherein M represents a suitable metal or metalloid derivative (e.g. a boronic acid or ester, a trialkyltin group, a suitably substituted silyl group, a zinc derivative or a magnesium halide), by reaction with a compound A-LG—see reaction scheme 5 below.

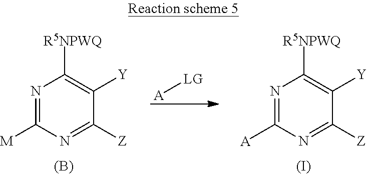

For example, a compound of formula (I) may be prepared from a compound of formula (B) in which M is a boronic acid group by reaction with a compound A-LG in the presence of a metal catalyst (e.g. a palladium derivative such as Pd₂(dba)₃), optionally with a suitable ligand (e.g. a phosphine such as X-Phos) and a base (e.g. potassium phosphate or caesium fluoride) in a suitable solvent.

Compounds of formula (B) may be prepared from other compounds of formula (B) using a transmetallation reaction. For example, a compound of formula (B) wherein M is a boronic acid may be prepared from a compound of formula (B) where M is a magnesium halide by reaction with a trialkylboronate, followed by hydrolysis (for example under acidic conditions).

Alternatively compounds of formula (B) may be prepared from compounds of formula (A) (reaction scheme 6).

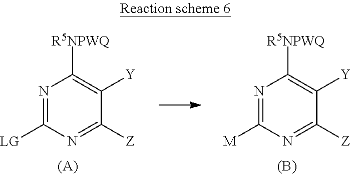

For example, a compound of formula (B) wherein M is a boronate ester or a trialkylstannane may be prepared from a compound of formula (A) by treating it with a suitable M-containing reagent (e.g. pinacolborane, bispinacolatodiboron, hexa-alkyldi-tin) in the presence of a metal catalyst (e.g. a palladium species, such as bis(diphenylphosphine)palladium dichloride) in a suitable solvent (e.g. dioxane).

Alternatively, a compound of formula (B) where M is a magnesium halide may be prepared from a compound of formula (A) by treatment with a suitable Grignard reagent (e.g. an isopropylmagnesium halide such as isopropylmagnesium chloride) in a suitable solvent.

Compounds of formula (A) may be prepared from compounds of formula (C) wherein LG′ is a second leaving group which may be the same as or different to LG (reaction scheme 7).

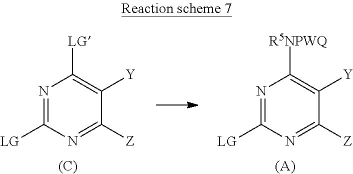

For example, a compound of formula (A) may be prepared from a compound of formula (C) by reaction with a reagent R⁵(QWP)N—H or R⁵(QWP)N in a suitable solvent (e.g. an ether solvent, such as tetrahydrofuran). The reagent R⁵(QWP)N⁻ may be generated in situ by addition of a suitable base to R⁵(QWP)N—H, or it may be prepared separately and added as a suitable salt.

As a further example, a compound of formula (A) may be prepared from a compound of formula (C) by treatment with a reagent R⁵(QWP)N—H in the presence of a suitable catalyst (e.g. a metal catalyst, such as a palladium source) and optionally a suitable ligand (e.g. a phosphine ligand, such as Josiphos) in a suitable solvent.

Compounds of formula (C1) (i.e. compounds of formula C in which LG is the same as LG′ and is, for example, a halogen atom or a sulphonate) may be prepared from compounds of formula (D) (reaction scheme 8).

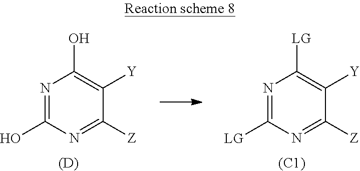

For example, a compound of formula (C1) in which LG is a halogen atom may be prepared from a compound of formula (D) by treatment with a suitable reagent (e.g. a phosphoryl halide such as phosphorous oxychloride) and optionally a suitable base (e.g. an organic base, such as N,N-diethylaniline).

Compounds of formula C2 (i.e. compounds of formula (C) in which LG and LG′ are different) may be prepared from compounds of formula (E) wherein B* represents a suitable precursor group to LG′ (reaction scheme 9).

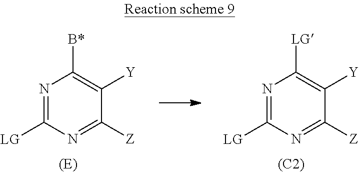

For example, a compound of formula (C2) in which LG′ is a sulphone may be prepared from a compound of formula (E1) (i.e. a compound of formula (E) in which B* is a thioether group) by reaction with a suitable oxidising agent, for example a peracid such as meta-chloroperbenzoic acid (reaction scheme 10).

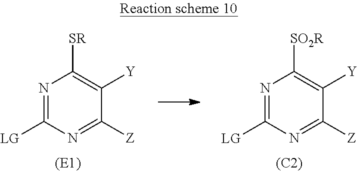

Compounds of formula (E) may be prepared from compounds of formula (C1) by treatment with a reagent B*—H or B*⁻.

For example, a compound of formula (E1) may be prepared from a compound of formula (C1) by reaction with a thiol or thiolate anion, optionally in the presence of a suitable base, in a suitable solvent (see reaction scheme 11).

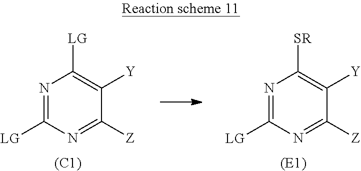

Compounds of formula (D) may be prepared from compounds of formula (F) by reaction with a suitable source of electropositive Y (reaction scheme 12).

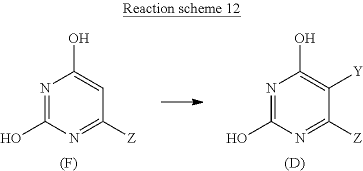

For example a compound of formula (D) in which Y is a halogen may be prepared from a compound of formula (F) by reaction with an N-halosuccinimide (e.g. N-chlorosuccinimide) in a suitable solvent (e.g. dimethylformamide).

Compounds of formula (F) are well known in the literature, or can be made readily from compounds known in the literature by routine methods that are familiar to the skilled man.

Compounds of formula (I) may also be prepared from compounds of formula (G) (reaction scheme 13).

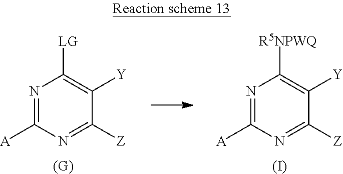

For example, a compound of formula (I) may be prepared from a compound of formula (G) by reaction with a reagent R⁵(QWP)N—H or R⁵(QWP)N⁻ in a suitable solvent (e.g. methanol or water). The reagent R⁵(QWP)N⁻ may be generated in situ by addition of a suitable base to R⁵(QWP)N—H, or it may be prepared separately and added as a suitable salt.

As a further example a compound of formula (I) may be prepared from a compound of formula (G) by treatment with a reagent R⁵(QWP)N—H in the presence of a suitable catalyst (e.g. a metal catalyst, such as a palladium source) and optionally a suitable ligand (for example a phosphine ligand, such as Josiphos) in a suitable solvent.

Compounds of formula (G) may be prepared from compounds of formula (H) as shown in reaction scheme 14 below.

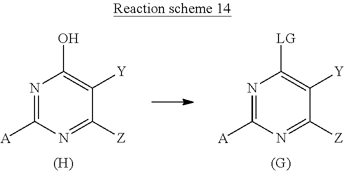

For example, a compound of formula (G) in which LG is a halogen atom may be prepared from a compound of formula (H) by treatment with a suitable reagent (e.g. a phosphoryl halide, such as phosphorous oxychloride) and optionally a suitable base (e.g. an organic base, such as N,N-diethylaniline).

Compounds of formula (H) may be prepared from compounds of formula (i) by reaction with a suitable source of electropositive Y, as shown in reaction scheme 15 below.

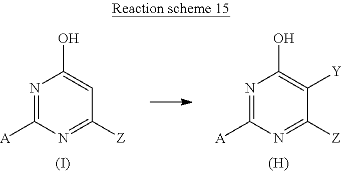

For example a compound of formula (H) in which Y is a halogen may be prepared from a compound of formula (i) by reaction with an N-halosuccinimide (e.g. N-chlorosuccinimide) in a suitable solvent (e.g. dimethylformamide), or with a metal hypohalite (e.g. sodium hypochlorite) in a suitable solvent e.g. acidic water).

Compounds of formula (i) may be prepared from amidines of formula (J) by reaction with a suitable ketoester of formula (K) as shown in the scheme below.

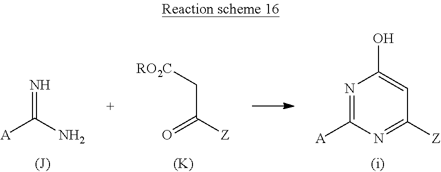

For example, a compound of formula (i1) (i.e. a compound of formula (i) in which Z is a carboxylic acid or ester) may be prepared by the reaction of an amidine of formula (J) with an oxaloacetate diester of formula (K1) (i.e. a compound of formula (K) in which Z is CO₂R), optionally in the presence of a suitable base (for example an inorganic base, such as sodium hydroxide), in a suitable solvent (for example water), see reaction scheme 17 below. The diester (K1) may also be used in the form of a salt, for example the sodium salt.

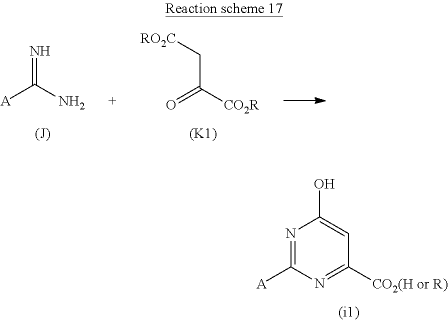

In an additional example, a compound of formula (i2) (i.e. a compound of formula (i) in which Z is an acetal group) may be prepared by the condensation of an amidine of formula (J) with a ketoester of formula (K2) (i.e. a compound of formula (K) in which Z is CH(OR)₂) in the presence of a base (for example an alkoxide base, such as sodium methoxide) in a suitable solvent (for example an alcohol, such as methanol) (reaction scheme 18).

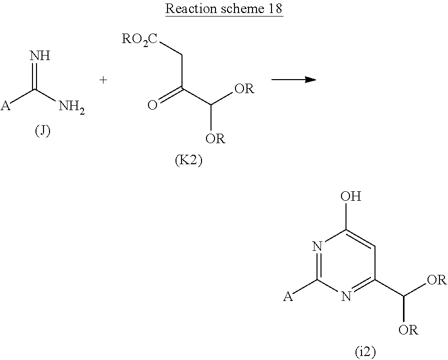

Amidines (J) and di-esters (K) are known in the literature or can be made readily from compounds known in the literature by routine methods that are familiar to the skilled man.

Compounds of formula (H) may be prepared from amidines of formula (J) by reaction with a suitable ketoester of formula (L) (reaction scheme 19).

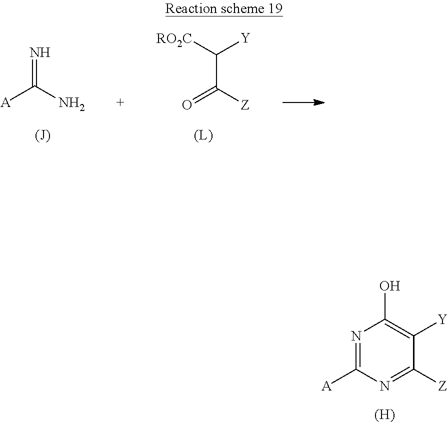

For example, a compound of formula (H1) (i.e. a compound of formula (H) in which Z is a carboxylic acid or ester) may be prepared by the reaction of an amidine of formula (J) with an oxaloacetate diester of formula (L1) (i.e. a compound of formula (L) in which Z is CO₂R), optionally in the presence of a suitable base (e.g. an inorganic base, such as sodium hydroxide), in a suitable solvent (e.g. water), see reaction scheme 20. The diester (L1) may also be used in the form of a salt, e.g. the sodium salt.

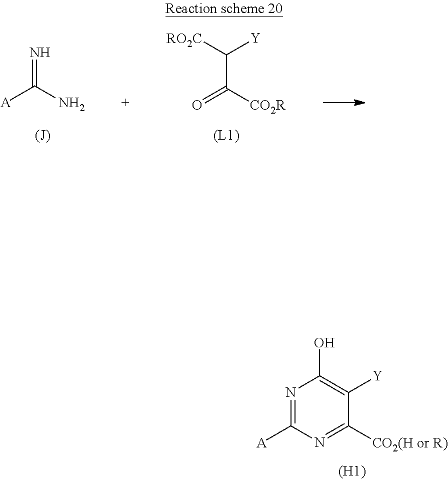

Compounds of formula (L) are well known in the literature, or can be made readily from compounds of formula (K) by standard methods that are familiar to the skilled man (reaction scheme 21).

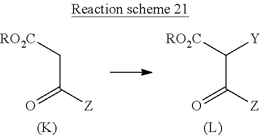

Compounds of formula (H) may be prepared by the condensation of amidines of formula (J) with substituted acetate esters of formula (M) and esters of formula (N) wherein Z is as defined hereinbefore in the presence of a base (e.g. an alkoxide base, such as sodium ethoxide) in a suitable solvent (e.g. an alcohol, such as ethanol) (reaction scheme 22).

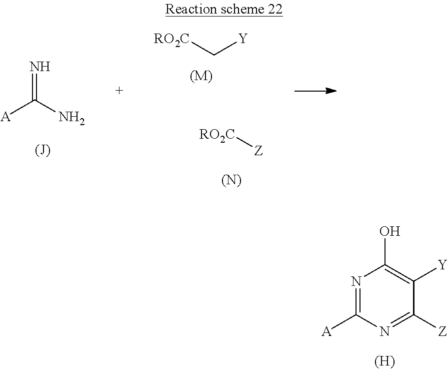

For example a compound of formula (H1) may be prepared by the reaction of an amidine of formula (J) with an acetate ester of formula (M) and a diethyl oxalate of formula (N1) (i.e. a compound of formula N in which Z is a carboxylate ester).

Compounds of formula (M) and (N) are well known in the literature.

Compounds of formula (I) may also be prepared by the reaction of compounds of formula (O) with a suitable source of electrophilic Y (reaction scheme 23).

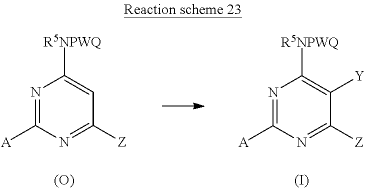

For example a compound of formula (I) in which Y is a halogen may be prepared from a compound of formula (O) by reaction with an N-halosuccinimide (e.g. N-chlorosuccinimide) in a suitable solvent (e.g. dimethylformamide).

Compounds of formula (O) may also be prepared from compounds of formula (P) (reaction scheme 24).

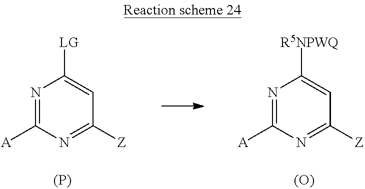

For example, a compound of formula O may be prepared from a compound of formula O by reaction with a reagent R⁵(QWP)N—H or R⁵(QWP)N⁻ in a suitable solvent (e.g. an ether solvent, such as tetrahydrofuran). The reagent R⁵(QWP)N⁻ may be generated in situ by addition of a suitable base to R⁵(QWP)N—H, or it may be prepared separately and added as a suitable salt.

As a further example a compound of formula (O) may be prepared from a compound of formula (P) by treatment with the reagent R⁵(QWP)N—H in the presence of a suitable catalyst (e.g. a metal catalyst, such as a palladium source) and optionally a suitable ligand (e.g. a phosphine ligand, such as Josiphos) in a suitable solvent.

Compounds of formula (P) may be prepared from compounds of formula (i) (as defined above) according to the scheme below.

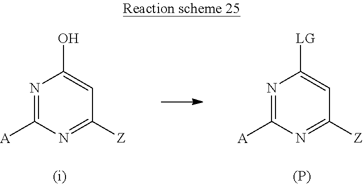

For example, a compound of formula (P) in which LG is a halogen atom may be prepared from a compound of formula (i) by treatment with a suitable reagent (e.g. a phosphoryl halide such as phosphorous oxychloride) and optionally a suitable base (e.g. an organic base, such as N,N-diethylaniline).

Compounds of formula (I) in which A is a ring may also be prepared from compounds of formula (Q) in which D represents a suitable cyclisation precursor by reactions in which ring A is formed (see reaction scheme 26). Examples of suitable cyclisation precursors include groups containing carbon-carbon and carbon-heteroatom double or triple bonds, such as alkenes, carbonyl groups, oximes and alkynes.

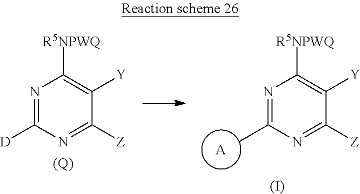

For example, a compound of formula (I) in which A is a 5-membered heteroaromatic ring may be formed from a compound of formula (Q1), which is a compound of formula (Q) in which D is an alkyne, by reaction with a suitable 1,3-dipolar species (R) in which one or more of E, F and G are heteroatoms, such as an azide or nitrile oxide (reaction scheme 27).

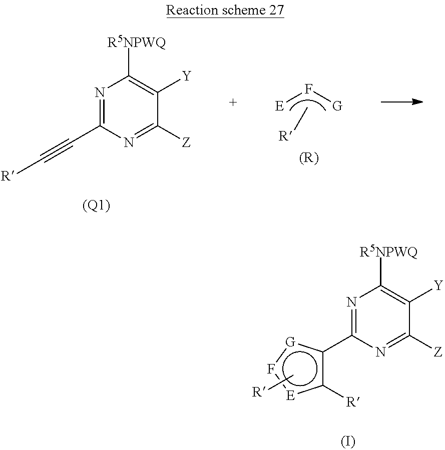

In a further example, a compound of formula (I) in which A is an unsaturated 6-membered ring may be prepared from a compound of formula (Q1) by reaction with a suitable diene (S), in which I, J, K and L are carbon atoms or one of I, J, K and L is a heteroatom (reaction scheme 28).

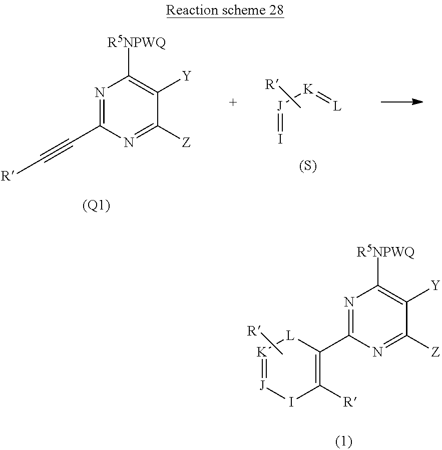

In a further example, a compound of formula (I), in which A is cyclopropane group, an epoxide group or an aziridine ring, may be prepared from a compound of formula (Q2) (i.e. a compound of formula (I) in which A is an alkene) by reaction with a suitable cyclopropanation reagent (e.g. a reagent combination that generates a carbene, such as diiodomethane and diethyl zinc), epoxidation reagent (e.g. a peracid such as meta-chloroperbenzoic acid) or aziridination reagent (e.g. a combination of Chloramine T and N-bromosuccinimide), as appropriate (see reaction scheme 29).

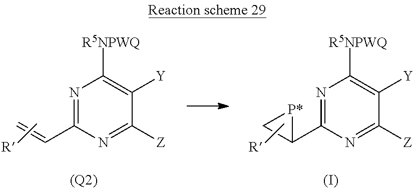

In a further example, a compound of formula (I) in which A is a 4-membered ring may be prepared from a compound of formula (Q2) by reaction with suitable reagent (T) containing a carbon-carbon or carbon-heteroatom double bond, wherein one or both of R* and R** is a carbon atom, for example an alkene or isocyanate (reaction scheme 30).

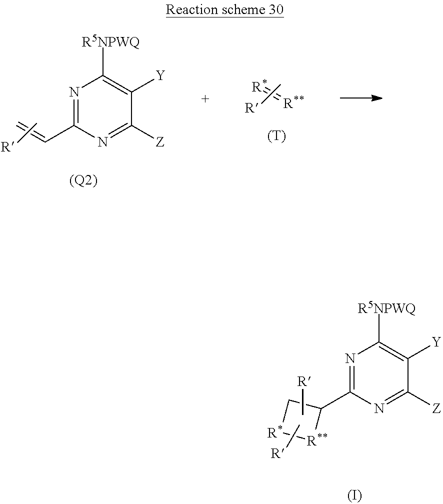

In another example a compound of formula (I) in which A is a 5-membered non-aromatic ring may be formed from a compound of formula (Q2) by reaction with a suitable 1,3-dipolar species (R) in which none, one or more of E, F and G are heteroatoms, such as a trimethylenemethane species, an azide or nitrone (reaction scheme 31).

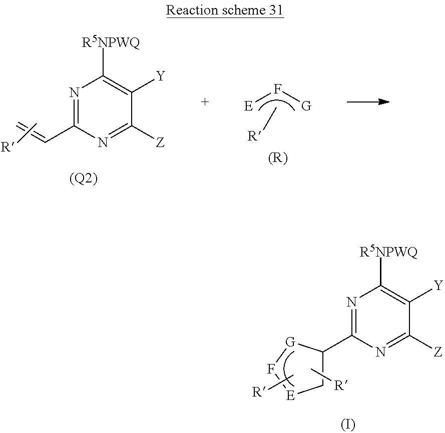

As a further example a compound of formula (I) in which A is an unsaturated 6-membered ring may be prepared from a compound of formula (Q2) by reaction with a suitable diene (S), in which I, J, K and L are carbon atoms or one of I, J, K and L is a heteroatom (reaction scheme 32).

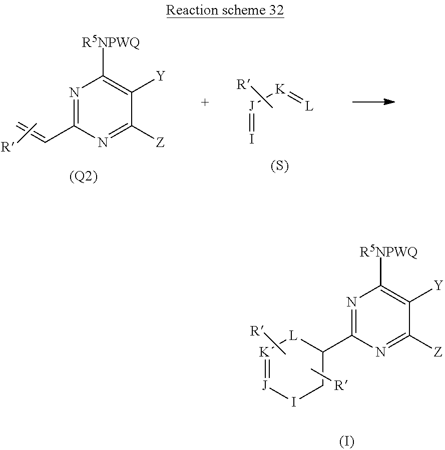

In a further example, a compound of formula (I), in which A is a 5-8 membered saturated ring containing at least two oxygen atoms, may be prepared from a compound of formula (Q3) i.e. a compound of formula (Q) in which D is a alkanediol group, by reaction with a suitable bifunctional reagent (e.g. a dihalide such as phosgene or diiodoethane, or a ketone or aldehyde such as acetone) under standard conditions known to the skilled man and readily available in the literature (shown schematically in reaction scheme 33).

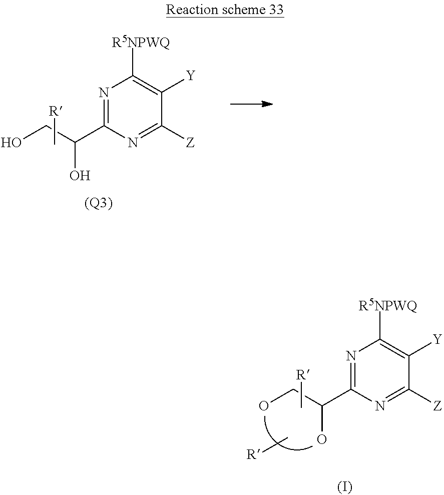

In another example, a compounds of formula (I) in which A is an acetal or related ring system, e.g. a dithioketal, may be prepared from a compound of formula (Q4) i.e. a compound of formula Q in which D is a aldehyde or ketone, by reaction with a reagent of formula U, in which each of AA and BB are independently oxygen, sulphur or nitrogen groups which are joined by a 2-5 atom chain (wherein one of the atoms in the chain may be a heteroatom) under suitable conditions, for example in the presence of an acid catalyst such as toluenesulphonic acid, in a suitable solvent (e.g. toluene), see reaction scheme 34.

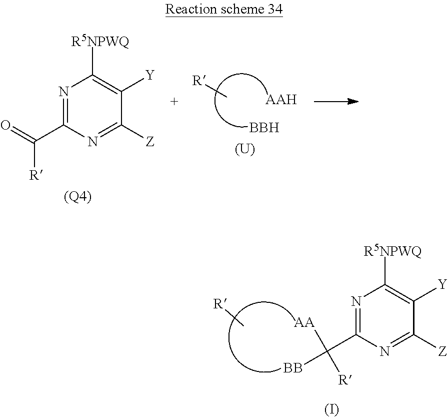

For a further example a compound of formula (I) in which A is an epoxide ring may be prepared from a compound of formula (Q4) by reaction with a suitable epoxidation reagent (e.g. a sulphonium ylide such as that formed by treatment of a trialkylsulphonium salt with a suitable base)—reaction scheme 35.

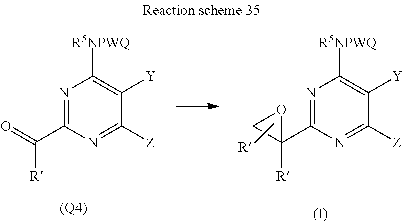

In a further example, a compound of formula (I) in which A is a 4-membered heterocyclic ring may be prepared from a suitable compound of formula (Q5) (i.e. a compound of formula Q in which CC represents a group bonded by a heteroatom, e.g. an imine) by a cycloaddition reaction with a compound of formula (V) under thermal or photolytic conditions.

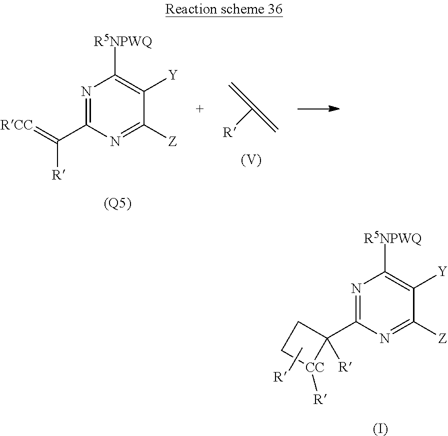

As a further example a compound of formula (I) in which A is an unsaturated heterocyclic 6-membered ring may be prepared from a compound of formula (Q5) by reaction with a suitable diene (W) under thermal conditions or in the presence of a Lewis acid (reaction scheme 37).

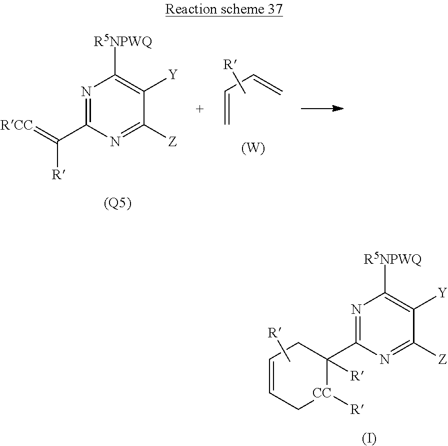

In a further example a compound of formula (I) in which A is an isoxazoline may be prepared by reaction of an oxime of formula (Q6) (i.e. a compound of formula (Q) in which D is an aldoxime) first with an oxidising reagent (e.g. an N-halosuccinimide) in the presence of a base, and then with an alkene of formula (V) under suitable conditions (reaction scheme 38).

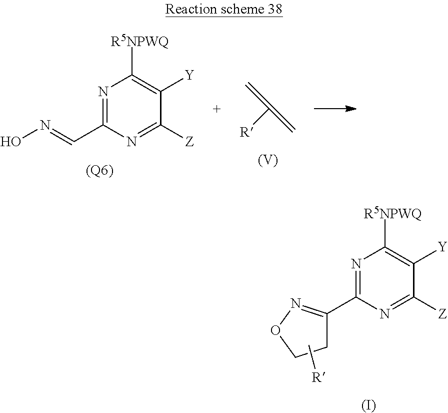

In a further example a compound of formula (I) in which A is an isoxazole may be prepared by reaction of an oxime of formula (Q6) first with an oxidising reagent (e.g. an N-halosuccinimide) in the presence of a base, and then with an alkyne of formula (X) under suitable conditions (reaction scheme 39).

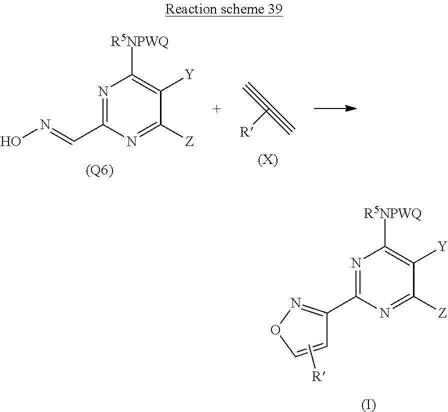

In yet another example, a compound of formula (I) in A is a heteroaromatic ring may be formed by the reaction of a compound of formula (Q7), i.e. a compound of formula (Q) in which D is a ketone containing a leaving group such as a halogen atom or a sulphonate, with an acid derivative of formula (Y), wherein DD is N, O or S (reaction scheme 40).

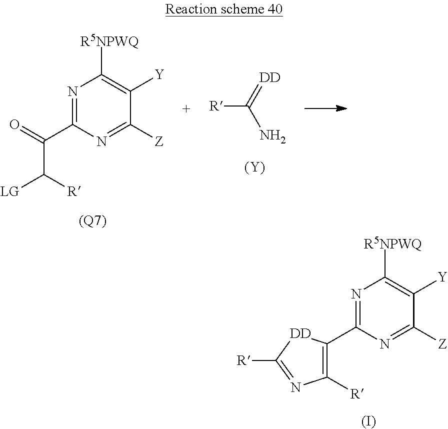

For example a compound of formula (I) in which A is an imidazole ring may be prepared by the reaction of a compound of formula (Q7) with an amidine of formula (Y1) i.e. a compound of formula Y in which DD is nitrogen), see reaction scheme 41.

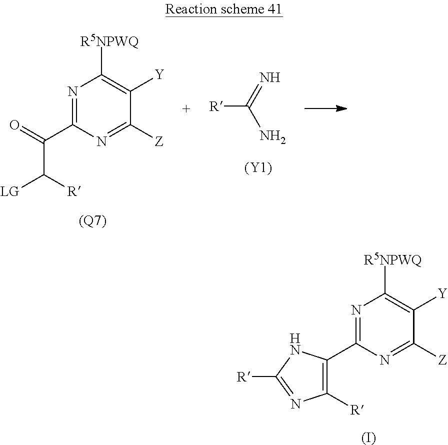

Compounds of formulae (R), (S), (T), (U), (V), (W), (X) and (Y) are known or can be made readily using methods that are well known in art.

Compounds of formula (Q1) may be prepared from compounds of formula (A) by a Sonogashira-type reaction (see reaction scheme 42) with an alkyne (X1) i.e. an alkyne of formula (X) wherein at least one substituent R′ is a hydrogen atom.

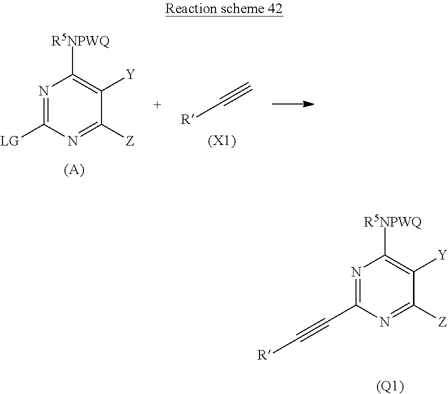

Compounds of formula (I) in which A is an alkene may be prepared from compounds of formula (Q4) by olefination reactions (see reaction scheme 43) that are well known in the literature, for example using the Wittig, Peterson, Tebbe or Petasis reactions.

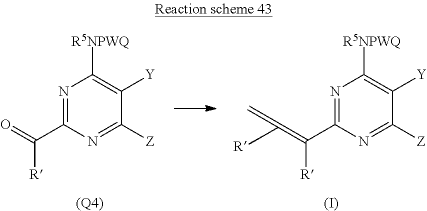

Compounds of formula (Q4) may be prepared from compounds of formula (Q2) by oxidative cleavage of the double bond (reaction scheme 44), e.g. by treatment with ozone.

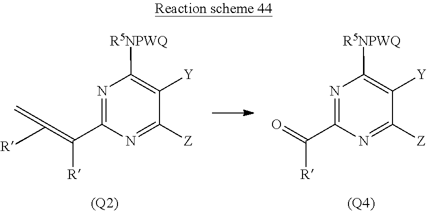

Alternatively compounds of formula (Q4) may be prepared from compounds of formula (Q3) by treatment with a suitable oxidising agent (see reaction scheme 45), e.g. sodium periodate.

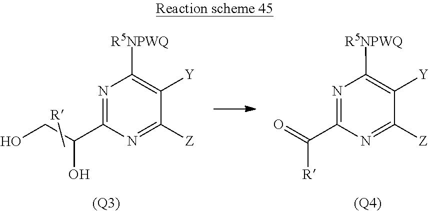

Compounds of formula (Q3) may be prepared from compounds of formula (Q2) by reaction with a suitable dihydroxylation reagent (e.g. an osmium species such as osmium tetroxide), see reaction scheme 46.

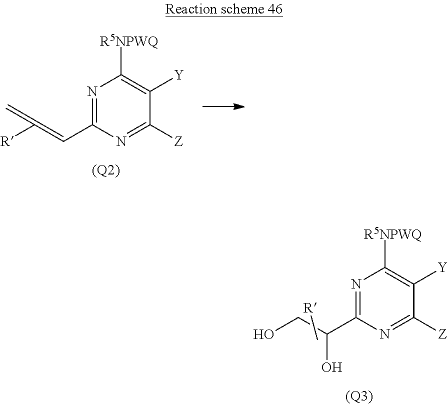

Compounds of formula (Q7) may be prepared from compounds of (Q4) (see reaction scheme 47) in which R′ is an alkyl group by reaction with a suitable oxidising reagent (e.g. an N-halosuccinimide when LG is a halogen atom) optionally in the presence of a suitable base.

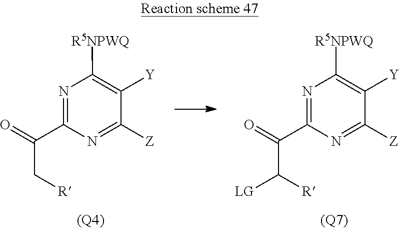

Compounds of formula (I) in which A is a heteroaromatic ring may be prepared by the condensation of compounds of formula (Z) wherein FF is a carboxylic acid derivative such as an amide or thioamide, with suitably functionalised carbonyl containing compounds (see reaction scheme 48 below).

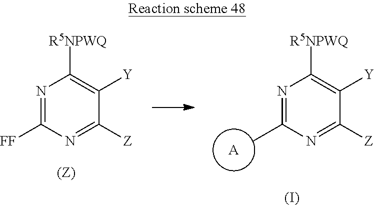

For example a compound of formula (I) in which ring A is a thiazole may be prepared from a thioamide of formula (Z1), i.e. a compound of formula (Z) wherein FF is a thioamide group, by reaction with a compound of formula (AA) wherein LG is a leaving group, e.g. a halogen or sulphonate,

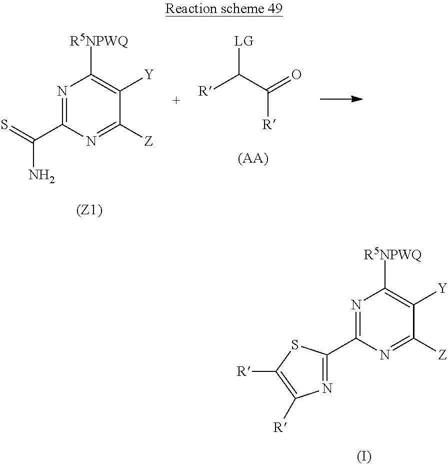

Compounds of formula (AA) are known compounds or may be prepared using routine methods well known in the art.

Compounds of formula (Z) may be prepared from compounds of formula (B) (as defined above) by reaction with an electrophilic source of the group FF (see reaction scheme 50 below).

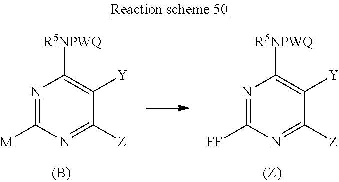

For example, a compound of formula (Z2) i.e. a compound of formula Z wherein FF is a carboxylic acid, may be prepared from a compound of formula (B1) (i.e. a compound of formula (B) in which M is Mg-Hal) by reaction with carbon dioxide in a suitable solvent (reaction scheme 51).

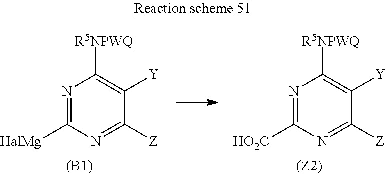

In a further example, a compound of formula (Z3), i.e. a compound of formula (Z) in which FF is a formyl group, may be prepared by the reaction of a compound of formula (B1) with a formamide such as dimethylformamide (reaction scheme 52).

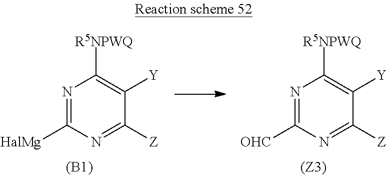

Compounds of formula (Z) may be prepared from other compounds of formula (Z) using transformations that are well known in the art and with which the skilled man will be familiar. For example, a compound of formula (Z1) may be prepared from a compound of formula (Z2) by first forming an amide of formula (Z4) (a compound of formula (Z) in which FF is an amide group) under known conditions, followed by reaction with a suitable thionating reagent (reaction scheme 53) such as phosphorus pentasulphide or Lawesson's reagent.

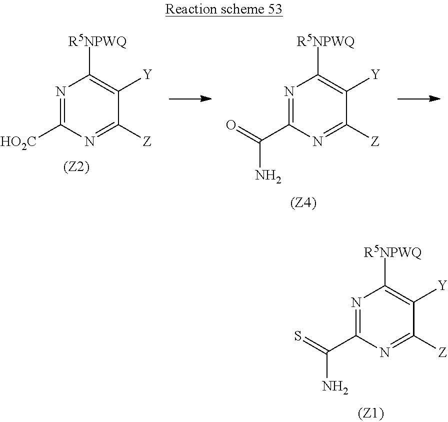

Compounds of formula (I) may be prepared from compounds of formula (AB) by methods readily available in the art (reaction scheme 54).

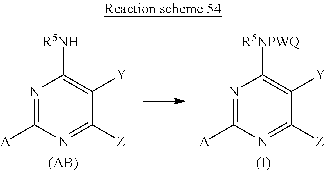

For example, a compound of formula (I) may be prepared by the alkylation of a compound of formula (AB) with a reagent LG-(PWQ) where LG is a suitable leaving group such as a halide or sulphonate.

Alternatively a compound of formula (I) may be prepared from a compound of formula (AB) by reductive amination, involving first a condensation with a carbonyl compound such as an aldehyde or ketone and then reduction of the intermediate imine or iminium ion with a suitable reducing agent such as a metal hydride (e.g. sodium cyanoborohydride).

Compounds of formula (AB1), i.e. compounds of formula (AB) in which R⁵ is hydrogen, may be prepared by the condensation of amidines of formula (J) (as defined previously) with substituted acetonitriles of formula (AC) and esters of formula (N) in the presence of a base (e.g. an alkoxide base, such as sodium ethoxide) in a suitable solvent (e.g. an alcohol, such as ethanol), see reaction scheme 55.

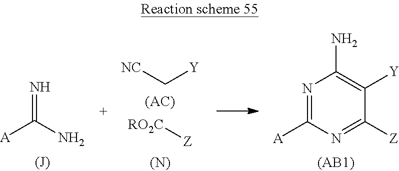

For example, a compound of formula (AB1) in which Z is CO₂R may be prepared by the reaction of an amidine of formula (J) with a substituted acetonitrile of formula (AC) and an oxalate diester of formula (N1).

Compounds of formula (AC) are known in the literature.

Alternatively compounds of formula (AB) may also be prepared from compounds of formula (G), as shown in reaction scheme 56.

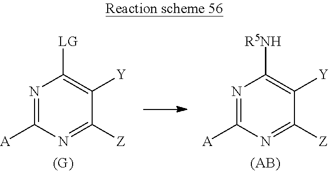

For example, a compound of formula (AB) may be prepared from a compound of formula (G) by reaction with a reagent R⁵NH₂ or R⁵NH⁻ in a suitable solvent (e.g. an ether, such as tetrahydrofuran). The reagent R⁵NH⁻ may be generated in situ by addition of a suitable base to R⁵NH₂, or it may be prepared separately and added as a suitable salt.

As an additional example a compound of formula (AB) may be prepared from a compound of formula (G) by treatment with a reagent R⁵NH₂ in the presence of a suitable catalyst (e.g. a metal catalyst, such as a palladium source) in a suitable solvent, and optionally a suitable ligand (e.g. a phosphine ligand, such as Josiphos).

Compounds of formula (AB) may additionally be prepared by reaction of compounds of formula (AD) with a suitable source of electrophilic Y (as shown in reaction scheme 57).

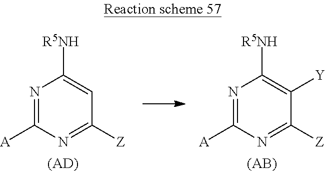

For example a compound of formula (AB) in which Y is a halogen may be prepared from a compound of formula (AD) by reaction with an N-halosuccinimide (e.g. N-chlorosuccinimide).

Compounds of formula (AD1), i.e. compounds of formula (AD) in which R⁵ is hydrogen, may be prepared by the reaction of amidines of formula (J) with cyanoketones of formula (AE) (see reaction scheme 58) in the presence of a base (e.g. an alkoxide base, such as sodium methoxide) in a suitable solvent (e.g. an alcohol, such as ethanol).

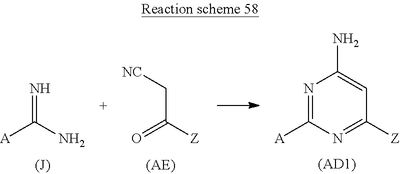

For example a compound of formula (AD1) in which Z is CO₂R may be prepared by the reaction of an amidine of formula (J) with a cyanopyruvate ester of formula (AE1) (i.e. a compound of formula (AE) in which Z is CO₂R). In one example, shown in reaction scheme 59 below, the compound of formula (AE1) may be reacted first with an alkylating agent (e.g. a methylating agent, such as dimethyl sulphate) in the presence of a base (e.g. an inorganic base, such as sodium bicarbonate) to form an enol ether, which is then reacted with amidine (J) in the presence of a base (e.g. an alkoxide base, such as sodium methoxide).

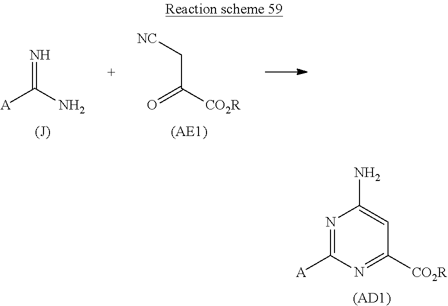

Compounds of formula (AE) are known or may be prepared using routine methods with which the skilled man is familiar.

Alternatively compounds of formula (AD) may also be prepared from compounds of formula (P), (reaction scheme 60).

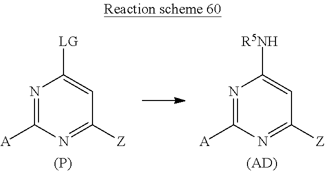

For example, a compound of formula (AD) may be prepared from a compound of formula (P) by reaction with a reagent R⁵NH₂ or R⁵NH⁻ in a suitable solvent (e.g. an alcohol or water). The reagent R⁵NH⁻ may be generated in situ by addition of a suitable base to R⁵NH₂, or it may be prepared separately and added as a suitable salt.

As a further example, a compound of formula (AD) may be prepared from a compound of formula (P) by treatment with a reagent R⁵NH₂ in the presence of a suitable catalyst (e.g. a metal catalyst, such as a palladium source) in a suitable solvent and optionally a suitable ligand (e.g a phosphine ligand, such as Josiphos). Compounds of formula (I) may be prepared from compounds of formula (AF), wherein LG is a suitable leaving group, such as a halogen atom or sulphonate (see reaction scheme 61).

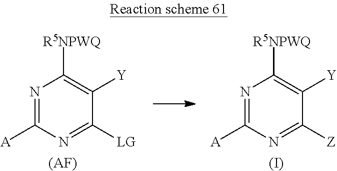

For example a compound of formula (I) in which Z is CO₂R may be prepared from a compound of formula (AF) by reaction with an alcohol ROH and carbon monoxide in the presence of a suitable metal catalyst (e.g. a palladium reagent, such as bis(triphenylphosphine)palladium dichloride) and a suitable base (e.g. an organic base, such as triethylamine), see reaction scheme 62. It may conveniently be conducted under an atmosphere of carbon monoxide gas at atmospheric or raised pressure.

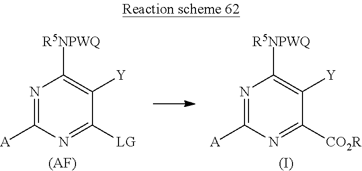

Alternatively a compound of formula (I) in which Z is T-(U)ₙ—V (as defined hereinbefore) may be prepared from a compound of formula (AF) by reaction with a compound of formula (AG) in the presence of a base (e.g. sodium hydride) in a suitable solvent (e.g. an ether, such as tetrahydrofuran).

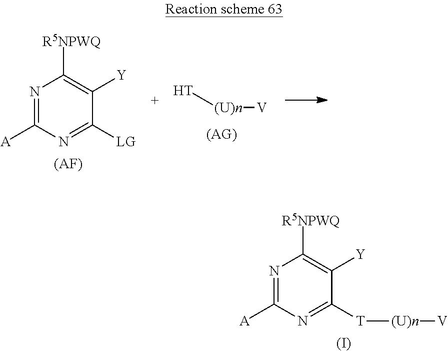

Compounds of formula (AG) are known compounds or may be prepared from known compounds using methods that are well known in the literature.

Compounds of formula (AF) may be prepared from compounds of formula (AH) (see reaction scheme 64) in which LG and LG′ may be the same or different and are leaving groups such as a halogen atoms or sulphonates.

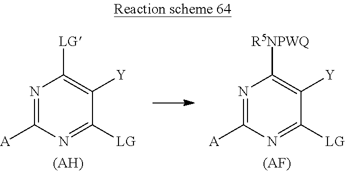

Compounds of formula (AH1), i.e. compounds of formula (AH) in which LG and LG′ are the same, may be prepared from compounds of formula (AI) by reaction with a suitable reagent, e.g. a phosphoryl halide or sulphonyl anhydride (reaction scheme 65).

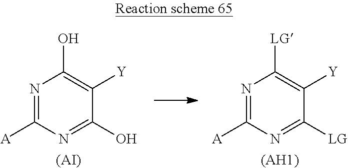

For example, a compound of formula (AH1) in which LG and LG′ are halogen atoms may be prepared by reaction of a compound of formula (AI) with a halogenating agent (e.g. a phosphoryl halide such as phosphorus oxychloride) in the presence of a suitable base (e.g. an organic base, such as N,N-diethylaniline).

Compounds of formula (AI) may be prepared by the reaction of amidines of formula (J) with malonyl diesters of formula (AJ) shown schematically below in reaction scheme 66, in the presence of a suitable base (e.g. an inorganic base, such as potassium carbonate, or an alkoxide base, such as sodium methoxide) in a suitable solvent (e.g. an ether, such as diglyme, or an alcohol, such as ethanol).

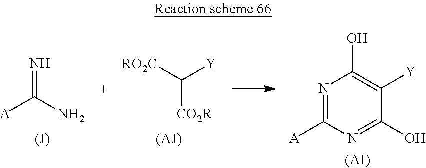

Diesters of formula (AJ) are known in the literature or may be prepared by methods known in the literature.

Alternatively, compounds of formula (AI) may be prepared by reaction of compounds of formula (AK) with a suitable source of electropositive Y (reaction scheme 67).

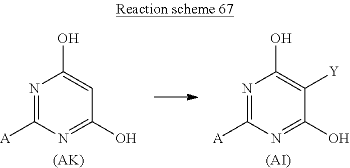

For example a compound of formula (AI) in which Y is a halogen may be prepared from a compound of formula (AK) by reaction with a halogenating agent (e.g. an N-halosuccinimide such as N-chlorosuccinimide, or an elemental halogen such as bromine).

Compounds of formula (AK) may be prepared from amidines of formula (J) and malonyl diesters of formula (AL)—reaction scheme 68.

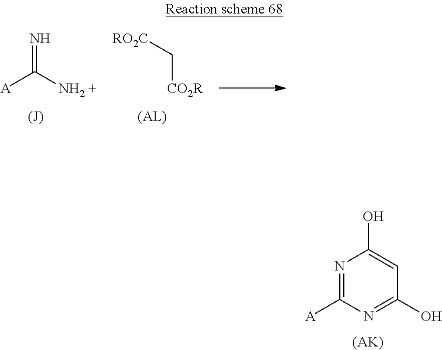

Diesters of formula (A) are well known in the literature.

Compounds of formula (I) in which P is CH(CONHR′) may be prepared by the reaction of compounds of formula (H) with an amine of formula R⁵NH₂, an aldehyde of formula Q-CHO and an isonitrile of formula R′NC.

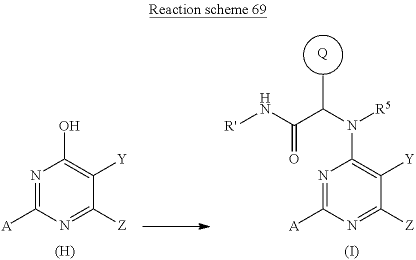

Compounds of formula (I) in which m is 0 and n is 2 may be prepared by the reaction of compounds of formula (AM) with a reagent that can functionalise the double bond (reaction scheme 70).

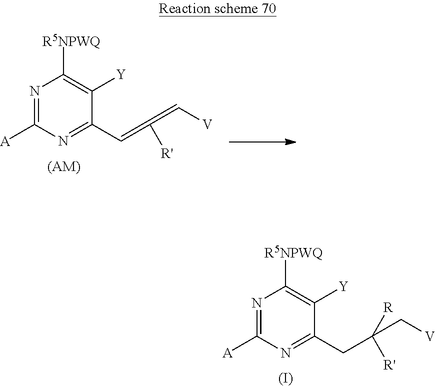

For example, a compound of formula (I) in which the groups R′ are hydrogen may be prepared from a compound of formula (AM) by reaction with a suitable reducing agent, (e.g. hydrogen gas in the presence of a metal catalyst, such as palladium supported on carbon), reaction scheme 71.

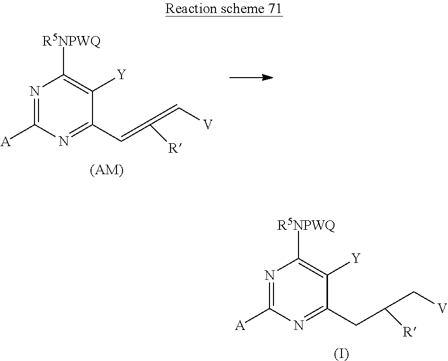

As yet a further example, a compound of formula (I) in which R′ represents vicinal hydroxyl groups may be prepared from a compound of formula (AM) by reaction with a dihydroxylation reagent (e.g. osmium tetroxide), reaction scheme 72.

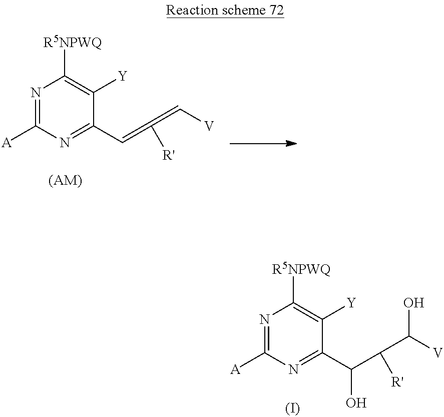

Compounds of formula (AM) may be prepared by the Heck reaction of compounds of formula (AF) with compounds of formula (AN) (see reaction scheme 73) in the presence of a suitable metal catalyst (e.g. a palladium species, such as palladium acetate) and a base (e.g. an organic base, such as triethylamine), in a suitable solvent.

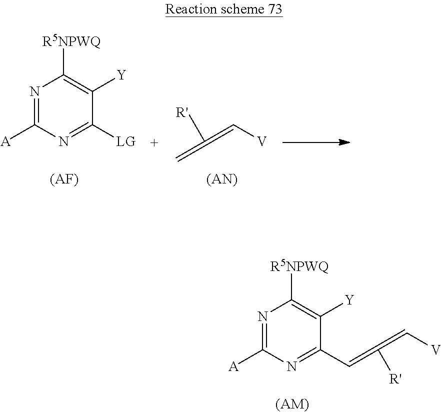

Alternatively, compounds of formula (AM) may be prepared by the reaction of compounds of formula (I) in which Z is CHO under the conditions of a suitable olefination reaction (see reaction scheme 74), for example a Wittig, Horner-Emmons or Peterson reaction.

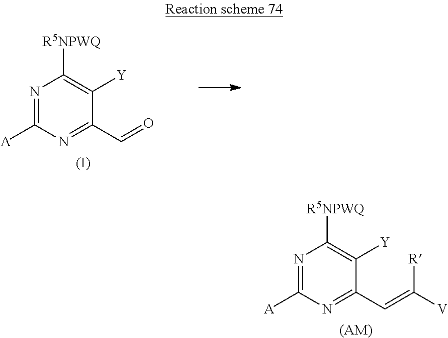

Compounds of formula (I) may be prepared from different compounds of formula (I) by the conversion of any of the substituents Y, Z, A, P, W and R⁵ or the ring Q into a different group Y, Z, A, P, W, R⁵ or Q using techniques well known to the skilled man.

For example, an unsaturated group A (e.g. an alkene or cycloalkene) may be reduced to form a saturated group (e.g. an alkyl or cycloalkyl group). When A is an unsaturated ring it may be oxidised to form an aromatic ring under standard conditions.

A further example is the conversion of a compound in which Y is a halogen or sulphonate (e.g. a bromine or chlorine atom) into a compound in which Y is a carbon-based group, e.g. an alkyl group or an optionally substituted phenyl or heteroaryl ring. Such a transformation may be carried out by reaction with a metal or metalloid derivative of the alkyl group or phenyl or heteroaryl ring (e.g. a boronic acid or boronate ester) in the presence of a base (e.g. an inorganic base, such as potassium phosphate or caesium fluoride, or an organic base, such as triethylamine), a metal source (e.g. a palladium source such as Pd₂(dba)₃) and, optionally, a ligand for the metal (e.g. a phosphine ligand, such as X-Phos) in a suitable solvent (e.g. a single solvent, such as acetonitrile, or a mixed solvent system, such as a mixture of dimethoxyethane and water). The metal catalyst and ligands may also be added as a single, pre-formed, complex (e.g. as a palladium/phosphine complex, such as palladium tetrakistriphenylphosphine, bis(triphenylphosphine)palladium dichloride or [1,1′-bis(diphenylphosphino)ferrocene]palladium dichloride).

A further compound of formula (I) may be prepared from a compound of formula (I), in which R⁵ is H by reaction with a suitable reagent R⁵-LG in which LG is a leaving group such as a halogen atom. Examples of such reagents R⁵-LG are alkyl halides and acid anhydrides. For example a compound of formula (I) in which R⁵ is COR may be prepared from a compound of formula (I) wherein R⁵ is hydrogen by reaction with an acylating agent such as an acyl chloride in the presence of a base (see reaction scheme 75).

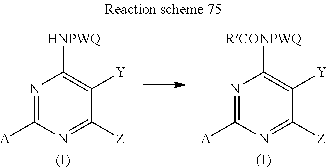

In an additional example a compound of formula (I) in which V is a carboxylic acid may be prepared from a compound of formula (I) in which V is a carboxylate ester, by hydrolysis under basic or acidic conditions, for example by treatment with aqueous sodium hydroxide. Alternatively this transformation may be achieved by treatment of the ester with a nucleophile, e.g. an alkyl thiolate, in a suitable solvent (both shown schematically in reaction scheme 76 below).

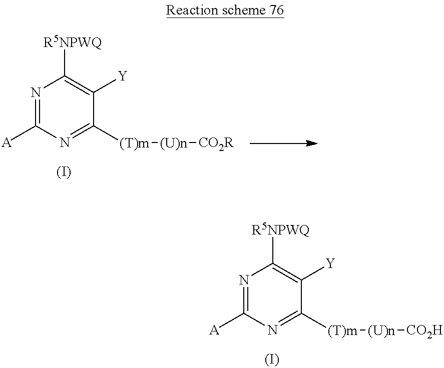

A compound of formula (I) in which V is a carboxylate ester may be prepared directly from a compound of formula (I) in which V is a carboxylic acid by esterification under standard conditions, for example by treatment with an alcohol ROH and an acid catalyst (e.g., thionyl chloride). Alternatively, this transformation may be achieved by first preparing an activated derivative of the acid group, e.g. an acyl halide, followed by reaction with an alcohol.

Other derivatives of the acid group in compounds of formula (I) in which V is a carboxylic acid may be prepared by standard methods found in the literature. For example a compound of formula (I) in which V is an amide group may be prepared from a compound of formula (I) in which V is a carboxylic acid by treatment with a suitable coupling reagent (e.g. a carbodiimide such as dicyclohexylcarbodiimide) and an amine RR′NH, optionally with a additive (e.g. dimethylaminopyridine), in a suitable solvent (e.g. dimethylformamide). Alternatively, this transformation may be performed by first preparing an activated derivative of the carboxylic acid group (e.g. an acyl halide such as an acid chloride), and then treating the activated derivative with an amine R′₂NH. Again, both transformations are shown schematically in reaction scheme 77 below.

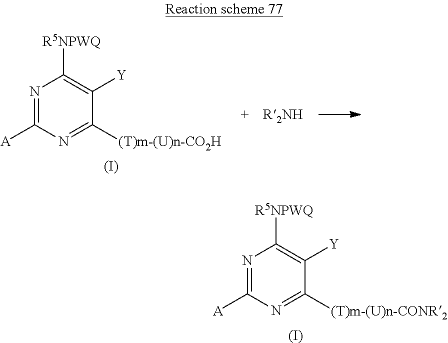

A compound of formula (I) in which Z is CO₂R may be prepared from a compound of formula (I) in which Z is CH(OR)₂ by treatment with a suitable oxidising agent, e.g. N-bromosuccinimide or a persulphate salt, optionally in the presence of an acid such as sulphuric acid (see reaction scheme 78).

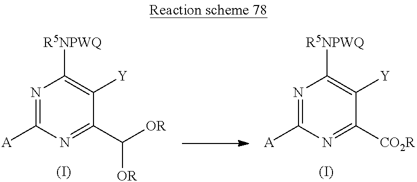

A compound of formula (I) wherein V is CHO may be prepared from a compound of formula (I) in which V is CO₂R by treatment with a suitable reducing agent (e.g. a hydride reducing agent such as DIBAL-H), see reaction scheme 79.

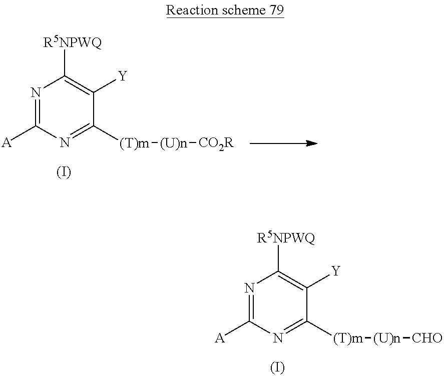

A compound of formula (I) in which V is CO₂H may be prepared from a compound of formula (I) in which V is CHO by treatment with a suitable oxidising agent (e.g. sodium hypochlorite), see reaction scheme 80.

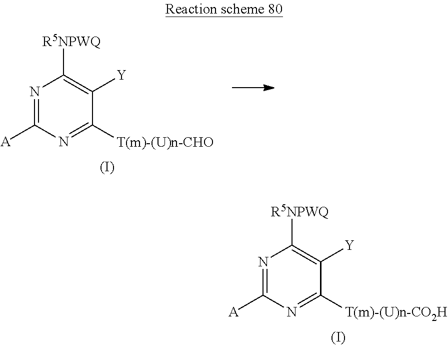

A compound of formula (I) in which V is CHO may be prepared by the removal of a suitable aldehyde protecting group (APG) from a compound of formula (I) in which V is a protected aldehyde (reaction scheme 81). Some examples of such protecting groups are acetals, thioacetals or hydrazones.

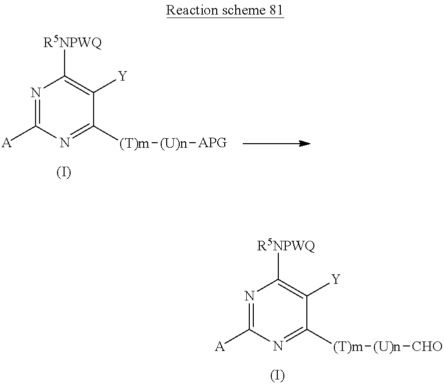

For example, a compound of formula (I) in which V is CHO may be prepared from a compound of formula (I) in which the aldehyde protecting group is an acetal, e.g. a dialkyl acetal such as a diethyl acetal) by treatment with acid (e.g. an inorganic acid such as aqueous hydrochloric acid), shown schematically below.

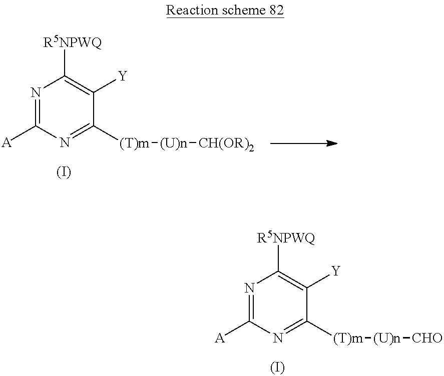

A compound of formula (I) in which V is CHO may also be prepared from a compound of formula (I) in which V is CH₂OH by oxidation under standard conditions, see reaction scheme 83, (e.g. using the Swern reaction).

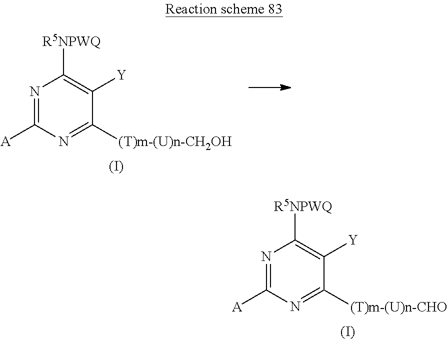

An aldehyde of formula (I) in which V is CHO may be converted into a derivative (e.g. an acetal or dithioacetal) of formula (I) under conditions that are known in the literature.

A compound of formula (I) in which V is CH₂OH may be prepared from a compound of formula (I) in which V is CHO by reaction with a suitable reducing agent (e.g. a metal hydride reducing agent, such as sodium borohydride), see reaction scheme 84.

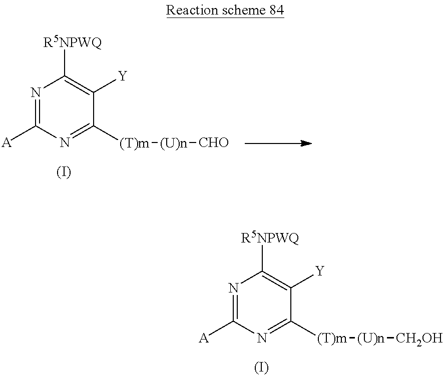

Alternatively, a compound of formula (I) in which V is CH₂OH may be prepared from a compound of formula (I) in which V is CO₂R by treatment with a suitable reducing agent (e.g: a metal hydride reducing agent, such as lithium aluminium hydride), see reaction scheme 85.

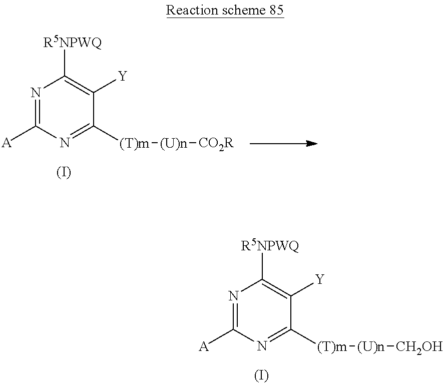

A compound of formula (I) in which V is CH₂OC(O)R may be prepared from a compound (I) in which V is CH₂OH under standard conditions, for example by treatment with an acyl halide in the presence of a base (reaction scheme 86).

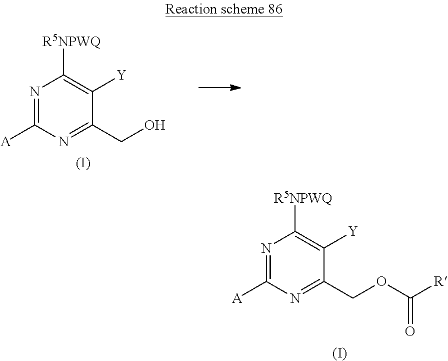

The skilled man will understand that transformations of this type may equally well be conducted at different stages of the synthetic route, for example converting one compound of formula (H) into a different compound of formula (H).

The skilled man will also understand that in certain instances more than one transformation can be conducted at one time utilising the same reaction conditions. For example a compound of formula (I) in which A and Y are the same may be prepared from a compound of formula (A1) (i.e. a compound of formula A in which Y is a leaving group such as a halogen atom or a sulphonate, which may be the same or different to LG) by reaction with an excess of a metal or metalloid derivative of A, such as a boronic acid, in the presence of a metal catalyst (e.g. a palladium derivative such as Pd₂(dba)₃), a ligand (e.g. a phosphine ligand such as X-Phos) and a base (such as potassium phosphate) in a suitable solvent (reaction scheme 87).

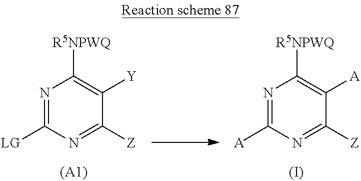

Another example is the preparation of a compound of formula (I) in which V is CONR⁵(PWQ) from a compound of formula (G1) (i.e. a compound of formula (G) in which V is CO₂R) by treatment with an excess of an amine R⁵(QWP)NH in a suitable solvent (reaction scheme 88).

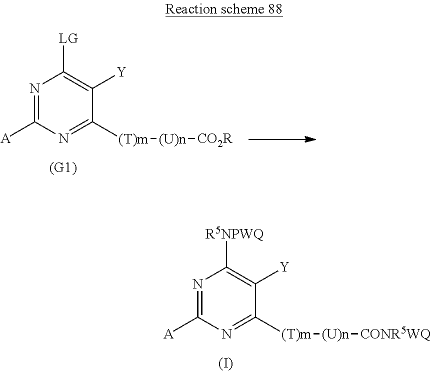

A further example (see reaction scheme 89) is the preparation of a compound of formula (H1) from a compound of formula (I2) by oxidation using a reagent that also provides a source of electropositive Y (e.g., N-bromosuccinimide for the case in which Y is Br).

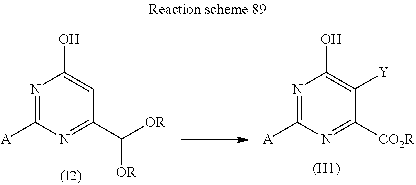

One skilled in the art will realise that it is often possible to alter the order in which the transformations described above are conducted, or to combine them in alternative ways to prepare a wide range of compounds of formula (1). All such variations are contemplated within the scope of the invention.

The skilled man will also be aware that some reagents will be incompatible with certain values or combinations of the substituents Y, Z, A, P, W, Q, R⁵, R and R′ as defined herein, and any additional steps, such as protection and/or deprotection steps, which are necessary to achieve the desired transformation will be clear to the skilled man.

Compounds of formula (I) may be used in unmodified form, i.e. as obtainable from synthesis, but preferably are formulated in any suitable manner using formulation adjuvants, such as carriers, solvents and surface-active substances, for example, as described hereinafter.

The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO Specifications for Plant Protection Products, 5th Edition, 1999. The formulations can be in the form of concentrates which are diluted prior to use, although ready-to-use formulations can also be made. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof. The active ingredients can also be contained in very fine microcapsules consisting of a polymer. Microcapsules usually have a diameter of from 0.1 to 500 microns. Typically, they will contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other known polymers. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxypropanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol (PEG), propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like. Water is generally the carrier of choice for diluting the concentrates. Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances, as described, for example, in CFR 180.1001. (c) & (d).

A large number of surface-active substances may advantageously be used in the formulations, especially in those formulations designed to be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in “McCutcheon's Detergents and Emulsifiers Annual” MC Publishing Corp., Ridgewood N.J., 1981.

Further adjuvants that can usually be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and also liquid and solid fertilisers.

The compositions according to the invention can additionally include an additive comprising an oil of vegetable or animal origin, a mineral oil, alkyl esters of such oils or mixtures of such oils and oil derivatives. The amount of oil additive in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil, olive oil or sunflower oil, emulsified vegetable oil, such as AMIGO® (Rhône-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈₋₂₂ fatty acids, especially the methyl derivatives of C₁₂₋₁₈ fatty acids, for example the methyl esters of lauric acid, palmitic acid and oleic acid, being of importance. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Another preferred adjuvant is Adigor® (Syngenta AG) which is a methylated rapeseed oil-based adjuvant.

The application and action of the oil additives can be further improved by combination with surface-active substances, such as non-ionic, anionic or cationic surfactants. Examples of suitable anionic, non-ionic and cationic surfactants are listed on pages 7 and 8 of WO97/34485. Preferred surface-active substances are anionic surfactants of the dodecylbenzylsulfonate type, especially the calcium salts thereof, and also non-ionic surfactants of the fatty alcohol ethoxylate type. Special preference is given to ethoxylated C₁₂₋₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltriloxanes which are commercially available e.g. as Silwet L-77®, and also perfluorinated surfactants. The concentration of the surface-active substances in relation to the total additive is generally from 1 to 30% by weight. Examples of oil additives consisting of mixtures of oil or mineral oils or derivatives thereof with surfactants are Edenor ME SU®, Turbocharge® (Syngenta AG, CH) or ActipronC (BP Oil UK Limited, GB).

If desired, it is also possible for the mentioned surface-active substances to be used in the formulations on their own, that is to say without oil additives.

Furthermore, the addition of an organic solvent to the oil additive/surfactant mixture may contribute to an additional enhancement of action. Suitable solvents are, for example, Solvesso® (ESSO) or Aromatic Solvent® (Exxon Corporation). The concentration of such solvents can be from 10 to 80% by weight of the total weight. Oil additives that are present in admixture with solvents are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada).

In addition to the oil additives listed above, for the purpose of enhancing the action of the compositions according to the invention it is also possible for formulations of alkylpyrrolidones (e.g. Agrimax®) to be added to the spray mixture. Formulations of synthetic lattices, e.g. polyacrylamide, polyvinyl compounds or poly-1-p-menthene (e.g. Bond®, Courier® or Emerald®) may also be used. It is also possible for solutions that contain propionic acid, for example Eurogkem Pen-e-trate®, to be added to the spray mixture as action-enhancing agent.

Herbicidal compositions of the invention generally comprise from 0.1 to 99% by weight, especially from 0.1 to 95% by weight, compounds of formula (I) and from 1 to 99.9% by weight of a formulation adjuvant which preferably includes from 0 to 25% by weight of a surface-active substance. Whereas commercial products will preferably be formulated as concentrates, the end user will normally employ dilute formulations.

Examples of preferred formulation types and their typical compositions are given below (% is percent by weight). Wettable powders as described herein are one particularly preferred type of formulation for use in the invention. In other preferred embodiments, in particular where the compound/composition/formulation of the invention is intended for use on turf, granular (inert or fertiliser) formulations as described herein are particularly suitable.

### Emulsifiable Concentrates:

| | |
|---|---|
| active ingredient: | 1 to 95%, preferably 60 to 90% |
| surface-active agent: | 1 to 30%, preferably 5 to 20% |
| liquid carrier: | 1 to 80%, preferably 1 to 35% |

### Dusts:

| | |
|---|---|
| active ingredient: | 0.1 to 10%, preferably 0.1 to 5% |
| solid carrier: | 99.9 to 90%, preferably 99.9 to 99% |

### Suspension Concentrates:

| | |
|---|---|
| active ingredient: | 5 to 75%, preferably 10 to 50% |
| water: | 94 to 24%, preferably 88 to 30% |
| surface-active agent: | 1 to 40%, preferably 2 to 30% |

### Wettable Powders:

| | |
|---|---|
| active ingredient: | 0.5 to 90%, preferably 1 to 80% |
| surface-active agent: | 0.5 to 20%, preferably 1 to 15% |
| solid carrier: | 5 to 95%, preferably 15 to 90% |

### Granules:

| | |
|---|---|
| active ingredient: | 0.1 to 30%, preferably 0.1 to 15% |
| solid carrier: | 99.5 to 70%, preferably 97 to 85% |

The following Examples further illustrate, but do not limit, the invention.

### Formulation Examples for Herbicides of Formula (I) (%=% by Weight)

| F1. Emulsifiable concentrates | a) | b) | c) | d) |
|---|---|---|---|---|
| active ingredient | 5% | 10% | 25% | 50% |
| calcium dodecylbenzenesulfonate | 6% | 8% | 6% | 8% |
| castor oil polyglycol ether | 4% | — | 4% | 4% |
| (36 mol of ethylene oxide) | | | | |
| octylphenol polyglycol ether | — | 4% | — | 2% |
| (7-8 mol of ethylene oxide) | | | | |
| NMP | — | — | 10% | 20% |
| arom. hydrocarbon mixture | 85% | 78% | 55% | 16% |
| (C₉-C₁₂) | | | | |

Emulsions of any desired concentration can be obtained from such concentrates by dilution with water.

| F2. Solutions | a) | b) | c) | d) |
|---|---|---|---|---|
| active ingredient | 5% | 10% | 50% | 90% |
| 1-methoxy-3-(3-methoxy- | — | 20% | 20% | — |
| propoxy)-propane | | | | |
| polyethylene glycol MW 400 | 20% | 10% | — | — |
| NMP | — | — | 30% | 10% |
| arom. hydrocarbon mixture | 75% | 60% | — | — |
| (C₉-C₁₂) | | | | |

The solutions are suitable for use in the form of microdrops.

| F3. Wettable powders | a) | b) | c) | d) |
|---|---|---|---|---|
| active ingredient | 5% | 25% | 50% | 80% |
| sodium lignosulfonate | 4% | — | 3% | — |
| sodium lauryl sulphate | 2% | 3% | — | 4% |
| sodium diisobutylnaphthalene- | — | 6% | 5% | 6% |
| sulfonate | | | | |
| octylphenol polyglycol ether | — | 1% | 2% | — |
| (7-8 mol of ethylene oxide) | | | | |
| highly dispersed silicic acid | 1% | 3% | 5% | 10% |
| kaolin | 88% | 62% | 35% | — |

The active ingredient is mixed thoroughly with the adjuvants and the mixture is thoroughly ground in a suitable mill, affording wettable powders which can be diluted with water to give suspensions of any desired concentration.

| F4. Coated granules | a) | b) | c) |
|---|---|---|---|
| active ingredient | 0.1% | 5% | 15% |
| highly dispersed silicic acid | 0.9% | 2% | 2% |
| inorganic carrier | 99.0% | 93% | 83% |
| (diameter 0.1-1 mm) | | | |
| e.g. CaCO₃ or SiO₂ | | | |

The active ingredient is dissolved in methylene chloride and applied to the carrier by spraying, and the solvent is then evaporated off in vacuo.

| F5. Coated granules | a) | b) | c) |
|---|---|---|---|
| active ingredient | 0.1% | 5% | 15% |
| polyethylene glycol MW 200 | 1.0% | 2% | 3% |
| highly dispersed silicic acid | 0.9% | 1% | 2% |
| inorganic carrier | 98.0% | 92% | 80% |
| (diameter 0.1-1 mm) | | | |
| e.g. CaCO₃ or SiO₂ | | | |

The finely ground active ingredient is uniformly applied, in a mixer, to the carrier moistened with polyethylene glycol. Non-dusty coated granules are obtained in this manner.

| F6. Extruder granules | a) | b) | c) | d) |
|---|---|---|---|---|
| active ingredient | 0.1% | 3% | 5% | 15% |
| sodium lignosulfonate | 1.5% | 2% | 3% | 4% |
| carboxymethylcellulose | 1.4% | 2% | 2% | 2% |
| kaolin | 97.0% | 93% | 90% | 79% |

The active ingredient is mixed and ground with the adjuvants, and the mixture is moistened with water. The mixture is extruded and then dried in a stream of air.

| F7. Dusts | a) | b) | c) |
|---|---|---|---|
| active ingredient | 0.1% | 1% | 5% |
| talcum | 39.9% | 49% | 35% |
| kaolin | 60.0% | 50% | 60% |

Ready-to-use dusts are obtained by mixing the active ingredient with the carriers and grinding the mixture in a suitable mill.

| F8. Suspension concentrates | a) | b) | c) | d) |
|---|---|---|---|---|
| active ingredient | 3% | 10% | 25% | 50% |
| ethylene glycol | 5% | 5% | 5% | 5% |
| nonylphenol polyglycol ether | — | 1% | 2% | — |
| (15 mol of ethylene oxide) | | | | |
| sodium lignosulfonate | 3% | 3% | 4% | 5% |
| carboxymethylcellulose | 1% | 1% | 1% | 1% |
| 37% aqueous formaldehyde | 0.2% | 0.2% | 0.2% | 0.2% |
| solution | | | | |
| silicone oil emulsion | 0.8% | 0.8% | 0.8% | 0.8% |
| water | 87% | 79% | 62% | 38% |

The finely ground active ingredient is intimately mixed with the adjuvants, giving a suspension concentrate from which suspensions of any desired concentration can be obtained by dilution with water.

Compounds of the invention (as well as mixtures and/or formulations containing the same) find utility as herbicides, and may thus be employed in methods of controlling plant growth. Such methods involve applying to the plants or to the locus thereof an herbicidally effective amount of said compound, or composition comprising the same (or mixture as described hereinafter). The invention thus also relates to a method of inhibiting plant growth which comprises applying to the plants or to the locus thereof a herbicidally effective amount of a compound of formula (I), composition, or mixture of the invention. In particular the invention provides a method of controlling weeds in crops of useful plants, which comprising applying to said weeds or the locus of said weeds, or to said crop of useful plants, a compound of formula I or a composition or mixture containing the same.

The term “locus” as used herein includes not only areas where weeds may already be growing, but also areas where weeds have yet to emerge, and also to areas under cultivation with respect to crops of useful plants. Areas under cultivation include land on which the crop plants are already growing and land intended for cultivation with such crop plants.

A compound, composition, and/or mixture of the invention may be used in a pre-emergence application and/or in a post-emergence application in order to mediate its effect.

Crops of useful plants in which compounds of formula (I), as well as formulations and/or mixtures containing the same, may be used according to the invention include perennial crops, such as citrus fruit, grapevines, nuts, oil palms, olives, pome fruit, stone fruit and rubber, and annual arable crops, such as cereals, for example barley and wheat, cotton, oilseed rape, maize, rice, soy beans, sugar beet, sugar cane, sunflowers, ornamentals and vegetables, especially cereals and maize.

Compounds of formula (I), formulations and/or mixtures containing the same may also be used on turf, pasture, rangeland, rights of way etc. In particular they may used on golf-courses, lawns, parks, sports-fields, race-courses and the like.

Crops are to be understood as also including those crops which have been rendered tolerant to herbicides or classes of herbicides (e.g. Auxines, ALS-, GS-, EPSPS-, PPO- and HPPD-inhibitors) by conventional methods of breeding or by genetic engineering. An example of a crop that has been rendered tolerant to imidazolinones, e.g. imazamox, by conventional methods of breeding is Clearfield® summer rape (canola). Examples of crops that have been rendered tolerant to herbicides by genetic engineering methods include e.g. glyphosate- and glufosinate-resistant maize varieties commercially available under the trade names RoundupReady® and LibertyLink®, as well as corn, soybean and cotton that have been engineered to be resistant to phenoxypropionic acids, pyridyloxyacetic acids and/or picolinate auxines.

Crops are also to be understood as being those which have been rendered resistant to harmful insects by genetic engineering methods, for example Bt maize (resistant to European corn borer), Bt cotton (resistant to cotton boll weevil) and also Bt potatoes (resistant to Colorado beetle). Examples of Bt maize are the Bt 176 maize hybrids of NK® (Syngenta Seeds). The Bt toxin is a protein that is formed naturally by Bacillus thuringiensis soil bacteria. Examples of toxins, or transgenic plants able to synthesise such toxins, are described in EP-A-451 878, EP-A-374 753, WO 93/07278, WO 95/34656, WO 03/052073 and EP-A-427 529. Examples of transgenic plants comprising one or more genes that code for an insecticidal resistance and express one or more toxins are KnockOut® (maize), Yield Gard® (maize), NuCOTIN33B® (cotton), Bollgard® (cotton), NewLeaf® (potatoes), NatureGard® and Protexcta®. Plant crops or seed material thereof can be both resistant to herbicides and, at the same time, resistant to insect feeding (“stacked” transgenic events). For example, seed can have the ability to express an insecticidal Cry3 protein while at the same time being tolerant to glyphosate.

Crops are also to be understood as being those which are obtained by conventional methods of breeding or genetic engineering and contain so-called output traits (e.g. improved storage stability, higher nutritional value and improved flavour).

The term “weeds” as used herein means any undesired plant, and thus includes not only agronomically important weeds as described below, but also volunteer crop plants.

Compounds of formula (I) may be used against a large number of agronomically important weeds. The weeds that may be controlled include both monocotyledonous and dicotyledonous weeds, such as, for example, Alisma spp, Leptochloa chinensis, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum, Rottboellia, Cyperus and especially Cyperus iria, Abutilon, Sida, Xanthium, Amaranthus, Chenopodium, Ipomoea, Chrysanthemum, Galium, Viola, Veronica, and Ischaemum spp.

The rates of application of compounds of formula (I) may vary within wide limits and depend on the nature of the soil, the method of application (pre- or post-emergence; seed dressing; application to the seed furrow; no tillage application etc.), the crop plant, or weed to be controlled, the prevailing climatic conditions, and other factors governed by the method of application, the time of application and the target crop. The compounds of formula I according to the invention are generally applied at a rate of from 10 to 2000 g/ha, especially from 50 to 1000 g/ha.

Any method of application to weeds/crop of useful plant, or locus thereof, which is routinely used in agriculture may be used, for example application by spray or broadcast method typically after suitable dilution of a compound of formula (I) (whether said compound is formulated and/or in combination with one or more further active ingredients and/or safeners, as described herein).

The compounds of formula (I) according to the invention can also be used in combination with other active ingredients, e.g. other herbicides, and/or insecticides, and/or acaricides, and/or nematocides, and/or molluscicides, and/or fungicides, and/or plant growth regulators. Such mixtures, and the use of such mixtures to control weeds and/or undesired plant growth form yet further aspects of the invention. For the avoidance of doubt, mixtures of invention also include mixtures of two or more different compounds of formula (I).

Where a compound of formula (I) is combined with at least one additional herbicide, the following mixtures of the compound of formula (I) are particularly preferred. Compound of formula (I)+acetochlor, compound of formula (I)+acifluorfen, compound of formula (I)+acifluorfen-sodium, compound of formula (I)+aclonifen, compound of formula (I)+acrolein, compound of formula (I)+alachlor, compound of formula (I)+alloxydim, compound of formula (I)+allyl alcohol, compound of formula (I)+ametryn, compound of formula (I)+amicarbazone, compound of formula (I)+amidosulfuron, compound of formula (I)+aminopyralid, compound of formula (I)+amitrole, compound of formula (I)+ammonium sulfamate, compound of formula (I)+anilofos, compound of formula (I)+asulam, compound of formula (I)+atrazine, formula (I)+aviglycine, formula (I)+azafenidin, compound of formula (I)+azimsulfuron, compound of formula (I)+BCPC, compound of formula (I)+beflubutamid, compound of formula (I)+benazolin, formula (I)+bencarbazone, compound of formula (I)+benfluralin, compound of formula (I)+benfuresate, compound of formula (I)+bensulfuron, compound of formula (I)+bensulfuron-methyl, compound of formula (I)+bensulide, compound of formula (I)+bentazone, compound of formula (I)+benzfendizone, compound of formula (I)+benzobicyclon, compound of formula (I)+benzofenap, compound of formula (I)+bifenox, compound of formula (I)+bilanafos, compound of formula (I)+bispyribac, compound of formula (I)+bispyribac-sodium, compound of formula (I)+borax, compound of formula (I)+bromacil, compound of formula (I)+bromobutide, formula (I)+bromophenoxim, compound of formula (I)+bromoxynil, compound of formula (I)+butachlor, compound of formula (I)+butafenacil, compound of formula (I)+butamifos, compound of formula (I)+butralin, compound of formula (I)+butroxydim, compound of formula (I)+butylate, compound of formula (I)+cacodylic acid, compound of formula (I)+calcium chlorate, compound of formula (I)+cafenstrole, compound of formula (I)+carbetamide, compound of formula (I)+carfentrazone, compound of formula (I)+carfentrazone-ethyl, compound of formula (I)+CDEA, compound of formula (I)+CEPC, compound of formula (I)+chlorflurenol, compound of formula (I)+chlorflurenol-methyl, compound of formula (I)+chloridazon, compound of formula (I)+chlorimuron, compound of formula (I)+chlorimuron-ethyl, compound of formula (I)+chloroacetic acid, compound of formula (I)+chlorotoluron, compound of formula (I)+chlorpropham, compound of formula (I)+chlorsulfuron, compound of formula (I)+chlorthal, compound of formula (I)+chlorthal-dimethyl, compound of formula (I)+cinidon-ethyl, compound of formula (I)+cinmethylin, compound of formula (I)+cinosulfuron, compound of formula (I)+cisanilide, compound of formula (I)+clethodim, compound of formula (I)+clodinafop, compound of formula (I)+clodinafop-propargyl, compound of formula (I)+clomazone, compound of formula (I)+clomeprop, compound of formula (I)+clopyralid, compound of formula (I)+cloransulam, compound of formula (I)+cloransulam-methyl, compound of formula (I)+CMA, compound of formula (I)+4-CPB, compound of formula (I)+CPMF, compound of formula (I)+4-CPP, compound of formula (I)+CPPC, compound of formula (I)+cresol, compound of formula (I)+cumyluron, compound of formula (I)+cyanamide, compound of formula (I)+cyanazine, compound of formula (I)+cycloate, compound of formula (I)+cyclosulfamuron, compound of formula (I)+cycloxydim, compound of formula (I)+cyhalofop, compound of formula (I)+cyhalofop-butyl, compound of formula (I)+2,4-D, compound of formula (I)+3,4-DA, compound of formula (I)+daimuron, compound of formula (I)+dalapon, compound of formula (I)+dazomet, compound of formula (I)+2,4-DB, compound of formula (I)+3,4-DB, compound of formula (I)+2,4-DEB, compound of formula (I)+desmedipham, formula (I)+desmetryn, compound of formula (I)+dicamba, compound of formula (I)+dichlobenil, compound of formula (I)+ortho-dichlorobenzene, compound of formula (I)+para-dichlorobenzene, compound of formula (I)+dichlorprop, compound of formula (I)+dichlorprop-P, compound of formula (I)+diclofop, compound of formula (I)+diclofop-methyl, compound of formula (I)+diclosulam, compound of formula (I)+difenzoquat, compound of formula (I)+difenzoquat metilsulfate, compound of formula (I)+diflufenican, compound of formula (I)+diflufenzopyr, compound of formula (I)+dimefuron, compound of formula (I)+dimepiperate, compound of formula (I)+dimethachlor, compound of formula (I)+dimethametryn, compound of formula (I)+dimethenamid, compound of formula (I)+dimethenamid-P, compound of formula (I)+dimethipin, compound of formula (I)+dimethylarsinic acid, compound of formula (I)+dinitramine, compound of formula (I)+dinoterb, compound of formula (I)+diphenamid, formula (I)+dipropetryn, compound of formula (I)+diquat, compound of formula (I)+diquat dibromide, compound of formula (I)+dithiopyr, compound of formula (I)+diuron, compound of formula (I)+DNOC, compound of formula (I)+3,4-DP, compound of formula (I)+DSMA, compound of formula (I)+EBEP, compound of formula (I)+endothal, compound of formula (I)+EPTC, compound of formula (I)+esprocarb, compound of formula (I)+ethalfluralin, compound of formula (I)+ethametsulfuron, compound of formula (I)+ethametsulfuron-methyl, formula (I)+ethephon, compound of formula (I)+ethofumesate, compound of formula (I)+ethoxyfen, compound of formula (I)+ethoxysulfuron, compound of formula (I)+etobenzanid, compound of formula (I)+fenoxaprop, compound of formula (I)+fenoxaprop-P, compound of formula (I)+fenoxaprop-ethyl, compound of formula (I)+fenoxaprop-P-ethyl, compound of formula (I)+fentrazamide, compound of formula (I)+ferrous sulfate, compound of formula (I)+flamprop-M, compound of formula (I)+flazasulfuron, compound of formula (I)+florasulam, compound of formula (I)+fluazifop, compound of formula (I)+fluazifop-butyl, compound of formula (I)+fluazifop-P, compound of formula (I)+fluazifop-P-butyl, formula (I)+fluazolate, compound of formula (I)+flucarbazone, compound of formula (I)+flucarbazone-sodium, compound of formula (I)+flucetosulfuron, compound of formula (I)+fluchloralin, compound of formula (I)+flufenacet, compound of formula (I)+flufenpyr, compound of formula (I)+flufenpyr-ethyl, formula (I)+flumetralin, compound of formula (I)+flumetsulam, compound of formula (I)+flumiclorac, compound of formula (I)+flumiclorac-pentyl, compound of formula (I)+flumioxazin, formula (I)+flumipropin, compound of formula (I)+fluometuron, compound of formula (I)+fluoroglycofen, compound of formula (I)+fluoroglycofen-ethyl, formula (I)+fluoxaprop, formula (I)+flupoxam, formula (I)+flupropacil, compound of formula (I)+flupropanate, compound of formula (I)+flupyrsulfuron, compound of formula (I)+flupyrsulfuron-methyl-sodium, compound of formula (I)+flurenol, compound of formula (I)+fluridone, compound of formula (I)+flurochloridone, compound of formula (I)+fluroxypyr, compound of formula (I)+flurtamone, compound of formula (I)+fluthiacet, compound of formula (I)+fluthiacet-methyl, compound of formula (I)+fomesafen, compound of formula (I)+foramsulfuron, compound of formula (I)+fosamine, compound of formula (I)+glufosinate, compound of formula (I)+glufosinate-ammonium, compound of formula (I)+glyphosate, compound of formula (I)+halosulfuron, compound of formula (I)+halosulfuron-methyl, compound of formula (I)+haloxyfop, compound of formula (I)+haloxyfop-P, compound of formula (I)+HC-252, compound of formula (I)+hexazinone, compound of formula (I)+imazamethabenz, compound of formula (I)+imazamethabenz-methyl, compound of formula (I)+imazamox, compound of formula (I)+imazapic, compound of formula (I)+imazapyr, compound of formula (I)+imazaquin, compound of formula (I)+imazethapyr, compound of formula (I)+imazosulfuron, compound of formula (I)+indanofan, compound of formula (I)+iodomethane, compound of formula (I)+iodosulfuron, compound of formula (I)+iodosulfuron-methyl-sodium, compound of formula (I)+ioxynil, compound of formula (I)+isoproturon, compound of formula (I)+isouron, compound of formula (I)+isoxaben, compound of formula (I)+isoxachlortole, compound of formula (I)+isoxaflutole, formula (I)+isoxapyrifop, compound of formula (I)+karbutilate, compound of formula (I)+lactofen, compound of formula (I)+lenacil, compound of formula (I)+linuron, compound of formula (I)+MAA, compound of formula (I)+MAMA, compound of formula (I)+MCPA, compound of formula (I)+MCPA-thioethyl, compound of formula (I)+MCPB, compound of formula (I)+mecoprop, compound of formula (I)+mecoprop-P, compound of formula (I)+mefenacet, compound of formula (I)+mefluidide, compound of formula (I)+mesosulfuron, compound of formula (I)+mesosulfuron-methyl, compound of formula (I)+mesotrione, compound of formula (I)+metam, compound of formula (I)+metamifop, compound of formula (I)+metamitron, compound of formula (I)+metazachlor, compound of formula (I)+methabenzthiazuron, formula (I)+methazole, compound of formula (I)+methylarsonic acid, compound of formula (I)+methyldymron, compound of formula (I)+methyl isothiocyanate, compound of formula (I)+metobenzuron, formula (I)+metobromuron, compound of formula (I)+metolachlor, compound of formula (I)+S-metolachlor, compound of formula (I)+metosulam, compound of formula (I)+metoxuron, compound of formula (I)+metribuzin, compound of formula (I)+metsulfuron, compound of formula (I)+metsulfuron-methyl, compound of formula (I)+MK-616, compound of formula (I)+molinate, compound of formula (I)+monolinuron, compound of formula (I)+MSMA, compound of formula (I)+naproanilide, compound of formula (I)+napropamide, compound of formula (I)+naptalam, formula (I)+NDA-402989, compound of formula (I)+neburon, compound of formula (I)+nicosulfuron, formula (I)+nipyraclofen, formula (I)+n-methyl glyphosate, compound of formula (I)+nonanoic acid, compound of formula (I)+norflurazon, compound of formula (I)+oleic acid (fatty acids), compound of formula (I)+orbencarb, compound of formula (I)+orthosulfamuron, compound of formula (I)+oryzalin, compound of formula (I)+oxadiargyl, compound of formula (I)+oxadiazon, compound of formula (I)+oxasulfuron, compound of formula (I)+oxaziclomefone, compound of formula (I)+oxyfluorfen, compound of formula (I)+paraquat, compound of formula (I)+paraquat dichloride, compound of formula (I)+pebulate, compound of formula (I)+pendimethalin, compound of formula (I)+penoxsulam, compound of formula (I)+pentachlorophenol, compound of formula (I)+pentanochlor, compound of formula (I)+pentoxazone, compound of formula (I)+pethoxamid, compound of formula (I)+petrolium oils, compound of formula (I)+phenmedipham, compound of formula (I)+phenmedipham-ethyl, compound of formula (I)+picloram, compound of formula (I)+picolinafen, compound of formula (I)+pinoxaden, compound of formula (I)+piperophos, compound of formula (I)+potassium arsenite, compound of formula (I)+potassium azide, compound of formula (I)+pretilachlor, compound of formula (I)+primisulfuron, compound of formula (I)+primisulfuron-methyl, compound of formula (I)+prodiamine, compound of formula (I)+profluazol, compound of formula (I)+profoxydim, formula (I)+prohexadione-calcium, compound of formula (I)+prometon, compound of formula (I)+prometryn, compound of formula (I)+propachlor, compound of formula (I)+propanil, compound of formula (I)+propaquizafop, compound of formula (I)+propazine, compound of formula (I)+propham, compound of formula (I)+propisochlor, compound of formula (I)+propoxycarbazone, compound of formula (I)+propoxycarbazone-sodium, compound of formula (I)+propyzamide, compound of formula (I)+prosulfocarb, compound of formula (I)+prosulfuron, compound of formula (I)+pyraclonil, compound of formula (I)+pyraflufen, compound of formula (I)+pyraflufen-ethyl, formula (I)+pyrasulfotole, compound of formula (I)+pyrazolynate, compound of formula (I)+pyrazosulfuron, compound of formula (I)+pyrazosulfuron-ethyl, compound of formula (I)+pyrazoxyfen, compound of formula (I)+pyribenzoxim, compound of formula (I)+pyributicarb, compound of formula (I)+pyridafol, compound of formula (I)+pyridate, compound of formula (I)+pyriftalid, compound of formula (I)+pyriminobac, compound of formula (I)+pyriminobac-methyl, compound of formula (I)+pyrimisulfan, compound of formula (I)+pyrithiobac, compound of formula (I)+pyrithiobac-sodium, formula (I)+pyroxasulfone, formula (I)+pyroxulam, compound of formula (I)+quinclorac, compound of formula (I)+quinmerac, compound of formula (I)+quinoclamine, compound of formula (I)+quizalofop, compound of formula (I)+quizalofop-P, compound of formula (I)+quizalofop-ethyl, compound of formula (I)+quizalofop-P-ehtyl, compound of formula (I)+rimsulfuron, compound of formula (I)+sethoxydim, compound of formula (I)+siduron, compound of formula (I)+simazine, compound of formula (I)+simetryn, compound of formula (I)+SMA, compound of formula (I)+sodium arsenite, compound of formula (I)+sodium azide, compound of formula (I)+sodium chlorate, compound of formula (I)+sulcotrione, compound of formula (I)+sulfentrazone, compound of formula (I)+sulfometuron, compound of formula (I)+sulfometuron-methyl, compound of formula (I)+sulfosate, compound of formula (I)+sulfosulfuron, compound of formula (I)+sulfuric acid, compound of formula (I)+tar oils, Compound of formula (I)+2,3,6-TBA, compound of formula (I)+TCA, compound of formula (I)+TCA-sodium, formula (I)+tebutam, compound of formula (I)+tebuthiuron, formula (I)+tefuryltrione, compound of formula 1 +tembotrione, compound of formula (I)+tepraloxydim, compound of formula (I)+terbacil, compound of formula (I)+terbumeton, compound of formula (I)+terbuthylazine, compound of formula (I)+terbutryn, compound of formula (I)+thenylchlor, compound of formula (I)+thiazafluron, compound of formula (I)+thiazopyr, compound of formula (I)+thifensulfuron, compound of formula (I)+thiencarbazone, compound of formula (I)+thifensulfuron-methyl, compound of formula (I)+thiobencarb, compound of formula (I)+tiocarbazil, compound of formula (I)+topramezone, compound of formula (I)+tralkoxydim, compound of formula (I)+tri-allate, compound of formula (I)+triasulfuron, compound of formula (I)+triaziflam, compound of formula (I)+tribenuron, compound of formula (I)+tribenuron-methyl, compound of formula (I)+tricamba, compound of formula (I)+triclopyr, compound of formula (I)+trietazine, compound of formula (I)+trifloxysulfuron, compound of formula (I)+trifloxysulfuron-sodium, compound of formula (I)+trifluralin, compound of formula (I)+triflusulfuron, compound of formula (I)+triflusulfuron-methyl, compound of formula (I)+trifop, compound of formula (I)+trifop-methyl, compound of formula (I)+trihydroxytriazine, compound of formula (I)+trinexapac-ethyl, compound of formula (I)+tritosulfuron, compound of formula (I)+[3-[2-chloro-4-fluoro-5-(1-methyl-6-trifluoromethyl-2,4-dioxo-1,2,3,4-tetrahydropyrimidin-3-yl)phenoxy]-2-pyridyloxy]acetic acid ethyl ester (CAS RN 353292-31-6), compound of formula (I)+4-hydroxy-3-[[2-[(2-methoxyethoxy)methyl]-6-(trifluoromethyl)-3-pyridinyl]carbonyl]-bicyclo[3.2.1]oct-3-en-2-one (CAS RN 352010-68-5), and compound of formula (I)+4-hydroxy-3-[[2-(3-methoxypropyl)-6-(difluoromethyl)-3-pyridinyl]carbonyl]-bicyclo[3.2.1]oct-3-en-2-one.

Whilst two-way mixtures of a compound of formula (I) and another herbicide are explicitly disclosed above, the skilled man will appreciate that the invention extends to three-way, and further multiple combinations comprising the above two-way mixtures.

In preferred embodiments a compound of formula (I) is combined with an acetolactate inhibitor, (e.g. one or more of florasulam, metsulfuron, thifensulfuron, tribenuron, triasulfuron, flucarbazone, flupyrsulfuron, iodosulfuron, mesosulfuron, propoxicarbazone, sulfosulfuron, pyroxsulam and tritosulfuron, as well as salts or esters thereof), a synthetic auxine herbicide (e.g. one or more of clopyralid, 2,4-D, 2,4-DB, dicamba, dichlorprop, fluroxypyr, MCPA, MCPB, mecoprop and mecoprop-P), an ACCase-inhibiting herbicide (e.g. one or more of phenylpyrazolin; pinoxaden; an aryloxyphenoxypropionic herbicide such as clodinafop, cyhalofop, diclofop, fenoxaprop, fluazifop, haloxyfop, quizalofop, trifop and mixtures thereof, as well as the isomers thereof, for example, fenoxaprop-P, fluazifop-P, haloxyfop-P, quizalofop-P; and a cyclohexanedione herbicide such as alloxydim; butroxydim, clethodim, cycloxydim, profoxydim, sethoxydim, tepraloxydim and tralkoxydim, as well as salts or esters thereof), an EPSPS inhibitor (such as for example glyphosate or sulfosate), and/or an auxin transport inhibitor such as semicarbozone (e.g. diflufenzopyr, in particular the sodium salt) or phthalamate compound (e.g. naptalam).

Particularly preferred mixture partners for compounds of formula (I) are: florasulam, iodosulfuron-methyl-sodium, mesosulfuron-methyl, metsulfuron-methyl, thifensulfuron, triasulfuron, tribenuron-methyl or pyroxsulam; dicamba, fluroxypyr, MCPA, mecoprop or mecoprop-P; clodinafop-propargyl, cyhalofop-butyl, diclofop-methyl, fenoxaprop-ethyl, fenoxaprop-P-ethyl, fluazifop-butyl, fluazifop-P-butyl, haloxyfop-methyl, haloxyfop-P-methyl, pinoxaden, propaquizafop, quizalofop-ethyl, quizalofop-P-ethyl, tralkoxydim, trifop-methyl, glyphosate, diflufenzopyr-Na, and naptalam.

For the avoidance of doubt, even if not explicitly stated above, the mixing partners of the compound of formula (I) may also be in the form of any suitable agrochemically acceptable ester or salt, as mentioned e.g. in The Pesticide Manual, Thirteenth Edition, British Crop Protection Council, 2003.

The mixing ratio of the compound of formula (I) to the mixing partner is preferably from 1:100 to 1000:1.

The mixtures can advantageously be used in the above-mentioned formulations (in which case “active ingredient” relates to the respective mixture of compound of formula (I) with the mixing partner).

The compounds of formula (I) according to the invention can also be used in combination with one or more safeners. Likewise, mixtures of a compound of formula (I) according to the invention with one or more further active ingredients, in particular with one or more further herbicides, can also be used in combination with one or more safeners. Suitable safeners for use in combination with compounds of formula (I) include AD 67 (MON 4660), benoxacor, cloquintocet-mexyl, cyometrinil and the corresponding (Z) isomer, cyprosulfamide (CAS RN 221667-31-8), dichlormid, fenchlorazole-ethyl, fenclorim, flurazole, fluxofenim, furilazole and the corresponding R isomer, isoxadifen-ethyl, mefenpyr-diethyl, oxabetrinil, naphthalic anhydride (CAS RN 81-84-5) and N-isopropyl-4-(2-methoxy-benzoylsulfamoyl)-benzamide (CAS RN 221668-34-4). Particularly preferred safeners for use in the invention are cloquintocet-mexyl, cyprosulfamide, fenchlorazole-ethyl and mefenpyr-diethyl. The safeners of the compound of formula (I) may also be in the form of esters or salts, as mentioned e.g. in The Pesticide Manual, 13^{th} Edition supra. The reference to cloquintocet-mexyl also applies to a lithium, sodium, potassium, calcium, magnesium, aluminium, iron, ammonium, quaternary ammonium, sulfonium or phosphonium salt thereof as disclosed in WO02/34048, and the reference to fenchlorazole-ethyl also applies to fenchlorazole, etc.

Preferably the mixing ratio of compound of formula (I) to safener is from 100:1 to 1:10, especially from 20:1 to 1:1.

The mixtures can advantageously be used in the above-mentioned formulations (in which case “active ingredient” relates to the respective mixture of compound of formula (I) with the safener).

Preferred mixtures of a compound of formula (I) with further herbicides and safeners include: a compound of formula (I)+pinoxaden+cloquinctocet-mexyl, a compound of formula (I)+clodinafop+cloquintocet-mexyl, and a compound of formula (I)+clodinafop-propargyl+cloquintocet-mexyl.

Various aspects and embodiments of the present invention will now be illustrated in more detail by way of example. It will be appreciated that modification of detail may be made without departing from the scope of the invention.

For the avoidance of doubt, where a literary reference, patent application, or patent, is cited within the text of this application, the entire text of said citation is herein incorporated by reference.

### EXAMPLES

### Example 1

### Synthesis of 2-cyclopropyl-4,5-dichloro-6-methoxycarbonyl pyrimidine

### 1.1 Preparation of cyclopropylcarboxamidine hydrochloride salt

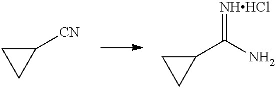

Hydrogen chloride gas was bubbled through a stirred solution of cyclopropylcarbonitrile (10.0 g, 0.15 mol) and methanol (6 ml, 0.15 mol) in dry ether (60 ml) at 0° C. for 2 hours. The reaction mixture was evaporated under reduced pressure and the residue dissolved in methanol (125 ml). The solution was added to an ice-cold mixture of methanol (125 ml) and liquid ammonia (15 ml) and the mixture stirred for 1 hour. The resulting clear solution was evaporated to leave cyclopropylcarboxamidine hydrochloride salt as a white solid (12.0 g, 67%). Characterising data for the compound are as follows: ¹H nmr (400 MHz, d₆-DMSO) δ_{H} 8.75 (2H, s), 8.64 (2H, s), 1.81 (1H, quintet), 1.11 (4H, s) ppm.

### 1.2 Preparation of 2-cyclopropyl-4-hydroxypyrimidine-6-carboxylic acid

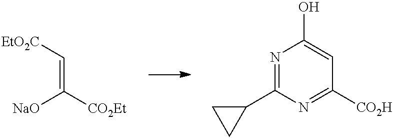

A solution of sodium hydroxide (2.85 g, 71.3 mmol) in water (3 ml) was added to a stirred solution of diethyl oxaloacetate sodium salt (8.7 g, 50 mmol) in water (50 ml) and the mixture stirred for 20 minutes. Cyclopropylcarboxamidine hydrochloride salt (5.0 g, 40 mmol) was added to the solution and the mixture was heated at 70° C. overnight, then cooled to ambient temperature and acidified to pH1 by the cautious addition of concentrated hydrochloric acid. The precipitate was isolated by filtration and dried to yield 2-cyclopropyl-4-hydroxypyrimidine-6-carboxylic acid (4.7 g, 63%). Characterising data for the compound are as follows: ¹H nmr (400 MHz, d₆-DMSO) δ_{H} 13.30 (1H, br s), 12.97 (1H, br s), 6.59 (1H, s), 1.94 (1H, quintet), 1.04 (4H, m) ppm.

### 1.3 Preparation of 5-chloro-2-cyclopropyl-4-hydroxypyrimidine-6-carboxylic acid

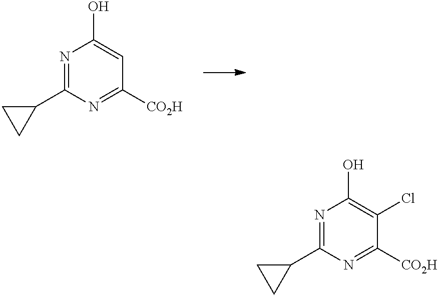

Aqueous sodium hypochlorite (470 ml) was added to a solution of 2-cyclopropyl-4-hydroxypyrimidine-6-carboxylic acid (68.7 g, 0.38 mol) in concentrated hydrochloric acid (280 ml) and water (412 ml), maintaining the temperature below 15° C. during the course of the addition. The reaction mixture was stirred for 12 hours at ambient temperature, then sodium metabisulphite (6.87 g) and sodium hydroxide (50% aqueous solution; 29.0 g) were added, maintaining the temperature below 15° C. during the addition. The precipitate was removed by filtration and dried to provide 5-chloro-2-cyclopropyl-4-hydroxypyrimidine-6-carboxylic acid (54 g, 66%). Characterising data for the compound are as follows: ¹H nmr (400 MHz, d₆-DMSO) δ_{H} 14.10 (1H, br s), 13.43 (1H, br s), 1.95 (1H, m), 1.08 (2H, m), 1.04 (2H, m) ppm.

### 1.4 Preparation of 2-cyclopropyl-4,5-dichloropyrimidine-6-carboxylic acid

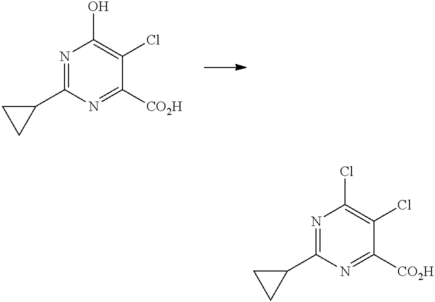

A mixture of 5-chloro-2-cyclopropyl-4-hydroxypyrimidine-6-carboxylic acid (10.0 g, 40 mmol) and phosphorus oxychloride (21.5 ml) was heated at reflux for 12 hours. The mixture was allowed to cool to ambient temperature and then added carefully to iced water and the resulting mixture extracted with ether. The combined ether layers were washed successively with water and brine, dried over sodium sulphate, filtered and the filtrate evaporated under reduced pressure to provide 2-cyclopropyl-4,5-dichloropyrimidine-6-carboxylic acid as a brown solid (7.0 g, 64%). Characterising data for the compound are as follows: ¹H nmr (400 MHz, d₆-DMSO) δ_{H} 3.37 (1H, br s), 2.24 (1H, m), 1.16 (2H, m), 1.08 (2H, m) ppm.

### 1.5 Preparation of 2-cyclopropyl-4,5-dichloro-6-methoxycarbonylpyrimidine

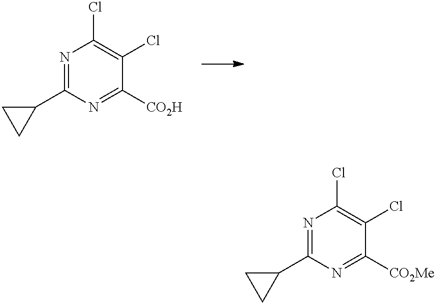

An excess of freshly prepared diazomethane was added to a solution of 2-cyclopropyl-4,5-dichloropyrimidine-6-carboxylic acid (50.0 g, 0.21 mol) in ether (1.5 I) at 0° C. After stirring for 15 minutes the reaction mixture was concentrated under reduced pressure to leave a brown oil. The residue was purified by column chromatography on silica using 10% ethyl acetate in hexane as eluent to provide 2-cyclopropyl-4,5-dichloro-6-methoxycarbonylpyrimidine (45.0 g, 85%) as a yellow oil, which solidified upon cooling. Characterising data for the compound is as follows: ¹H nmr (400 MHz, d₆-DMSO) δ_{H} 3.95 (3H, s), 2.25 (1H, quintet), 1.17 (2H, m), 1.03 (2H, m) ppm.

Further examples of compounds that were prepared using this method are listed below in Table 4.

TABLE 4
Compounds made according to the method described in Example 1
above. Characteristic data provided is ¹H-nmr data (400 MHz, d₆-
DMSO) δ_{H} ppm

| Name | Structure | Characteristic data |
|---|---|---|
| 2-(4-chloro-2-fluoro-3- methoxyphenyl)-4,5- dichloro-6- methoxycarbonyl- pyrimidine | 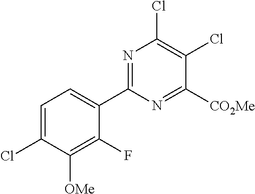 | 7.78 (1H, t), 7.48 (1H, d), 4.00 (3H, s), 3.94 (3H, s) |

### Example 2

### Synthesis of 5-chloro-2-cyclopropyl-6-methoxycarbonyl-4-phenylmethylaminopyrimidine (Compound 4-60) and 5-chloro-2-cyclopropyl-4-phenylmethylamino-6-phenylmethylaminocarbonyl pyrimidine (Compound 265-1)

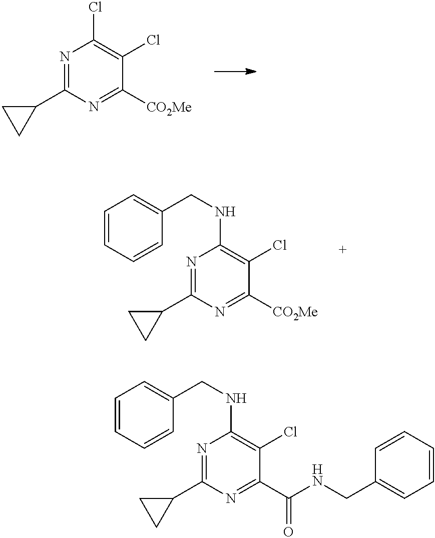

Triethylamine (0.3 ml, 2.1 mmol), followed by benzylamine (0.32 g, 2.16 mmol), were added to a stirred solution of 2-cyclopropyl-4,5-dichloro-6-methoxycarbonylpyrimidine (prepared as described in example 1) (0.26 g, 1.06 mmol) in dichloromethane (3.5 ml). The reaction mixture was stirred at ambient temperature for 66 hours, the solvent evaporated under reduced pressure and the residue purified by column chromatography on silica using 20% ethyl acetate in hexane as eluent to provide 5-chloro-2-cyclopropyl-6-methoxycarbonyl-4-phenylmethylaminopyrimidine as a white solid (71 mg, 21%). Characterising data for this compound are as follows: ¹H nmr (400 MHz, CDCl₃) δ_{H} 7.31 (5H, m), 5.90 (1H, br s), 4.66 (2H, d), 3.98 (3H, s), 2.10 (1H, m), 1.04 (2H, m), 0.80 (2H, m) ppm.

Further elution with 25% ethyl acetate in hexane provided 5-chloro-2-cyclopropyl-4-phenylmethylamino-6-phenylmethylaminocarbonylpyrimidine as a gum (150 mg, 36%). Characterising data for this compound are as follows: ¹H nmr (400 MHz, CDCl₃) δ_{H} 8.32 (1H, br t), 7.32 (10H, m), 6.13 (1H, br t), 4.67 (2H, d), 4.61 (2H, d), 2.02 (1H, m), 1.01 (2H, m), 0.95 (2H, m) ppm.

Further examples of compounds that were prepared using this method are listed below in Table 5. Characteristic data provided is either melting point (° C.), ¹H-nmr data (400 MHz, CDCl₃) δ_{H} ppm or LCMS [retention time (RT, recorded in minutes) and the molecular ion, typically the cation M+H⁺]. Compounds characterised by HPLC-MS used a Waters 2777 injector with a 1525 micro pump HPLC equipped with a Waters Atlantis dC18 IS column (column length 20 mm, internal diameter of column 3 mm, particle size 3 micron), Waters 2996 photodiode array, Waters 2420 ELSD and Micromass ZQ2000. The analysis was conducted using a three minute run time, according to the following gradient table:

| Time | Solvent A | Solvent B | Flow (ml/ |
|---|---|---|---|
| (minutes) | (%) | (%) | minute) |
| 0.00 | 95 | 5 | 1.3 |
| 2.50 | 0 | 100 | 1.3 |
| 2.80 | 0 | 100 | 1.3 |
| 2.90 | 95 | 5 | 1.3 |

Solvent A: H₂O with 0.05% TFA
Solvent B: CH₃CN with 0.05% TFA

TABLE 5
Compounds made according to the method described in Example 2
above.

| Compound | | | Characteristic |
|---|---|---|---|
| Number | Name | Structure | data |
| 4-35 | 5-Chloro-4- cyclohexylamino-2- cyclopropyl-6- methoxycarbonyl- pyrimidine | 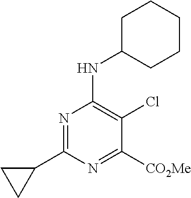 | 89-91 |
| 4-1 | 5-Chloro-2- cyclopropyl-4- cyclopropylamino- 6-methoxycarbonyl- pyrimidine | 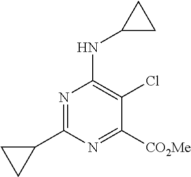 | 5.69 (1H, br s), 3.96 (3H, s), 2.81 (1H, m), 2.14 (1H, m), 1.15 (2H, m), 1.00 (2H, m), 0.87 (2H, m), 0.58 (2H, m) |
| 4-189 | 5-Chloro-2- cyclopropyl-6- methoxycarbonyl-4- (1-methyl-1-phenyl- ethylamino)- pyrimidine | 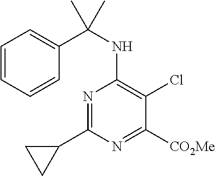 | 98-100 |
| 4-63 | 5-Chloro-4-(4- chlorophenylmethyl- amino)-2- cyclopropyl-6- methoxycarbonyl- pyrimidine | 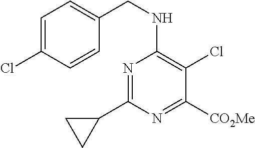 | 118-120 |
| 4-125 | 5-Chloro-2- cyclopropyl-4- (furan-2-ylmethyl- amino)-6-methoxy- carbonylpyrimidine | 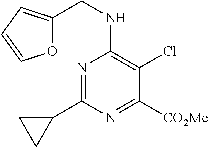 | 88-89; 7.37 (1H, m), 6.33 (1H, m), 6.24 (1H, m), 5.85 (1H, br t), 4.65 (2H, d), 3.97 (3H, s), 2.12 (1H, m), 1.10 (2H, m), 1.00 (2H, m) |
| 4-140 | 5-Chloro-2- cyclopropyl-6- methoxycarbonyl-4- (thiophen-2- ylmethylamino)- pyrimidine | 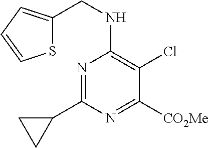 | 86-87; 7.23 (1H, m), 7.00 (1H, m), 6.97 (1H, m), 5.90 (1H, br t), 4.84 (2H, d), 3.97 (3H, s), 2.14 (1H, m), 1.15 (2H, m), 1.01 (2H, m) |
| 265-3 | 5-Chloro-2- cyclopropyl-4- (furan-2- ylmethylamino)-6- (furan-2-yl- methylamino- carbonyl)- pyrimidine | 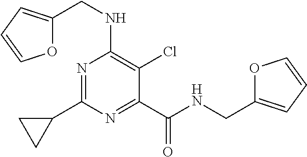 | 8.27 (1H, br t), 7.36 (2H, m), 6.33 (2H, m), 6.28 (1H, m), 6.24 (1H, m), 6.07 (1H, br t), 4.65 (2H, d), 4.57 (2H, d), 2.04 (1H, m), 1.07 (2H, m), 0.99 (2H, m) |
| 265-4 | 5-Chloro-2- cyclopropyl-4- (thiophen-2- ylmethylamino)-6- (thiophen-2-yl- methylamino- carbonyl)- pyrimidine | 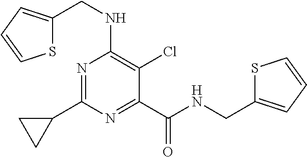 | 8.30 (1H, br t), 7.23 (2H, m), 7.04 (1H, m), 7.00 (1H, m), 6.96 (2H, m), 6.13 (1H, br t), 4.84 (2H, d), 4.76 (2H, d), 2.04 (1H, m), 1.10 (2H, m), 0.99 (2H, m) |
| 4-32 | 5-Chloro-4- cyclobutylamino-2- cyclopropyl-6- methoxycarbonyl- pyrimidine | 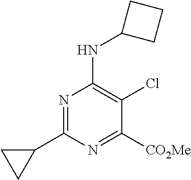 | 5.64 (1H, br d), 4.49 (1H, s), 3.97 (3H, s), 2.41 (2H, m), 2.09 (1H, m), 1.94 (2H, m), 1.83 (2H, m), 1.07 (2H, m), 0.08 (2H, m) |
| 4-33 | 5-Chloro-4- cyclopentylamino- 2-cyclopropyl-6- methoxycarbonyl- pyrimidine | 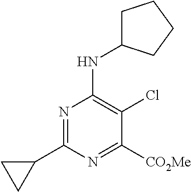 | 69-71 |
| 4-43 | 5-Chloro-2- cyclopropyl-4- cyclopropylmethyl- amino-6- methoxycarbonyl- pyrimidine | 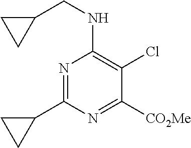 | 5.67 (1H, br s), 3.95 (3H, s), 3.31 (2H, m), 2.10 (1H, m), 1.08 (3H, m), 0.98 (2H, m), 0.59 (2H, m), 0.29 (2H, m) |
| 6-60 | 5-Chloro-2- cyclopropyl-4-([N- ethyl-N- phenylmethyl]- amino)-6- methoxycarbonyl- pyrimidine | 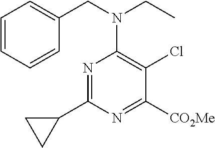 | 7.32 (2H, m), 7.24 (3H, m), 4.83 (2H, s), 3.97 (3H, s), 3.64 (2H, q), 2.05 (1H, quin), 1.26 (3H, t), 0.91 (4H, m) |
| 4-174 | 5-Chloro-2- cyclopropyl-6- methoxycarbonyl-4- (1-phenylethyl- amino)pyrimidine | 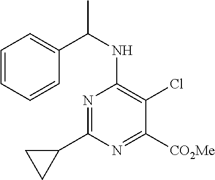 | 101-103 |
| 5-125 | 5-Chloro-2- cyclopropyl-4-(N- furan-2-ylmethyl-N- methyl-amino)-6- methoxycarbonyl- pyrimidine | 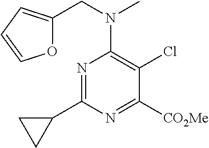 | 7.36 (1H, m), 6.33 (1H, m); 6.23 (1H, m), 4.75 (2H, s), 3.96 (3H, s), 3.21 (3H, s), 2.08 (1H, m), 1.06 (2H, m), 0.97 (2H, m) |
| 4-53 | 5-Chloro-2- cyclopropyl-6- methoxy carbonyl- 4-(tetrahydrofuran- 2-ylmethylamino)- pyrimidine | 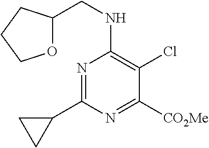 | 5.90 (1H, br t), 4.07 (1H, m), 3.97 (3H, s), 3.90 (1H, m), 3.80 (1H, m), 3.72 (1H, m), 3.47 (1H, m), 2.10 (1H, m), 2.01 (1H, m), 1.92 (2H, m), 1.59 (1H, m), 1.06 (2H, m), 0.97 (2H, m) |
| 4-135 | 5-Chloro-2- cyclopropyl-6- methoxy carbonyl- 4-(5-methylfuran-2- ylmethylamino)- pyrimidine | 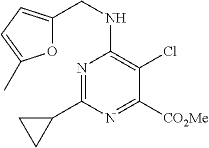 | 63-65 |
| 4-210 | 5-Chloro-2- cyclopropyl-6- methoxycarbonyl-4- (1-methoxy- carbonyl-1-phenyl- methylamino)- pyrimidine | 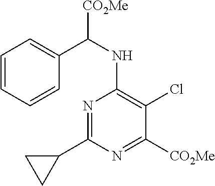 | 7.39 (5H, m), 6.55 (1H, br d), 5.54 (1H, d), 3.97 (3H, s), 3.76 (3H, s), 2.06 (1H, m), 1.00 (2H, m), 0.90 (1H, m), 0.83 (1H, m) |
| 4-6 | 5-Chloro-2- cyclopropyl-4-[1- (2,4-dichlorophenyl) cyclopropyl]-6- methoxycarbonyl- pyrimidine | 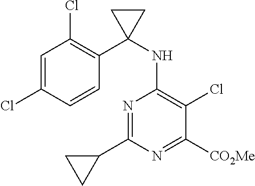 | 7.58 (1H, m), 7.36 (1H, m), 7.20 (1H, m), 6.57 (1H, br s), 3.94 (3H, s), 2.13 (1H, m), 1.28 (4H, s), 1.11 (2H, m), 1.03 (2H, m) |
| 4-42 | 5-Chloro-2- cyclopropyl-4-(3,4- dichlorophenyl- amino)-6- methoxycarbonyl- pyrimidine | 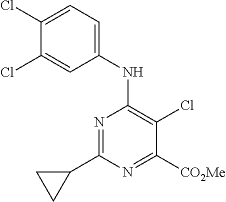 | 7.96 (1H, m), 7.42 (1H, d), 7.38 (1H, m), 7.36 (1H, br s), 4.01 (3H, s), 2.20 (1H, m), 1.10 (4H, m) |
| 4-64 | 5-Chloro-2- cyclopropyl-4-(2- fluorophenylmethyl- amino)-6- methoxycarbonyl- pyrimidine | 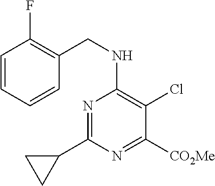 | RT 1.50, MH⁺ 336, 338 |
| 4-61 | 5-Chloro-4-(2- chlorophenyl- methylamino)-2- cyclopropyl-6- methoxycarbonyl- pyrimidine | 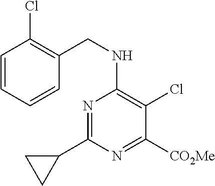 | RT 1.61, MH⁺ 352, 354, 356 |
| 4-103 | 5-Chloro-2- cyclopropyl-4-(2,4- dichlorophenyl- methylamino)-6- methoxycarbonyl- pyrimidine | 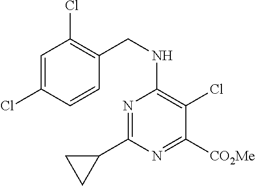 | RT 1.77, MH⁺ 386, 388, 390, 392 |
| 4-62 | 5-Chloro-3-(3- chlorophenyl- methylamino)-2- cyclopropyl-6- methoxycarbonyl- pyrimidine | 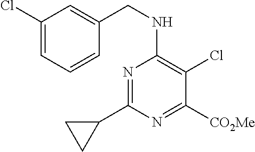 | RT 1.59, MH⁺ 352, 354, 356 |
| 4-66 | 5-Chloro-2- cyclopropyl-4-(4- fluorophenyl- methylamino)-6- methoxy- carbonylpyrimidine | 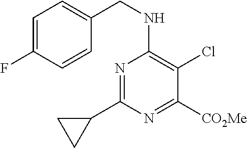 | RT 1.49, MH⁺ 336, 338 |
| 4-116 | 5-Chloro-4-(4- chloro-3-methoxy- phenylmethyl- amino)-2- cyclopropyl-6- methoxycarbonyl- pyrimidine | 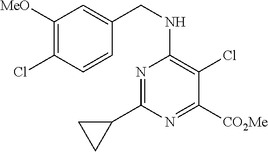 | 6.86 (1H, d), 6.81 (1H, m), 5.86 (1H, m), 5.75 (1H, br t), 4.57 (2H, d), 3.95 (3H, s), 3.87 (3H, s), 2.11 (1H, m), 1.03 (2H, m), 0.96 (2H, m) |
| 4-77 | 5-Chloro-2- cyclopropyl-6- methoxycarbonyl-4- (4-trifluoromethyl- phenylmethyl- amino)pyrimidine | 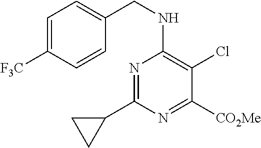 | RT 1.66, MH⁺ 386, 388 |
| 4-74 | 5-Chloro-2- cyclopropyl-4-(4- isopropylphenyl- methlamino)-6- methoxycarbonyl- pyrimidine | 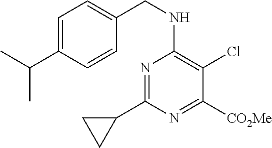 | RT 1.73, MH⁺ 360, 362 |
| 4-121 | 5-Chloro-2- cyclopropyl-4-(2,4- dichloro-6- methylphenylmethyl- amino)-6-methoxy- carbonylpyrimidine | 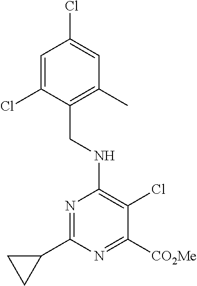 | RT 1.91, MH⁺ 400, 402, 404, 406 |
| 4-111 | 5-Chloro-2- cyclopropyl-4-(2,6- dimethoxyphenyl- methylamino)-6- methoxycarbonyl- pyrimidine | 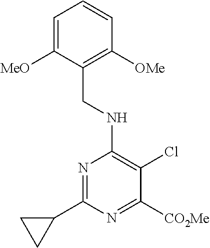 | RT 1.53, MH⁺ 378, 380 |
| 4-104 | 5-Chloro-2- cyclopropyl-4-(2,6- dichlorophenyl- methylamino)-6- methoxycarbonyl- pyrimidine | 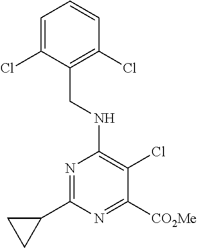 | 124-126 |
| 4-85 | 4-(2-Aminophenyl- methylamino)-5- chloro-2-cyclo- propyl-6-methoxy- carbonylpyrimidine | 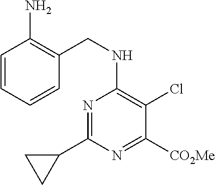 | RT 1.10, MH⁺ 333, 335 |
| 4-82 | 4-(4-ⁿButoxyphenyl- methylamino)-5- chloro-2-cyclo- propyl-6-methoxy- carbonylpyrimidine | 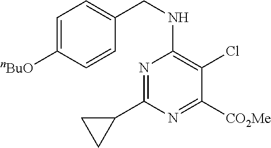 | RT 1.77, MH⁺ 390, 392 |
| 4-112 | 5-Chloro-2- cyclopropyl-4-(3,4- dimethoxyphenyl- methylamino)-6- methoxycarbonyl- pyrimidine | 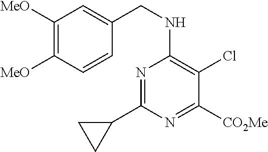 | RT 1.33, MH⁺ 378, 380 |
| 4-100 | 5-Chloro-2- cyclopropyl-4-(3,5- difluorophenylmeth- ylamino)-6- methoxy- carbonylpyrimidine | 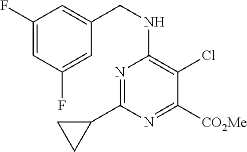 | RT 1.56, MH⁺ 354, 356 |
| 4-72 | 5-Chloro-2- cyclopropyl-6- methoxycarbonyl-4- (3-methylphenyl- methylamino)- pyrimidine | 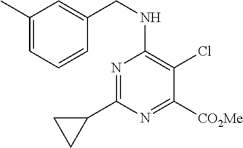 | RT 1.54, MH⁺ 332, 334 |
| 4-80 | 5-Chloro-2- cyclopropyl-6- methoxycarbonyl-4- (4-methoxyphenyl- methylamino)- pyrimidine | 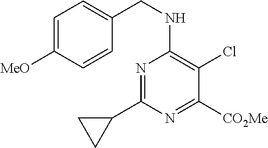 | RT 1.43, MH⁺ 348, 350 |
| 4-75 | 5-Chloro-2- cyclopropyl-6- methoxycarbonyl-4- (2-trifluoromethyl- phenylmethyl- amino)-pyrimidine | 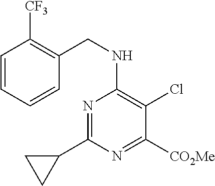 | RT 1.68, MH⁺ 386, 388 |
| 4-76 | 5-Chloro-2- cyclopropyl-6- methoxycarbonyl-4- (3-trifluoromethyl- phenylmethyl- amino)pyrimidine | 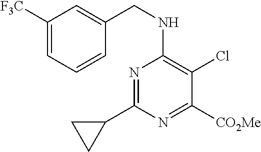 | RT 1.64, MH⁺ 386, 388 |
| 4-115 | 4-(Benzo[1,3]- dioxol-5-ylmethyl- amino)-5-chloro-2- cyclopropyl-6- methoxycarbonyl- pyrimidine | 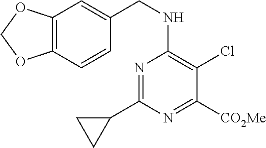 | RT 1.43, MH⁺ 362, 364 |
| 4-78 | 5-Chloro-2- cyclopropyl-6- methoxycarbonyl-4- (2-methoxyphenyl- methylamino)- pyrimidine | 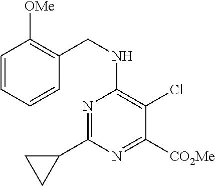 | RT 1.49, MH⁺ 348, 350 |
| 4-91 | 5-Chloro-2- cyclopropyl-6- methoxycarbonyl-4- (3-nitrophenyl- methylamino)- pyrimidine | 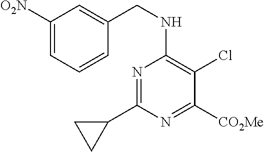 | RT 1.49, MH⁺ 363, 365 |
| 4-109 | 5-Chloro-2- cyclopropyl-4-(2,3- dimethoxyphenyl- methylamino)-6- methoxycarbonyl- pyrimidine | 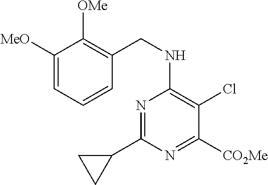 | RT 1.45, MH⁺ 378, 380 |
| 4-86 | 4-(4-Aminophenyl- methylamino)-5- chloro-2- cyclopropyl-6- methoxycarbonyl- pyrimidine | 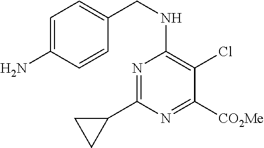 | RT 0.97, MH⁺ 333, 335 |
| 4-122 | 5-Chloro-2- cyclopropyl-6- methoxycarbonyl-4- (3,4,5-trimethoxy- phenylmethyl- amino)pyrimidine | 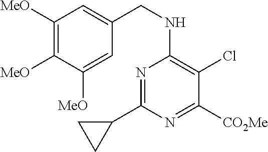 | RT 1.34, MH⁺ 408, 410 |
| 4-87 | 5-Chloro-2- cyclopropyl-4-(4- dimethylamino- phenylmethyl- amino)-6- methoxycarbonyl- pyrimidine | 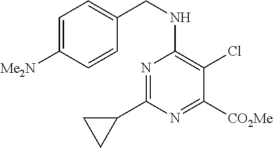 | RT 1.02, MH⁺ 361, 363 |
| 4-83 | 5-Chloro-2- cyclopropyl-6- methoxycarbonyl-4- (4-trifluoromethoxy- phenylmethyl- amino)pyrimidine | 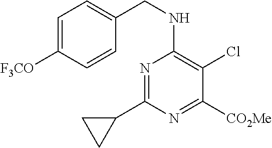 | RT 1.69, MH⁺ 402, 404 |
| 4-73 | 5-Chloro-2- cyclopropyl-6- methoxycarbonyl-4- (4-methylphenyl- methylamino)- pyrimidine | 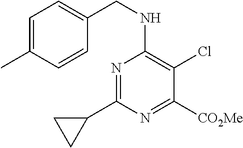 | RT 1.55, MH⁺ 332, 334 |
| 4-68 | 4-(3-Bromophenyl- methylamino)5- chloro-2- cyclopropyl-6- methoxycarbonyl- pyrimidine | 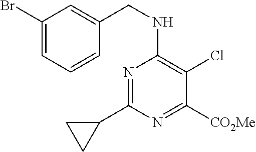 | RT 1.62, MH⁺ 396, 398, 400 |
| 4-70 | 5-Chloro-2- cyclopropyl-4-(3- iodophenylmethyl- amino)-6-methoxy- carbonylpyrimidine | 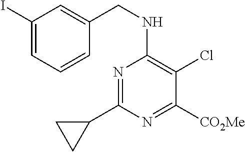 | RT 1.66, MH⁺ 444, 446 |
| 4-71 | 5-Chloro-2- cyclopropyl-6- methoxycarbonyl-4- (2-methylphenyl- methylamino)- pyrimidine | 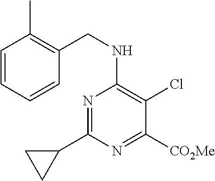 | RT 1.55, MH⁺ 332, 334 |
| 4-65 | 5-Chloro-2- cyclopropyl-4-(3- fluorophenylmethyl- amino)-6- methoxycarbonyl- pyrimidine | 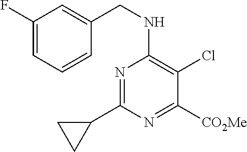 | RT 1.50, MH⁺ 336, 338 |
| 4-107 | 5-Chloro-2- cyclopropyl-4-(3- fluoro-5-trifluoro- methylphenyl- methylamino)-6- methoxycarbonyl- pyrimidine | 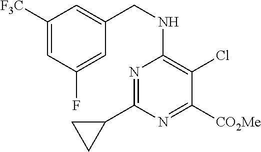 | RT 1.70, MH⁺ 404, 406 |
| 4-108 | 4-(3,5-Bis-trifluoro- methylphenyl- methylamino)-5- chloro-2-cyclo- propyl-6-methoxy- carbonylpyrimidine | 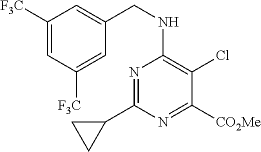 | RT 1.82, MH⁺ 454, 456 |
| 4-95 | 5-Chloro-2- cyclopropyl-6- methoxycarbonyl-4- (3-phenylphenyl- methylamino)- pyrimidine | 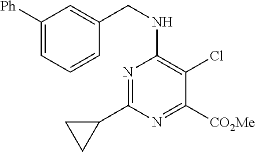 | RT 1.74, MH⁺ 394, 396 |
| 4-99 | 5-Chloro-2- cyclopropyl-4-(2,6- difluorophenylmeth- ylamino)-6- methoxycarbonyl- pyrimidine | 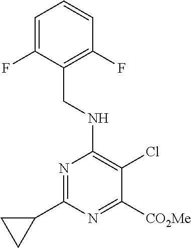 | RT 1.52, MH⁺ 354, 356 |
| 4-67 | 4-(2-Bromophenyl- methylamino)-5- chloro-2- cyclopropyl-6- methoxycarbonyl- pyrimidine | 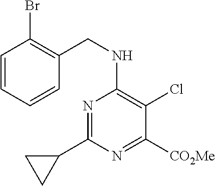 | RT 1.65, MH⁺ 396, 398, 400 |
| 4-97 | 5-Chloro-2- cyclopropyl-4-(2,4- difluorophenyl- methylamino)-6- methoxycarbonyl- pyrimidine | 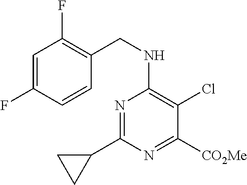 | RT 1.55, MH⁺ 354, 356 |
| 4-98 | 5-Chloro-2- cyclopropyl-4-(2,5- difluorophenyl- methyl-amino)-6- methoxycarbonyl- pyrimidine | 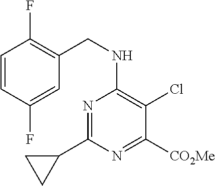 | RT 1.54, MH⁺ 354, 356 |
| 4-90 | 5-Chloro-2- cyclopropyl-6- methoxycarbonyl-4- (2-nitrophenyl- methylamino)- pyrimidine | 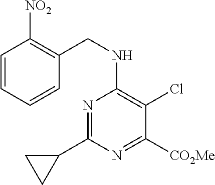 | RT 1.51, MH⁺ 363, 365 |
| 4-96 | 5-Chloro-2- cyclopropyl-4-(2,3- difluorophenyl- methylamino)-6- methoxycarbonyl- pyrimidine | 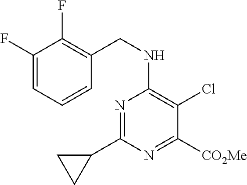 | RT 1.55, MH⁺ 354, 356 |
| 4-101 | 5-Chloro-4-(4- chloro-2-fluoro- phenylmethyl- amino)-2- cyclopropyl-6- methoxycarbonyl- pyrimidine | 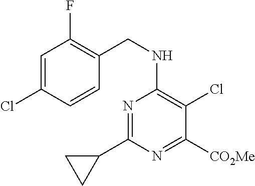 | RT 1.66, MH⁺ 370, 372, 374 |
| 4-118 | 5-Chloro-2- cyclopropyl-6- methoxycarbonyl-4- (2,3,6-trifluoro- phenylmethyl- amino)pyrimidine | 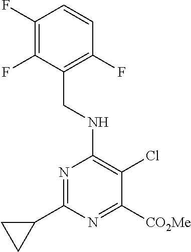 | RT 1.56, MH⁺ 372, 374 |
| 4-120 | 5-Chloro-2- cyclopropyl-6- methoxycarbonyl-4- (2,4,5-trifluoro- phenylmethyl- amino)pyrimidine | 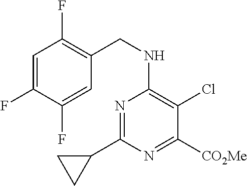 | RT 1.60, MH⁺ 372, 374 |
| 4-94 | 5-Chloro-2- cyclopropyl-6- methoxycarbonyl-4- (4-methoxy- carbonylphenyl- methylamino)- pyrimidine | 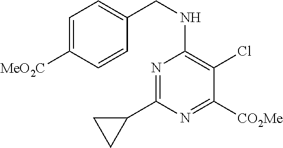 | RT 1.46, MH⁺ 376, 378 |
| 4-88 | 5-Chloro-2- cyclopropyl-6- methoxycarbonyl-4- (2-morpholin-4-yl- phenylmethyl- amino)-pyrimidine | 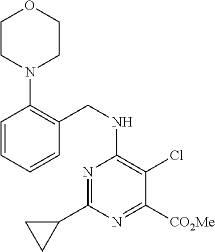 | RT 1.48, MH⁺ 403, 405 |
| 4-110 | 5-Chloro-2- cyclopropyl-4-(2,4- dimethoxyphenyl- methylamino)-6- methoxycarbonyl- pyrimidine | 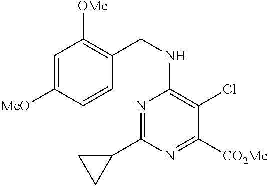 | RT 1.49, MH⁺ 378, 380 |
| 5-61 | 5-Chloro-4-[N-(2- chlorophenyl- methyl)-N-methyl- amino]-2- cyclopropyl-6- methoxycarbonyl- pyrimidine | 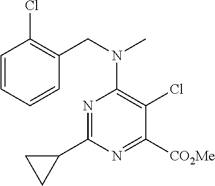 | RT 1.88, MH⁺ 366, 368, 370 |
| 161-60 | 5-Chloro-4-[N-(2- cyanoethyl)-N- phenylmethyl- amino]-2- cyclopropyl-6- methoxycarbonyl- pyrimidine | 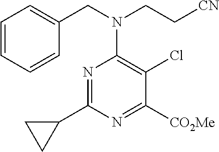 | RT 1.65, MH⁺ 371, 373 |
| 162-60 | 5-Chloro-2- cyclopropyl-4-[N-(2- ethoxycarbonyl- ethyl)-N- phenylmethyl- amino]-6- methoxycarbonyl- pyrimidine | 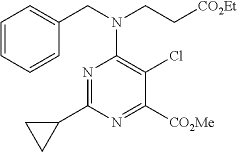 | RT 1.82, MH⁺ 418, 420 |
| 150-60 | 5-Chloro-2- cyclopropyl-6- methoxycarbonyl-4- (N-phenylmethyl-N- trimethylsilylmethyl- amino)pyrimidine | 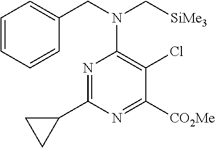 | RT 2.07, MH⁺ 405, 407 |
| 5-77 | 5-Chloro-2- cyclopropyl-6- methoxycarbonyl-4- [N-methyl-N-(4- trifluoromethyl- phenyl)methyl- amino]pyrimidine | 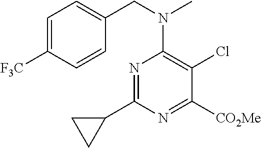 | RT 1.90, MH⁺ 400, 402 |
| 5-73 | 5-Chloro-2- cyclopropyl-6- methoxycarbonyl-4- [N-methyl-N-(4- methylphenyl)- methylamino]- pyrimidine | 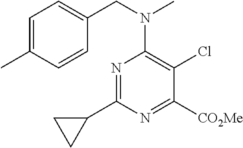 | RT 1.85, MH⁺ 346, 348 |
| 5-80 | 5-Chloro-2- cyclopropyl-6- methoxycarbonyl-4- [N-(4-methoxy- phenyl)methyl-N- methyl-amino]- pyrimidine | 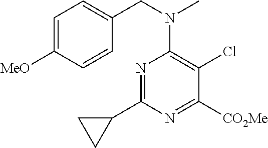 | RT 1.72, MH⁺ 362, 364 |
| 151-64 | 5-Chloro-2-cyclo- propyl-4-[N(2- fluorophenyl)methyl- N-prop-2-enyl- amino]-6-methoxy- carbonylpyrimidine | 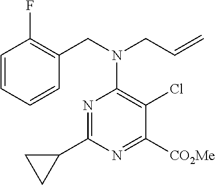 | RT 1.92, MH⁺ 376, 378 |
| 154-60 | 4-(N-But-3-enyl-N- phenylmethyl- amino)-5-chloro-2- cyclopropyl-6- methoxycarbonyl- pyrimidine | 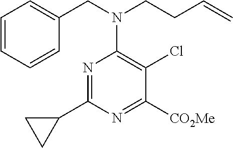 | RT 1.96, MH⁺ 372, 374 |
| 6-64 | 5-Chloro-2- cyclopropyl-4-[N- ethyl-N-(2-fluoro- phenyl)methyl- amino]-6- methoxycarbonyl- pyrimidine | 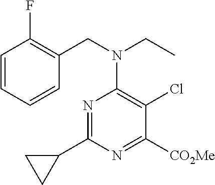 | RT 1.86, MH⁺ 364, 366 |
| 4-175 | 5-Chloro-2- cyclopropyl-4-[1-(4- fluorophenyl)ethyl- amino]-6-methoxy- carbonylpyrimidine | 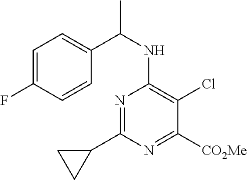 | RT 1.59, MH⁺ 350, 352 |
| 4-176 | 5-Chloro-4-[1-(4- chlorophenyl)ethyl- amino]-2-cyclo- propyl-6-methoxy- carbonylpyrimidine | 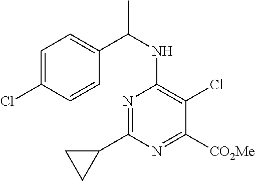 | RT 1.70, MH⁺ 366, 368, 370 |
| 4-194 | 5-Chloro-2- cyclopropyl-6- methoxycarbonyl-4- (1-phenylpropyl- amino)pyrimidine | 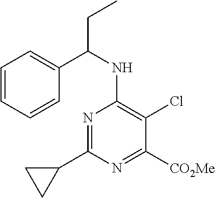 | RT 1.65, MH⁺ 346, 348 |
| 4-178 | 5-Chloro-2- cyclopropyl-6- methoxycarbonyl-4- [1-(4-n-pentyl- phenyl)ethylamino]- pyrimidine | 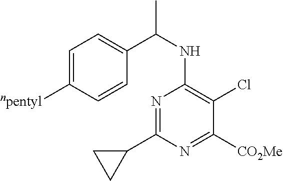 | RT 2.07, MH⁺ 402, 404 |
| 4-278 | 5-Chloro-4-[(4- chlorophenyl)- (phenyl)- methylamino]-2- cyclopropyl-6- methoxycarbonyl- pyrimidine | 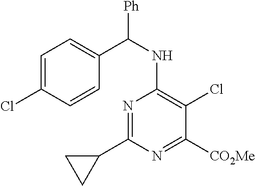 | RT 1.95, MH⁺ 428, 430, 432 |
| 4-183 | 5-Chloro-2- cyclopropyl-4-[1- (2,4-dimethyl- phenyl)ethylamino]- 6-methoxycarbonyl- pyrimidine | 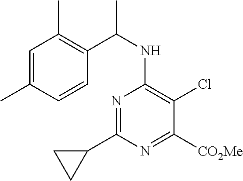 | RT 1.73, MH⁺ 360, 362 |
| 4-180 (S-isomer) | (S)-5-Chloro-2- cyclopropyl-6- methoxycarbonyl-4- [1-(3-methoxy- phenyl) ethylamino]- pyrimidine | 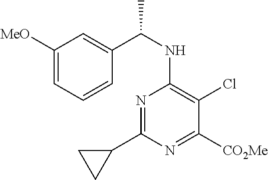 | RT 1.54, MH⁺ 362, 364 |
| 4-181 (S-isomer) | (S)-5-Chloro-2- cyclopropyl-6- methoxycarbonyl-4- [1-(4-methoxy- phenyl)ethylamino]- pyrimidine | 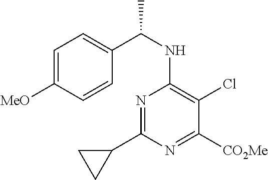 | RT 1.53, MH⁺ 362, 364 |
| 4-277 | 5-Chloro-2- cyclopropyl-4- (diphenylmethyl- amino)-6-methoxy- carbonylpyrimidine | 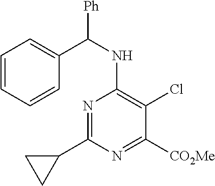 | RT 1.82, MH⁺ 394, 396 |
| 4-197 | 5-Chloro-2- cyclopropyl-6- methoxycarbonyl-4- (1-phenylprop-2-en- 1-yl-amino)- pyrimidine | 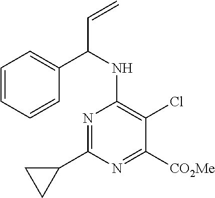 | RT 1.66, MH⁺ 344, 346 |
| 4-198 | 5-Chloro-4-[1-(4- chlorophenyl)but-3- en-1-yl-amino]-2- cyclopropyl-6- methoxycarbonyl- pyrimidine | 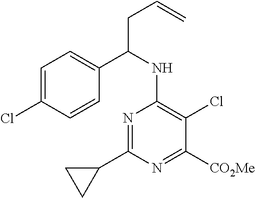 | RT 1.85, MH⁺ 392, 394, 396 |
| 4-5 | 5-Chloro-4-[1-(4- chlorophenyl)cyclo- propylamino]-2- cyclopropyl-6- methoxycarbonyl- pyrimidine | 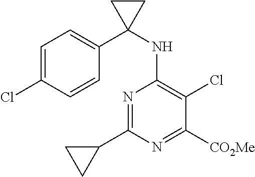 | RT 1.85, MH⁺ 378, 380, 382 |
| 4-177 | 4-[1-(4- Bromophenyl)ethyl- amino]-5-chloro-2- cyclopropyl-6- methoxycarbonyl- pyrimidine | 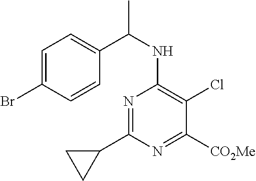 | RT 1.73, MH⁺ 410, 412, 414 |
| 4-200 | 5-Chloro-4-[1-(4- chlorophenyl)-1- (cyclopropyl)methyl- amino]-2- cyclopropyl-6- methoxycarbonyl- pyrimidine | 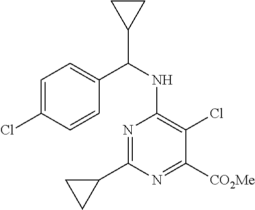 | RT 1.77, MH⁺ 392, 394, 396 |
| 4-179 | 5-Chloro-2- cyclopropyl-6- methoxycarbonyl-4- [1-(3-trifluoro- methylphenyl)ethyl- amino]pyrimidine | 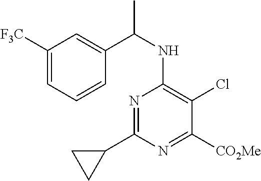 | RT 1.73, MH⁺ 400, 402 |
| 4-214 | 5-Chloro-2- cyclopropyl-4-[(1- ethoxycarbonyl-1- phenyl)-methyl- amino]-6-methoxy- carbonylpyrimidine | 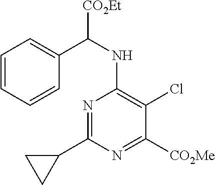 | RT 1.70, MH⁺ 390, 392 |
| 4-151 | 5-Chloro-2- cyclopropyl-6- methoxycarbonyl-4- (pyrid-2-yl- methylamino)- pyrimidine | 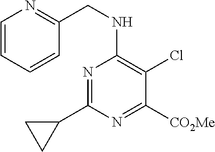 | RT 1.02, MH⁺ 319, 321 |
| 4-160 | 5-Chloro-2- cyclopropyl-6- methoxycarbonyl-4- (pyrid-4-yl-methyl- amino)pyrimidine | 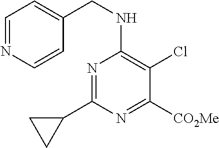 | RT 0.91, MH⁺ 319, 321 |
| 4-156 | 5-Chloro-2- cyclopropyl-6- methoxycarbonyl-4- (pyrid-3-ylmethyl- amino)pyrimidine | 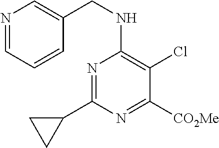 | RT 1.55, MH⁺ 319, 321 |
| 4-155 | 5-Chloro-4-(3- chloro-5- trifluoromethylpyrid- 2-ylmethylamino)-2- cyclopropyl-6- methoxycarbonyl- pyrimidine | 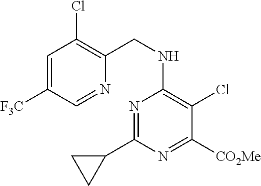 | RT 1.70, MH⁺ 421, 423, 425 |
| 4-159 | 5-Chloro-2- cyclopropyl-6- methoxycarbonyl-4- (2-trifluoromethyl- pyrid-5-yl-methyl- amino)-pyrimidine | 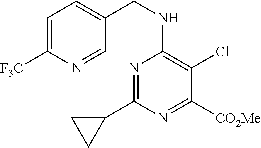 | RT 1.45, MH⁺ 387, 389 |
| 4-163 | 5-Chloro-2- cyclopropyl-6- methoxycarbonyl-4- (5-methylpyrazin-2- ylmethylamino)- pyrimidine | 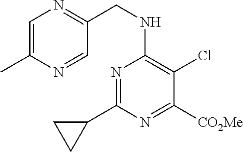 | RT 1.11, MH⁺ 334, 336 |
| 4-146 | 5-Chloro-4-(2- chlorothiazol-5- ylmethylamino)-2- cyclopropyl-6- methoxycarbonyl- pyrimidine | 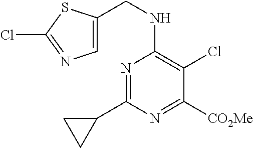 | RT 1.45, MH⁺ 359, 361, 363 |
| 4-166 | 4-(Benzimidazol-2- ylmethylamino)-5- chloro-2- cyclopropyl-6- methoxycarbonyl- pyrimidine | 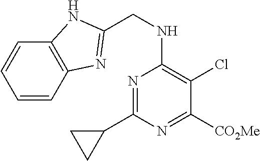 | RT 1.45, MH⁺ 358, 360 |
| 4-143 | 4-(2- Bromothiophen-3- ylmethylamino)-5- chloro-2- cyclopropyl-6- methoxycarbonyl- pyrimidine | 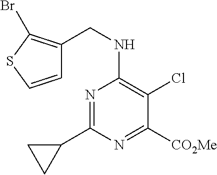 | RT 1.62, MH⁺ 402, 404, 406 |
| 4-144 | 5-Chloro-2- cyclopropyl-4-(1,5- dimethylpyrazol-3- ylmethylamino)-6- methoxycarbonyl- pyrimidine | 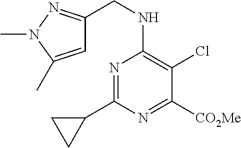 | RT 1.15, MH⁺ 336, 338 |
| 4-168 | 4-(Benzothiazol-2- ylmethylamino)-5- chloro-2- cyclopropyl-6- methoxycarbonyl- pyrimidine | 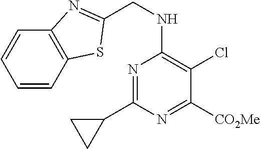 | RT 1.49, MH⁺ 375, 377 |
| 4-152 | 5-Chloro-4-(3- chloropyrid-2- ylmethylamino)-2- cyclopropyl-6- methoxycarbonyl- pyrimidine | 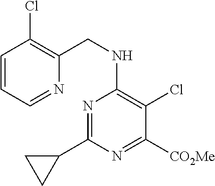 | RT 1.41, MH⁺ 353, 355, 357 |
| 4-153 | 5-Chloro-2- cyclopropyl-6- methoxycarbonyl-4- (3-methylpyrid-2- ylmethylamino)- pyrimidine | 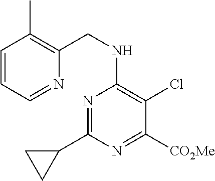 | 120-121 |
| 4-184 (R-isomer) | (R)-5-Chloro-2- cyclopropyl-6- methoxycarbonyl-4- [1-(naphth-1- yl)ethylamino]- pyrimidine | 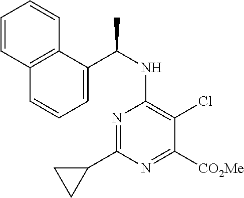 | RT 1.71, MH⁺ 382, 384 |
| 4-185 (R-isomer) | (R)-5-Chloro-2- cyclopropyl-6- methoxycarbonyl-4- [1-(naphtha-2- yl)ethylamino]- pyrimidine | 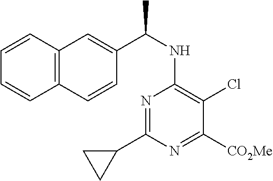 | RT 1.74, MH⁺ 382, 384 |
| 4-10 | 5-Chloro-2- cyclopropyl-6- methoxycarbonyl-4- (1-pyrid-2-ylcyclo- propylamino)- pyrimidine | 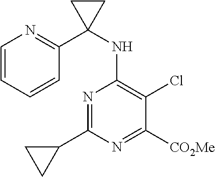 | RT 0.99, MH⁺ 345, 347 |
| 4-188 | 5-Chloro-2- cyclopropyl-6- methoxycarbonyl-4- (1-pyrid-2-yl- ethylamino)- pyrimidine | 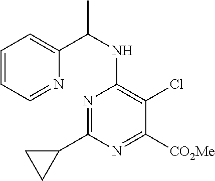 | RT 0.96, MH⁺ 333, 335 |
| 4-199 | 5-Chloro-2- cyclopropyl-6- methoxycarbonyl-4- [1-(pyrid-2-yl)-but- 3-enylamino]- pyrimidine | 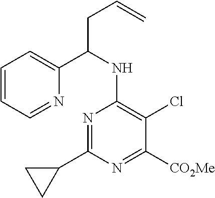 | RT 1.09, MH⁺ 359, 361 |
| 4-13 | 5-Chloro-2- cyclopropyl-6- methoxycarbonyl-4- [1-(6-methylpyrid-2- yl)-cyclopropyl- amino]-pyrimidine | 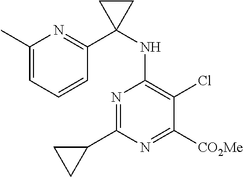 | RT 0.99, MH⁺ 359, 361 |
| 146-60 | 5-Chloro-2- cyclopropyl-4-[(N- isopropyl-N- phenylmethyl)- amino]-6- methoxycarbonyl- pyrimidine | 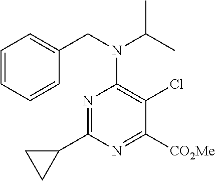 | RT 1.89, MH⁺ 360, 362 |
| 155-41 | 5-Chloro-2- cyclopropyl-6- methoxycarbonyl-4- {[N-(4- methoxyphenyl)-N- phenylmethyl]- amino}-pyrimidine | 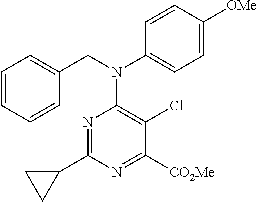 | RT 1.92, MH⁺ 424, 426 |
| 4-39 | 5-Chloro-2- cyclopropyl-6- methoxycarbonyl-4- phenylamino- pyrimidine | 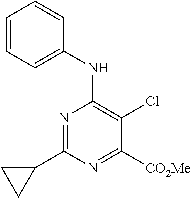 | 86-87 |
| 4-171 | 5-Chloro-2- cyclopropyl-4-(1- cyclopropylethyl- amino)-6-methoxy- carbonylpyrimidine | 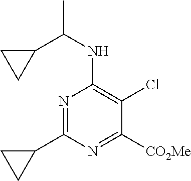 | 5.55 (1H, br d), 3.96 (3H, s), 3.59 (1H, m), 2.08 (1H, m), 1.27 (3H, d), 1.02 (2H, m), 0.94 (3H, m), 0.56 (1H, m), 0.48 (1H, m), 0.34 (1H, m), 0.27 (1H, m) |
| 4-283 | 5-Chloro-2- cyclopropyl-6- methoxycarbonyl-4- (1-methoxy- carbonyl-2- phenylethylamino)- pyrimidine | 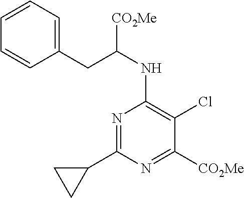 | 83-84 |
| 4-17 | 5-Chloro-2- cyclopropyl-6- methoxycarbonyl-4- [1-(2-phenylethyl)- cyclopropylamino]- pyrimidine | 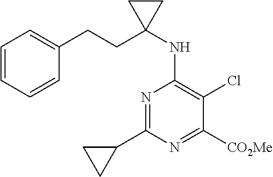 | 7.24 (2H, m), 7.17 (1H, m), 7.13 (2H, m), 5.75 (1H, br s), 3.97 (3H, s), 2.70 (2H, m), 2.13 (1H, m), 1.94 (2H, m), 1.10 (2H, m), 1.00 (2H, m), 0.74 (4H, m) |
| 4-148 | 5-Chloro-2- cyclopropyl-6- methoxycarbonyl-4- (oxazol-2- ylmethylamino)- pyrimidine | 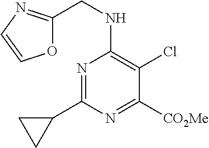 | 114-115 |
| 4-279 | 5-Chloro-2- cyclopropyl-4-(1,2- diphenylethyl- amino)-6-methoxy- carbonylpyrimidine | 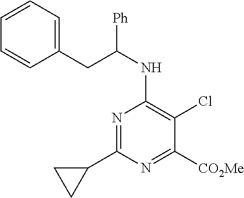 | RT 1.79, MH⁺ 408, 410 |
| 4-231 | 5-Chloro-2- cyclopropyl-4-[2- (indol-3-yl)-1- methyl-ethylamino]- 6-methoxy- carbonylpyrimidine | 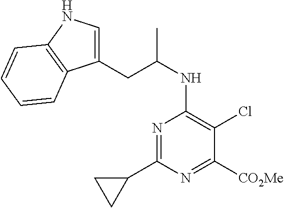 | RT 1.48, MH⁺ 385, 387 |
| 4-230 | 5-Chloro-4-(2- cyclopentyl-1- methyl-ethylamino)- 2-cyclopropyl-6- methoxycarbonyl- pyrimidine | 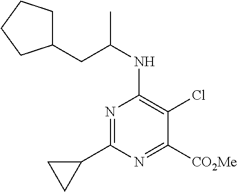 | RT 1.70, MH⁺ 338, 340 |
| 4-268 | 5-Chloro-2- cyclopropyl-6- methoxycarbonyl-4- (2-phenoxy-1- methylethylamino)- pyrimidine | 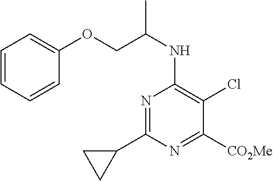 | RT 1.61, MH⁺ 362, 364 |
| 4-269 | 5-Chloro-4-[2-(4- cyanophenoxy)-1- methyl-ethylamino]- 2-cyclopropyl-6- methoxycarbonyl- pyrimidine | 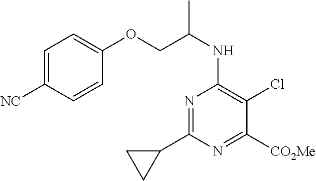 | RT 1.54, MH⁺ 387, 389 |
| 4-252 | 5-Chloro-2- cyclopropyl-6- methoxycarbonyl-4- (3-phenyl-1-methyl- propylamino)- pyrimidine | 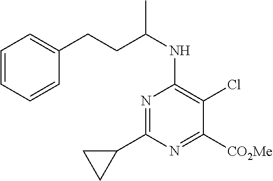 | RT 1.60, MH⁺ 360, 362 |
| 4-173 (S isomer) | 5-Chloro-4-[(1S)-1- (cyclohexyl)ethyl- amino]-2- cyclopropyl-6- methoxycarbonyl- pyrimidine | 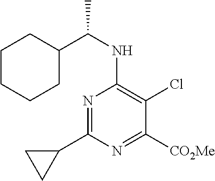 | RT 1.70, MH⁺ 338, 340 |
| 4-290 | 5-Chloro-2- cyclopropyl-4-(2- indol-3-yl-1- methoxycarbonyl- ethylamino)-6- methoxycarbonyl- pyrimidine | 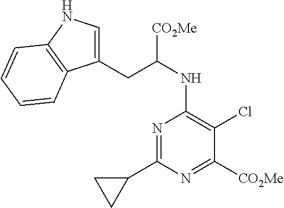 | RT 1.56, MH⁺ 429, 431 |
| 4-292 (R isomer) | 5-Chloro-2- cyclopropyl-4-[(1R)- 1-ethoxycarbonyl-2- phenylthio- ethylamino]-6- methoxycarbonyl- pyrimidine | 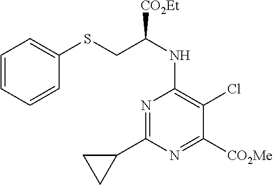 | RT 1.77, MH⁺ 450, 452 |
| 4-287 (S isomer) | 5-Chloro-2- cyclopropyl-4-[(1S)- 2-(4- hydroxyphenyl)-1- methoxycarbonyl- ethylamino]-6- methoxycarbonyl- pyrimidine | 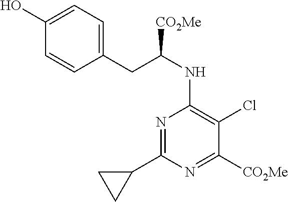 | RT 1.36, MH⁺ 406, 408 |
| 4-272 (S isomer) | 5-Chloro-2- cyclopropyl-6- methoxycarbonyl-4- [(1S)-1- (phenylmethoxy- carbonyl)ethylamino]- pyrimidine | 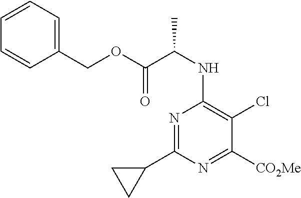 | RT 1.59, MH⁺ 390, 392 |
| 4-293 (S isomer) | 4-[(1S)-1- ^{t}Butoxycarbonyl-2- phenyl-ethylamino]- 5-chloro-2- cyclopropyl-6- methoxycarbonyl- pyrimidine | 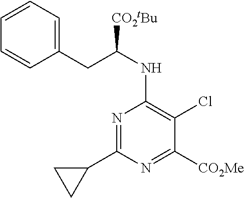 | RT 1.89, MH⁺ 432, 434 |
| 4-285 (S isomer) | 5-Chloro-4-[(1S)- 2-(4-chlorophenyl)- 1-methoxycarbonyl- ethylamino]-2- cyclopropyl-6- methoxycarbonyl- pyrimidine | 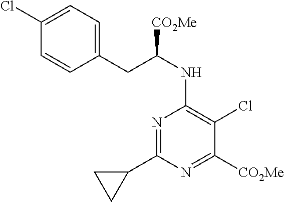 | RT 1.71, MH⁺ 424, 426, 428 |
| 5-283 (S isomer) | 5-Chloro-2- cyclopropyl-6- methoxycarbonyl-4- {N-[(1S)-1- methoxycarbonyl-2- phenyl-ethyl]-N- methyl-amino}- pyrimidine | 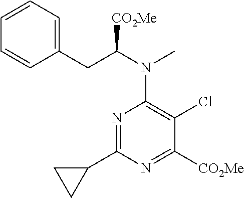 | RT 1.69, MH⁺ 404, 406 |
| 4-284 | 5-Chloro-2- cyclopropyl-4-[2-(4- fluorophenyl)-1- methoxycarbonyl- ethylamino]-6- methoxycarbonyl- pyrimidine | 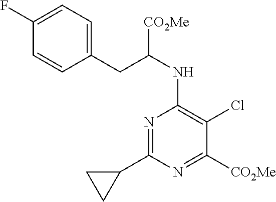 | RT 1.61, MH⁺ 408, 410 |
| 4-16 | 5-Chloro-4-[1-(4- chlorophenoxy- methyl)cyclo- propylamino]-2- cyclopropyl-6- methoxycarbonyl- pyrimidine | 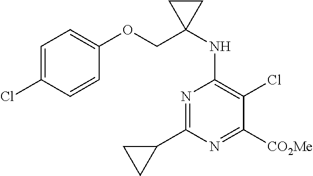 | RT 1.73, MH⁺ 408, 410, 412 |
| 4-286 (S isomer) | 5-Chloro-2- cyclopropyl-6- methoxycarbonyl-4- [(1S)-1-methoxy- carbonyl-2-(3- trifluoromethyl- phenyl)- ethylamino]- pyrimidine | 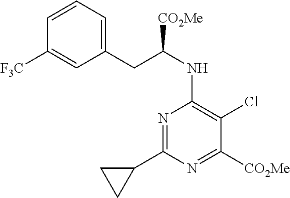 | RT 1.74, MH⁺ 458, 460, 462 |
| 4-282 (S isomer) | 5-Chloro-4-[(1S)-2- cyclohexyl-1- methoxycarbonyl- ethylamino]-2- cyclopropyl-6- methoxycarbonyl- pyrimidine | 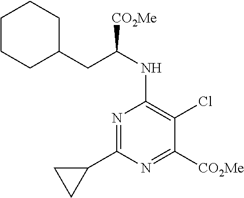 | RT 1.84, MH⁺ 396, 398 |
| 4-288 (S isomer) | 4-[(1S)-2-(4- ^{t}Butoxyphenyl)-1- methoxycarbonyl- ethylamino]-5- chloro-2- cyclopropyl-6- methoxycarbonyl- pyrimidine | 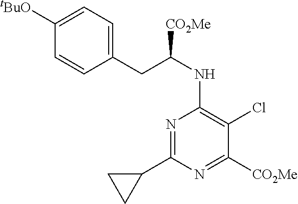 | RT 1.76, MH⁺ 462, 464 |
| 4-261 | 5-Chloro-2- cyclopropyl-6- methoxycarbonyl-4- [2-(naphth-1-yl- amino)ethylamino]- pyrimidine | 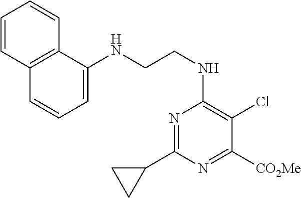 | RT 1.59, MH⁺ 397, 399 |
| 4-226 | 5-Chloro-2- cyclopropyl-4-[2- (indol-3-yl) ethylamino]-6- methoxycarbonyl- pyrimidine | 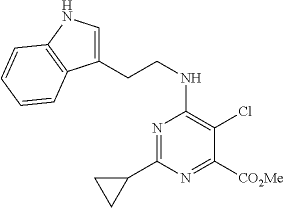 | RT 1.43, MH⁺ 371, 373 |
| 4-260 | 5-Chloro-2- cyclopropyl-6- methoxycarbonyl-4- [2-(phenylamino)- ethylamino)- pyrimidine | 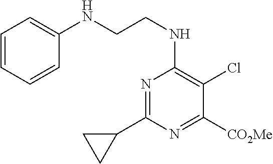 | RT 1.22, MH⁺ 347, 349 |
| 4-221 | 5-Chloro-2- cyclopropyl-6- methoxycarbonyl-4- (2-phenylethyl- amino)-pyrimidine | 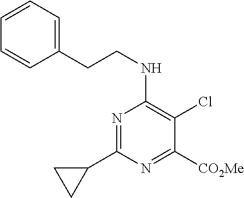 | RT 1.48, MH⁺ 332, 334 |
| 5-221 | 5-Chloro-2- cyclopropyl-6- methoxycarbonyl-4- [(N-methyl)-N-(2- phenylethyl)- amino]pyrimidine | 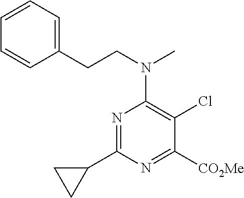 | RT 1.76, MH⁺ 346, 348 |
| 4-266 | 5-Chloro-2- cyclopropyl-6- methoxycarbonyl-4- [2-(phenylmethoxy- carbonyl)ethyl- amino]-pyrimidine | 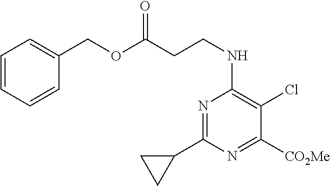 | RT 1.47, MH⁺ 390, 392 |
| 4-267 | 5-Chloro-2- cyclopropyl-4-[2,2- (diphenyl)- ethylamino]-6- methoxycarbonyl- pyrimidine | 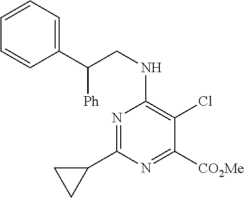 | RT 1.73, MH⁺ 408, 410 |
| 4-227 | 5-Chloro-2- cyclopropyl-6- methoxycarbonyl-4- [2-(5-methoxyindol- 3-yl)-ethylamino]- pyrimidine | 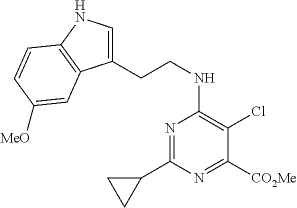 | RT 1.39, MH⁺ 401, 403 |
| 4-253 | 5-Chloro-2- cyclopropyl-6- methoxycarbonyl-4- (4-phenylbutyl- amino)pyrimidine | 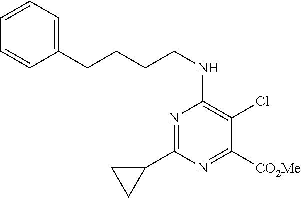 | RT 1.62, MH⁺ 360, 362 |
| 4-49 | 5-Chloro-4- cyclohexylmethyl- amino-2-cyclo- propyl-6-methoxy- carbonylpyrimidine | 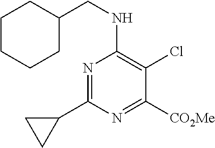 | RT 1.59, MH⁺ 324, 326 |
| 4-247 | 5-Chloro-2- cyclopropyl-6- methoxycarbonyl-4- [3-(2-methyl- piperidin-1-yl)- propylamino]- pyrimidine | 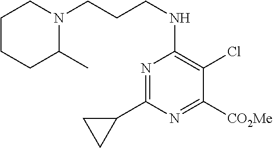 | RT 0.97, MH⁺ 367, 369 |
| 4-249 | 5-Chloro-2- cyclopropyl-6- methoxycarbonyl-4- (3-phenylpropyl- amino)pyrimidine | 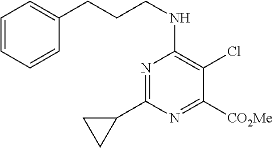 | RT 1.53, MH⁺ 346, 348 |
| 4-233 | 5-Chloro-4-[2-(4- chlorophenyl)propyl- amino]-2- cyclopropyl-6- methoxycarbonyl- pyrimidine | 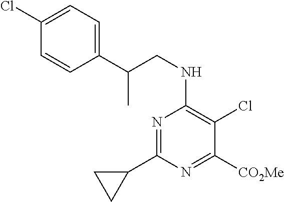 | RT 1.69, MH⁺ 380, 382, 384 |
| 4-263 | 5-Chloro-2- cyclopropyl-4-{2- [(2,6-dichloro- phenyl)methylthio]- ethylamino}-6- methoxycarbonyl- pyrimidine | 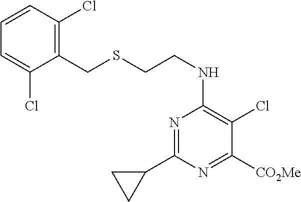 | RT 1.78, MH⁺ 446, 448, 450, 452 |
| 4-232 | 5-Chloro-2- cyclopropyl-6- methoxycarbonyl-4- [2- (phenyl)propyl- amino]-pyrimidine | 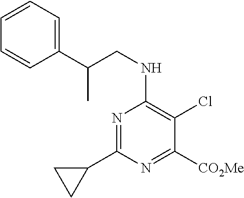 | RT 1.56, MH⁺ 346, 348 |
| 4-250 | 5-Chloro-2- cyclopropyl-4-[3- (imidazol-1- yl)propylamino]-6- methoxycarbonyl- pyrimidine | 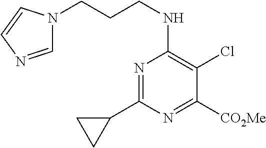 | RT 0.88, MH⁺ 336, 338 |
| 4-228 | 5-Chloro-2- cyclopropyl-6- methoxycarbonyl-4- [2-(pyrid-2- yl)ethylamino]- pyrimidine | 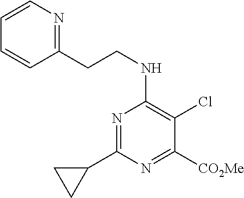 | RT 0.91, MH⁺ 333, 335 |
| 4-224 | 5-Chloro-2- cyclopropyl-6- methoxycarbonyl-4- [2-(thiophen-2- yl)ethylamino]- pyrimidine | 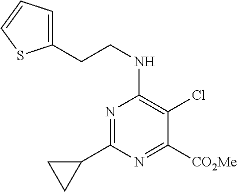 | RT 1.47, MH⁺ 338, 340 |
| 4-246 | 5-Chloro-2- cyclopropyl-6- methoxycarbonyl-4- [3-(2-oxo-pyrrolidin- 1-yl)propylamino]- pyrimidine | 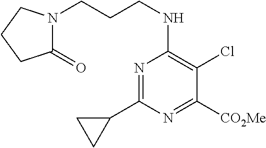 | RT 1.05, MH⁺ 353, 355 |
| 5-54 | 5-Chloro-2- cyclopropyl-4-[N- (1,3-dioxolan-2- yl)methyl-N-methyl- amino]-6- methoxycarbonyl- pyrimidine | 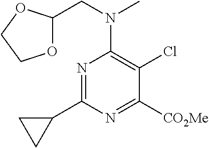 | RT 1.40, MH⁺ 328, 330 |
| 4-220 | 5-Chloro-2- cyclopropyl-6- methoxycarbonyl-4- [2-(morpholin-4- yl)ethylamino]- pyrimidine | 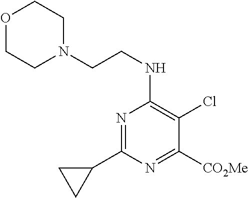 | RT 0.87, MH⁺ 341, 343 |
| 5-228 | 5-Chloro-2- cyclopropyl-6- methoxycarbonyl-4- [N-methyl-N-(2- pyrid-2-ylethyl)- amino]pyrimidine | 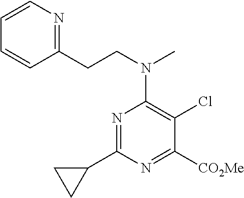 | RT 1.04, MH⁺ 347, 349 |
| 4-216 | 5-Chloro-2- cyclopropyl-4-[2- (1,3-dioxolan-2- yl)ethylamino]-6- methoxycarbonyl- pyrimidine | 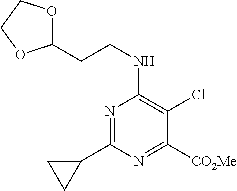 | RT 1.13, MH⁺ 328, 330 |
| 4-52 | 5-Chloro-4- cyclooctylmethyl- amino-2-cyclo- propyl-6-methoxy- carbonylpyrimidine | 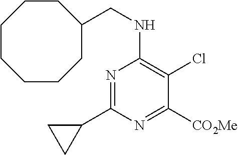 | RT 1.77, MH⁺ 352, 354 |
| 4-265 | 5-Chloro-2- cyclopropyl-6- methoxycarbonyl-4- (phenylmethoxy- carbonylmethyl- amino)pyrimidine | 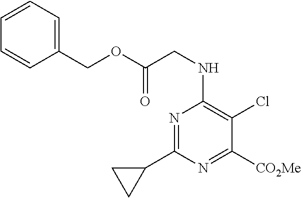 | RT 1.51, MH⁺ 376, 378 |
| 4-51 | 5-Chloro-4-[(4- cyanocyclohexyl) methylamino]-2- cyclopropyl-6- methoxycarbonyl- pyrimidine | 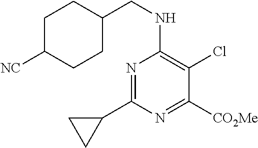 | RT 1.32, MH⁺ 349, 351 |
| 4-222 | 4-[2-(4-Amino- sulphonylphenyl)- ethylamino]-5- chloro-2-cyclo- propyl-6-methoxy- carbonylpyrimidine | 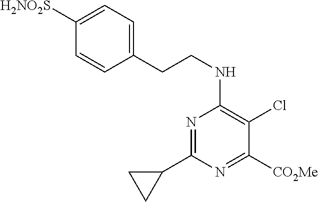 | RT 1.17, MH⁺ 411, 413 |
| 4-262 | 5-Chloro-2- cyclopropyl-6- methoxycarbonyl-4- [2-(phenylmethyl- thio)ethylamino]- pyrimidine | 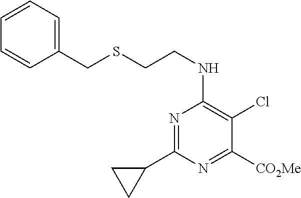 | RT 1.60, MH⁺ 378, 380 |
| 4-58 | 4-[(1- ^{t}Butoxypiperidin-4- yl)methylamino]-5- chloro-2-cyclo- propyl-6-methoxy- carbonylpyrimidine | 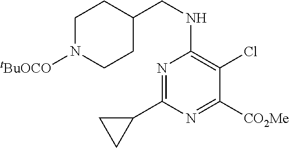 | RT 1.50, MH⁺ 425, 427 |
| 4-251 | 5-Chloro-2- cyclopropyl-6- methoxycarbonyl-4- [3-(pyrid-3- yl)propylamino]- pyrimidine | 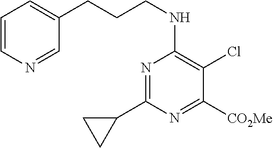 | RT 0.92, MH⁺ 347, 349 |
| 4-219 | 5-Chloro-2- cyclopropyl-6- methoxycarbonyl-4- [2-(2-oxo- imidazolidin-1- yl)ethylamino]- pyrimidine | 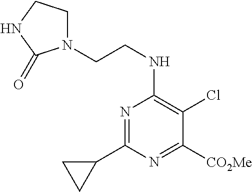 | RT 0.96, MH⁺ 340, 342 |
| 4-229 | 5-Chloro-4-[2-(6- chloro-4- trifluoromethyl- pyrid-2- yl)ethylamino]-2- cyclopropyl-6- methoxycarbonyl- pyrimidine | 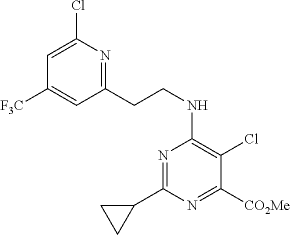 | RT 1.62, MH⁺ 435, 437, 439 |
| 4-239 | 5-Chloro-2- cyclopropyl-4-[2- (2,4- dichlorophenyl)-2- fluoro-ethylamino]- 6-methoxycarbonyl- pyrimidine | 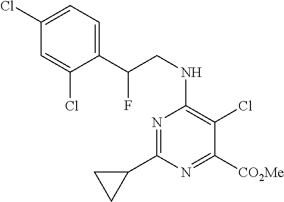 | RT 1.81, MH⁺ 418, 420, 422, 424 |
| 4-235 | 4-[2-(3- Bromophenyl)-2- fluoro-ethylamino]- 5-chloro-2- cyclopropyl-6- methoxycarbonyl- pyrimidine | 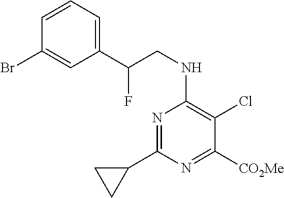 | RT 1.70, MH⁺ 428, 430, 432 |
| 4-242 | 5-Chloro-2- cyclopropyl-4-[2- fluoro-2-(3- trifluoromethyl- phenyl)propyl- amino]-6-methoxy- carbonylpyrimidine | 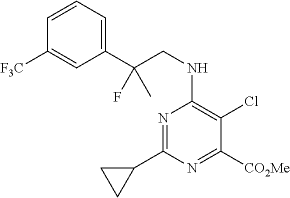 | RT 1.75, MH⁺ 432, 434 |
| 4-244 | 5-Chloro-2- cyclopropyl-4-[2- (2,4-dichloro- phenyl)-2-fluoro- pentylamino]-6- methoxycarbonyl- pyrimidine | 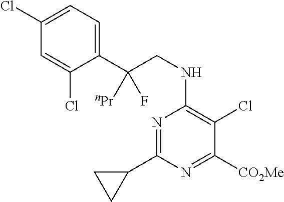 | RT 2.10, MH⁺ 460, 462, 464, 466 |
| 4-236 | 4-[2-(4- Bromophenyl)-2- fluoro-ethylamino]- 5-chloro-2- cyclopropyl-6- methoxycarbonyl- pyrimidine | 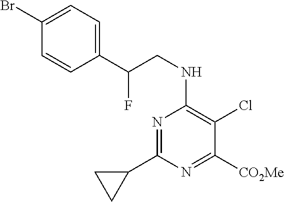 | RT 1.71, MH⁺ 428, 430, 432 |
| 4-237 | 5-Chloro-2- cyclopropyl-4-[2- fluoro-2-(3- trifluoromethyl- phenyl)ethylamino]- 6-methoxy- carbonylpyrimidine | 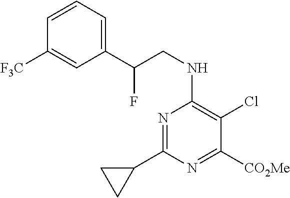 | RT 1.71, MH⁺ 418, 420 |
| 4-234 | 4-[2-(2- Bromophenyl)-2- fluoro-ethylamino]- 5-chloro-2- cyclopropyl-6- methoxycarbonyl- pyrimidine | 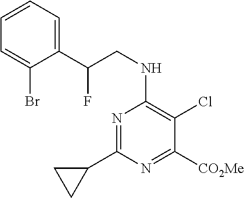 | RT 1.68, MH⁺ 428, 430, 432 |
| 4-241 | 5-Chloro-4-[2-(3- chlorophenyl)-2- fluoro- propylamino]-2- cyclopropyl-6- methoxycarbonyl- pyrimidine | 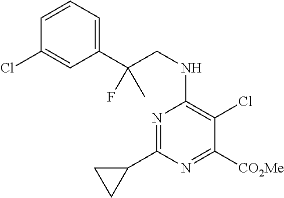 | RT 1.72, MH⁺ 398, 400, 402 |
| 4-238 | 5-Chloro-2- cyclopropyl-4-[2- (2,4-difluoro- phenyl)-2-fluoro- ethylamino]-6- methoxycarbonyl- pyrimidine | 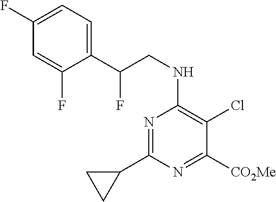 | RT 1.61, MH⁺ 386, 388 |
| 4-240 | 4-[2-(4-Bromo-2- fluorophenyl)-2- fluoro-ethylamino]- 5-chloro-2- cyclopropyl-6- methoxycarbonyl- pyrimidine | 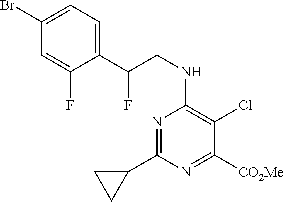 | RT 1.74, MH⁺ 446, 448, 450 |
| 4-243 | 5-Chloro-2- cyclopropyl-4-[2- (2,4- dichlorophenyl)-2- fluoro- propylamino]-6- methoxycarbonyl- pyrimidine | 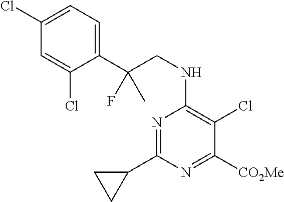 | RT 1.91, MH⁺ 432, 434, 436, 438 |
| 4-19 (E isomer) | 5-Chloro-2- cyclopropyl-6- methoxycarbonyl-4- [1-(2-phenyl- ethenyl)-cyclo- propylamino]- pyrimidine | 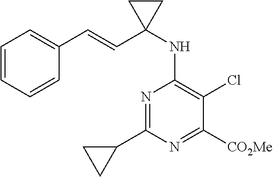 | RT 1.63, MH⁺ 370, 372 |
| 4-20 | 5-Chloro-2- cyclopropyl-4-{1-[2- (4-fluorophenyl)- ethenyl]-cyclo- propylamino}-6- methoxycarbonyl- pyrimidine | 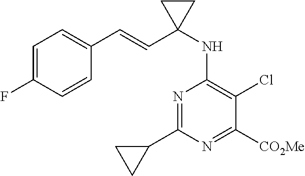 | RT 1.66, MH⁺ 388, 390 |
| 4-21 | 5-Chloro-4-{1-[2-(4- chlorophenyl)- ethenyl]- cyclopropylamino}- 2-cyclopropyl-6- methoxycarbonyl- pyrimidine | 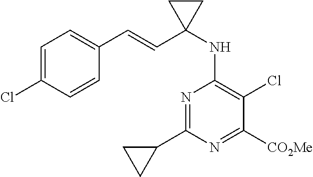 | RT 1.76, MH⁺ 404, 406, 408 |
| 4-22 | 4-{1-[2-(3,5-Bis- trifluoromethylphen- yl)-ethenyl]- cyclopropylamino}- 5-chloro-2- cyclopropyl-6- methoxycarbonyl- pyrimidine | 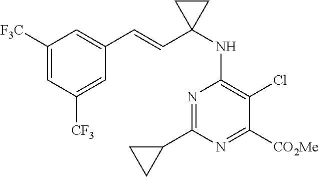 | RT 1.93, MH⁺ 506, 508 |
| 4-23 | 5-Chloro-2- cyclopropyl-6- methoxycarbonyl-4- [1-(2-thiophen-2- ylethenyl)- cyclopropylamino]- pyrimidine | 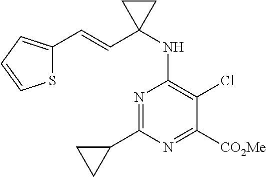 | RT 1.61, MH⁺ 376, 378 |
| 4-24 | 5-Chloro-4-{1-[2-(2- chlorophenyl)- ethenyl]- cyclopropylamino}- 2-cyclopropyl-6- methoxycarbonyl- pyrimidine | 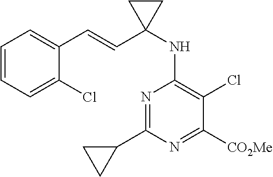 | RT 1.73, MH⁺ 404, 406, 408 |
| 4-25 | 5-Chloro-2- cyclopropyl-6- methoxycarbonyl-4- {1-[2-(4- methylphenyl)- ethenyl]- cyclopropylamino}- pyrimidine | 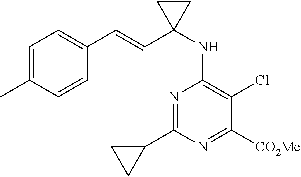 | RT 1.72, MH⁺ 384, 386 |
| 4-26 | 5-Chloro-2- cyclopropyl-4-{1-[2- (2,6-difluoro- phenyl)ethenyl]- cyclopropylamino}- 6-methoxy- carbonylpyrimidine | 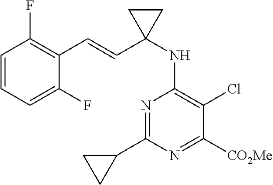 | RT 1.69, MH⁺ 406, 408 |
| 4-27 | 5-Chloro-2- cyclopropyl-4-{1-[2- (2-fluorophenyl)- ethenyl]-cyclo- propylamino}-6- methoxycarbonyl- pyrimidine | 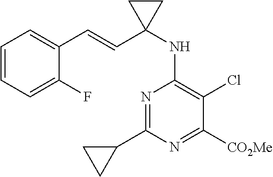 | RT 1.66, MH⁺ 388, 390 |
| 4-29 | 5-Chloro-2- cyclopropyl-4-{1-[2- (2,3-dichloro- phenyl)ethenyl]- cyclopropylamino}- 6-methoxycarbonyl- pyrimidine | 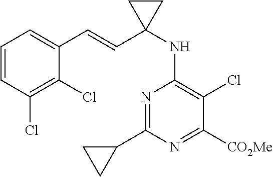 | RT 1.82, MH⁺ 438, 440, 442, 444 |
| 4-28 | 5-Chloro-2- cyclopropyl-4-{1-[2- (2,4-dichloro- phenyl)ethenyl]- cyclopropyl-amino}- 6-methoxy- carbonylpyrimidine | 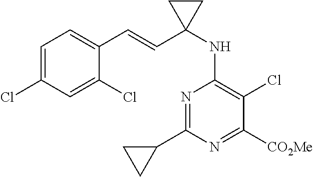 | RT 1.88, MH⁺ 438, 440, 442, 444 |
| 4-30 | 5-Chloro-4-{1-[2-(3- chlorophenyl)- etenyl]- cyclopropylamino}- 2-cyclopropyl-6- methoxycarbonyl- pyrimidine | 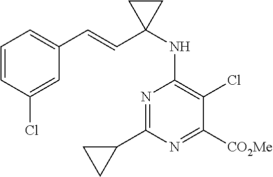 | RT 1.75, MH⁺ 404, 406, 408 |
| 4-31 | 5-Chloro-2- cyclopropyl-4-{1-[2- (2,4-difluoro- phenyl)-ethenyl]- cyclopropylamino}- 6-methoxy- carbonylpyrimidine | 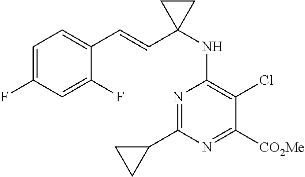 | RT 1.70, MH⁺ 406, 408 |
| 4-56 | 5-Chloro-2- cyclopropyl-4-[(3,4- dihydro-2H-pyran- 2-yl)methylamino]- 6-methoxy- carbonylpyrimidine | 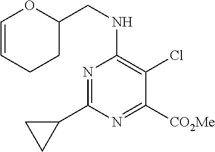 | RT 1.42, MH⁺ 324, 326 |
| 4-215 | 5-Chloro-4-[2- (cyclohex-1- enyl)ethylamino]-2- cyclopropyl-6- methoxycarbonyl- pyrimidine | 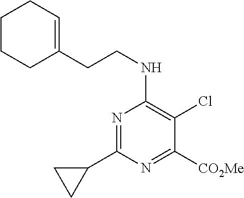 | RT 1.66, MH⁺ 336, 338 |
| 4-55 | 5-Chloro-2- cyclopropyl-4-[(2,5- dihydro-2,5- dimethoxy-furan-2- yl)methylamino]-6- methoxycarbonyl- pyrimidine | 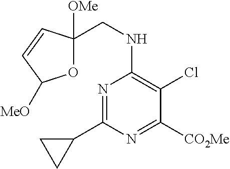 | RT 1.28, MH⁺ 370, 372 |
| 4-19 (Z isomer) | 5-Chloro-2- cyclopropyl-6- methoxycarbonyl- [(Z)-1-(2- phenylethenyl)- cyclopropylamino]- pyrimidine | 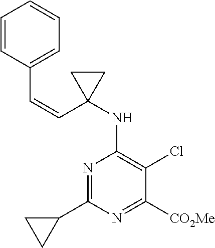 | RT 1.60, MH⁺ 370, 372 |
| 146-255 | 5-Chloro-2- cyclopropyl-4-{N- isopropyl-N-[3-(2- methoxyphenyl)- prop-2-enyl]- amino}-6- methoxycarbonyl- pyrimidine | 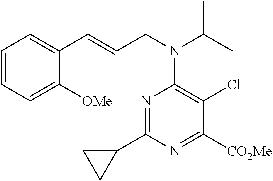 | RT 1.99, MH⁺ 416, 418 |
| 160-256 | 5-Chloro-2- cyclopropyl-4-[N-(3- furan-2-yl-prop-2- enyl)-N-2- methoxyethyl- amino]-6- methoxycarbonyl- pyrimidine | 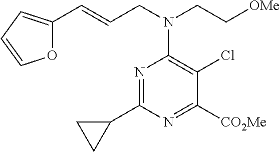 | RT 1.75, MH⁺ 392, 394 |
| 5-84 | 5-Chloro-2- cyclopropyl-6- methoxycarbonyl-4- [N-methyl-N-(4- phenoxyphenyl)- methyl-amino]- pyrimidine | 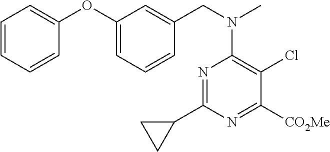 | 83-84 |
| 4-93 | 4-(4- Aminosulphonyl- phenyl)methylamino-5- chloro-2- cyclopropyl-6- methoxycarbonyl- pyrimidine | 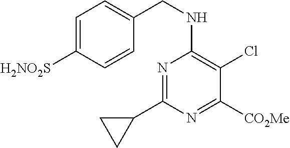 | >220 |
| 4-164 | 4-(5-Bromo- benzofuran-2- yl)methylamino-5- chloro-2- cyclopropyl-6- methoxycarbonyl- pyrimidine | 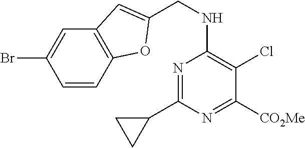 | 103-105 |
| 4-274 | 5-Chloro-4-[2-(4- chlorophenyl)-1- hydroxymethyl- ethylamino]-2- cyclopropyl-6- methoxycarbonyl- pyrimidine | 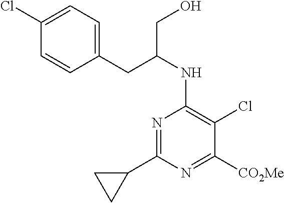 | RT 1.43, MH⁺ 396, 398, 400 |
| 4-275 (R, R isomer) | 5-Chloro-2- cyclopropyl-4-[(1R, 2R)-2-hydroxy-1- hydroxymethyl-2- (4-nitrophenyl)- ethylamino]-6- methoxycarbonyl- pyrimidine | 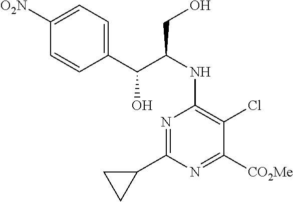 | RT 1.21, MH⁺ 423, 425 |
| 4-275 (S, S isomer) | 5-Chloro-2- cyclopropyl-4-[(1S, 2S)-2-hydroxy-1- hydroxymethyl-2- (4-nitrophenyl)- ethylamino]-6- methoxycarbonyl- pyrimidine | 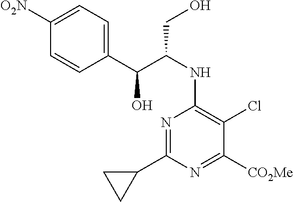 | RT 1.21, MH⁺ 423, 425 |
| 5-270 | 5-Chloro-2- cyclopropyl-4-[N-(2- hydroxy-1-methyl- 2-phenyl-ethyl)-N- methyl-amino]-6- methoxycarbonyl- pyrimidine | 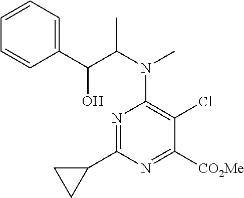 | RT 1.62, MH⁺ 376, 378 |
| 4-203 (R isomer) | 5-Chloro-2- cyclopropyl-4-[(1R)- 2-hydroxy-1- phenyl-ethylamino]- 6-methoxycarbonyl- pyrimidine | 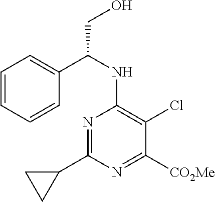 | RT 1.28, MH⁺ 348, 350 |
| 4-273 (R isomer) | 5-Chloro-2- cyclopropyl-4-[(1R)- 1-hydroxymethyl-2- phenyl-ethylamino]- 6-methoxycarbonyl- pyrimidine | 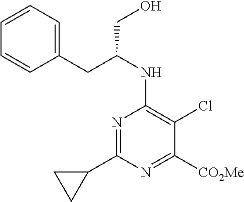 | RT 1.29, MH⁺ 362, 364 |
| 155-257 | 5-Chloro-2- cyclopropyl-4-[N-(2- hydroxy-2- phenylethyl)-N- phenylmethyl- amino]-6- methoxycarbonyl- pyrimidine | 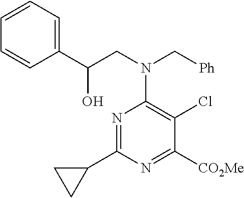 | RT 1.88, MH⁺ 438, 440 |
| 4-280 (1S, 2R isomer) | 5-Chloro-2- cyclopropyl-4-[(1S, 2R)-1,2-diphenyl-2- hydroxy- ethylamino]-6- methoxycarbonyl- pyrimidine | 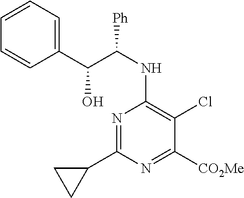 | RT 1.63, MH⁺ 424, 426 |
| 4-257 | 5-Chloro-2- cyclopropyl-4-(2- hydroxy-2-phenyl- ethylamino)-6- methoxycarbonyl- pyrimidine | 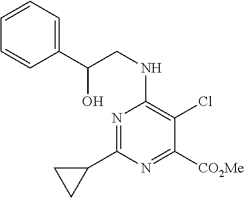 | RT 1.29, MH⁺ 348, 350 |
| 4-37 (1S, 2R isomer) | 5-Chloro-2- cyclopropyl-4-[(1S, 2R)-2-hydroxy- indan-1-ylamino]-6- methoxycarbonyl- pyrimidine | 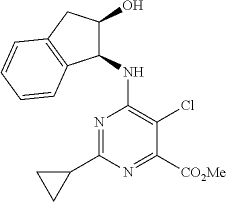 | RT 1.44, MH⁺ 360, 362 |
| 4-59 | 5-Chloro-2- cyclopropyl-4-[(1- hydroxy-6- methoxyindan-1- yl)methylamino]-6- methoxycarbonyl- pyrimidine | 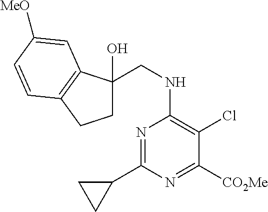 | RT 1.37, MH⁺ 404, 406 |
| 4-280 (1R, 2S isomer) | 5-Chloro-2- cyclopropyl-4-[(1R, 2S)-1,2-diphenyl-2- hydroxy-ethyl- amino]-6-methoxy- carbonylpyrimidine | 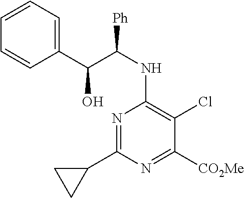 | RT 1.64, MH⁺ 424, 426 |
| 4-276 (R isomer) | 5-Chloro-2- cyclopropyl-4-[(1R)- 1-hydroxymethyl-2- phenylmethylthio- ethylamino]-6- methoxycarbonyl- pyrimidine | 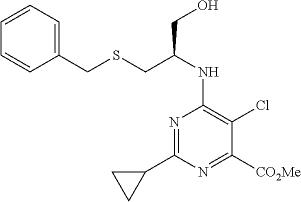 | RT 1.45, MH⁺ 408, 410 |
| 4-37 (1R, 2S isomer) | 5-Chloro-2- cyclopropyl-4-[(1R, 2S)-2-hydroxy- indan-1-ylamino]-6- methoxycarbonyl- pyrimidine | 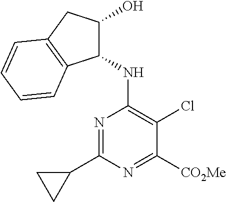 | RT 1.44, MH⁺ 360, 362 |
| 4-38 (S, S isomer) | 5-Chloro-2- cyclopropyl-4-[(3S, 4S)-4-hydroxytetra- hydrofuran-3- ylamino]-6- methoxycarbonyl- pyrimidine | 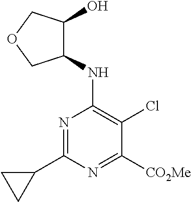 | RT 1.02, MH⁺ 314, 316 |
| 4-203 (S isomer) | 5-Chloro-2- cyclopropyl-4-[(1S)- 2-hydroxy-1- phenyl-ethylamino]- 6-methoxycarbonyl- pyrimidine | 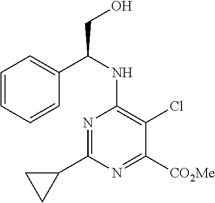 | RT 1.28, MH⁺ 348, 350 |
| 4-202 | 5-Chloro-2- cyclopropyl-4-(2- hydroxy-1- tetrahydropyran-4- yl-ethylamino)-6- methoxycarbonyl- pyrimidine | 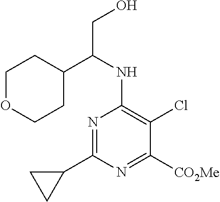 | RT 0.97, MH⁺ 356, 358 |
| 4-264 | 5-Chloro-4-[3-(4- chlorophenoxy)-2- hydroxy- propylamino]-2- cyclopropyl-6- methoxycarbonyl- pyrimidine | 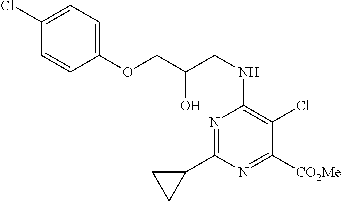 | RT 1.48, MH⁺ 412, 414, 416 |
| 4-34 | 5-Chloro-2- cyclopropyl-4-(2- hydroxycyclopentyl amino)-6- methoxycarbonyl- pyrimidine | 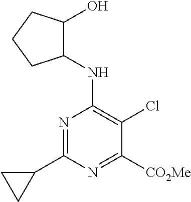 | RT 1.06, MH⁺ 312, 314 |
| 4-203 | 5-Chloro-2- cyclopropyl-4-(2- hydroxy-1-phenyl- ethylamino)-6- methoxycarbonyl- pyrimidine | 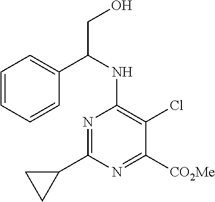 | RT 1.28, MH⁺ 348, 350 |
| 157-156 | 5-Chloro-2- cyclopropyl-4-(N-2- hydroxyethyl-N- pyrid-3-ylmethyl- - amino)-6-methoxy- carbonylpyrimidine | 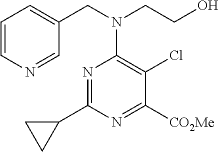 | RT 0.93, MH⁺ 363, 365 |
| 4-50 | 5-Chloro-2- cyclopropyl-4-[(1- hydroxycyclohex-1- yl)methylamino]-6- methoxycarbonyl- pyrimidine | 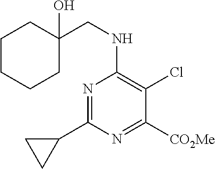 | RT 1.30, MH⁺ 340, 342 |
| 5-257 | 5-Chloro-2- cyclopropyl-4-[N-(2- hydroxy-2-phenyl- ethyl)-N-methyl- amino]-6-methoxy- carbonylpyrimidine | 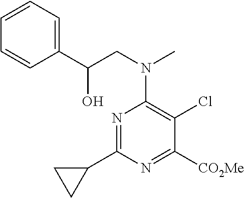 | RT 1.53, MH⁺ 362, 364 |
| 4-201 | 5-Chloro-2- cyclopropyl-4-(2- hydroxy-1- tetrahydrofuran-3- yl-ethylamino)-6- methoxycarbonyl- pyrimidine | 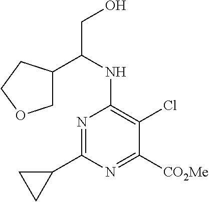 | RT 0.94, MH⁺ 342, 244 |
| 157-157 | 5-Chloro-4-[N-(6- chloropyrid-3- ylmethyl)-N-2- hydroxyethyl- amino]-2- cyclopropyl-6- methoxycarbonyl- pyrimidine | 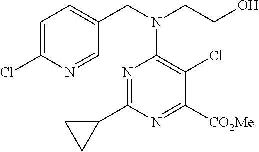 | RT 1.36, MH⁺ 397, 399, 401 |
| 4-259 | 5-Chloro-2- cyclopropyl-4-[2- hydroxy-2-(4- hydroxyphenyl)- ethylamino]-6- methoxycarbonyl- pyrimidine | 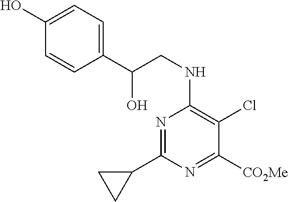 | RT 1.06, MH⁺ 364, 366 |
| 4-271 | 5-Chloro-2- cyclopropyl-4-[2- hydroxy-2-(4- hydroxyphenyl)-1- methyl-ethylamino]- 6- methoxycarbonyl- pyrimidine | 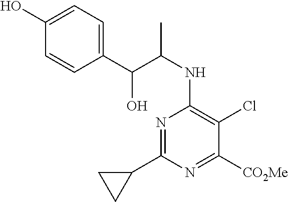 | RT 1.12, MH⁺ 378, 380 |
| 4-258 | 5-Chloro-2- cyclopropyl-4-[2- hydroxy-2-(3- hydroxyphenyl)- ethylamino]-6- methoxycarbonyl- pyrimidine | 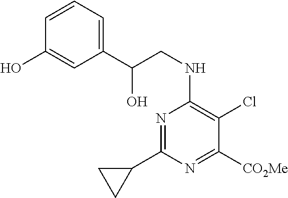 | RT 1.09, MH⁺ 364, 366 |
| 4-273 (S isomer) | 5-Chloro-2- cyclopropyl-4-[(1S)- 1-hydroxymethyl-2- phenyl-ethylamino]- 6-methoxy- carbonylpyrimidine | 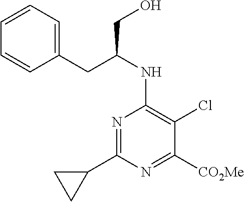 | RT 1.29, MH⁺ 362, 364 |
| 4-270 (1S, 2R isomer) | 5-Chloro-2- cyclopropyl-4-[(1S, 2R)-2-hydroxy-1- methyl-2-phenyl- ethylamino]-6- methoxycarbonyl- pyrimidine | 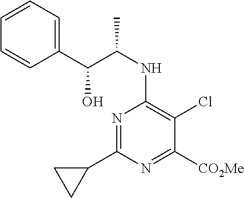 | RT 1.40, MH⁺ 362, 364 |
| 4-36 (trans isomer) | 5-Chloro-2- cyclopropyl-4- (trans-2- hydroxycyclohexyl- amino)-6-methoxy- carbonylpyrimidine | 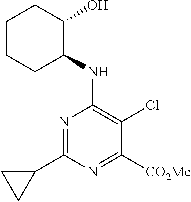 | RT 1.12, MH⁺ 326, 328 |
| 4-270 (erythro isomer) | 5-Chloro-2- cyclopropyl-4- (erythro-2-hydroxy- 1-methyl-2-phenyl- ethylamino)-6- methoxycarbonyl- pyrimidine | 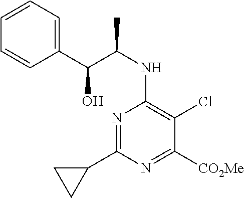 | RT 1.40, MH⁺ 362, 364 |
| 4-205 | 5-Chloro-2- cyclopropyl-6- methoxycarbonyl-4- [1-(4-methoxy- phenyl)-2- methylsulphonyl- ethylamino]- pyrimidine | 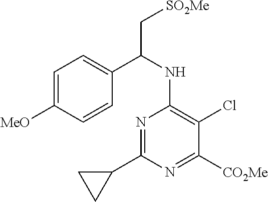 | RT 1.37, MH⁺ 440, 442 |
| 155-60 | 5-Chloro-2- cyclopropyl-4- dibenzylamino-6- methoxycarbonyl- pyrimidine | 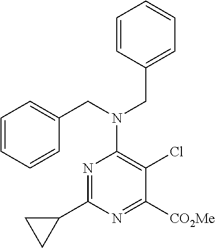 | 7.26 (10 H, m), 4.81 (4 H, m), 3.95 (3 H, s), 2.06 (1 H, m), 0.88 (4 H, m) |
| 156-60 | 4-(N-carboxy- methyl-N-phenyl- methyl-amino)-5- chloro-2-cyclo- propyl-6-methoxy- carbonylpyrimidine | 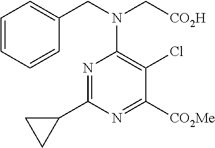 | 7.35 (5 H, m), 5.03 (2 H, s), 4.08 (2 H, s), 3.96 (3 H, s), 2.09 (1 H, m), 0.98 (4 H, m) |
| 6-35 | 5-Chloro-4-(N- cyclohexyl-N-ethyl- amino)-2-cyclo- propyl-6-methoxy- carbonylpyrimidine | 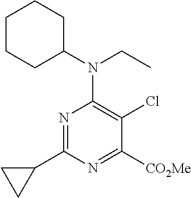 | 4.21 (1 H, m), 3.96 (3 H, s), 3.53 (2 H, q), 2.08 (1 H, m), 1.82 (4 H, m), 1.67 (2 H, d), 1.54 (2 H, dq), 1.32 (2 H, qt), 1.13 (3 H, t), 1.02 (2 H, m), 0.96 (2 H, m) |
| 6-125 | 5-Chloro-2- cyclopropyl-4-(N- ethyl-N-furan-2- ylmethyl-amino)-6- methoxycarbonyl- pyrimidine | 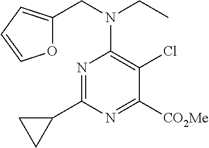 | 7.34 (1 H, d), 6.32 (1 H, dd), 6.22 (1 H, d), 4.76 (2 H, s), 3.95 (3 H, s), 3.69 (2 H, q), 2.07 (1 H, m), 1.22 (3 H, t), 1.03 (2 H, m), 0.95 (2 H, m) |
| 6-140 | 5-Chloro-2- cyclopropyl-4-(N- ethyl-N-thiophen-2- ylmethyl-amino)6- methoxycarbonyl- pyrimidine | 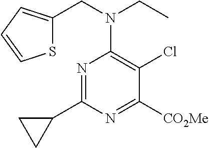 | 7.21 (1 H, dd), 6.98 (1 H, d), 6.93 (1 H, dd), 4.91 (2 H, s), 3.95 (3 H, s), 3.63 (2 H, q), 2.12 (1 H, m), 1.24 (3 H, t), 1.12 (2 H, m), 0.97 (2 H, m) |
| 6-142 | 4-[N-(5- bromothiophen-2- ylmethyl)-N-ethyl- amino]-5-chloro-2- cyclopropyl-6- methoxycarbonyl- pyrimidine | 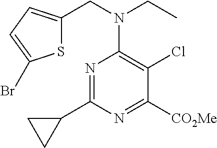 | 6.88 (1 H, d), 6.76 (1 H, d), 4.79 (2 H, s), 3.96 (3 H, s), 3.62 (2 H, q), 2.13 (1 H, m), 1.25 (3 H, t), 1.12 (2 H, m), 1.01 (2 H, m) |
| 6-135 | 5-Chloro-2- cyclopropyl-4-[N- ethyl-N-(5- methylfuran-2- ylmethyl)-amino]-6- methoxycarbonyl- pyrimidine | 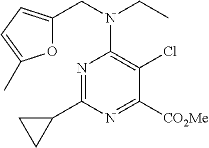 | 6.10 (1 H, d), 5.89 (1 H, dd), 4.70 (2 H, s), 3.95 (3 H, s), 3.69 (2 H, q), 2.25 (3 H, s), 2.08 (1 H, m), 1.22 (3 H, t), 1.06 (2 H, m), 0.95 (2 H, m) |
| 6-112 | 5-Chloro-2- cyclopropyl-4-[N- (3,4-dimethoxy- phenylmethyl)-N- ethyl-amino]-6- methoxycarbonyl- pyrimidine | 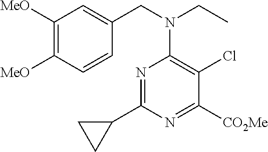 | 6.85 (1 H, d), 6.82 (1 H, s), 6.81 (1 H, d), 4.77 (2 H, s), 3.96 (3 H, s), 3.87 (3 H, s), 3.85 (3 H, s), 3.61 (2 H, q), 2.07 (1 H, m), 1.24 (3 H, t), 1.00 (2 H, m), 0.95 (2 H, m) |
| 6-66 | 5-Chloro-2- cyclopropyl-4-[N- ethyl-N-(4- fluorophenylmethyl)- amino]-6- methoxycarbonyl- pyrimidine | 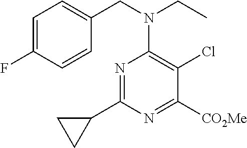 | 7.22 (2 H, dd), 7.01 (2 H, t), 4.79 (2 H, s), 3.96 (3 H, s), 3.62 (2 H, q), 2.05 (1 H, m), 1.25 (3 H, t), 0.92 (4 H, d) |
| 6-161 | 5-Chloro-2- cyclopropyl-4-[N- ethyl-N-(2- methylpyrimidin-4- ylmethyl)-amino]-6- methoxycarbonyl- pyrimidine | 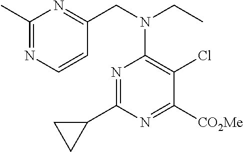 | 8.55 (1 H, d), 6.97 (1 H, d), 4.79 (2 H, s), 3.97 (3 H, s), 3.76 (2 H, q), 2.73 (3 H, s), 2.00 (1 H, m), 1.32 (3 H, t), 0.89 (2 H, m), 0.73 (2 H, m) |
| 6-102 | 5-Chloro-4-[N-(2- chloro-6- fluorophenylmethyl)- N-ethyl-amino]-2- cyclopropyl-6- methoxycarbonyl- pyrimidine | 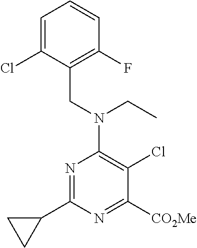 | 7.21 (2 H, m), 6.97 (1 H, m), 5.02 (2 H, s), 3.97 (3 H, s), 3.63 (2 H, q), 2.08 (1 H, m), 1.12 (3 H, t), 1.02 (2 H, m), 0.93 (2 H, m) |
| 148-60 | 4-(N-But-2-yl-N- phenylmethyl- amino)-5-chloro-2- cyclopropyl-6- methoxycarbonyl- pyrimidine | 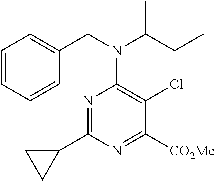 | RT 2.00, MH⁺ 374. 376 |
| 146-135 | 5-Chloro-2- cyclopropyl-6- methoxycarbonyl-4- [N-(5-methylfuran- 2-ylmethyl)-N-prop- 2-yl-amino]- pyrimidine | 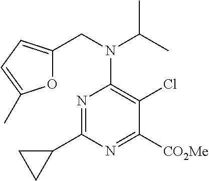 | RT 1.90, MH⁺ 364, 366 |
| 6-65 | 5-Chloro-2- cyclopropyl-4-[N- ethyl-N-(3- fluorophenylmethyl)- amino]-6- methoxycarbonyl- pyrimidine | 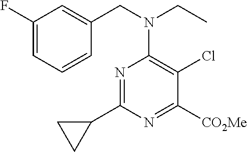 | RT 1.83, MH⁺ 364, 366 |
| 151-48 | 5-Chloro-4-(N- cyclopentyl-N-prop- 2-en-1-yl-amino)-2- cyclopropyl-6- methoxycarbonyl- pyrimidine | 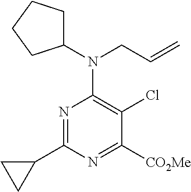 | RT 1.94, MH⁺ 336, 338 |
| 151-117 | 5-Chloro-4-[N-(5- chloro-2- methoxyphenylmeth- yl)-N-prop-2-en-1- yl-amino]-2- cyclopropyl-6- methoxycarbonyl- pyrimidine | 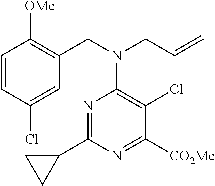 | RT 2.02, MH⁺ 422, 424, 426 |
| 151-140 | 5-Chloro-2- cyclopropyl-6- methoxycarbonyl-4- (N-prop-2-en-1-yl- N-thiophen-2- ylmethyl-amino)- pyrimidine | 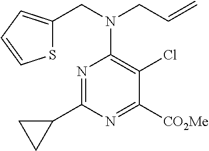 | RT 1.90, MH⁺ 364, 366 |
| 151-141 | 5-Chloro-4-[N-(5- chlorothiophen-2- ylmethyl)-N-prop-2- en-1-yl-amino]-2- cyclopropyl-6- methoxycarbonyl- pyrimidine | 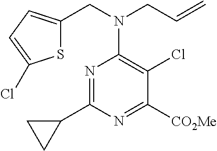 | RT 2.09, MH⁺ 398, 400, 402 |
| 151-60 | 5-Chloro-2- cyclopropyl-6- methoxycarbonyl-4- (N-phenylmethyl-N- prop-2-en-1-yl- amino)-pyrimidine | 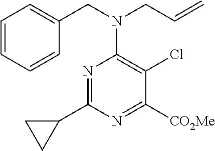 | RT 1.91, MH⁺ 358, 360 |
| 151-109 | 5-Chloro-2- cyclopropyl-4-[N- (2,3- dimethoxyphenyl- methyl)-N-prop-2-en- 1-yl-amino]-6- methoxycarbonyl- pyrimidine | 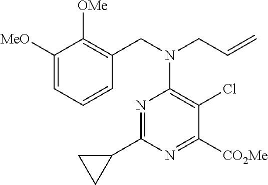 | RT 1.86, MH⁺ 418, 420 |
| 145-73 | 5-Chloro-2- cyclopropyl-6- methoxycarbonyl-4- [N-(4-methyl- phenylmethyl)-N- prop-1-yl-amino]- pyrimidine | 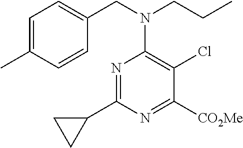 | RT 2.03, MH⁺ 374, 376 |
| 145-64 | 5-Chloro-2- cyclopropyl-4-[N-(2- fluorophenylmethyl)- N-prop-1-yl- amino]-6-methoxy- carbonylpyrimidine | 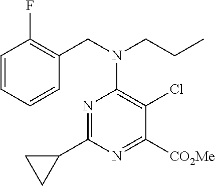 | RT 1.96, MH⁺ 378, 380 |
| 5-156 | 5-Chloro-2- cyclopropyl-6- methoxycarbonyl-4- (N-methyl-N- pyridin-3-ylmethyl- amino)pyrimidine | 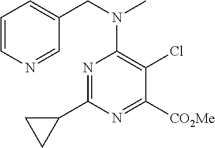 | 8.55 (2 H, d), 7.60 (1 H, d), 7.29 (1 H, dd), 4.82 (2 H, s), 3.97 (3 H, s), 3.19 (3 H, s), 2.08 (1 H, m), 0.97 (4 H, m) |
| 5-160 | 5-Chloro-2- cyclopropyl-6- methoxycarbonyl-4- (N-methyl-N- pyridin-4-ylmethyl- amino)pyrimidine | 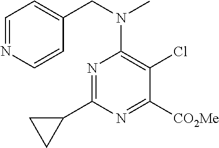 | RT 1.06, MH⁺ 333, 335 |
| 161-125 | 5-Chloro-4-[N-(2- cyanoethyl)-N- furan-2-ylmethyl- amino]-2-cyclo- propyl-6-methoxy- carbonylpyrimidine | 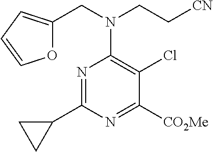 | RT 1.57, MH⁺ 361, 363 |
| 5-91 | 5-Chloro-2- cyclopropyl-6- methoxycarbonyl-4- [N-methyl-N-(3- nitrophenylmethyl)- amino]pyrimidine | 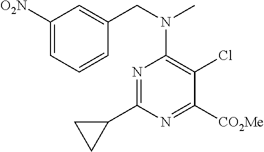 | RT 1.74, MH⁺ 377, 379 |
| 5-76 | 5-Chloro-2- cyclopropyl-6- methoxycarbonyl-4- [N-methyl-N-(3- trifluoromethyl- phenylmethyl)- amino]pyrimidine | 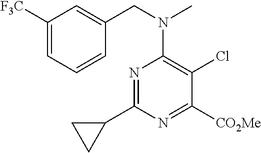 | RT 1.88, MH⁺ 400, 402 |
| 5-63 | 5-Chloro-4-[N-(4- chlorophenyl- methyl)-N-methyl- amino]-2-cyclo- propyl-6-methoxy- carbonylpyrimidine | 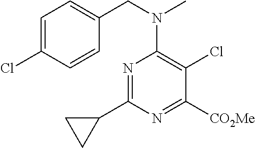 | RT 1.90, MH⁺ 366, 368, 370 |
| 5-66 | 5-Chloro-2- cyclopropyl-4-[N-(4- fluorophenylmethyl)- N-methyl-amino]- 6-methoxy- carbonylpyrimidine | 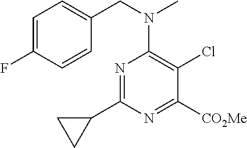 | RT 1.77, MH⁺ 350, 352 |
| 5-69 | 4-[N-(4-Bromo- phenylmethyl)-N- methyl-amino]-5- chloro-2-cyclo- propyl-6-methoxy- carbonylpyrimidine | 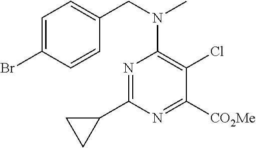 | RT 1.91, MH⁺ 410, 412, 414 |
| 5-62 | 5-Chloro-4-[N-(3- chlorophenyl- methyl)-N-methyl- amino]-2-cyclo- propyl-6-methoxy- carbonylpyrimidine | 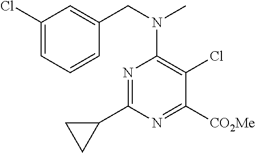 | RT 1.87, MH⁺ 366, 368, 370 |
| 5-65 | 5-Chloro-2- cyclopropyl-4-[N-(3- fluorophenylmethyl)- N-methyl-amino]- 6-methoxy- carbonylpyrimidine | 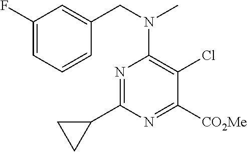 | RT 1.79, MH⁺ 350, 352 |
| 5-79 | 5-Chloro-2- cyclopropyl-6- methoxycarbonyl-4- [N-(3-methoxy- phenylmethyl)-N- methyl-amino]- pyrimidine | 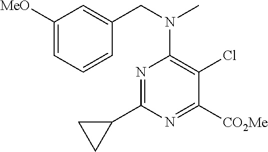 | RT 1.73, MH⁺ 362, 364 |
| 5-105 | 5-Chloro-2- cyclopropyl-4-[N- (3,4-dichlorophenyl- methyl)-N-methyl- amino]-6-methoxy- carbonylpyrimidine | 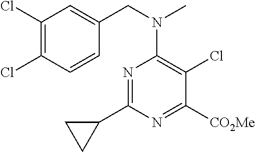 | RT 1.99, MH⁺ 400, 402, 404, 406 |
| 151-66 | 5-Chloro-2- cyclopropyl-4-[N-(4- fluorophenylmethyl)- N-prop-2-en-1-yl- amino]-6-methoxy- carbonylpyrimidine | 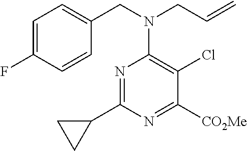 | RT 1.95, MH⁺ 376, 378 |
| 145-60 | 5-Chloro-2- cyclopropyl-6- methoxycarbonyl-4- (N-phenyl-N-prop- 1-yl-amino)- pyrimidine | 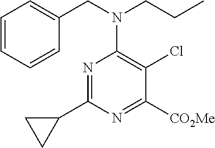 | RT 1.95, MH⁺ 360, 362 |
| 5-114 | 5-Chloro-2- cyclopropyl-4-[N- (3,4-diethoxy- phenylmethyl)-N- methyl-amino]-6- methoxycarbonyl- pyrimidine | 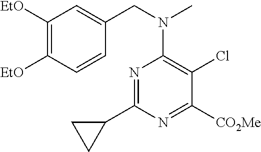 | RT 1.83, MH⁺ 420, 422 |
| 5-90 | 5-Chloro-2- cyclopropyl-6- methoxycarbonyl-4- [N-methyl-N-(2- nitrophenylmethyl)- amino]pyrimidine | 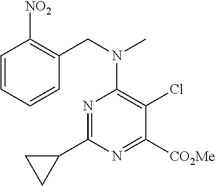 | RT 1.72, MH⁺ 377, 379 |
| 5-64 | 5-Chloro-2-cyclo- propyl-4-[N-(2- fluorophenylmethyl)- N-methyl]-6- methoxy- carbonylpyrimidine | 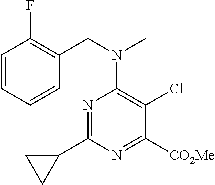 | RT 1.79, MH⁺ 350, 352 |
| 5-135 | 5-Chloro-2- cyclopropyl-6- methoxycarbonyl-4- [N-methyl-N-(5- methylfuran-2- ylmethyl)-amino]- pyrimidine | 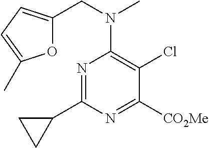 | RT 1.76, MH⁺ 336, 368 |
| 5-104 | 5-Chloro-2- cyclopropyl-4-[N- (2,6-dichlorophenyl- methyl)-N-methyl- amino]-6-methoxy- carbonylpyrimidine | 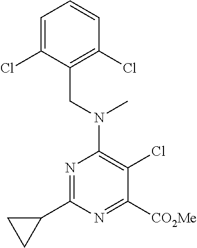 | RT 2.02, MH⁺ 400, 402, 404, 406 |
| 5-149 | 5-Chloro-2- cyclopropyl-6- methoxycarbonyl-4- [N-methyl-N-(4- methyl-1,2,5- oxadiazol-3- ylmethyl)-amino]- pyrimidine | 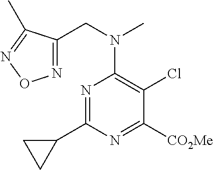 | RT 1.57, MH⁺ 338, 340 |
| 5-113 | 5-Chloro-2- cyclopropyl-4-[N- (3,5-dimethoxy- phenylmethyl)-N- methyl-amino]-6- methoxycarbonyl- pyrimidine | 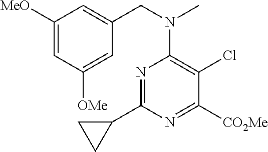 | RT 1.75, MH⁺ 392, 394 |
| 5-86 | 4-[N-(4- aminophenylmethyl)- N-methyl-amino]- 5-chloro-2- cyclopropyl-6- methoxycarbonyl- pyrimidine | 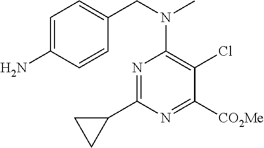 | 7.04 (2 H, d), 6.65 (2 H, d), 4.70 (2 H, s), 3.96 (3 H, s), 3.69 (2 H, br s), 3.09 (3 H, s), 2.08 (1 H, m), 1.03 (2 H, m), 0.94 (2 H, m) |
| 152-75 | 5-Chloro-2- cyclopropyl-6- methoxycarbonyl-4- [N-(3-methylbut-2- en-1-yl)-N-(2- trifluoromethyl- phenylmethyl)- amino]pyrimidine | 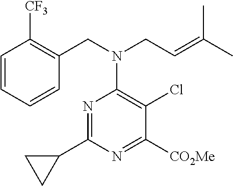 | RT 2.24, MH⁺ 454, 456 |
| 5-92 | 5-Chloro-2- cyclopropyl-6- methoxycarbonyl-4- [N-methyl-N-(4- nitrophenylmethyl)- amino]-pyrimidine | 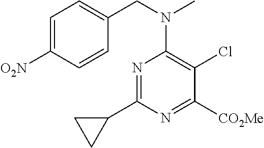 | RT 1.73, MH⁺ 377, 379 |
| 147-60 | 4-(N-But-1-yl-N- phenylmethyl- amino)-5-chloro-2- cyclopropyl-6- methoxycarbonyl- pyrimidine | 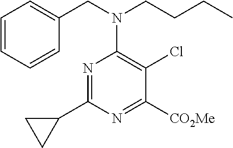 | RT 2.03, MH⁺ 374, 376 |
| 4-137 | 5-Chloro-2- cyclopropyl-4- (furan-3- ylmethylamino)-6- methoxycarbonyl- pyrimidine | 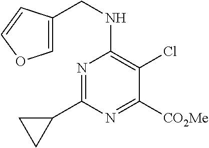 | 7.40 (2H, m), 6.39 (1H, m), 5.70 (1H, br t), 4.50 (2H, d), 3.96 (3H, s), 2.13 (1H, m), 1.10 (2H, m), 1.00 (2H, m) |
| 4-186 | 5-Chloro-2- cyclopropyl-4-[(1- furan-2- yl)ethylamino]-6- methoxycarbonyl- pyrimidine | 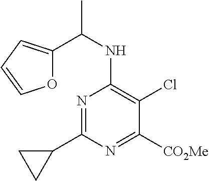 | 52-54 |
| 112-186 | 5-Chloro-2-(4- chloro-2-fluoro-3- methoxyphenyl)-4- [(1-furan-2- yl)ethylamino]-6- methoxycarbonyl- pyrimidine | 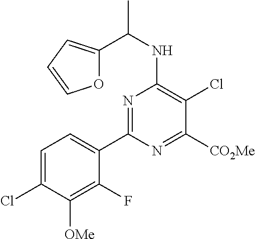 | 80-81 |
| 6-160 | 5-Chloro-2- cyclopropyl-4-(N- ethyl-N-pyridin-4- ylmethyl-amino)-6- methoxycarbonyl- pyrimidine | 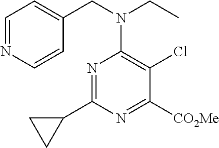 | 8.60 (2H, d), 7.20 (2H, d), 5.80 (2H, s), 4.00 (3H, s), 3.70 (2H, q), 2.00 (1H, m), 1.40 (3H, t), 0.90 (2H, m), 0.80 (2H, m) |
| 4-15 | 5-Chloro-2- cyclopropyl-6- methoxycarbonyl-4- [1-(2-methylpyridin- 5-yl)cyclopropyl- amino]pyrimidine | 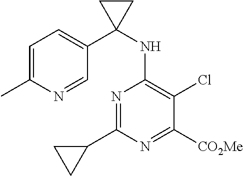 | 8.40 (d, 1H), 7.50 (1H, dd), 7.10 (1H, d), 6.30 (1H, br s), 3.90 (3H, s), 2.50 (3H, s), 2.10 (1H, m), 1.40 (4H, m), 0.90 (4H, m) |
| 4-128 | 4-[(3-bromofuran-2- yl)methylamino]-5- chloro-2-cyclo- propyl-6-methoxy- carbonylpyrimidine | 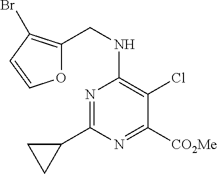 | 7.35 (1H, d), 6.41 (1H, d), 5.85 (1H, br t), 4.71 (2H, d), 3.97 (3H, s), 2.14 (1H, m), 1.12 (2H, m), 0.98 (2H, m) |
| 4-14 | 5-Chloro-4-[1-(2- chloropyridin-5-yl)- cyclopropylamino]- 2-cyclopropyl-6- methoxycarbonyl- pyrimidine | 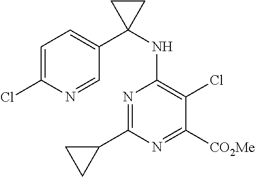 | 8.30 (1H, d), 7.60 (1H, dd), 7.30 (1H, d), 6.30 (1H, br s), 4.00 (3H, s), 2.10 (1H, m), 1.40 (4H, m), 0.90 (4H, m) |
| 114-60 | 5-Chloro-2-(4- chloro-2-fluoro-3- methoxyphenyl)-4- (N-ethyl-N-phenyl- methylamino)-6- methoxycarbonyl- pyrimidine | 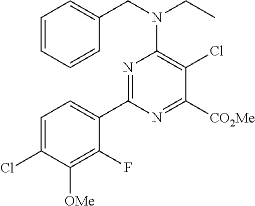 | 7.63 (1H, t), 7.29 (5H, m), 7.16 (1H, d), 4.99 (2H, d), 4.00 (3H, s), 3.96 (3H, s), 3.73 (2H, q), 1.30 (3H, t) |
| 4-294 (S isomer) | 5-Chloro-2- cyclopropyl-4-[(1S)- 2-indol-3-yl-1- hydroxymethyl- ethylamino)-6- methoxycarbonyl- pyrimidine | 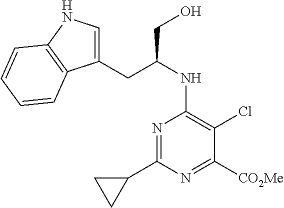 | 152-156 |

### Example 3

### Synthesis of 4-(5-bromofuran-2-ylmethylamino)-5-chloro-2-cyclopropyl-6-methoxycarbonyl-pyrimidine (Compound 4-129)

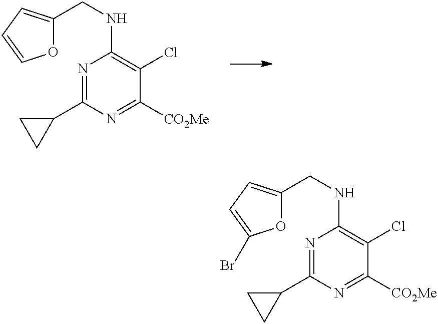

N-Bromosuccinimide (79 mg, 0.44 mmol) was added to a stirred solution of 5-chloro-2-cyclopropyl-4-(furan-2-ylmethylamino)-6-methoxycarbonylpyrimidine (prepared as described in example 2) (90 mg, 0.29 mmol) in dichloromethane at ambient temperature. The mixture was stirred in the dark for 24 hours then further N-bromosuccinimide (26 mg, 0.15 mmol) was added and stirring continued for 3 hours. Ethyl acetate and brine were added, the phases separated and the organic layer dried over magnesium sulphate, filtered and evaporated under reduced pressure to leave a brown solid. The residue was purified by column chromatography on silica using ethyl acetate:hexane (1:2) as eluent to provide 4-(5-bromofuran-2-ylmethylamino)-5-chloro-2-cyclopropyl-6-methoxycarbonyl-pyrimidine as a yellow gum (31 mg, 28%). Characterising data for this compound are as follows: ¹H nmr (400 MHz, CDCl₃) δ_{H} 6.25 (2H, m), 5.88 (1H, br t), 4.61 (2H, d), 3.97 (3H, s), 2.11 (1H, m), 1.09 (2H, m), 1.00 (2H, m) ppm.

### Example 4

### Synthesis of 6-methoxycarbonyl-2,4,5-trichloropyrimidine

### 4.1 Preparation of 2,4-dihydroxy-6-methoxycarbonylpyrimidine(methyl orotate)

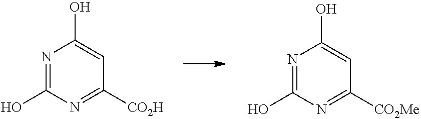

Thionyl chloride (500 ml), pyridine (2.5 ml) and a few drops of dimethylformamide were added to orotic acid monohydrate (78 g, 0.44 mol). The reaction mixture was stirred at RT for 5 days and then heated under reflux for an additional 14 hours. After cooling the solid material was allowed to settle and the supernatant decanted. The solid residue was washed with hexane and dried. Methanol (700 ml) was added dropwise with agitation to the solid. Once the rate of the gas formation slowed, the mixture was heated at reflux overnight and then cooled to 4-5° C. The solid was removed by filtration and washed with methanol and ether to provide methyl orotate (73 g, 97%). Characterising data for this compound are as follows: ¹H nmr (400 MHz, d₆-DMSO) δ_{H} 11.41 (1H, s), 11.26 (1H, s), 6.04 (1H, s), 3.84 (3H, s) ppm.

### 4.2 Preparation of 5-chloro-2,4-dihydroxy-6-methoxycarbonylpyrimidine

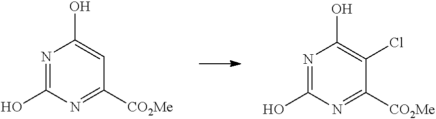

A catalytic quantity of ferric chloride was added to a solution of methyl orotate (34 g, 0.20 mol) in acetic anhydride (5% solution in glacial acetic acid, 500 ml). The mixture was heated to 90-95° C. and sulphuryl chloride (54 g, 0.40 mol) was added dropwise. After the addition was complete, the solution was slowly brought to reflux with agitation and heating was continued overnight. The solution was cooled to 18° C. and the solid was removed by filtration. The solid was washed with acetic acid and then with water, and dried to give 5-chloro-2,4-dihydroxy-6-methoxycarbonylpyrimidine (36.0 g, 89%). Characterising data for this compound are as follows: ¹H nmr (400 MHz, d₆-DMSO) δ_{H} 11.86 (1H, s), 11.62 (1H, s), 3.88 (3H, s) ppm.

### 4.3 Preparation of 6-methoxycarbonyl-2,4,5-trichloropyrimidine

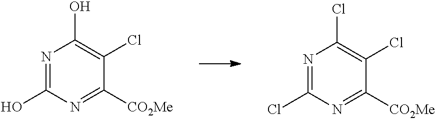

Phosphorus oxychloride (993 ml) was added to 5-chloro-2,4-dihydroxy-6-methoxycarbonylpyrimidine (30.0 g, 0.146 mol) at 10° C. and the resulting solution cooled to 0° C. N,N-Diethyl aniline (30.9 ml, 0.193 mol) was added dropwise to the stirred solution. After the addition was complete, the reaction mixture was allowed to warm slowly to ambient temperature and was then heated at reflux overnight. The resulting solution was cooled and concentrated under reduced pressure. The residue was poured onto crushed ice (600 g) and extracted with cold ether. The ether extracts were washed with brine, dried over sodium sulphate, filtered and evaporated under reduced pressure to give a light brown solid. This was triturated with warm hexane to yield 6-methoxycarbonyl-2,4,5-trichloropyrimidine (28 g, 82%). Characterising data for this compound are as follows: ¹H nmr (400 MHz, CDCl₃) δ_{H} 4.02 (3H, s) ppm.

### Example 5

### Synthesis of 2,5-dichloro-4-(furan-2-ylmethylamino)-6-methoxy carbonylpyrimidine (Compound 207-48)

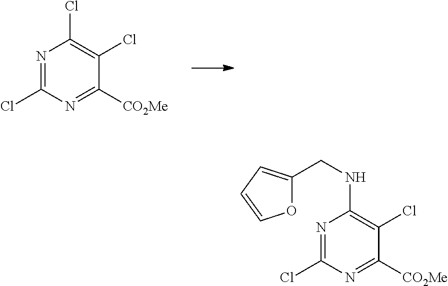

Furfurylamine (0.155 g, 1.60 mmol) was added to a stirred solution of 6-methoxycarbonyl-2,4,5-trichloropyrimidine (prepared as described in example 4) (0.193 g, 0.80 mmol) and triethylamine (0.24 ml, 1.7 mmol) in dichloromethane (3 ml). The solution was stirred at ambient temperature for 18 hours, and then added to a mixture of ethyl acetate and brine. The organic phase was dried over magnesium sulphate, filtered and evaporated under reduced pressure to give an orange solid. This was purified by column chromatography on silica using ethyl acetate:hexane (1:2) as eluent to provide 2,5-dichloro-4-(furan-2-ylmethylamino)-6-methoxycarbonylpyrimidine as a pale yellow solid (0.195 g, 81%). Characterising data for this compound are as follows: m.p. 110-112° C.; ¹H nmr (400 MHz, CDCl₃) δ_{H} 7.40 (1H, m), 6.35 (2H, m), 6.16 (1H, br s), 4.72 (2H, d), 3.98 (3H, s) ppm.

Further examples of compounds that were prepared using this method are listed below in Table 6.

TABLE 6
Compounds made according to the method described in Example 5 above.
Characteristic data provided is melting point (° C.)

| Compound | | | Characteristic |
|---|---|---|---|
| Number | Name | Structure | data |
| 207-30 | 4-Cyclopropylamino- 2,5-dichloro-6- methoxycarbonyl- pyrimidine | 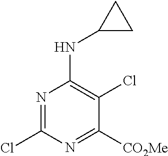 | 88-89 |
| 207-35 | 2,5-Dichloro-6- methoxycarbonyl-4- phenylmethylamino- pyrimidine | 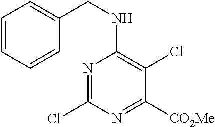 | 82-83 |
| 207-31 | 4-Cyclopropylmethyl- amino-2,5-dichloro-6- methoxycarbonyl- pyrimidine | 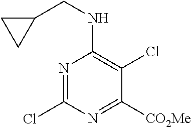 | 92-93 |
| 207-50 | 2,5-Dichloro-6- methoxycarbonyl-4-(5- methylfuran-2- ylmethylamino)- pyrimidine | 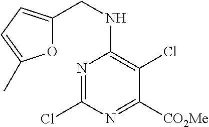 | 104-105 |

### Example 6

### Synthesis of 5-chloro-2-(4-chlorophenyl)-4-(furan-2-ylmethylamino)-6-methoxycarbonylpyrimidine (Compound 40-125)

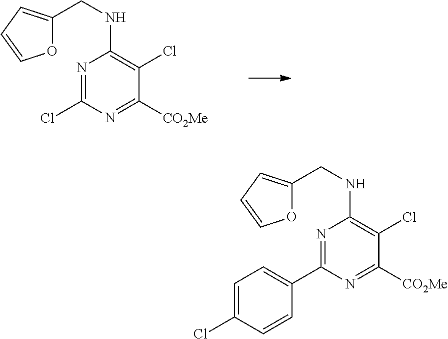

A solution of 4-chlorophenyl boronic acid pinacol ester (110 mg, 0.50 mmol), 2,5-dichloro-4-(furan-2-ylmethylamino)-6-methoxycarbonylpyrimidine (prepared as described in Example 5) (146 mg, 0.50 mmol), caesium fluoride (151 mg, 1.0 mmol) and [1,1-bis(diphenylphosphino)-ferrocene]dichloropalladium (II) complex with dichloromethane (1:1) (41 mg, 0.05 mmol) in dimethoxyethane (1 ml) and water (1 ml) was heated in a microwave reactor at 140° C. for 10 minutes. The reaction mixture was allowed to cool, and then added to mixture of ethyl acetate (10 ml) and brine (10 ml). The organic phase was dried over magnesium sulphate, filtered and evaporated under reduced pressure to leave a brown gum. This was purified by chromatography on silica with 20% ethyl acetate in hexane as eluent to provide 5-chloro-2-(4-chlorophenyl)-4-(furan-2-ylmethylamino)-6-methoxycarbonylpyrimidine as a pale yellow solid (104 mg, 57%). Characterising data for this compound are as follows: m.p. 132-134° C.; ¹H nmr (400 MHz, CDCl₃) δ_{H} 8.36 (2H, d), 7.42 (1H, m), 7.41 (2H, d), 6.36 (2H, m), 5.99 (1H, br t), 4.86 (2H, d), 4.03 (3H, s) ppm.

Further examples of compounds that were prepared using this method are listed below in Table 7. Characteristic data provided is either melting point (° C.), ¹H-nmr data (400 MHz, CDCl₃) δ_{H} ppm or LCMS [retention time (RT, recorded in minutes) and the molecular ion, typically the cation M+H⁺]. Compounds characterised by HPLC-MS used a Waters 2777 injector with a 1525 micro pump HPLC equipped with a Waters Atlantis dC18 IS column (column length 20 mm, internal diameter of column 3 mm, particle size 3 micron), Waters 2996 photodiode array, Waters 2420 ELSD and Micromass ZQ2000. The analysis was conducted using a three minute run time, according to the following gradient table:

| Time | Solvent A | Solvent B | Flow (ml/ |
|---|---|---|---|
| (minutes) | (%) | (%) | minute) |
| 0.00 | 95 | 5 | 1.3 |
| 2.50 | 0 | 100 | 1.3 |
| 2.80 | 0 | 100 | 1.3 |
| 2.90 | 95 | 5 | 1.3 |

Solvent A: H₂O with 0.05% TFA
Solvent B: CH₃CN with 0.05% TFA

TABLE 7
Compounds made according to the method described in Example 6 above.

| Compound | | | |
|---|---|---|---|
| Number | Name | Structure | Characteristic data |
| 40-1 | 5-Chloro-2-(4-chloro- phenyl)-4-cyclo- propylamino-6- methoxycarbonyl- pyrimidine | 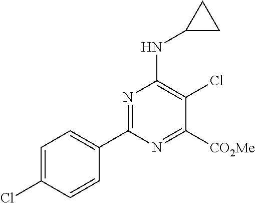 | 74-76 |
| 40-60 | 5-Chloro-2-(4- chlorophenyl)-6- methoxycarbonyl-4- (phenylmethylamino) pyrimidine | 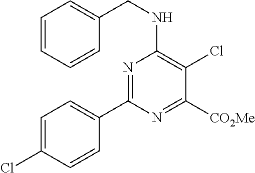 | 107-109 |
| 262-30 | 5-Chloro-2-(5-chloro- thiophen-2-yl)-4- cyclopropylamino-6- methoxycarbonyl- pyrimidine | 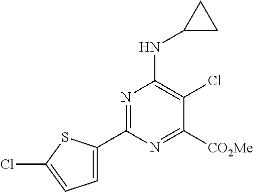 | 100-101 |
| 262-48 | 5-Chloro-2-(5- chlorothiophen-2-yl)- 4-(furan-2-yl- methylamino)-6- methoxycarbonyl- pyrimidine | 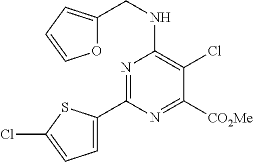 | 7.74 (1 H, d), 7.40 (1 H, s), 6.92 (1 H, d), 6.36 (2 H, m), 5.97 (1 H, br t), 4.77 (2 H, d), 3.99 (3 H, s), 1.27 (4 H, m), 1.11 (2 H, m), 1.04 (2 H, m) |
| 112-125 | 5-Chloro-2-(4-chloro- 2-fluoro-3- methoxyphenyl)-4- (furan-2-yl-methyl- amino)-6-methoxy- carbonylpyrimidine | 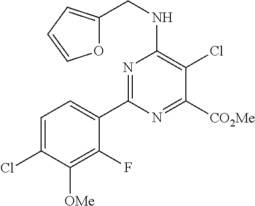 | 135-137 |
| 112-43 | 5-Chloro-2-(4-chloro- 2-fluoro-3-methoxy- phenyl)-4-cyclo- propylmethylamino- 6-methoxycarbonyl- pyrimidine | 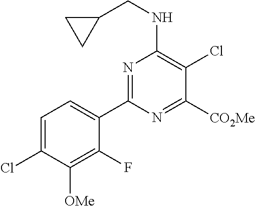 | 113-115 |
| 261-48 | 5-Chloro-2-(4-chloro- 3-dimethylamino-2- fluorophenyl)-4- (furan-2-yl-methyl- amino)-6-methoxy- carbonylpyrimidine | 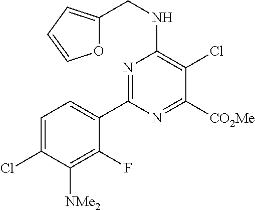 | 108-110 |
| 112-135 | 5-Chloro-2-(4-chloro- 2-fluoro-3- methoxyphenyl)-6- methoxycarbonyl-4- [(5-methylfuran-2- yl)methylamino)- pyrimidine | 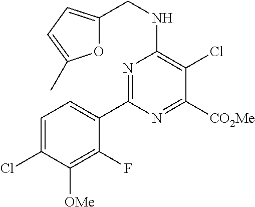 | 92-94 |
| 212-48 | 5-Chloro-2-(2,4- dichlorophenyl)-4- (furan-2-ylmethyl- amino)-6-methoxy- carbonylpyrimidine | 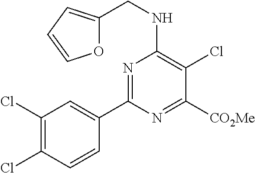 | RT 2.09, MH⁺ 412, 414, 416, 418 |
| 213-48 | 5-Chloro-2-(4-chloro- 2-methylphenyl)-4- (furan-2-yl-methyl- amino)-6-methoxy- carbonylpyrimidine | 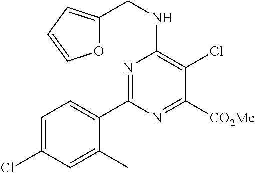 | RT 1.93, MH⁺ 392, 394, 396 |
| 214-48 | 5-Chloro-2-(4-chloro- 2-methoxyphenyl)-4- (furan-2-yl-methyl- amino)-6-methoxy- carbonylpyrimidine | 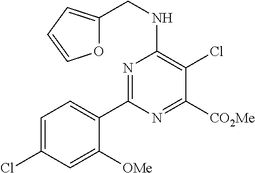 | RT 1.58, MH⁺ 408, 410, 412 |
| 215-48 | 5-Chloro-2-(4-chloro- 2,6-difluorophenyl)-4- (furan-2-yl-methyl- amino)-6-methoxy- carbonylpyrimidine | 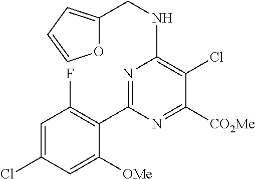 | RT 1.90, MH⁺ 414, 416, 418 |
| 216-48 | 5-Chloro-2-(4-chloro- 3-methylphenyl)-4- (furan-2-ylmethyl- amino)-6-methoxy- carbonylpyrimidine | 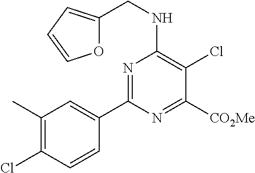 | RT 2.04, MH⁺ 392, 394, 396 |
| 217-48 | 5-Chloro-2-(4-chloro- 3-trifluoromethyl- phenyl)-4-(furan-2- ylmethylamino)-6- methoxycarbonyl- pyrimidine | 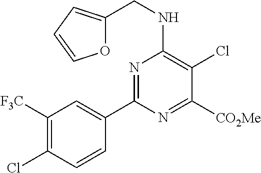 | RT 2.09, MH⁺ 446, 448, 450 |
| 218-48 | 5-Chloro-2-(4-chloro- 2-fluorophenyl)-4- (furan-2-yl-methyl- amino)-6-methoxy- carbonylpyrimidine | 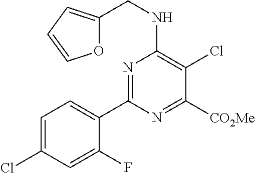 | RT 1.87, MH⁺ 396, 398, 400 |
| 219-48 | 5-Chloro-2-(4-chloro- 3-nitrophenyl)-4- (furan-2-yl-methyl- amino)-6- methoxycarbonyl- pyrimidine | 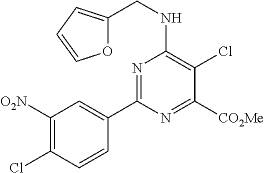 | RT 1.92, MH⁺ 423, 425, 427 |
| 220-48 | 5-Chloro-2-(4-chloro- 2-trifluoromethyl- phenyl)-4-(furan-2- ylmethylamino)-6- methoxycarbonyl- pyrimidine | 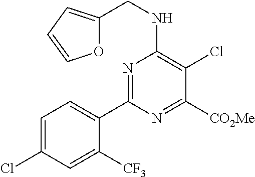 | RT 1.90, MH⁺ 446, 448, 450 |
| 221-48 | 5-Chloro-2-(4-chloro- 3-cyanophenyl)-4- (furan-2-yl-methyl- amino)-6-methoxy- carbonylpyrimidine | 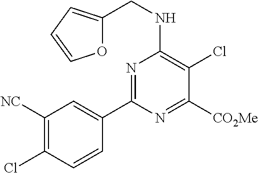 | RT 1.87, MH⁺ 403, 405, 407 |
| 222-48 | 5-Chloro-2-(4-chloro- 3-fluorophenyl)-4- (furan-2-ylmethyl- amino)-6- methoxycarbonyl- pyrimidine | 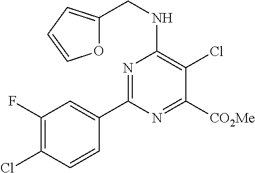 | RT 1.98, MH⁺ 396, 398, 400 |
| 224-48 | 5-Chloro-2-(2,4- dichloro-3- fluorophenyl)-4- (furan-2-ylmethyl- amino)-6-methoxy- carbonylpyrimidine | 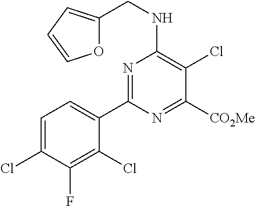 | RT 1.91, MH⁺ 430, 432, 434, 436 |
| 225-48 | 5-Chloro-2-(4-chloro- 2,3-difluorophenyl)-4- (furan-2-ylmethyl- amino)-6-methoxy- carbonylpyrimidine | 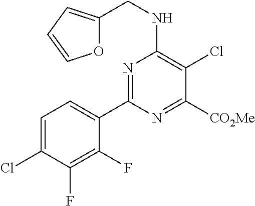 | RT 1.91, MH⁺ 414, 416, 418 |
| 226-48 | 5-Chloro-2-(3,4- dichloro-2- fluorophenyl)-4- (furan-2-ylmethyl- amino)-6-methoxy- carbonylpyrimidine | 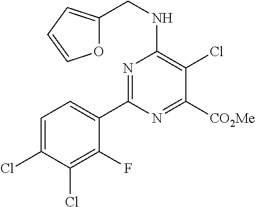 | RT 1.99, MH⁺ 430, 432, 434, 436 |
| 228-48 | 5-Chloro-4-(furan-2- ylmethylamino)-6- methoxycarbonyl-2- (2,4,5-trichloro- phenyl)pyrimidine | 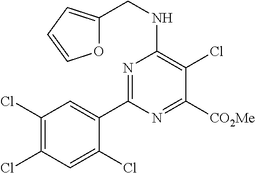 | RT 2.05, MH⁺ 446, 448, 450, 452, 454 |
| 229-48 | 5-Chloro-2-(4-chloro- 3-methoxyphenyl)-4- (furan-2-ylmethyl- amino)-6-methoxy- carbonylpyrimidine | 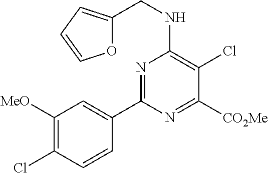 | RT 1.90, MH⁺ 408, 410, 412 |
| 230-48 | 5-Chloro-2-(4-chloro- 2-fluoro-3-trifluoro- methylphenyl)-4- (furan-2-ylmethyl- amino)-6-methoxy- carbonylpyrimidine | 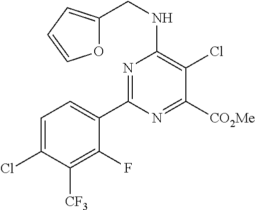 | RT 1.99, MH⁺ 464, 466, 468 |
| 231-48 | 5-Chloro-4-(furan-2- ylmethylamino)-6- methoxycarbonyl-2- (2,3,4-trichloro- phenyl)pyrimidine | 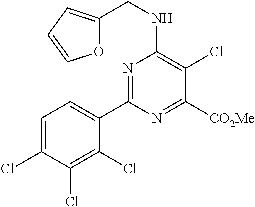 | RT 1.97, MH⁺ 446, 448, 450, 452, 454 |
| 232-48 | 5-Chloro-2-(4-chloro- 3,5-difluorophenyl)-4- (furan-2-ylmethyl- amino)-6-methoxy- carbonylpyrimidine | 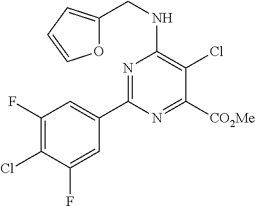 | RT 2.05, MH⁺ 414, 416, 418 |
| 233-48 | 5-Chloro-4-(furan-2- ylmethylamino)-6- methoxycarbonyl-2- (4-trifluoromethyl- phenyl)pyrimidine | 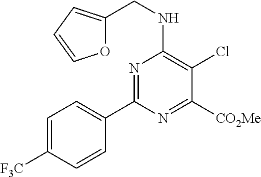 | RT 1.97, MH⁺ 412, 414 |
| 234-48 | 2-[2,4-Bis(trifluoro- methyl)phenyl]-5- chloro-4-(furan-2-yl- methylamino)-6- methoxycarbonyl- pyrimidine | 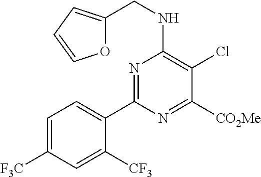 | RT 1.92, MH⁺ 480, 482 |
| 235-48 | 5-Chloro-2-(3-chloro- 4-trifluoromethyl- phenyl)-4-(furan-2- ylmethylamino)-6- methoxycarbonyl- pyrimidine | 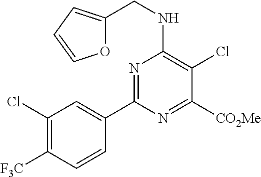 | RT 2.11, MH⁺ 446, 448, 450 |
| 236-48 | 5-Chloro-2-(2-chloro- 4-trifluoromethyl- phenyl)-4-(furan-2- ylmethylamino)-6- methoxycarbonyl- pyrimidine | 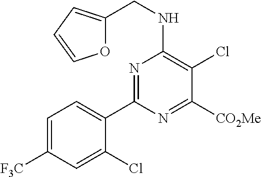 | RT 1.92, MH⁺ 446, 448, 450 |
| 237-48 | 5-Chloro-4-(furan-2- ylmethylamino)-6- methoxycarbonyl-2- (4-methylphenyl)- pyrimidine | 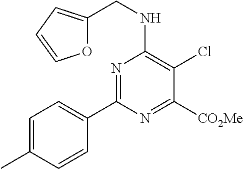 | RT 1.86, MH⁺ 358, 360 |
| 238-48 | 5-Chloro-2-(2-chloro- 4-methylphenyl)-4- (furan-2-yl-methyl- amino)-6-methoxy- carbonylpyrimidine | 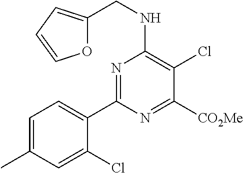 | RT 1.82, MH⁺ 392, 394, 396 |
| 239-48 | 5-Chloro-2-(3-chloro- 4-methylphenyl)-4- (furan-2-yl-methyl- amino)-6-methoxy- carbonylpyrimidine | 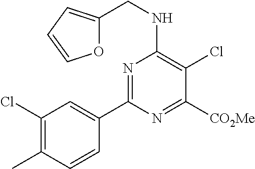 | RT 2.05, MH⁺ 392, 394, 396 |
| 240-48 | 5-Chloro-2-(3-fluoro- 4-methylphenyl)-4- (furan-2-yl-methyl- amino)-6-methoxy- carbonylpyrimidine | 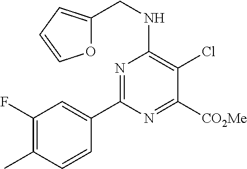 | RT 1.93, MH⁺ 376, 378 |
| 241-48 | 5-Chloro-4-(furan-2- ylmethylamino)-6- methoxycarbony-2- (2,4,5-trifluoro- phenyl)pyrimidine | 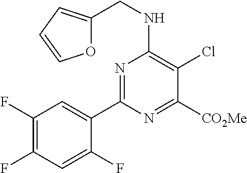 | RT 1.82, MH⁺ 398, 400 |
| 242-48 | 5-Chloro-2-(4- fluorophenyl)-4- (furan-2-yl-methyl- amino)-6-methoxy- carbonylpyrimidine | 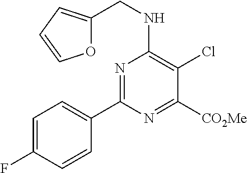 | RT 1.83, MH⁺ 362, 364 |
| 243-48 | 5-Chloro-2-(4-fluoro- 3-methoxyphenyl)-4- (furan-2-yl-methyl- amino)-6-methoxy- carbonylpyrimidine | 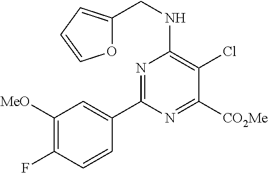 | RT 1.80, MH⁺ 392, 394 |
| 244-48 | 5-Chloro-2-(4-fluoro- 3-trifluoromethyl- phenyl)-4-(furan-2- ylmethylamino)-6- methoxycarbonyl- pyrimidine | 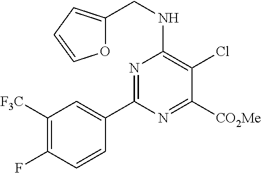 | RT 2.00, MH⁺ 430, 432 |
| 245-48 | 5-Chloro-2-(3-chloro- 4-fluorophenyl)-4- (furan-2-yl-methyl- amino)-6-methoxy- carbonylpyrimidine | 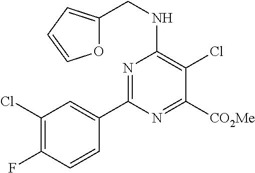 | RT 1.98, MH⁺ 396, 398, 400 |
| 247-48 | 5-Chloro-4-(furan-2- ylmethylamino)-6- methoxycarbonyl-2- (2,3,4-trifluoro- phenyl)pyrimidine | 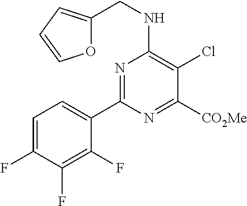 | RT 1.82, MH⁺ 398, 400 |
| 248-48 | 5-Chloro-4-(furan-2- ylmethylamino)-6- methoxycarbonyl-2- (4-trifluoromethoxy- phenyl)pyrimidine | 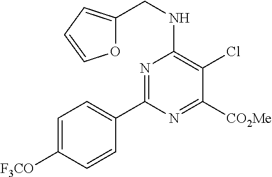 | RT 2.01, MH⁺ 428, 430 |
| 249-48 | 5-Chloro-2-(2-fluoro- 3-methoxyphenyl)-4- (furan-2-ylmethyl- amino)-6-methoxy- carbonylpyrimidine | 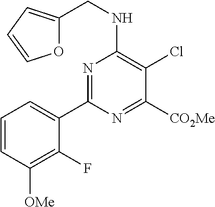 | RT 1.67, MH⁺ 392, 394 |
| 250-48 | 5-Chloro-4-(furan-2- ylmethylamino)-2-(4- methoxyphenyl)-6- methoxycarbonyl- pyrimidine | 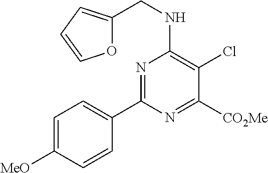 | RT 1.77, MH⁺ 374, 376 |
| 251-48 | 5-Chloro-4-(furan-2- yl-methylamino)-2-(4- methoxy-2-methyl- pheny)-6-methoxy- carbonylpyrimidine | 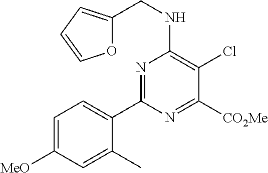 | RT 1.76, MH⁺ 388, 390 |
| 252-48 | 5-Chloro-2-(2,4- dimethoxyphenyl)-4- (furan-2-yl-methyl- amino)-6-methoxy- carbonylpyrimidine | 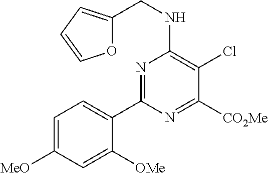 | RT 1.33, MH⁺ 404, 406 |
| 253-48 | 5-Chloro-2-(3-fluoro- 4-methoxyphenyl)-4- (furan-2-yl-methyl- amino)-6-methoxy- carbonylpyrimidine | 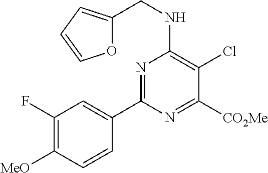 | RT 1.80, MH⁺ 392, 394 |
| 254-48 | 5-Chloro-2-(3-chloro- 4-methoxyphenyl)-4- (furan-2-yl-methyl- amino)-6-methoxy- carbonylpyrimidine | 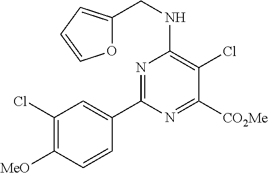 | RT 1.88, MH⁺ 408, 410, 412 |
| 255-48 | 5-Chloro-2-(3,4- dimethylphenyl)-4- (furan-2-yl-methyl- amino)-6-methoxy- carbonylpyrimidine | 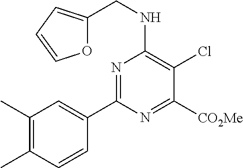 | RT 1.94, MH⁺ 372, 374 |
| 256-48 | 5-Chloro-4-(furan-2- ylmethylamino)-6- methoxycarbonyl-2- (4-methyl-3- nitrophenyl)- pyrimidine | 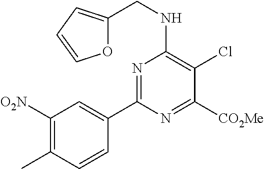 | RT 1.88, MH⁺ 403, 405 |
| 257-48 | 5-Chloro-2-(2-fluoro- 4-methylphenyl)-4- (furan-2- ylmethylamino)-6- methoxycarbonyl- pyrimidine | 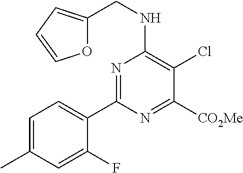 | RT 1.79, MH⁺ 376, 378 |
| 258-48 | 5-Chloro-2-(2-fluoro- 4-methoxyphenyl)-4- (furan-2-ylmethyl- amino)-6-methoxy- carbonylpyrimidine | 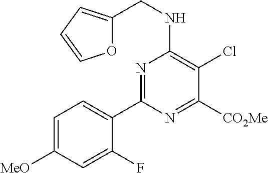 | RT 1.73, MH⁺ 392, 394 |
| 260-48 | 5-Chloro-2-(3,4- dimethoxyphenyl)-4- (furan-2-ylmethyl- amino)-6-methoxy- carbonylpyrimidine | 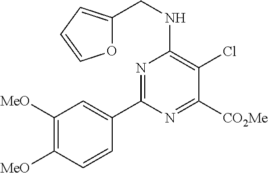 | RT 1.64, MH⁺ 404, 406 |

### Example 7

### Synthesis of 2-cyclopropyl-5-ethenyl-4-(furan-2-ylmethylamino)-6-methoxycarbonylpyrimidine (Compound 31-125)

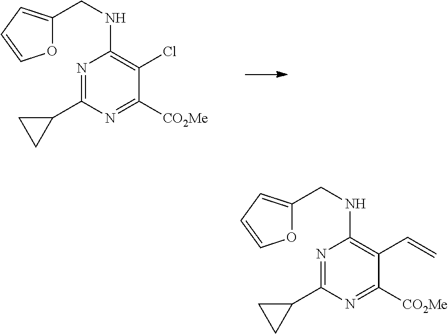

A solution of ethenyl boronic acid pinacol ester (174 mg, 1.13 mmol), 5-chloro-2-cyclopropyl-4-(furan-2-ylmethylamino)-6-methoxycarbonylpyrimidine (prepared as described in Example 2) (230 mg, 0.75 mmol), caesium fluoride (226 mg, 1.49 mmol) and [1,1′-bis(diphenylphosphino)-ferrocene]dichloropalladium (II) complex with dichloromethane (1:1) (61 mg, 0.075 mmol) in dimethoxyethane (1.5 ml) and water (1.5 ml) was heated in a microwave reactor at 140° C. for 20 minutes. The reaction mixture was allowed to cool, and then added to mixture of ethyl acetate (10 ml) and water (10 ml). The organic phase was dried over magnesium sulphate, filtered and evaporated under reduced pressure to leave a brown gum. This was purified by chromatography on silica with 20% ethyl acetate in hexane as eluent to provide 2-cyclopropyl-5-ethenyl-4-(furan-2-ylmethylamino)-6-methoxycarbonylpyrimidine as a pale yellow solid (115 mg, 52%). Characterising data for this compound are as follows: m.p. 85-86° C.; ¹H nmr (400 MHz, CDCl₃) δ_{H} 7.36 (1H, m), 6.70 (1H, q), 6.34 (1H, m), 6.21 (1H, m), 5.68 (1H, br t), 5.58 (1H, d), 5.48 (1H, d), 4.10 (2H, d), 3.90 (3H, s), 2.14 (1H, m), 1.12 (2H, m), 0.98 (2H, m) ppm. Further examples of compounds that were prepared using this method are listed below in Table 8.

TABLE 8
Compounds made according to the method described in Example 7 above.
Characteristic data provided is either melting point (° C.) and/or
¹H-nmr data (400 MHz, CDCl₃) δ_{H} ppm

| Compound | | | |
|---|---|---|---|
| Number | Name | Structure | Characteristic data |
| 67-125 | 2-(4-Chlorophenyl)- 5-ethenyl-4-(furan-2- ylmethylamino)-6- methoxycarbonyl- pyrimidine | 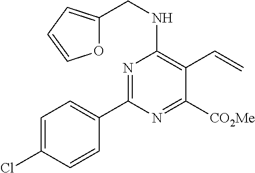 | 108-110 |
| 187-125 | 2-Cyclopropyl-5-(4- ethenylphenyl)-4- (furan-2- ylmethylamino)-6- methoxycarbonyl- pyrimidine | 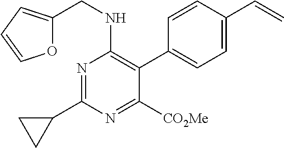 | 141-143 |
| 139-125 | 2-(4-Chloro-2-fluoro- 3-methoxyphenyl)-5- ethenyl-4-(furan-2- ylmethylamino)-6- methoxycarbonyl- pyrimidine | 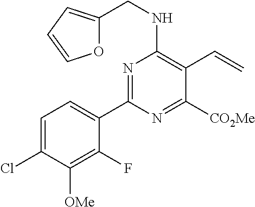 | 100-102 |
| 32-156 | 2-Cyclopropyl-5- ethenyl-6- methoxycarbonyl-4- (N-methyl-N-pyridin- 3-ylmethyl-amino)- pyrimidine | 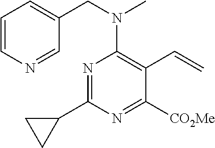 | 8.55 (1 H, d), 8.50 (1 H, s), 7.50 (1 H, d), 7.30 (1 H, m), 6.80 (1 H, dd), 5.30 (2 H, dd), 4.80 (2 H, s), 3.90 (3 H, s), 3.00 (3 H, s), 2.10 (1 H, m), 0.90 (4 H, m) |
| 32-160 | 2-Cyclopropyl-5- ethenyl-6- methoxycarbonyl-4- (N-methyl-N-pyridin- 4-ylmethyl-amino)- pyrimidine | 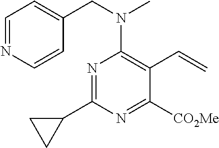 | 8.60 (2 H, d), 7.10 (2 H, m), 6.80 (1 H, dd), 5.30 (2 H, dd), 4.80 (2 H, s), 3.90 (3 H, s), 3.10 (3 H, s), 2.10 (1 H, m), 0.90 (4 H, m) |
| 33-160 | 2-Cyclopropyl-5- ethenyl-4-(N-ethyl-N- pyridin-4-ylmethyl- amino)-6-methoxy- carbonylpyrimidine | 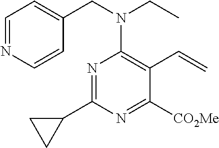 | 8.60 (2 H, d), 7.10 (2 H, m), 6.70 (1 H, dd), 5.30 (2 H, dd), 4.70 (2 H, s), 3.90 (3 H, s), 3.60 (2 H, q), 2.10 (1 H, m), 1.30 (3 H, t), 0.90 (4 H, m) |

### Example 8

### Synthesis of 5-chloro-2-cyclopropyl-4-(furan-2-ylmethylamino)-pyrimidine-6-carboxylic acid (Compound 1-125)

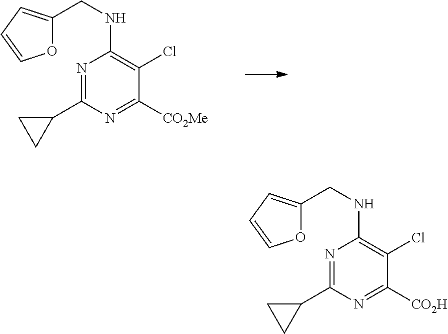

Sodium hydroxide (76 mg) was added to a stirred solution of 5-chloro-2-cyclopropyl-4-(furan-2-ylmethylamino)-6-methoxycarbonylpyrimidine (prepared by the method described in example 2) (275 mg, 0.90 mmol) in tetrahydrofuran (25 ml) and water (10 ml). The reaction mixture was allowed to stand at ambient temperature for 66 hours, then washed with ethyl acetate. The aqueous phase was acidified to pH 2-3 by the addition of 2N hydrochloric acid and the resulting precipitate removed by filtration and dried to provide 5-chloro-2-cyclopropyl-4-(furan-2-ylmethylamino)-pyrimidine-6-carboxylic acid. Characterising data for this compound are as follows: m.p. 150-152° C.; ¹H nmr (400 MHz, CDCl₃) δ_{H} 8.12 (1H, t), 7.55 (1H, m), 6.37 (1H, m), 6.21 (1H, m), 4.54 (2H, d), 1.96 (1H, m), 0.93 (4H, m) ppm (acid proton not observed).

Further examples of compounds that were prepared using this method are listed below in Table 9.

TABLE 9
Compounds made according to the method described in Example 8 above.
Characteristic data provided is either melting point (° C.) and/or
¹H-nmr data (400 MHz, D₂O) δ_{H} ppm

| Compound | | | |
|---|---|---|---|
| Number | Name | Structure | Characteristic data |
| 1-1 | 5-Chloro-2- cyclopropyl-4- cyclopropylamino- pyrimidine-6- carboxylic acid | 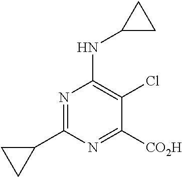 | 168-170 |
| 1-60 | 5-Chloro-2- cyclopropyl-4- phenylmethyamino pyrimidtne-6- carboxylic acid | 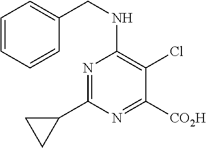 | 143-144 |
| 1-35 | 5-Chloro-4- cyclohexylamino-2- cyclopropyl- pyrimidine-6- carboxylic acid | 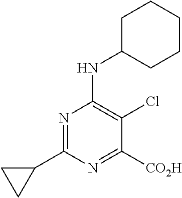 | 134-135 |
| 37-125 | 5-Chloro-2-(4- chlorophenyl)-4- (furan-2- ylmethylamino)- pyrimidine-6- carboxylic acid | 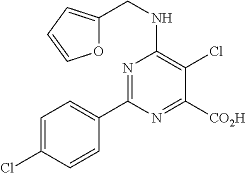 | 151-153 |
| 1-66 | 5-Chloro-2- cyclopropyl-4-(4- fluorophenylmethyl- amino)pyrimidine-6- carboxylic acid | 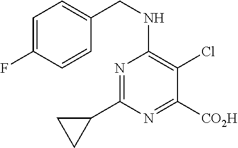 | 132-134 |
| 1-135 | 5-Chloro-2- cyclopropyl-4-(5- methylfuran-2- ylmethylamino)- pyrimidine-6- carboxylic acid | 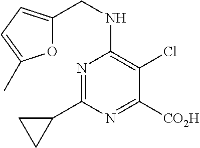 | 153-154 |
| 1-104 | 5-Chloro-2- cyclopropyl-4-(2,6- dichlorophenyl- methylamino)- pyrimidine-6- carboxylic acid | 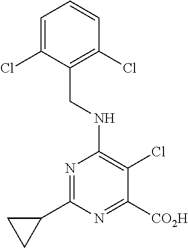 | 150-151 |
| 1-110 | 5-Chloro-2- cyclopropyl-4-(2,4- dimethoxyphenyl- methylamino)- pyrimidine-6- carboxylic acid | 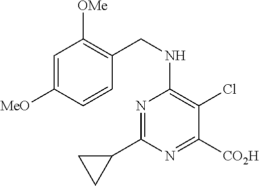 | 128-129 |
| 109-43 | 5-Chloro-2-(4- chloro-2-fluoro-3- methoxyphenyl)-4- cyclopropylmethyl- aminopyrimidine-6- carboxylic acid | 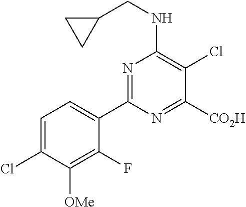 | 145-147 |
| 1-176 | 5-Chloro-4-[1-(4- chlorophenyl)ethyl- amino]-2- cyclopropyl- pyrimidine-6- carboxylic acid | 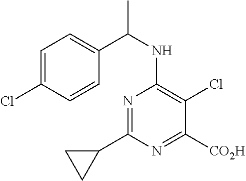 | >230 |
| 109-125 | 5-Chloro-2-(4- chloro-2-fluoro-3- methoxyphenyl)-4- (furan-2-yl-methyl- amino)pyrimidine-6- carboxylic acid | 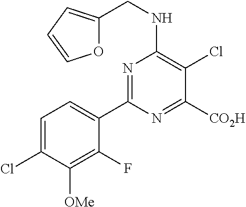 | 144-145 |
| 1-73 | 5-Chloro-2-cyclo- propyl-4-(4-methyl- phenylmethyl- amino)pyrimidine-6- carboxylic acid | 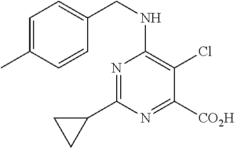 | 130-133 |
| 1-10 | 5-Chloro-2- cyclopropyl-4-[(1- pyridin-2-yl)cyclo- propylamino]- pyrimidine-6- carboxylic acid | 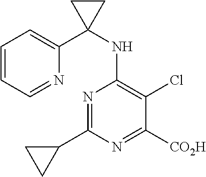 | 173-174 |
| 1-153 | 5-Chloro-2- cyclopropyl-4-(3- methylpyridin-2- ylmethylamino)- pyrimidine-6- carboxylic acid | 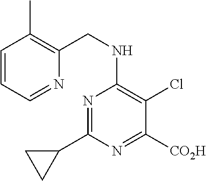 | 152-154 |
| 1-33 | 5-Chloro-2- cyclopropyl-4- cyclopentylamino- pyrimidine-6- carboxylic acid | 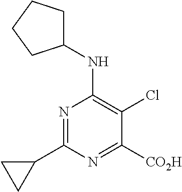 | 145-147 |
| 1-186 | 5-Chloro-2- cyclopropyl-4-[1- (furan-2- yl)ethylamino]- pyrimidine-6- carboxylic acid | 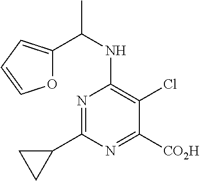 | 131-132 |
| 2-156 | 5-Chloro-2- cyclopropyl-4-(N- methyl-N-pyridin-3- ylmethyl-amino)- pyrimidine-6- carboxylic acid | 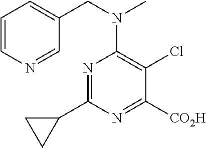 | 8.85 (1 H, s), 8.80 (1 H, d), 8.60 (1 H, d). 8.10 (1 H, dd), 5.20 (2 H, s), 3.60 (3 H, s), 2.20 (1 H, m), 1.20 (2 H, m), 0.90 (2 H, m) |
| 2-160 | 5-Chloro-2- cyclopropyl-4-(N- methyl-N-pyridin-4- ylmethyl-amino)- pyrimidine-6- carboxylic acid | 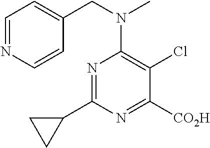 | 8.70 (2 H, d), 8.10 (2 H, dd), 5.20 (2 H, s), 3.60 (3 H, s), 2.00 (1 H, m), 1.10 (2 H, m), 0.80 (2 H, m) |
| 1-152 | 5-Chloro-4-(3- chloropyridin-2- ylmethylamino)-2- cyclopropyl- pyrimidine-6- carboxylic acid | 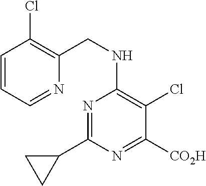 | 147-148 |

### Example 9

### Synthesis of 5-chloro-2-cyclopropyl-6-ethoxycarbonyl-4-phenylmethyl-aminopyrimidine (Compound 7-60)

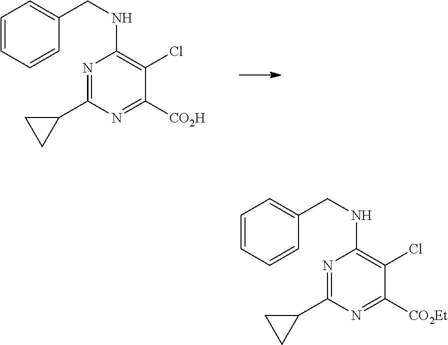

Thionyl chloride (0.05 ml) was added dropwise to a stirred suspension of 5-chloro-2-cyclopropyl-4-phenylmethylaminopyrimidine-6-carboxylic acid (prepared using the method described in example 8) (200 mg, 0.67 mmol) in ethanol (1.5 ml). The resulting solution was heated at 70° C. for 15 hours, cooled and neutralised by the addition of aqueous sodium bicarbonate. The mixture was extracted with ethyl acetate and the organic extract dried over magnesium sulphate, filtered and evaporated under reduced pressure to leave an orange gum, which was purified by column chromatography on silica using 20% ethyl acetate in hexane as eluent to provide 5-chloro-2-cyclopropyl-6-ethoxycarbonyl-4-phenylmethylaminopyrimidine as a solid (75 mg, 34%). Characterising data for this compound are as follows: m.p. 59-61° C.; ¹H nmr (400 MHz, CDCl₃) δ_{H} 7.35 (2H, m), 7.29 (3H, m), 5.57 (1H, br t), 4.67 (2H), 4.44 (2H, q), 2.10 (1H, m), 1.41 (3H, t), 1.05 (2H, m), 0.97 (2H, m) ppm.

Further examples of compounds that were prepared using this method are listed below in Table 10.

TABLE 10
Compounds made according to the method described in Example 9 above.
Characteristic data provided is either melting point (° C.) and/or
¹H-nmr data (400 MHz, CDCl₃) δ_{H} ppm

| Compound | | | |
|---|---|---|---|
| Number | Name | Structure | Characteristic data |
| 7-125 | 5-Chloro-2- cyclopropyl-6- ethoxycarbonyl-4- (2-furanylmethyl- amino)pyrimidine | 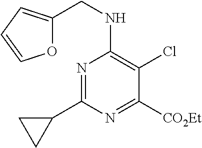 | 79-80 |
| 197-125 | 5-Chloro-2-cyclo- propyl-4-(2- furanylmethyl- amino)-6-iso- propoxycarbonyl- pyrimidine | 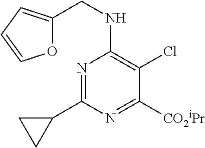 | 7.37 (1 H, m), 6.34 (1 H, m), 6.24 (1 H, m), 5.78 (1 H, br t), 5.30 (1 H, septet), 4.66 (2 H, d), 2.12 (1 H, m), 1.40 (6 H, d), 1.10 (2 H, m), 0.99 (2 H, m) |

### Example 10

### Synthesis of 5-chloro-2-cyclopropyl-4-[(furan-2-ylmethyl)-amino]-6-ⁿpropoxycarbonylpyrimidine (Compound 198-125)

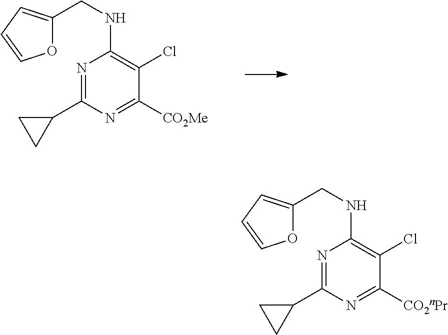

A solution of 5-chloro-2-cyclopropyl-4-(furan-2-ylmethylamino)-6-methoxycarbonylpyrimidine (prepared by the method described in example 2) (100 mg, 0.325 mmol), 1-hydroxy-3-isothionato-1,1,3,3-tetrabutyl distannoxane (18 mg, 0.0325 mmol), and propan-1-ol (250 ul, 3.25 mmol) in toluene (4 ml) was heated at reflux for 3 hours. The reaction was allowed to cool and evaporated under reduced pressure to give a pale brown oil, which was purified on a Biotage SP4 system using a 12S column and a gradient elution from 12% ethyl acetate in isohexane to ethyl acetate gradient to provide 5-chloro-2-cyclopropyl-4-[(furan-2-ylmethyl)-amino]-6-npropoxycarbonylpyrimidine as a colourless oil (88 mg, 81%). Characterising H 7.35 (1H, d), □data for this compound are as follows: 1H NMR (400 MHz, CDCl3) 6.35 (1H, dd), 6.25 (1H, d), 5.80 (1H, br t), 4.65 (2H, d), 4.35 (2h, t), 2.15 (1H, m), 1.80 (2H, m), 1.10 (2H, m), 1.05-0.95 (5H, m).

Further examples of compounds that were prepared using this method are listed below in Table 11.

TABLE 11
Compounds made according to the method described in Example 10 above.
Characteristic data provided is either melting point (° C.) and/or
¹H-nmr data (400 MHz, CDCl₃) δ_{H} ppm

| Compound | | | |
|---|---|---|---|
| Number | Name | Structure | Characteristic data |
| 200-125 | 5-Chloro-2- cyclopropyl-4-(2- furanylmethyl- amino)-6-phenyl- methylcarbonyl- pyrimidine | 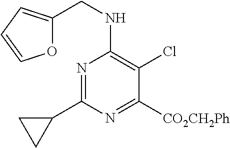 | 7.50 (2 H, m), 7.45- 7.35 (4 H, m), 6.40 (1 H, dd), 6.30 (1 H, d), 6.10 (1 H, br t), 5.45 (2 H, s), 4.70 (2 H, d), 2.25 (1 H, m), 1.20 (2 H, m), 1.10 (2 H, m) |
| 199-125 | 5-Chloro-2- cyclopropyl-4-(2- furanylmethyl- amino)-6-iso- butoxycarbonyl- pyrimidine | 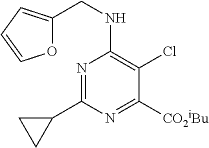 | 7.40 (1 H, d), 6.40 (1 H, dd), 6.30 (1 H, d), 5.85 (1 H, br t), 4.70 (2 H, d), 4.20 (2 H, d), 2.20-2.10 (2 H, m), 1.15 (2 H, m), 1.05-1.00 (8 H, m) |

### Example 11

### Synthesis of 6-aminocarbonyl-5-chloro-2-cyclopropyl-4-(furan-2-ylmethylamino)-pyrimidine (Compound 201-125)

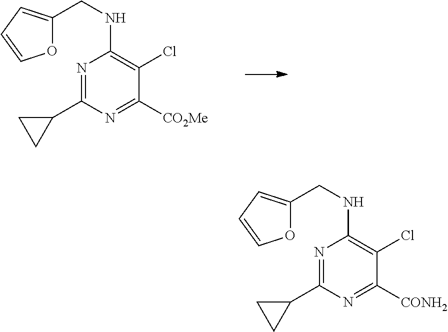

A solution of 5-chloro-2-cyclopropyl-4-(furan-2-ylmethylamino)-6-methoxy-carbonylpyrimidine (prepared by the method described in example 2) (300 mg, 0.97 mmol) in methanolic ammonia (7M; 2 ml, 14 mmol) was stirred at ambient temperature for 6 hours and then allowed to stand for a further 18 hours. The mixture was partitioned between ethyl acetate and water and the organic phase dried over magnesium sulphate, filtered and evaporated under reduced pressure to provide 6-aminocarbonyl-5-chloro-2-cyclopropyl-4-(furan-2-ylmethylamino)-pyrimidine as a beige solid (252 mg, 88%). Characterising data for this compound are as follows: M.p. 126-128° C.; ¹H nmr (400 MHz, CDCl₃) δ_{H} 7.71 (1H, br s), 7.38 (1H, m), 6.33 (1H, m), 6.26 (1H, m), 6.06 (1H, br t), 5.64 (1H, br s), 4.67 (2H, d), 2.07 (1H, m), 1.07 (2H, m), 1.00 (2H, m) ppm.

### Example 12

### Synthesis of 2-cyclopropyl-4-hydroxy-6-methoxycarbonylmethoxy-5-methylpyrimidine

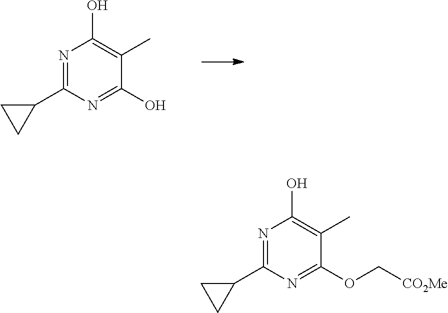

A mixture of 2-cyclopropyl-4,6-dihydroxy-5-methylpyrimidine (1.6 g, 9.6 mmol), methyl bromoacetate (1.5 g, 9.8 mmol), potassium carbonate (1.4 g, 10 mmol) and dimethylformamide (7.5 ml) was heated at 90° C. for 6 hours, then allowed to cool and stood at ambient temperature for 65 hours. Water was added and the resulting precipitate washed with ether and dried to provide a white solid. This was washed with ethyl acetate to provide 2-cyclopropyl-4-hydroxy-6-methoxycarbonylmethoxy-5-methylpyrimidine as a white solid (450 mg, 19%).

¹H nmr (400 MHz, CDCl₃) δ_{H} 13.80 (1H, br s), 4.70 (2H, s), 3.70 (3H, s), 1.90 (4H, m), 1.05 (4H, m) ppm.

Further examples of compounds that were prepared using this method are listed below in Table 12.

TABLE 12
Compounds made according to the method described in Example 12 above.
Characteristic data provided is ¹H-nmr data (400 MHz, CDCl₃) δ_{H} ppm

| Name | Structure | Characteristic data |
|---|---|---|
| 6-^{t}Butoxycarbonylmethoxy-2- cyclopropyl-4-hydroxy-5- methylpyrimidine | 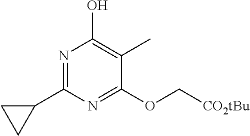 | 4.70 (2 H, s), 2.00 (1 H, m), 2.00 (3 H, s), 1.45 (9 H, s), 1.10 (4 H, m) (OH not observed) |

### Example 13

### Synthesis of 2-cyclopropyl-4-cyclopropylmethylamino-6-cyclopropylmethylaminocarbonylmethoxy-5-methylpyrimidine (Compound 266-20)

### 13.1 Preparation of 4-chloro-2-cyclopropyl-6-methoxycarbonylmethoxy-5-methylpyrimidine

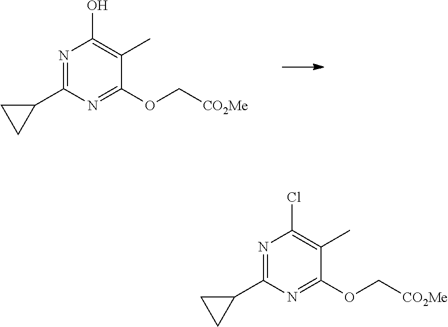

A stirred suspension of 2-cyclopropyl-4-hydroxy-6-methoxycarbonylmethoxy-5-methylpyrimidine (prepared as described in example 12) (450 mg, 1.9 mmol) in phosphorus oxychloride (3 ml) was heated at 120° C. for 5 hours. The reaction mixture was allowed to cool and evaporated under reduced pressure. The residue was dissolved in dichloromethane and the solution washed with saturated aqueous sodium bicarbonate, dried over magnesium sulphate, filtered and evaporated under reduced pressure. The residue was purified by column chromatography on silica using diethyl ether as eluent to provide 4-chloro-2-cyclopropyl-6-methoxycarbonylmethoxy-5-methylpyrimidine as a solid (350 mg, 70%).

¹H nmr (400 MHz, CDCl₃) δ_{H} 4.85 (2H, s), 3.75 (3H, s), 2.20 (3H, s), 2.10 (1H, m), 1.00 (4H, m) ppm.

### 13.2 Preparation of 2-cyclopropyl-4-cyclopropylmethylamino-6-cyclopropylmethylaminocarbonylmethoxy-5-methylpyrimidine

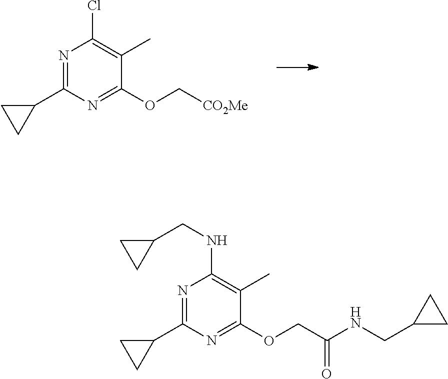

A solution of 4-chloro-2-cyclopropyl-6-methoxycarbonylmethoxy-5-methylpyrimidine (50 mg, 0.19 mmol) and cyclopropylmethylamine (0.5 ml, 5.8 mmol) in dichloroethane was heated at reflux for 4 hours, then allowed to cool. Water was added and the resulting mixture extracted with ethyl acetate. The combined organic phases were dried over magnesium sulphate, filtered and evaporated under reduced pressure to provide 2-cyclopropyl-4-cyclopropylmethylamino-6-cyclopropylmethylaminocarbonylmethoxy-5-methylpyrimidine as an oil (12 mg, 21%).

¹H nmr (400 MHz, CDCl₃) δ_{H} 6.60 (1H, br s), 4.80 (2H, s), 4.50 (1H, br t), 3.35 (2H, m), 3.20 (2H, m), 1.90 (3H, s), 1.90 (1H, m), 1.70 (1H, br s), 1.05 (1H, m), 1.00 (2H, m), 0.80 (2H, m), 0.50 (4H, m), 0.30 (2H, m), 0.25 (2H, m) ppm.

### Example 14

### Synthesis of 6-^{t}butoxycarbonylmethoxy-2-cyclopropyl-4-cyclopropylmethylamino-5-methylpyrimidine (Compound 266-19)

### 14.1 Preparation of 6-^{t}butoxycarbonylmethoxy-2-cyclopropyl-5-methyl-4-(trifluoromethylsulphonyloxy)-pyrimidine

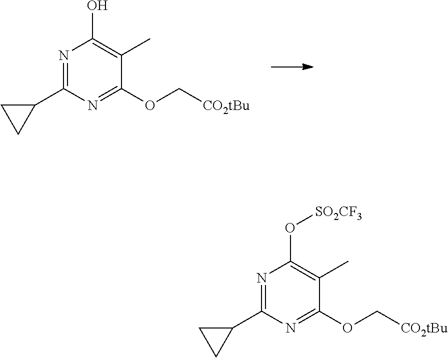

A solution of trifluoromethanesulphononic anhydride (150 mg, 0.53 mmol) in dichloromethane (3 ml) was added to a stirred solution of 6-^{t}butoxycarbonylmethoxy-2-cyclopropyl-4-hydroxy-5-methylpyrimidine (prepared as described in example 12) (136 mg, 0.48 mmol) and pyridine (50 mg, 0.22 mmol) in dichloromethane (10 ml). The reaction mixture was stirred at ambinent temperature for 2 hours, the solvent evaporated under reduced pressure and ether added. The resulting solution was washed with water, dried over magnesium sulphate, filtered and evaporated under reduced pressure. The residue was purified by column chromatography on silica to provide 6-^{t}butoxycarbonylmethoxy-2-cyclopropyl-5-methyl-4-(trifluoromethylsulphonyloxy)-pyrimidine as an oil (115 mg, 58%).

¹H nmr (400 MHz, CDCl₃) δ_{H} 4.80 (2H, s), 2.15 (3H, s), 2.05 (1H, m), 1.45 (9H, s), 1.05 (4H, m) ppm.

### 14.2 Preparation of 6-^{t}butoxycarbonylmethoxy-2-cyclopropyl-4-cyclopropylmethylamino-5-methylpyrimidine

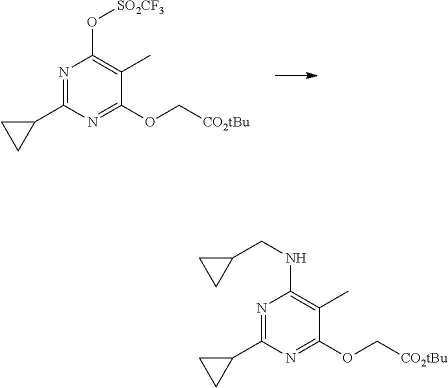

A solution of 6-^{t}butoxycarbonylmethoxy-2-cyclopropyl-5-methyl-4-(trifluoromethylsulphonyloxy)-pyrimidine (120 mg, 0.29 mmol) and cyclopropylmethylamine (100 mg, 1.4 mmol) in dimethoxyethane (10 ml) was heated at reflux for 90 minutes, then allowed to cool and evaporated under reduced pressure. The residue was purified by column chromatography on silica using diethyl ether as eluent to provide 6-^{t}butoxycarbonylmethoxy-2-cyclopropyl-4-cyclopropylmethylamino-5-methyl-pyrimidine as an off-white solid (97 mg, 100%).

¹H nmr (400 MHz, CDCl₃) δ_{H} 4.70 (2H, s), 4.40 (1H, br t), 3.35 (2H, t), 1.95 (3H, s), 1.90 (1H, m), 1.45 (9H, s), 1.05 (1H, m), 1.00 (2H, m), 0.85 (2H, m), 0.50 (2H, m), 0.25 (2H, m) ppm.

### Example 15

### Synthesis of 6-carboxymethoxy-2-cyclopropyl-4-cyclopropylmethylamino-5-methylpyrimidine (Compound 266-17) trifluoroacetate salt

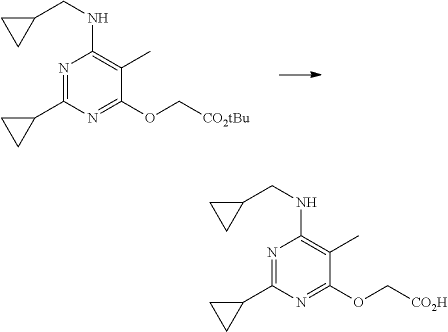

A solution of 6-^{t}butoxycarbonylmethoxy-2-cyclopropyl-4-cyclopropylmethylamino-5-methylpyrimidine (prepared as described in example 14) (75 mg, 0.23 mmol) and trifluoroacetic acid (0.5 ml) in dichloroethane (5 ml) was heated at reflux for 6 hours then allowed to cool. The resulting solution was azeotroped with toluene (3×50 ml) to provide 6-carboxymethoxy-2-cyclopropyl-4-cyclopropylmethylamino-5-methylpyrimidine trifluoroacetate salt as an oil (88 mg, 100%).

¹H nmr (400 MHz, CDCl₃) δ_{H} 4.87 (2H, s), 3.45 (2H, d), 2.35 (1H, pentet), 2.04 (3H, s), 1.10 (4H, m), 0.88 (1H, m), 0.64 (2H, m), 0.33 (2H, m) ppm (exchangeable protons not observed).

### Example 16

### Synthesis of 2-cyclopropyl-4-cyclopropylmethylamino-6-methoxycarbonylmethoxy-5-methylpyrimidine (Compound 266-18)

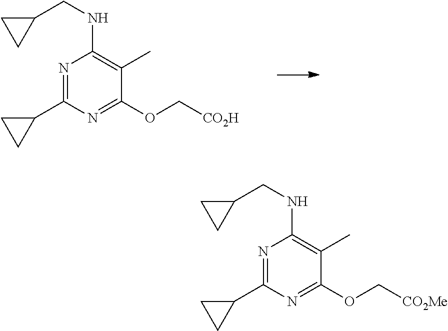

A solution of 6-carboxymethoxy-2-cyclopropyl-4-cyclopropylmethylamino-5-methylpyrimidine trifluoroacetate salt (prepared as described in example 15) (50 mg, 0.23 mmol) and trifluoroacetic acid (0.3 ml) in methanol (2 ml) was heated in a microwave reactor at 135° C. for 45 minutes, then allowed to cool and the solvent evaporated under reduced pressure. The residue was purified by column chromatography on silica using diethyl ether as eluent to provide 2-cyclopropyl-4-cyclopropylmethylamino-6-methoxycarbonylmethoxy-5-methylpyrimidine as an oil (32 mg, 86%).

¹H nmr (400 MHz, CDCl₃) δ_{H} 4.80 (2H, s), 3.80 (3H, s), 3.40 (2H, d), 2.10 (1H, m), 2.00 (3H, s), 1.10 (1H, m), 1.00 (4H, m), 0.55 (2H, m), 0.30 (2H, m) ppm (NH not observed).

### Example 17

### Pre-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retoflexus (AMARE) and Ipomea hederaceae (IPOHE) were sown in standard soil in pots. After cultivation for one day under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethelyene sorbitan monolaurate, CAS RN 9005-64-5) to give a final dose of 1000 or 250 g/ha of test compound.

The test plants were then grown under controlled conditions in the glasshouse (at 24/16° C., day/night; 14 hours light; 65% humidity) and watered twice daily. After 13 days the test was evaluated (100=total damage to plant; 0=no damage to plant). Results are shown below in Table B1.

TABLE B1
Percentage damage caused to weed species by compounds of the
invention when applied pre-emergence.

| Compound | Rate | Species | | | | | |
|---|---|---|---|---|---|---|---|
| Number | (g/ha) | SOLNI | AMARE | SETFA | ALOMY | ECHCG | IPOHE |
| 1-1 | 1,000 | 90 | 100 | 0 | 20 | 20 | 90 |
| 1-33 | 1,000 | 80 | 80 | 50 | 30 | 30 | 70 |
| 1-35 | 1,000 | 20 | 50 | 0 | 10 | 10 | 0 |
| 1-60 | 1,000 | 90 | 90 | 0 | 20 | 20 | 100 |
| 1-66 | 1,000 | 60 | 50 | 10 | 10 | 10 | 90 |
| 1-73 | 1,000 | 80 | 70 | 30 | 10 | 10 | 60 |
| 1-104 | 1,000 | 0 | 0 | 0 | 0 | 0 | 0 |
| 1-110 | 1,000 | 60 | 100 | 30 | 40 | 20 | 90 |
| 1-125 | 1,000 | 100 | 100 | 70 | 60 | 80 | 100 |
| 1-135 | 1,000 | 100 | 100 | 90 | 80 | 90 | 100 |
| 1-153 | 1,000 | 90 | 100 | 70 | 40 | 50 | 100 |
| 1-176 | 1,000 | 0 | 0 | 0 | 0 | 0 | 0 |
| 4-1 | 1,000 | 100 | 100 | 60 | 40 | 50 | 90 |
| 4-5 | 1,000 | 0 | 40 | 0 | 0 | 0 | 40 |
| 4-6 | 1,000 | 20 | 40 | 0 | 0 | 0 | 10 |
| 4-10 | 1,000 | 20 | 30 | 0 | 0 | 0 | 80 |
| 4-13 | 1,000 | 20 | 20 | 0 | 0 | 0 | 50 |
| 4-16 | 1,000 | 10 | 30 | 0 | 0 | 0 | 10 |
| 4-17 | 1,000 | 10 | 50 | 0 | 10 | 20 | 70 |
| 4-19 (Z) | 1,000 | 40 | 90 | 0 | 20 | 20 | 90 |
| 4-19 (E) | 1,000 | 60 | 40 | 20 | 20 | 20 | 80 |
| 4-20 | 1,000 | 20 | 50 | 0 | 0 | 0 | 50 |
| 4-21 | 1,000 | 50 | 20 | 0 | 0 | 0 | 70 |
| 4-22 | 1,000 | 20 | 70 | 10 | 20 | 20 | 80 |
| 4-23 | 1,000 | 60 | 70 | 0 | 10 | 0 | 70 |
| 4-24 | 1,000 | 90 | 100 | 0 | 10 | 0 | 40 |
| 4-25 | 1,000 | 60 | 100 | 0 | 10 | 10 | 30 |
| 4-26 | 1,000 | 20 | 20 | 0 | 10 | 0 | 50 |
| 4-27 | 1,000 | 10 | 40 | 0 | 10 | 0 | 80 |
| 4-28 | 1,000 | 40 | 90 | 10 | 10 | 0 | 70 |
| 4-29 | 1,000 | 30 | 30 | 10 | 10 | 0 | 70 |
| 4-30 | 1,000 | 0 | 20 | 10 | 0 | 0 | 40 |
| 4-31 | 1,000 | 20 | 80 | 0 | 0 | 0 | 70 |
| 4-32 | 1,000 | 80 | 80 | 80 | 50 | 50 | 80 |
| 4-33 | 1,000 | 20 | 50 | 10 | 0 | 20 | 50 |
| 4-34 | 1,000 | 80 | 100 | 50 | 30 | 20 | 90 |
| 4-35 | 1,000 | 0 | 0 | 0 | 0 | 0 | 0 |
| 4-36 (trans) | 1,000 | 70 | 80 | 20 | 10 | 20 | 60 |
| 4-37 (1R,2S) | 1,000 | 100 | 100 | 80 | 50 | 60 | 100 |
| 4-37 (1S,2R) | 1,000 | 90 | 100 | 60 | 30 | 40 | 80 |
| 4-38 (S,S) | 1,000 | 80 | 100 | 30 | 20 | 30 | 70 |
| 4-39 | 1,000 | 50 | 60 | 0 | 0 | 20 | 80 |
| 4-42 | 1,000 | 0 | 30 | 10 | 10 | 20 | 0 |
| 4-43 | 1,000 | 90 | 90 | 70 | 70 | 70 | 80 |
| 4-49 | 1,000 | 30 | 10 | 0 | 0 | 0 | 40 |
| 4-50 | 1,000 | 100 | 100 | 10 | 10 | 10 | 100 |
| 4-51 | 1,000 | 10 | 30 | 0 | 0 | 0 | 50 |
| 4-52 | 1,000 | 30 | 40 | 0 | 0 | 0 | 0 |
| 4-53 | 1,000 | 40 | 60 | 0 | 10 | 0 | 60 |
| 4-55 | 1,000 | 10 | 100 | 0 | 0 | 0 | 60 |
| 4-56 | 1,000 | 90 | 100 | 20 | 40 | 30 | 100 |
| 4-58 | 1,000 | 30 | 80 | 0 | 0 | 0 | 20 |
| 4-59 | 1,000 | 80 | 80 | 10 | 20 | 40 | 80 |
| 4-60 | 1,000 | 50 | 60 | 30 | 0 | 0 | 60 |
| 4-62 | 1,000 | 90 | 100 | 10 | 10 | 20 | 60 |
| 4-63 | 1,000 | 60 | 60 | 20 | 20 | 20 | 60 |
| 4-65 | 1,000 | 80 | 80 | 40 | 20 | 60 | 70 |
| 4-66 | 1,000 | 90 | 90 | 80 | 40 | 50 | 90 |
| 4-67 | 250 | 20 | 40 | 0 | 0 | 0 | 0 |
| 4-68 | 1,000 | 20 | 50 | 10 | 10 | 10 | 60 |
| 4-70 | 1,000 | 30 | 40 | 20 | 10 | 30 | 60 |
| 4-71 | 1,000 | 20 | 90 | 20 | 20 | 20 | 70 |
| 4-72 | 1,000 | 40 | 60 | 10 | 10 | 40 | 40 |
| 4-73 | 1,000 | 60 | 70 | 20 | 10 | 30 | 80 |
| 4-74 | 1,000 | 70 | 80 | 10 | 10 | 30 | 60 |
| 4-75 | 1,000 | 30 | 50 | 10 | 10 | 0 | 20 |
| 4-76 | 1,000 | 60 | 60 | 50 | 40 | 60 | 70 |
| 4-77 | 1,000 | 40 | 50 | 0 | 0 | 30 | 40 |
| 4-78 | 1,000 | 90 | 90 | 40 | 20 | 60 | 100 |
| 4-80 | 1,000 | 90 | 90 | 60 | 40 | 50 | 90 |
| 4-82 | 1,000 | 90 | 90 | 80 | 40 | 60 | 100 |
| 4-83 | 1,000 | 40 | 60 | 10 | 0 | 0 | 50 |
| 4-86 | 1,000 | 100 | 100 | 80 | 60 | 70 | 100 |
| 4-87 | 1,000 | 100 | 100 | 90 | 90 | 90 | 100 |
| 4-88 | 1,000 | 70 | 70 | 20 | 40 | 50 | 100 |
| 4-90 | 1,000 | 100 | 100 | 80 | 50 | 60 | 100 |
| 4-91 | 1,000 | 50 | 50 | 20 | 10 | 20 | 60 |
| 4-93 | 1,000 | 0 | 30 | 0 | 0 | 0 | 40 |
| 4-94 | 1,000 | 80 | 70 | 10 | 10 | 10 | 50 |
| 4-95 | 1,000 | 30 | 60 | 10 | 10 | 10 | 30 |
| 4-96 | 1,000 | 60 | 60 | 30 | 10 | 20 | 50 |
| 4-97 | 1,000 | 50 | 40 | 40 | 20 | 40 | 60 |
| 4-98 | 1,000 | 60 | 50 | 30 | 30 | 20 | 60 |
| 4-99 | 1,000 | 60 | 60 | 50 | 30 | 50 | 60 |
| 4-100 | 1,000 | 60 | 70 | 20 | 20 | 40 | 50 |
| 4-101 | 1,000 | 30 | 70 | 10 | 10 | 30 | 70 |
| 4-103 | 1,000 | 10 | 40 | 0 | 10 | 0 | 30 |
| 4-104 | 1,000 | 30 | 40 | 0 | 10 | 20 | 20 |
| 4-108 | 1,000 | 0 | 40 | 10 | 0 | 0 | 40 |
| 4-109 | 1,000 | 50 | 70 | 30 | 20 | 30 | 80 |
| 4-110 | 1,000 | 90 | 100 | 40 | 40 | 80 | 100 |
| 4-111 | 1,000 | 60 | 90 | 40 | 20 | 50 | 80 |
| 4-112 | 1,000 | 90 | 70 | 80 | 50 | 40 | 90 |
| 4-115 | 1,000 | 90 | 100 | 60 | 30 | 60 | 90 |
| 4-116 | 1,000 | 100 | 100 | 80 | 50 | 70 | 100 |
| 4-118 | 1,000 | 40 | 40 | 0 | 20 | 50 | 70 |
| 4-120 | 1,000 | 50 | 60 | 10 | 10 | 20 | 40 |
| 4-121 | 1,000 | 0 | 20 | 0 | 0 | 0 | 20 |
| 4-122 | 1,000 | 30 | 60 | 20 | 10 | 30 | 90 |
| 4-125 | 1,000 | 90 | 100 | 80 | 70 | 80 | 100 |
| 4-129 | 1,000 | 100 | 100 | 40 | 30 | 70 | 100 |
| 4-135 | 1,000 | 100 | 100 | 90 | 60 | 100 | 100 |
| 4-140 | 1,000 | 100 | 100 | 50 | 20 | 40 | 80 |
| 4-143 | 1,000 | 50 | 70 | 0 | 0 | 20 | 80 |
| 4-146 | 1,000 | 40 | 70 | 0 | 50 | 50 | 100 |
| 4-148 | 1,000 | 90 | 80 | 20 | 20 | 30 | 100 |
| 4-151 | 1,000 | 90 | 100 | 50 | 40 | 60 | 90 |
| 4-152 | 1,000 | 80 | 70 | 40 | 20 | 70 | 100 |
| 4-153 | 1,000 | 100 | 100 | 60 | 50 | 60 | 100 |
| 4-155 | 1,000 | 70 | 90 | 20 | 20 | 80 | 90 |
| 4-156 | 1,000 | 30 | 50 | 0 | 0 | 0 | 80 |
| 4-159 | 1,000 | 20 | 0 | 0 | 10 | 30 | 70 |
| 4-160 | 1,000 | 90 | 70 | 50 | 20 | 60 | 100 |
| 4-164 | 1,000 | 50 | 50 | 30 | 30 | 30 | 100 |
| 4-168 | 1,000 | 40 | 20 | 0 | 20 | 20 | 50 |
| 4-171 | 1,000 | 40 | 90 | 40 | 30 | 70 | 100 |
| 4-173 (S) | 1,000 | 0 | 20 | 0 | 0 | 0 | 0 |
| 4-174 | 1,000 | 70 | 80 | 20 | 20 | 30 | 50 |
| 4-175 | 1,000 | 10 | 50 | 0 | 10 | 10 | 70 |
| 4-176 | 1,000 | 0 | 50 | 0 | 0 | 20 | 60 |
| 4-177 | 1,000 | 0 | 20 | 0 | 0 | 0 | 20 |
| 4-178 | 1,000 | 30 | 60 | 10 | 10 | 10 | 40 |
| 4-179 | 250 | 0 | 50 | 0 | 0 | 0 | 0 |
| 4-180 (S) | 1,000 | 10 | 20 | 0 | 0 | 0 | 80 |
| 4-181 (S) | 1,000 | 60 | 60 | 0 | 20 | 50 | 80 |
| 4-183 | 1,000 | 0 | 30 | 0 | 0 | 0 | 20 |
| 4-184 (R) | 1,000 | 0 | 0 | 0 | 0 | 0 | 0 |
| 4-185 (R) | 1,000 | 0 | 0 | 0 | 0 | 0 | 70 |
| 4-189 | 1,000 | 50 | 60 | 0 | 20 | 0 | 40 |
| 4-194 | 1,000 | 60 | 100 | 10 | 10 | 20 | 90 |
| 4-197 | 1,000 | 90 | 90 | 20 | 20 | 40 | 100 |
| 4-198 | 1,000 | 20 | 30 | 0 | 0 | 0 | 70 |
| 4-199 | 1,000 | 0 | 0 | 0 | 0 | 0 | 40 |
| 4-200 | 1,000 | 0 | 20 | 0 | 0 | 0 | 50 |
| 4-201 | 1,000 | 100 | 100 | 10 | 0 | 20 | 100 |
| 4-202 | 1,000 | 70 | 60 | 30 | 20 | 30 | 100 |
| 4-203 | 1,000 | 80 | 100 | 60 | 30 | 60 | 100 |
| 4-203 (S) | 1,000 | 80 | 70 | 40 | 20 | 20 | 80 |
| 4-203 (R) | 1,000 | 80 | 90 | 30 | 20 | 20 | 100 |
| 4-205 | 1,000 | 0 | 0 | 0 | 0 | 0 | 0 |
| 4-210 | 1,000 | 90 | 100 | 20 | 20 | 50 | 100 |
| 4-214 | 1,000 | 90 | 100 | 40 | 20 | 60 | 100 |
| 4-215 | 1,000 | 90 | 100 | 20 | 10 | 20 | 90 |
| 4-216 | 1,000 | 90 | 100 | 40 | 30 | 40 | 100 |
| 4-219 | 1,000 | 30 | 60 | 0 | 0 | 0 | 90 |
| 4-220 | 1,000 | 40 | 60 | 0 | 0 | 30 | 50 |
| 4-221 | 1,000 | 90 | 100 | 60 | 60 | 60 | 100 |
| 4-222 | 1,000 | 0 | 30 | 0 | 0 | 0 | 10 |
| 4-224 | 1,000 | 30 | 20 | 0 | 0 | 0 | 70 |
| 4-226 | 1,000 | 60 | 70 | 0 | 0 | 10 | 100 |
| 4-227 | 1,000 | 30 | 20 | 0 | 0 | 0 | 50 |
| 4-228 | 1,000 | 40 | 70 | 0 | 0 | 0 | 40 |
| 4-229 | 1,000 | 20 | 30 | 10 | 0 | 0 | 20 |
| 4-230 | 1,000 | 0 | 10 | 0 | 0 | 0 | 40 |
| 4-231 | 1,000 | 10 | 30 | 0 | 0 | 0 | 70 |
| 4-232 | 1,000 | 80 | 90 | 30 | 10 | 30 | 50 |
| 4-233 | 1,000 | 20 | 40 | 0 | 0 | 0 | 10 |
| 4-234 | 1,000 | 0 | 10 | 0 | 0 | 0 | 0 |
| 4-235 | 1,000 | NC | NC | 0 | 0 | 0 | 0 |
| 4-236 | 1,000 | 60 | 0 | 0 | 0 | 0 | 0 |
| 4-237 | 1,000 | 0 | 30 | 0 | 0 | 0 | 10 |
| 4-238 | 1,000 | 50 | 10 | 0 | 0 | 0 | 0 |
| 4-239 | 1,000 | 0 | 20 | 0 | 0 | 0 | 10 |
| 4-240 | 1,000 | 10 | 30 | 0 | 0 | 0 | 0 |
| 4-241 | 1,000 | 0 | 30 | 0 | 0 | 0 | 0 |
| 4-242 | 1,000 | 0 | 10 | 0 | 0 | 0 | 10 |
| 4-243 | 1,000 | 10 | 40 | 0 | 0 | 0 | 0 |
| 4-244 | 1,000 | 0 | 30 | 0 | 0 | 0 | 0 |
| 4-246 | 1,000 | 20 | 80 | 0 | 0 | 0 | 30 |
| 4-247 | 1,000 | 70 | 30 | 10 | 10 | 10 | 20 |
| 4-249 | 1,000 | 80 | 60 | 50 | 20 | 50 | 100 |
| 4-250 | 1,000 | 40 | 90 | 0 | 0 | 0 | 80 |
| 4-251 | 1,000 | 20 | 40 | 0 | 0 | 0 | 20 |
| 4-252 | 1,000 | 0 | 20 | 0 | 0 | 0 | 0 |
| 4-253 | 1,000 | 80 | 50 | 30 | 10 | 20 | 100 |
| 4-257 | 1,000 | 90 | 100 | 80 | 40 | 60 | 100 |
| 4-258 | 1,000 | 100 | 100 | 40 | 30 | 30 | 100 |
| 4-259 | 1,000 | 90 | 100 | 30 | 10 | 30 | 100 |
| 4-260 | 1,000 | 90 | 100 | 20 | 10 | 20 | 90 |
| 4-262 | 1,000 | 10 | 40 | 0 | 0 | 0 | 50 |
| 4-263 | 1,000 | 10 | 30 | 0 | 0 | 0 | 30 |
| 4-264 | 1,000 | 90 | 80 | 80 | 40 | 60 | 90 |
| 4-265 | 1,000 | 90 | 100 | 30 | 10 | 30 | 90 |
| 4-266 | 1,000 | 100 | 100 | 60 | 50 | 60 | 100 |
| 4-267 | 1,000 | 60 | 90 | 10 | 10 | 10 | 60 |
| 4-268 | 1,000 | 10 | 10 | 0 | 0 | 0 | 10 |
| 4-269 | 1,000 | 0 | 40 | 0 | 0 | 0 | 0 |
| 4-270 (erythro) | 1,000 | 80 | 80 | 30 | 30 | 30 | 100 |
| 4-270 (1S,2R) | 1,000 | 80 | 70 | 30 | 20 | 20 | 100 |
| 4-271 | 1,000 | 90 | 100 | 30 | 20 | 20 | 100 |
| 4-272 (S) | 1,000 | 80 | 70 | 20 | 10 | 30 | 70 |
| 4-273 (S) | 1,000 | 90 | 100 | 80 | 40 | 60 | 90 |
| 4-273 (R) | 1,000 | 80 | 80 | 70 | 50 | 60 | 100 |
| 4-274 | 1,000 | 80 | 60 | 40 | 40 | 30 | 80 |
| 4-275 (S,S) | 1,000 | 100 | 100 | 60 | 30 | 30 | 100 |
| 4-275 (R,R) | 1,000 | 90 | 100 | 70 | 70 | 60 | 100 |
| 4-276 (R) | 1,000 | 90 | 100 | 30 | 20 | 30 | 80 |
| 4-277 | 1,000 | 0 | 0 | 0 | 0 | 0 | 40 |
| 4-278 | 1,000 | 10 | 40 | 10 | 0 | 10 | 20 |
| 4-279 | 1,000 | 70 | 20 | 0 | 10 | 0 | 60 |
| 4-280 (1R,2S) | 1,000 | 80 | 60 | 10 | 10 | 20 | 90 |
| 4-280 (1S,2R) | 1,000 | 90 | 100 | 30 | 20 | 40 | 80 |
| 4-282 (S) | 1,000 | 90 | 90 | 20 | 10 | 30 | 80 |
| 4-283 | 1,000 | 100 | 100 | 80 | 40 | 70 | 100 |
| 4-284 | 1,000 | 90 | 100 | 30 | 20 | 30 | 100 |
| 4-285 (S) | 1,000 | 90 | 90 | 20 | 20 | 30 | 70 |
| 4-286 (S) | 1,000 | 80 | 90 | 20 | 10 | 20 | 100 |
| 4-287 (S) | 1,000 | 80 | 80 | 20 | 20 | 10 | 80 |
| 4-288 (S) | 1,000 | 40 | 80 | 0 | 0 | 0 | 60 |
| 4-290 | 1,000 | 80 | 80 | 30 | 20 | 40 | 90 |
| 4-292 (R) | 1,000 | 80 | 100 | 10 | 20 | 20 | 100 |
| 4-293 (S) | 1,000 | 50 | 40 | 0 | 10 | 20 | 60 |
| 5-54 | 1,000 | 0 | 50 | 0 | 0 | 0 | 30 |
| 5-61 | 1,000 | 20 | 70 | 40 | 30 | 40 | 70 |
| 5-62 | 1,000 | 20 | 100 | 10 | 0 | 0 | 60 |
| 5-63 | 1,000 | 30 | 100 | 20 | 10 | 0 | 70 |
| 5-64 | 1,000 | 70 | 70 | 10 | 10 | 20 | 80 |
| 5-65 | 1,000 | 90 | 100 | 20 | 20 | 10 | 100 |
| 5-66 | 1,000 | 60 | 100 | 60 | 30 | 40 | 70 |
| 5-69 | 1,000 | 20 | 70 | 0 | 10 | 10 | 50 |
| 5-73 | 1,000 | 20 | 80 | 0 | 0 | 0 | 30 |
| 5-76 | 1,000 | 40 | 80 | 30 | 10 | 20 | 60 |
| 5-77 | 1,000 | 20 | 90 | 0 | 0 | 0 | 20 |
| 5-79 | 1,000 | 40 | 70 | 0 | 0 | 0 | 50 |
| 5-80 | 1,000 | 30 | 100 | 20 | 0 | 0 | 60 |
| 5-84 | 1,000 | 0 | 60 | 0 | 0 | 0 | 20 |
| 5-86 | 1,000 | 100 | 100 | 30 | 40 | 50 | 100 |
| 5-90 | 1,000 | 90 | 100 | 30 | 20 | 30 | 90 |
| 5-91 | 1,000 | 60 | 70 | 10 | 10 | 0 | 30 |
| 5-92 | 1,000 | 60 | 90 | 10 | 20 | 10 | 70 |
| 5-104 | 1,000 | 10 | 30 | 0 | 0 | 0 | 40 |
| 5-105 | 1,000 | 60 | 80 | 30 | 10 | 10 | 60 |
| 5-113 | 1,000 | 10 | 70 | 0 | 0 | 0 | 60 |
| 5-114 | 1,000 | 20 | 70 | 20 | 0 | 0 | 40 |
| 5-125 | 1,000 | 90 | 100 | 80 | 40 | 70 | 100 |
| 5-135 | 1,000 | 90 | 100 | 10 | 30 | 0 | 100 |
| 5-149 | 1,000 | 40 | 80 | 10 | 30 | 20 | 80 |
| 5-156 | 1,000 | 20 | 90 | 0 | 10 | 0 | 60 |
| 5-160 | 1,000 | 40 | 80 | 0 | 0 | 20 | 70 |
| 5-221 | 1,000 | 30 | 30 | 0 | 0 | 0 | 40 |
| 5-228 | 1,000 | 70 | 80 | 0 | 0 | 0 | 0 |
| 5-257 | 1,000 | 100 | 100 | 10 | 10 | 30 | 50 |
| 5-270 | 1,000 | 50 | 90 | 0 | 10 | 0 | 20 |
| 5-283 (S) | 1,000 | 40 | 80 | 0 | 0 | 0 | 30 |
| 6-35 | 1,000 | 60 | 100 | 0 | 10 | 0 | 30 |
| 6-60 | 1,000 | 100 | 100 | 30 | 20 | 20 | 20 |
| 6-64 | 1,000 | 20 | 60 | 30 | 10 | 20 | 50 |
| 6-65 | 1,000 | 40 | 70 | 30 | 20 | 20 | 70 |
| 6-66 | 1,000 | 20 | 50 | 10 | 20 | 0 | 80 |
| 6-102 | 1,000 | 60 | 100 | 0 | 0 | 0 | 60 |
| 6-112 | 1,000 | 10 | 60 | 0 | 10 | 10 | 60 |
| 6-125 | 1,000 | 80 | 90 | 30 | 50 | 40 | 100 |
| 6-135 | 1,000 | 80 | 100 | 40 | 40 | 60 | 100 |
| 6-140 | 1,000 | 30 | 80 | 0 | 10 | 20 | 80 |
| 6-142 | 1,000 | 10 | 50 | 10 | 0 | 0 | 60 |
| 6-161 | 1,000 | 20 | 100 | 0 | 0 | 0 | 80 |
| 7-60 | 1,000 | 90 | 70 | 50 | 30 | 50 | 70 |
| 7-125 | 1,000 | 100 | 100 | 80 | 60 | 90 | 100 |
| 31-125 | 1,000 | 80 | 90 | 20 | 20 | 20 | 100 |
| 37-125 | 1,000 | 100 | 100 | 90 | 50 | 90 | 40 |
| 40-1 | 1,000 | 40 | 100 | 30 | 40 | 50 | 30 |
| 40-60 | 1,000 | 30 | 90 | 0 | 20 | 50 | 0 |
| 40-125 | 1,000 | 80 | 100 | 40 | 40 | 50 | 20 |
| 67-125 | 1,000 | 20 | 100 | 10 | 50 | 20 | 40 |
| 109-43 | 1,000 | 90 | 100 | 60 | 10 | 30 | 70 |
| 109-125 | 1,000 | 100 | 100 | 90 | 70 | 90 | 100 |
| 112-43 | 1,000 | 70 | 60 | 30 | 40 | 60 | 80 |
| 112-125 | 1,000 | 90 | 100 | 90 | 70 | 100 | 100 |
| 112-135 | 1,000 | 100 | 100 | 90 | 70 | 90 | 100 |
| 139-125 | 1,000 | 100 | 100 | 80 | 70 | 80 | 90 |
| 145-60 | 1,000 | 70 | 100 | 10 | 0 | 0 | 40 |
| 145-64 | 1,000 | 70 | 70 | 0 | 0 | 0 | 70 |
| 145-73 | 1,000 | 70 | 100 | 20 | 30 | 30 | 20 |
| 146-135 | 1,000 | 100 | 100 | 40 | 40 | 10 | 100 |
| 146-255 | 1,000 | 10 | 100 | 20 | 10 | 10 | 70 |
| 147-60 | 1,000 | 80 | 100 | 0 | 40 | 20 | 30 |
| 148-60 | 1,000 | 80 | 80 | 0 | 0 | 0 | 30 |
| 150-60 | 1,000 | 70 | 80 | 20 | 20 | 40 | 90 |
| 151-60 | 1,000 | 10 | 40 | 10 | 20 | 20 | 60 |
| 151-64 | 1,000 | 0 | 30 | 0 | 0 | 0 | 30 |
| 151-66 | 1,000 | 0 | 40 | 0 | 0 | 0 | 50 |
| 151-109 | 1,000 | 20 | 60 | 10 | 0 | 0 | 20 |
| 151-117 | 1,000 | 30 | 40 | 10 | 10 | 0 | 30 |
| 151-140 | 1,000 | 70 | 100 | 10 | 0 | 0 | 20 |
| 151-141 | 1,000 | 0 | 50 | 0 | 10 | 0 | 40 |
| 152-75 | 1,000 | 20 | 80 | 40 | 20 | 10 | 80 |
| 154-60 | 1,000 | 0 | 40 | 0 | 0 | 0 | 0 |
| 155-60 | 1,000 | 0 | 30 | 0 | 0 | 0 | 40 |
| 155-257 | 1,000 | 10 | 30 | 20 | 10 | 10 | 30 |
| 157-156 | 1,000 | 20 | 40 | 0 | 0 | 0 | 40 |
| 157-157 | 1,000 | 100 | 100 | 30 | 10 | 20 | 40 |
| 160-256 | 1,000 | 10 | 20 | 0 | 10 | 0 | 10 |
| 161-60 | 1,000 | 0 | 0 | 0 | 0 | 0 | 0 |
| 161-125 | 1,000 | 30 | 100 | 0 | 20 | 20 | 80 |
| 162-60 | 1,000 | 20 | 40 | 0 | 10 | 20 | 70 |
| 187-125 | 1,000 | 0 | 0 | 0 | 0 | 0 | 0 |
| 197-125 | 1,000 | 90 | 100 | 70 | 50 | 70 | 100 |
| 198-125 | 1,000 | 100 | 100 | 80 | 40 | 80 | 100 |
| 199-125 | 1,000 | 100 | 100 | 80 | 40 | 50 | 100 |
| 200-125 | 1,000 | 100 | 100 | 70 | 50 | 70 | 100 |
| 201-125 | 1,000 | 70 | 70 | 0 | 20 | 0 | 40 |
| 207-30 | 1,000 | 0 | 0 | 0 | 0 | 0 | 0 |
| 207-31 | 1,000 | 10 | 20 | 0 | 0 | 0 | 0 |
| 207-35 | 1,000 | 0 | 30 | 0 | 10 | 10 | 10 |
| 207-48 | 1,000 | 40 | 30 | 20 | 20 | 10 | 40 |
| 207-50 | 1,000 | 80 | 60 | 0 | 0 | 0 | 40 |
| 212-48 | 1,000 | 40 | 30 | 20 | 10 | 20 | 0 |
| 213-48 | 1,000 | 0 | 70 | 10 | 10 | 0 | 0 |
| 214-48 | 1,000 | 20 | 100 | 10 | 20 | 0 | 0 |
| 215-48 | 1,000 | 10 | 40 | 30 | 40 | 0 | 0 |
| 216-48 | 1,000 | 30 | 30 | 30 | 0 | 30 | 0 |
| 217-48 | 1,000 | 50 | 20 | 20 | 20 | 0 | 0 |
| 218-48 | 1,000 | 90 | 100 | 70 | 70 | 50 | 50 |
| 219-48 | 1,000 | 0 | 20 | 0 | 0 | 0 | 0 |
| 220-48 | 1,000 | 0 | 20 | 0 | 0 | 0 | 0 |
| 221-48 | 1,000 | 0 | 60 | 0 | 0 | 0 | 0 |
| 222-48 | 1,000 | 100 | 100 | 20 | 30 | 50 | 70 |
| 224-48 | 1,000 | 90 | 100 | 50 | 60 | 0 | 70 |
| 225-48 | 1,000 | 90 | 100 | 80 | 60 | 30 | 70 |
| 226-48 | 1,000 | 100 | 100 | 40 | 50 | 30 | 30 |
| 228-48 | 1,000 | 0 | 30 | 0 | 0 | 0 | 0 |
| 229-48 | 1,000 | 90 | 100 | 30 | 10 | 0 | 0 |
| 230-48 | 1,000 | 70 | 70 | 50 | 30 | 20 | 50 |
| 231-48 | 1,000 | 40 | 100 | 20 | 40 | 30 | 10 |
| 232-48 | 1,000 | 0 | 0 | 0 | 0 | 0 | 0 |
| 233-48 | 1,000 | 100 | 100 | 20 | 30 | 20 | 60 |
| 234-48 | 1,000 | 80 | 90 | 10 | 20 | 10 | 100 |
| 235-48 | 1,000 | 20 | 10 | 0 | 0 | 0 | 20 |
| 237-48 | 1,000 | 0 | 100 | 0 | 20 | 20 | 30 |
| 238-48 | 1,000 | 0 | 100 | 10 | 20 | 0 | 0 |
| 239-48 | 1,000 | 0 | 0 | 0 | 0 | 0 | 0 |
| 240-48 | 1,000 | 50 | 100 | 0 | 0 | 0 | 0 |
| 241-48 | 1,000 | 0 | 80 | 0 | 0 | 0 | 10 |
| 242-48 | 1,000 | 20 | 100 | 0 | 10 | 0 | 20 |
| 243-48 | 1,000 | 10 | 60 | 40 | 10 | 0 | 0 |
| 244-48 | 1,000 | 30 | 10 | 60 | 30 | 20 | 0 |
| 245-48 | 1,000 | 10 | 80 | 30 | 30 | 30 | 20 |
| 247-48 | 1,000 | 40 | 90 | 20 | 10 | 10 | 40 |
| 248-48 | 1,000 | 20 | 60 | 0 | 0 | 0 | 0 |
| 249-48 | 1,000 | 30 | 70 | 20 | 0 | 0 | 20 |
| 250-48 | 1,000 | 0 | 30 | 20 | 0 | 0 | 0 |
| 251-48 | 1,000 | 0 | 0 | 0 | 0 | 0 | 0 |
| 252-48 | 1,000 | 0 | 0 | 20 | 0 | 0 | 0 |
| 253-48 | 1,000 | NC | NC | 0 | 0 | 0 | 0 |
| 254-48 | 1,000 | 0 | 0 | 0 | 0 | 0 | 0 |
| 255-48 | 1,000 | 0 | 20 | 0 | 0 | 0 | 10 |
| 256-48 | 1,000 | 0 | 80 | 0 | 0 | 0 | 0 |
| 257-48 | 1,000 | 10 | 90 | 10 | 20 | 40 | 30 |
| 258-48 | 1,000 | 0 | 70 | 20 | 30 | 0 | 30 |
| 260-48 | 1,000 | 0 | 0 | 0 | 0 | 0 | 0 |
| 261-48 | 1,000 | 40 | 100 | 90 | 50 | 90 | 30 |
| 262-30 | 1,000 | 0 | 0 | 0 | 0 | 0 | 0 |
| 262-48 | 1,000 | 20 | 80 | 0 | 0 | 0 | 0 |
| 265-1 | 1,000 | 10 | 30 | 0 | 0 | 0 | 0 |
| 265-3 | 1,000 | 60 | 60 | 40 | 20 | 30 | 90 |
| 265-4 | 1,000 | 0 | 20 | 0 | 0 | 0 | 0 |
| 266-20 | 1,000 | 50 | 100 | 0 | 0 | 0 | 40 |

NC = data not collected

### Example 18

### Post-Emergence Biological Efficacy

Seeds of Alopecurus myosuroides (ALOMY), Setaria faberi (SETFA), Echinochloa crus-galli (ECHCG), Solanum nigrum (SOLNI), Amaranthus retoflexus (AMARE) and Ipomea hederaceae (IPOHE) were sown in standard soil in pots. After cultivation for 8 days under controlled conditions in a glasshouse (at 24/16° C., day/night; 14 hours light; 65% humidity), the plants were sprayed with an aqueous spray solution derived from the formulation of the technical active ingredient in acetone/water (50:50) solution containing 0.5% Tween 20 (polyoxyethelyene sorbitan monolaurate, CAS RN 9005-64-5) to give a final dose of 1000 or 250 g/ha of test compound.

The test plants were then grown on under controlled conditions in a glasshouse (at 24/16° C., day/night; 14 hours light; 65% humidity) and watered twice daily. After 13 days the test was evaluated (100=total damage to plant; 0=no damage to plant). Results are shown below in Table B2.

TABLE B2
Percentage damage caused to weed species by compounds of the
invention when applied post-emergence.

| Compound | Rate | Species | | | | | |
|---|---|---|---|---|---|---|---|
| Number | (g/ha) | SOLNI | AMARE | SETFA | ALOMY | ECHCG | IPOHE |
| 1-1 | 1,000 | 90 | 100 | 80 | 40 | 40 | 80 |
| 1-33 | 1,000 | 80 | 100 | 80 | 30 | 20 | 70 |
| 1-35 | 1,000 | 30 | 50 | 0 | 10 | 10 | 50 |
| 1-60 | 1,000 | 90 | 100 | 90 | 50 | 70 | 100 |
| 1-66 | 1,000 | 90 | 100 | 80 | 10 | 50 | 80 |
| 1-73 | 1,000 | 80 | 100 | 50 | 10 | 30 | 80 |
| 1-104 | 1,000 | 50 | 60 | 0 | 0 | 0 | 60 |
| 1-110 | 1,000 | 80 | 100 | 80 | 30 | 50 | 80 |
| 1-125 | 1,000 | 100 | 100 | 90 | 60 | 70 | 100 |
| 1-135 | 1,000 | 100 | 100 | 80 | 80 | 70 | 100 |
| 1-153 | 1,000 | 90 | 100 | 90 | 50 | 60 | 70 |
| 1-176 | 1,000 | 60 | 40 | 20 | 10 | 10 | 40 |
| 4-1 | 1,000 | 100 | 100 | 80 | 60 | 60 | 90 |
| 4-5 | 1,000 | 80 | 100 | 40 | 20 | 0 | 70 |
| 4-6 | 1,000 | 40 | 100 | 0 | 20 | 20 | 60 |
| 4-10 | 1,000 | 40 | 60 | 0 | 20 | 0 | 50 |
| 4-13 | 1,000 | 50 | 60 | 0 | 20 | 0 | 60 |
| 4-16 | 1,000 | 90 | 100 | 60 | 20 | 40 | 100 |
| 4-17 | 1,000 | 70 | 100 | 60 | 20 | 30 | 60 |
| 4-19 (Z) | 1,000 | 90 | 100 | 60 | 10 | 30 | 100 |
| 4-19 (E) | 1,000 | 80 | 100 | 70 | 20 | 40 | 100 |
| 4-20 | 1,000 | 80 | 100 | 70 | 20 | 50 | 100 |
| 4-21 | 1,000 | 80 | 100 | 60 | 20 | 50 | 80 |
| 4-22 | 1,000 | 100 | 100 | 80 | 30 | 40 | 100 |
| 4-23 | 1,000 | 80 | 100 | 70 | 20 | 60 | 100 |
| 4-24 | 1,000 | 80 | 100 | 60 | 10 | 30 | 100 |
| 4-25 | 1,000 | 80 | 100 | 60 | 40 | 60 | 100 |
| 4-26 | 1,000 | 70 | 100 | 60 | 20 | 20 | 90 |
| 4-27 | 1,000 | 80 | 100 | 70 | 10 | 60 | 100 |
| 4-28 | 1,000 | 80 | 100 | 50 | 10 | 30 | 100 |
| 4-29 | 1,000 | 100 | 90 | 60 | 10 | 30 | 100 |
| 4-30 | 1,000 | 70 | 100 | 60 | 10 | 30 | 100 |
| 4-31 | 1,000 | 90 | 100 | 80 | 10 | 30 | 80 |
| 4-32 | 1,000 | 90 | 100 | 90 | 50 | 70 | 100 |
| 4-33 | 1,000 | 70 | 80 | 70 | 30 | 60 | 70 |
| 4-34 | 1,000 | 80 | 100 | 60 | 20 | 60 | 60 |
| 4-35 | 1,000 | 40 | 30 | 20 | 30 | 0 | 60 |
| 4-36 (trans) | 1,000 | 70 | 80 | 20 | 20 | 10 | 40 |
| 4-37 (1R,2S) | 1,000 | 100 | 100 | 70 | 30 | 60 | 100 |
| 4-37 (1S,2R) | 1,000 | 100 | 100 | 60 | 50 | 60 | 50 |
| 4-38 (S,S) | 1,000 | 80 | 100 | 60 | 30 | 10 | 30 |
| 4-39 | 1,000 | 80 | 100 | 60 | 50 | 70 | 70 |
| 4-42 | 1,000 | 50 | 50 | 0 | 10 | 10 | 60 |
| 4-43 | 1,000 | 90 | 100 | 100 | 60 | 70 | 100 |
| 4-49 | 1,000 | 70 | 100 | 40 | 10 | 40 | 70 |
| 4-50 | 1,000 | 80 | 100 | 50 | 10 | 10 | 70 |
| 4-51 | 1,000 | 50 | 50 | 50 | 10 | 20 | 60 |
| 4-52 | 1,000 | 60 | 30 | 20 | 0 | 20 | 50 |
| 4-53 | 1,000 | 90 | 100 | 40 | 20 | 30 | 100 |
| 4-55 | 1,000 | 80 | 100 | 30 | 10 | 0 | 50 |
| 4-56 | 1,000 | 80 | 100 | 40 | 20 | 30 | 80 |
| 4-58 | 1,000 | 70 | 70 | 50 | 0 | 40 | 50 |
| 4-59 | 1,000 | 100 | 100 | 80 | 50 | 60 | 100 |
| 4-60 | 1,000 | 100 | 100 | 90 | 20 | 90 | 100 |
| 4-62 | 1,000 | 100 | 100 | 90 | 30 | 70 | 100 |
| 4-63 | 1,000 | 90 | 100 | 80 | 40 | 50 | 80 |
| 4-65 | 1,000 | 100 | 100 | 100 | 20 | 80 | 100 |
| 4-66 | 1,000 | 100 | 100 | 90 | 20 | 70 | 100 |
| 4-67 | 250 | 90 | 90 | 70 | 20 | 50 | 80 |
| 4-68 | 1,000 | 90 | 100 | 90 | 20 | 60 | 100 |
| 4-70 | 1,000 | 100 | 100 | 90 | 20 | 70 | 100 |
| 4-71 | 1,000 | 90 | 100 | 80 | 20 | 60 | 100 |
| 4-72 | 1,000 | 100 | 100 | 30 | 10 | 50 | 100 |
| 4-73 | 1,000 | 100 | 100 | 80 | 20 | 60 | 100 |
| 4-74 | 1,000 | 100 | 90 | 80 | 20 | 70 | 100 |
| 4-75 | 1,000 | 100 | 100 | 90 | 20 | 50 | 100 |
| 4-76 | 1,000 | 100 | 100 | 90 | 40 | 70 | 100 |
| 4-77 | 1,000 | 100 | 100 | 90 | 20 | 90 | 100 |
| 4-78 | 1,000 | 90 | 100 | 80 | 30 | 70 | 100 |
| 4-80 | 1,000 | 100 | 100 | 90 | 30 | 60 | 100 |
| 4-82 | 1,000 | 100 | 100 | 80 | 30 | 60 | 100 |
| 4-83 | 1,000 | 100 | 100 | 90 | 20 | 60 | 100 |
| 4-86 | 1,000 | 100 | 100 | 90 | 50 | 70 | 100 |
| 4-87 | 1,000 | 100 | 100 | 100 | 60 | 60 | 100 |
| 4-88 | 1,000 | 80 | 100 | 60 | 50 | 60 | 80 |
| 4-90 | 1,000 | 100 | 100 | 100 | 50 | 80 | 100 |
| 4-91 | 1,000 | 100 | 100 | 90 | 50 | 80 | 100 |
| 4-93 | 1,000 | 50 | 30 | 0 | 0 | 0 | 70 |
| 4-94 | 1,000 | 90 | 100 | 30 | 10 | 10 | 100 |
| 4-95 | 1,000 | 80 | 90 | 60 | 20 | 60 | 100 |
| 4-96 | 1,000 | 100 | 100 | 90 | 20 | 80 | 100 |
| 4-97 | 1,000 | 100 | 100 | 90 | 20 | 80 | 100 |
| 4-98 | 1,000 | 100 | 100 | 100 | 40 | 80 | 100 |
| 4-99 | 1,000 | 100 | 100 | 100 | 50 | 100 | 100 |
| 4-100 | 1,000 | 100 | 100 | 100 | 30 | 70 | 100 |
| 4-101 | 1,000 | 100 | 100 | 90 | 30 | 50 | 100 |
| 4-103 | 1,000 | 80 | 100 | 70 | 20 | 60 | 90 |
| 4-104 | 1,000 | 90 | 100 | 90 | 20 | 90 | 100 |
| 4-108 | 1,000 | 90 | 50 | 20 | 10 | 10 | 70 |
| 4-109 | 1,000 | 90 | 100 | 80 | 20 | 60 | 100 |
| 4-110 | 1,000 | 80 | 100 | 80 | 70 | 80 | 100 |
| 4-111 | 1,000 | 100 | 100 | 80 | 30 | 60 | 100 |
| 4-112 | 1,000 | 100 | 100 | 90 | 40 | 90 | 100 |
| 4-115 | 1,000 | 100 | 100 | 90 | 60 | 80 | 100 |
| 4-116 | 1,000 | 100 | 100 | 90 | 60 | 100 | 100 |
| 4-118 | 1,000 | 80 | 100 | 80 | 60 | 80 | 90 |
| 4-120 | 1,000 | 100 | 100 | 90 | 20 | 60 | 100 |
| 4-121 | 1,000 | 90 | 100 | 70 | 20 | 60 | 100 |
| 4-122 | 1,000 | 100 | 100 | 90 | 20 | 90 | 100 |
| 4-125 | 1,000 | 100 | 100 | 90 | 60 | 80 | 100 |
| 4-129 | 1,000 | 90 | 100 | 90 | 40 | 70 | 100 |
| 4-135 | 1,000 | 100 | 100 | 90 | 60 | 90 | 100 |
| 4-140 | 1,000 | 90 | 90 | 70 | 30 | 60 | 90 |
| 4-143 | 1,000 | 90 | 90 | 70 | 20 | 60 | 100 |
| 4-146 | 1,000 | 90 | 90 | 70 | 20 | 50 | 80 |
| 4-148 | 1,000 | 80 | 100 | 50 | 20 | 40 | 60 |
| 4-151 | 1,000 | 80 | 100 | 80 | 60 | 80 | 80 |
| 4-152 | 1,000 | 90 | 100 | 90 | 50 | 80 | 100 |
| 4-153 | 1,000 | 100 | 100 | 90 | 70 | 80 | 100 |
| 4-155 | 1,000 | 90 | 100 | 70 | 30 | 70 | 90 |
| 4-156 | 1,000 | 90 | 100 | 70 | 30 | 50 | 70 |
| 4-159 | 1,000 | 80 | 90 | 70 | 30 | 80 | 80 |
| 4-160 | 1,000 | 80 | 100 | 80 | 40 | 70 | 90 |
| 4-164 | 1,000 | 100 | 100 | 70 | 30 | 60 | 80 |
| 4-168 | 1,000 | 70 | 90 | 60 | 30 | 40 | 60 |
| 4-171 | 1,000 | 90 | 100 | 90 | 40 | 60 | 90 |
| 4-173 (S) | 1,000 | 20 | 20 | 0 | 0 | 0 | 40 |
| 4-174 | 1,000 | 90 | 100 | 90 | 30 | 90 | 100 |
| 4-175 | 1,000 | 100 | 100 | 90 | 20 | 60 | 100 |
| 4-176 | 1,000 | 100 | 100 | 70 | 20 | 50 | 100 |
| 4-177 | 1,000 | 90 | 90 | 40 | 20 | 50 | 100 |
| 4-178 | 1,000 | 80 | 70 | 30 | 20 | 20 | 70 |
| 4-179 | 250 | 80 | 70 | 50 | 30 | 40 | 60 |
| 4-180 (S) | 1,000 | 70 | 80 | 30 | 20 | 50 | 80 |
| 4-181 (S) | 1,000 | 90 | 100 | 40 | 30 | 50 | 90 |
| 4-183 | 1,000 | 40 | 70 | 20 | 10 | 10 | 60 |
| 4-184 (R) | 1,000 | 60 | 80 | 20 | 10 | 40 | 70 |
| 4-185 (R) | 1,000 | 40 | 80 | 0 | 0 | 0 | 70 |
| 4-189 | 1,000 | 90 | 100 | 50 | 20 | 0 | 100 |
| 4-194 | 1,000 | 100 | 100 | 80 | 10 | 60 | 100 |
| 4-197 | 1,000 | 90 | 100 | 80 | 40 | 70 | 100 |
| 4-198 | 1,000 | 30 | 40 | 40 | 20 | 0 | 60 |
| 4-199 | 1,000 | 60 | 50 | 0 | 0 | 0 | 80 |
| 4-200 | 1,000 | 80 | 100 | 40 | 20 | 20 | 90 |
| 4-201 | 1,000 | 90 | 100 | 20 | 10 | 10 | 60 |
| 4-202 | 1,000 | 80 | 100 | 30 | 10 | 0 | 50 |
| 4-203 | 1,000 | 80 | 100 | 40 | 40 | 30 | 50 |
| 4-203 (S) | 1,000 | 80 | 100 | 50 | 30 | 30 | 70 |
| 4-203 (R) | 1,000 | 80 | 100 | 40 | 20 | 30 | 50 |
| 4-205 | 1,000 | 10 | 20 | 0 | 0 | 0 | 20 |
| 4-210 | 1,000 | 40 | 50 | 80 | 60 | 60 | 40 |
| 4-214 | 1,000 | 80 | 100 | 70 | 50 | 70 | 100 |
| 4-215 | 1,000 | 100 | NC | 50 | 20 | 30 | 80 |
| 4-216 | 1,000 | 70 | 100 | 60 | 20 | 20 | 80 |
| 4-219 | 1,000 | 50 | 50 | 10 | 0 | 0 | 50 |
| 4-220 | 1,000 | 90 | 100 | 40 | 20 | 30 | 100 |
| 4-221 | 1,000 | 80 | 100 | 70 | 30 | 40 | 100 |
| 4-222 | 1,000 | 20 | 30 | 0 | 0 | 0 | 60 |
| 4-224 | 1,000 | 70 | 100 | 30 | 10 | 10 | 50 |
| 4-226 | 1,000 | 80 | 100 | 50 | 30 | 10 | 60 |
| 4-227 | 1,000 | 80 | 70 | 40 | 10 | 30 | 70 |
| 4-228 | 1,000 | 80 | 90 | 50 | 10 | 40 | 70 |
| 4-229 | 1,000 | 90 | 100 | 70 | 10 | 20 | 60 |
| 4-230 | 1,000 | 40 | 40 | 0 | 0 | 0 | 40 |
| 4-231 | 1,000 | 70 | 90 | 30 | 10 | 10 | 60 |
| 4-232 | 1,000 | 70 | 100 | 50 | 20 | 30 | 100 |
| 4-233 | 1,000 | 80 | 100 | 50 | 10 | 20 | 60 |
| 4-234 | 1,000 | 70 | 90 | 30 | 0 | 20 | 60 |
| 4-235 | 1,000 | 60 | 100 | 20 | 0 | 30 | 70 |
| 4-236 | 1,000 | 70 | 100 | 40 | 0 | 0 | 60 |
| 4-237 | 1,000 | 70 | 100 | 40 | 0 | 30 | 60 |
| 4-238 | 1,000 | 70 | 90 | 0 | 10 | 0 | 60 |
| 4-239 | 1,000 | 60 | 90 | 20 | 0 | 0 | 50 |
| 4-240 | 1,000 | 70 | 100 | 30 | 10 | 0 | 60 |
| 4-241 | 1,000 | 70 | 60 | 30 | 0 | 10 | 60 |
| 4-242 | 1,000 | 60 | 90 | 10 | 0 | 0 | 50 |
| 4-243 | 1,000 | 70 | 80 | 20 | 0 | 10 | 60 |
| 4-244 | 1,000 | 20 | 20 | 0 | 0 | 0 | 50 |
| 4-246 | 1,000 | 50 | 100 | 30 | 0 | 0 | 60 |
| 4-247 | 1,000 | 50 | 100 | 20 | 10 | 20 | 50 |
| 4-249 | 1,000 | 100 | 100 | 70 | 30 | 50 | 90 |
| 4-250 | 1,000 | 70 | 100 | 50 | 0 | 30 | 100 |
| 4-251 | 1,000 | 80 | 100 | 50 | 10 | 40 | 50 |
| 4-252 | 1,000 | 60 | 100 | 30 | 10 | 0 | 60 |
| 4-253 | 1,000 | 100 | 100 | 50 | 10 | 40 | 70 |
| 4-257 | 1,000 | 90 | 100 | 80 | 60 | 70 | 100 |
| 4-258 | 1,000 | 80 | 100 | 70 | 30 | 60 | 90 |
| 4-259 | 1,000 | 80 | 100 | 80 | 20 | 30 | 70 |
| 4-260 | 1,000 | 80 | 100 | 60 | 20 | 40 | 100 |
| 4-262 | 1,000 | 60 | 80 | 30 | 10 | 10 | 60 |
| 4-263 | 1,000 | 60 | 90 | 30 | 0 | 0 | 50 |
| 4-264 | 1,000 | 100 | 100 | 70 | 30 | 50 | 60 |
| 4-265 | 1,000 | 70 | 100 | 40 | 20 | 20 | 60 |
| 4-266 | 1,000 | 80 | 100 | 70 | 60 | 50 | 100 |
| 4-267 | 1,000 | 80 | 90 | 20 | 10 | 10 | 70 |
| 4-268 | 1,000 | 60 | 30 | 10 | 10 | 10 | 50 |
| 4-269 | 1,000 | 70 | 100 | 60 | 20 | 0 | 90 |
| 4-270 (erythro) | 1,000 | 80 | 100 | 80 | 30 | 60 | 100 |
| 4-270 (1S,2R) | 1,000 | 100 | 100 | 80 | 40 | 30 | 100 |
| 4-271 | 1,000 | 90 | 100 | 80 | 30 | 70 | 100 |
| 4-272 (S) | 1,000 | 80 | 100 | 60 | 20 | 30 | 60 |
| 4-273 (S) | 1,000 | 100 | 100 | 70 | 50 | 60 | 90 |
| 4-273 (R) | 1,000 | 90 | 100 | 60 | 30 | 60 | 90 |
| 4-274 | 1,000 | 80 | 100 | 30 | 30 | 20 | 50 |
| 4-275 (S,S) | 1,000 | 100 | 100 | 80 | 40 | 60 | 70 |
| 4-275 (R,R) | 1,000 | 100 | 100 | 80 | 30 | 70 | 100 |
| 4-276 (R) | 1,000 | 80 | 100 | 40 | 20 | 30 | 40 |
| 4-277 | 1,000 | 50 | 90 | 50 | 30 | 50 | 70 |
| 4-278 | 1,000 | 30 | 90 | 40 | 10 | 20 | 60 |
| 4-279 | 1,000 | 70 | 90 | 40 | 10 | 0 | 60 |
| 4-280 (1R,2S) | 1,000 | 80 | 100 | 70 | 40 | 50 | 70 |
| 4-280 (1S,2R) | 1,000 | 100 | 100 | 80 | 40 | 60 | 100 |
| 4-282 (S) | 1,000 | 80 | 100 | 60 | 20 | 20 | 80 |
| 4-283 | 1,000 | 80 | 100 | 60 | 20 | 60 | 70 |
| 4-284 | 1,000 | 80 | 100 | 40 | 30 | 20 | 100 |
| 4-285 (S) | 1,000 | 90 | 100 | 40 | 20 | 20 | 90 |
| 4-286 (S) | 1,000 | 80 | 100 | 20 | 10 | 10 | 50 |
| 4-287 (S) | 1,000 | 80 | 100 | 40 | 20 | 60 | 70 |
| 4-288 (S) | 1,000 | 80 | 80 | 10 | 10 | 0 | 50 |
| 4-290 | 1,000 | 80 | 100 | 50 | 30 | 30 | 70 |
| 4-292 (R) | 1,000 | 70 | 100 | 50 | 20 | 30 | 60 |
| 4-293 (S) | 1,000 | 80 | 100 | 40 | 10 | 20 | 80 |
| 5-54 | 1,000 | 30 | 80 | 30 | 0 | 0 | 60 |
| 5-61 | 1,000 | 90 | 100 | 80 | 20 | 80 | 90 |
| 5-62 | 1,000 | 80 | 100 | 40 | 20 | 30 | 90 |
| 5-63 | 1,000 | 80 | 100 | 70 | 30 | 40 | 90 |
| 5-64 | 1,000 | 90 | 100 | 90 | 20 | 80 | 100 |
| 5-65 | 1,000 | 100 | 100 | 80 | 30 | 70 | 90 |
| 5-66 | 1,000 | 90 | 100 | 80 | 20 | 40 | 100 |
| 5-69 | 1,000 | 80 | 100 | 70 | 30 | 50 | 80 |
| 5-73 | 1,000 | 70 | 100 | 50 | 10 | 10 | 100 |
| 5-76 | 1,000 | 90 | 100 | 80 | 40 | 50 | 90 |
| 5-77 | 1,000 | 100 | 100 | 50 | 20 | 40 | 100 |
| 5-79 | 1,000 | 80 | 100 | 30 | 0 | 0 | 70 |
| 5-80 | 1,000 | 100 | 100 | 80 | 20 | 50 | 100 |
| 5-84 | 1,000 | 40 | 100 | 30 | 10 | 10 | 40 |
| 5-86 | 1,000 | 90 | 100 | 90 | 40 | 70 | 100 |
| 5-90 | 1,000 | 90 | 100 | 90 | 50 | 70 | 100 |
| 5-91 | 1,000 | 80 | 100 | 40 | 20 | 30 | 80 |
| 5-92 | 1,000 | 80 | 100 | 60 | 50 | 40 | 100 |
| 5-104 | 1,000 | 60 | 60 | 20 | 0 | 0 | 70 |
| 5-105 | 1,000 | 80 | 100 | 60 | 30 | 40 | 80 |
| 5-113 | 1,000 | 70 | 80 | 30 | 10 | 10 | 70 |
| 5-114 | 1,000 | 80 | 100 | 40 | 10 | 20 | 70 |
| 5-125 | 1,000 | 90 | 100 | 80 | 50 | 60 | 100 |
| 5-135 | 1,000 | 90 | 100 | 80 | 30 | 40 | 100 |
| 5-149 | 1,000 | 90 | 100 | 80 | 40 | 40 | 90 |
| 5-156 | 1,000 | 80 | 100 | 60 | 30 | 20 | 80 |
| 5-160 | 1,000 | 80 | 100 | 60 | 20 | 0 | 100 |
| 5-221 | 1,000 | 80 | 100 | 40 | 10 | 30 | 100 |
| 5-228 | 1,000 | 70 | 100 | 40 | 20 | 30 | 90 |
| 5-257 | 1,000 | 80 | 100 | 50 | 20 | 30 | 80 |
| 5-270 | 1,000 | 80 | 90 | 30 | 10 | 0 | 90 |
| 5-283 (S) | 1,000 | 70 | 100 | 40 | 10 | 10 | 50 |
| 6-35 | 1,000 | 70 | 70 | 40 | 0 | 0 | 80 |
| 6-60 | 1,000 | 90 | 100 | 90 | 30 | 70 | 100 |
| 6-64 | 1,000 | 100 | 100 | 90 | 30 | 70 | 100 |
| 6-65 | 1,000 | 90 | 100 | 90 | 10 | 70 | 100 |
| 6-66 | 1,000 | 90 | 100 | 90 | 40 | 60 | 100 |
| 6-102 | 1,000 | 90 | 100 | 80 | 20 | 70 | 90 |
| 6-112 | 1,000 | 80 | 100 | 80 | 30 | 70 | 80 |
| 6-125 | 1,000 | 90 | 100 | 90 | 40 | 60 | 100 |
| 6-135 | 1,000 | 90 | 100 | 90 | 30 | 60 | 90 |
| 6-140 | 1,000 | 80 | 100 | 70 | 40 | 40 | 100 |
| 6-142 | 1,000 | 90 | 100 | 90 | 30 | 60 | 100 |
| 6-161 | 1,000 | 70 | 70 | 30 | 0 | 0 | 80 |
| 7-60 | 1,000 | 90 | 100 | 90 | 30 | 70 | 90 |
| 7-125 | 1,000 | 90 | 100 | 90 | 90 | 80 | 100 |
| 31-125 | 1,000 | 80 | 90 | 10 | 10 | 0 | 50 |
| 37-125 | 1,000 | 100 | 100 | 100 | 70 | 90 | 100 |
| 40-1 | 1,000 | 100 | 100 | 90 | 40 | 50 | 70 |
| 40-60 | 1,000 | 70 | 60 | 0 | 0 | 0 | 100 |
| 40-125 | 1,000 | 90 | 100 | 90 | 50 | 70 | 90 |
| 67-125 | 1,000 | 90 | 100 | 0 | 30 | 40 | 80 |
| 109-43 | 1,000 | 100 | 100 | 90 | 90 | 100 | 80 |
| 109-125 | 1,000 | 90 | 100 | 90 | 70 | 80 | 100 |
| 112-43 | 1,000 | 90 | 100 | 80 | 40 | 80 | 70 |
| 112-125 | 1,000 | 100 | 100 | 100 | 80 | 90 | 90 |
| 112-135 | 1,000 | 90 | 100 | 90 | 70 | 90 | 80 |
| 139-125 | 1,000 | 90 | 100 | 90 | 80 | 80 | 80 |
| 145-60 | 1,000 | 80 | 90 | 70 | 20 | 40 | 80 |
| 145-64 | 1,000 | 80 | 90 | 40 | 0 | 30 | 70 |
| 145-73 | 1,000 | 50 | 80 | 40 | 10 | 20 | 70 |
| 146-135 | 1,000 | 90 | 100 | 90 | 40 | 80 | 100 |
| 146-255 | 1,000 | 80 | 100 | 80 | 20 | 30 | 100 |
| 147-60 | 1,000 | 80 | 70 | 40 | 10 | 10 | 80 |
| 148-60 | 1,000 | 80 | 100 | 40 | 10 | 0 | 90 |
| 150-60 | 1,000 | 90 | 100 | 70 | 40 | 60 | 90 |
| 151-60 | 1,000 | 80 | 100 | 80 | 20 | 40 | 70 |
| 151-64 | 1,000 | 90 | 100 | 90 | 10 | 40 | 100 |
| 151-66 | 1,000 | 80 | 80 | 50 | 20 | 30 | 70 |
| 151-109 | 1,000 | 30 | 30 | 0 | 0 | 0 | 30 |
| 151-117 | 1,000 | 30 | 30 | 20 | 0 | 0 | 60 |
| 151-140 | 1,000 | 50 | 60 | 50 | 10 | 10 | 70 |
| 151-141 | 1,000 | 40 | 40 | 60 | 10 | 10 | 70 |
| 152-75 | 1,000 | 60 | 50 | 10 | 10 | 0 | 60 |
| 154-60 | 1,000 | 50 | 60 | 30 | 20 | 20 | 80 |
| 155-60 | 1,000 | 80 | 70 | 40 | 10 | 10 | 80 |
| 155-257 | 1,000 | 80 | 90 | 50 | 20 | 0 | 50 |
| 157-156 | 1,000 | 40 | 40 | 0 | 0 | 0 | 40 |
| 157-157 | 1,000 | 30 | 50 | 0 | 0 | 0 | 50 |
| 160-256 | 1,000 | 60 | 90 | 40 | 0 | 0 | 50 |
| 161-60 | 1,000 | 80 | 90 | 50 | 20 | 50 | 70 |
| 161-125 | 1,000 | 50 | 80 | 40 | 0 | 10 | 40 |
| 162-60 | 1,000 | 60 | 80 | 0 | 10 | 0 | 70 |
| 187-125 | 1,000 | 50 | 70 | 0 | 0 | 0 | 40 |
| 197-125 | 1,000 | 100 | 100 | 100 | 50 | 70 | 100 |
| 198-125 | 1,000 | 90 | 100 | 90 | 40 | 80 | 100 |
| 199-125 | 1,000 | 80 | 100 | 70 | 20 | 70 | 100 |
| 200-125 | 1,000 | 90 | 100 | 80 | 30 | 70 | 90 |
| 201-125 | 1,000 | 90 | 90 | 50 | 20 | 20 | 70 |
| 207-30 | 1,000 | 10 | 0 | 0 | 10 | 0 | 20 |
| 207-31 | 1,000 | 60 | 50 | 40 | 10 | 0 | 10 |
| 207-35 | 1,000 | 60 | 50 | 30 | 30 | 20 | 10 |
| 207-48 | 1,000 | 60 | 30 | 10 | 10 | 10 | 30 |
| 207-50 | 1,000 | 80 | 40 | 0 | 0 | 0 | 70 |
| 212-48 | 1,000 | 90 | 100 | 60 | 10 | 20 | 60 |
| 213-48 | 1,000 | 10 | 100 | 10 | 0 | 0 | 0 |
| 214-48 | 1,000 | 70 | 100 | 10 | 30 | 0 | 30 |
| 215-48 | 1,000 | 40 | 90 | 40 | 30 | 30 | 40 |
| 216-48 | 1,000 | 60 | 100 | 10 | 10 | 30 | 30 |
| 217-48 | 1,000 | 70 | 80 | 20 | 10 | 0 | 30 |
| 218-48 | 1,000 | 90 | 100 | 80 | 60 | 60 | 90 |
| 219-48 | 1,000 | 30 | 20 | 20 | 0 | 0 | 0 |
| 220-48 | 1,000 | 20 | 40 | 0 | 20 | 0 | 10 |
| 221-48 | 1,000 | 80 | 80 | 30 | 10 | 30 | 40 |
| 222-48 | 1,000 | 90 | 100 | 40 | 30 | 40 | 80 |
| 224-48 | 1,000 | 90 | 100 | 80 | 60 | 40 | 70 |
| 225-48 | 1,000 | 90 | 100 | 80 | 60 | 60 | 90 |
| 226-48 | 1,000 | 90 | 100 | 80 | 50 | 60 | 60 |
| 228-48 | 1,000 | 30 | 70 | 20 | 20 | 0 | 10 |
| 229-48 | 1,000 | 80 | 100 | 60 | 10 | 30 | 30 |
| 230-48 | 1,000 | 80 | 100 | 70 | 30 | 90 | 60 |
| 231-48 | 1,000 | 80 | 100 | 70 | 50 | 30 | 70 |
| 232-48 | 1,000 | 60 | 20 | 0 | 0 | 0 | 10 |
| 233-48 | 1,000 | 80 | 80 | 10 | 20 | 30 | 70 |
| 234-48 | 1,000 | 80 | 100 | 40 | 10 | 30 | 100 |
| 235-48 | 1,000 | 80 | 80 | 10 | 10 | 0 | 60 |
| 237-48 | 1,000 | 60 | 100 | 0 | 0 | 0 | 70 |
| 238-48 | 1,000 | 30 | 100 | 0 | 10 | 0 | 60 |
| 239-48 | 1,000 | 30 | 20 | 0 | 0 | 0 | 30 |
| 240-48 | 1,000 | 30 | 100 | 10 | 0 | 20 | 70 |
| 241-48 | 1,000 | 50 | 80 | 10 | 10 | 0 | 60 |
| 242-48 | 1,000 | 50 | 100 | 20 | 20 | 20 | 60 |
| 243-48 | 1,000 | 60 | 100 | 60 | 10 | 40 | 10 |
| 244-48 | 1,000 | 80 | 50 | 80 | 30 | 60 | 30 |
| 245-48 | 1,000 | 90 | 100 | 70 | 20 | 40 | 60 |
| 247-48 | 1,000 | 90 | 100 | 40 | 30 | 60 | 60 |
| 248-48 | 1,000 | 70 | 70 | 0 | 0 | 40 | 50 |
| 249-48 | 1,000 | 70 | 70 | 0 | 0 | 0 | 30 |
| 250-48 | 1,000 | 40 | 90 | 50 | 0 | 30 | 30 |
| 251-48 | 1,000 | 20 | 0 | 0 | 0 | 0 | 0 |
| 252-48 | 1,000 | 20 | 30 | 0 | 0 | 0 | 30 |
| 253-48 | 1,000 | 40 | 70 | 0 | 0 | 0 | 20 |
| 254-48 | 1,000 | 30 | 20 | 0 | 0 | 0 | 10 |
| 255-48 | 1,000 | 40 | 70 | 0 | 0 | 0 | 10 |
| 256-48 | 1,000 | 50 | 30 | 10 | 0 | 0 | 20 |
| 257-48 | 1,000 | 40 | 100 | 0 | 20 | 10 | 60 |
| 258-48 | 1,000 | 50 | 90 | 40 | 10 | 0 | 30 |
| 260-48 | 1,000 | 30 | 20 | 0 | 0 | 0 | 0 |
| 261-48 | 1,000 | 80 | 100 | 90 | 50 | 70 | 60 |
| 262-30 | 1,000 | 90 | 80 | 10 | 0 | 10 | 50 |
| 262-48 | 1,000 | 80 | 100 | 0 | 20 | 20 | 60 |
| 265-1 | 1,000 | 50 | 80 | 0 | 10 | 0 | 70 |
| 265-3 | 1,000 | 70 | 70 | 20 | 20 | 30 | 70 |
| 265-4 | 1,000 | 50 | 50 | 20 | 20 | 20 | 50 |
| 266-20 | 1,000 | 80 | 90 | 0 | 0 | 0 | 70 |

NC = data not collected

1. Use of a compound of formula (I) 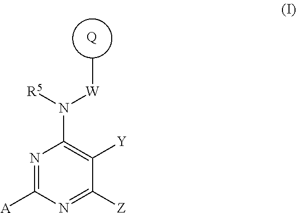
or salt or N-oxide thereof,
wherein:
A is halogen, optionally substituted alkyl, optionally substituted alkenyl, or an optionally substituted 3-8 membered ring optionally containing up to 4 heteroatoms;
R⁵ is hydrogen, optionally substituted alkyl, optionally substituted cycloalkyl, formyl, optionally substituted alkylcarbonyl, optionally substituted cycloalkylcarbonyl, optionally substituted alkenylcarbonyl, optionally substituted alkynylcarbonyl, optionally substituted phenylcarbonyl, optionally substituted heteroarylcarbonyl, optionally substituted aminocarbonyl, optionally substituted alkoxycarbonyl, optionally substituted alkylsulphonyl, optionally substituted cycloalkylsulphonyl, optionally substituted alkenylsulphonyl, optionally substituted alkynylsulphonyl, optionally substituted phenylsulphonyl, optionally substituted amino, hydroxyl, optionally substituted alkoxy, optionally substituted cycloalkoxy, optionally substituted alkylcarbonyloxy, optionally substituted alkoxycarbonyloxy, optionally substituted aminocarbonyloxy, optionally substituted phenoxy, optionally substituted phenyl, optionally substituted heteroaryl, optionally substituted alkenyl, optionally substituted alkynyl, N=CR^{g}R^{h}, cyano, optionally substituted silyl, or optionally substituted phosphonyl;
R^{g} is hydrogen, optionally substituted alkyl, optionally substituted cycloalkyl, optionally substituted phenyl, optionally substituted heteroaryl, optionally substituted amino, optionally substituted alkoxy, optionally substituted phenoxy, optionally substituted alkylthio, or optionally substituted phenylthio;
R^{h} is optionally substituted alkyl, optionally substituted cycloalkyl, optionally substituted phenyl, optionally substituted heteroaryl, optionally substituted amino, optionally substituted alkoxy, or optionally substituted alkylthio; or R^{g} and R^{h} together with the C atom to which they are joined form an optionally substituted ring;
W is a direct bond, an optionally substituted alkylene chain, an optionally substituted alkenylene chain, or an optionally substituted alkynylene chain;
Q is an optionally substituted 3-8 membered ring optionally containing up to 4 heteroatoms;
Y is halogen, cyano, nitro, hydroxyl, optionally substituted alkyl, optionally substituted cycloalkyl, optionally substituted alkenyl, optionally substituted alkynyl, optionally substituted phenyl, optionally substituted heteroaryl, optionally substituted alkoxy, optionally substituted amino, optionally substituted alkylthio, optionally substituted alkylsulphinyl, optionally substituted alkylsulphonyl, optionally substituted dialkyl phosphonyl, optionally substituted trialkylsilyl;
Z is (T)ₘ-(U)ₙ—V;
wherein
m is an integer of 0 to 1; n is an integer from 0 to 3, and n≧m;
T is an oxygen or sulphur atom;
U is CR^{w}R^{x};
each R^{w} is independently hydrogen, halogen, hydroxyl, optionally substituted alkyl, optionally substituted alkoxycarbonyl, or OR^{y} and each R^{y} is independently an optionally substituted alkyl or an optionally substituted alkylcarbonyl;
each R^{x} is independently hydrogen, halogen, optionally substituted alkyl, OR^{Z}, and each R^{Z} is independently an optionally substituted alkyl group;
or any geminal R^{w} and R^{x} together form a group selected from oxo, or ═NOR^{ca}, wherein R^{ca} is hydrogen or optionally substituted alkyl;
or any geminal, vicinal or non-adjacent R^{w} and/or R^{x} together with the C atom(s) to which they are attached and any intervening atom form an optionally substituted 3-6 membered ring;
or wherein when at least one R^{w} is OR^{y} and at least one R^{x} is OR^{Z}, said OR^{y} and OR^{Z} groups together with the C atom(s) to which they are attached and any intervening atom form an optionally substituted 5-6 membered heterocyclic ring;
V is C(O)R^{cb}, C(S)R^{cd}, C(═NR^{ce})R^{cf}, CHR^{cg}R^{ch}, CH(S[O]ₚR^{ci})(S[O]_{q}R^{cj}), CR^{ck}R^{cl}R^{cm}, or CH₂OR^{cn};
R^{cb} is hydrogen, hydroxyl, optionally substituted alkoxy, optionally substituted cycloalkoxy, optionally substituted alkylthio, or optionally substituted amino;
R^{cd} is optionally substituted alkoxy, optionally substituted cycloalkoxy, optionally substituted alkylthio, or optionally substituted amino;
R^{ce} is hydrogen, optionally substituted alkyl, optionally substituted alkoxy, optionally substituted cycloalkoxy, or optionally substituted amino;
R^{cf} is hydrogen, optionally substituted alkoxy, optionally substituted cycloalkoxy, optionally substituted alkylthio, or optionally substituted amino;
R^{cg} and R^{ch} are each independently an optionally substituted alkoxy group; or R^{cg} and R^{ch}, together with the carbon to which they are joined, form a dioxolane or dioxane ring, which ring is optionally substituted;
R^{ci} and R^{cj} are each independently an optionally substituted alkyl group; or R^{ci} and R^{cj}, together with the carbon and sulphur atoms to which they are joined, form an optionally substituted 5-6 membered ring;
p and q are each independently an integer of 0,1, or 2
R^{ck}, R^{cl}, and R^{cm} are each independently an optionally substituted alkoxy group; or R^{ck} and R^{cl} and R^{cm} together with the carbon to which they are attached form an optionally substituted trioxabicyclo[2.2.2]octane ring system;
and R^{cn} is hydrogen or an optionally substituted alkylcarbonyl group, as a herbicide.

2. A compound of formula (I) as defined in claim 1, or N-oxide or salt thereof, provided said compound is not:
6-methoxycarbonyl-4-[(4-methoxyphenyl)methylamino]-5-nitro-2-thiophen-2-yl-pyrimidine, 6-aminocarbonyl-4-[(2-aminophenyl)amino]-5-cyano-2-pyrrolidin-1-yl-pyrimidine, 6-dimethylaminocarbonyl-5-methyl-4-{[(N-methyl-N-phenyl-aminocarbonylymethyl]-amino}-2-pyridin-4-yl-pyrimidine, 5-methyl-4-{[(N-methyl-N-phenyl-aminocarbonyl)-methyl]-amino}-2-pyrimidin-4-yl-pyrimidine-6-carboxylic acid, 6-ethoxycarbonyl-5-methyl-4-{[(N-methyl-N-phenyl-aminocarbonylymethyl]-amino}-2-pyridin-4-yl-pyrimidine, 4-phenylamino-2-piperidin-1-yl-5-piperidin-1-ylmethyl-pyrimidine-6-carboxylic acid, or 4-[(2-aminophenyl)amino]-5-cyano-6-ethoxycarbonyl-2-pyrrolidin-1-yl-pyrimidine.

3. A compound according to claim 1, wherein:
A is halogen; alkyl optionally substituted by one or more groups R^{a}; alkenyl optionally substituted by one or more groups R^{b}; phenyl optionally substituted by 1-4 groups R¹; or a 5 or 6 membered heteroaromatic ring optionally substituted by 1-4 groups R¹, said heteroaromatic ring containing 1-4 heteroatoms selected from O, N and S, provided said heteroaromatic ring contains only one O and/or one S atom; a 3-8 membered non-aromatic ring containing 0-3 heteroatoms chosen from oxygen, sulphur and nitrogen, said ring being optionally substituted by 1-8 groups R²;
each R¹ is independently: halogen; cyano; nitro; azido; hydroxyl; alkyl optionally substituted by one or more groups R^{a}; alkenyl optionally substituted by one or more groups R^{b}; alkynyl optionally substituted by one or more R^{c}; cycloalkyl optionally substituted by one or more groups R^{d}; OR^{aa}; S(O)ₐR^{bb}; C(O)R^{cc}; NR^{dd}R^{ee}; SiR^{ff}R^{gg}R^{hh}; P(O)RⁱⁱR^{jj}, B(OR^{kk})(OR^{LL}); or 2 adjacent groups R¹ together with the atoms to which they are joined form a 5-7 membered ring, optionally containing 1 or 2 heteroatoms selected from O, N and S, said 5-7 membered ring being optionally substituted with 1-4 groups R³;
each R² is independently: halogen; cyano; nitro; hydroxyl; alkyl optionally substituted by one or more R^{a}; alkenyl optionally substituted by one or more R^{b}, alkynyl optionally substituted by one or more R^{c}; cycloalkyl optionally substituted by one or more R^{d}; phenyl optionally substituted by one or more R^{e}; heteroaryl optionally substituted by one or more R^{f}; OR^{aa}; S(O)ₐR^{bb}; C(O)R^{cc}; NR^{dd}R^{ee}; P(O)RⁱⁱR^{jj}; or any two geminal groups R² together form a group selected from oxo, ═CR^{mm}Rⁿⁿ, ═NOR^{oo}, and ═NNR^{pp}R^{qq}; or two groups R² are together with the atoms to which they are joined and any intervening atoms form a 3-7 membered ring system, wherein said ring system optionally contains 1-2 heteroatoms independently selected from O, S and N and is optionally substituted by 1-4 groups R⁴;
each R³ is independently: halogen; cyano; nitro; hydroxyl; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; SiR^{ff}R^{gg}R^{hh}; P(O)RⁱⁱR^{jj}; B(OR^{kk})(OR^{LL}); or any two geminal groups R³ together form a group selected from: oxo, ═CR^{mm}Rⁿⁿ, ═NOR^{oo}, and ═NNR^{pp}R^{qq};
each R⁴ is independently: halogen; cyano; nitro; hydroxyl; alkyl optionally substituted by one or more R^{a}; alkenyl optionally substituted by one or more R^{b}; alkynyl optionally substituted by one or more R^{c}; cycloalkyl optionally substituted by one or more R^{d}; OR^{aa}; S(O)ₐR^{bb}; C(O)R^{cc}; NR^{dd}R^{ee}; P(O)RⁱⁱR^{jj}; B(OR^{kk})(OR^{LL}); or any two geminal groups R⁴ together form a group selected from: oxo, ═CR^{mm}Rⁿⁿ, ═NOR^{oo}, and ═NNR^{pp}R^{qq};
each R^{a} is independently: halogen; cyano; nitro; hydroxyl; cycloalkyl; OR^{aa}; S(O)ₐR^{bb}; C(O)R^{cc}; or NR^{dd}R^{ee};
each R^{b} is independently: halogen; cyano; nitro; hydroxyl; alkoxy; S(O)₂R^{bb}; C(O)R^{cc}; or P(O)RⁱⁱR^{jj};
each R^{c} is independently: halogen; cyano; alkoxy; S(O)₂R^{bb}; C(O)R^{cc}; or SiR^{ff}R^{gg}R^{hh};
each R^{d} is independently: halogen; cyano; nitro; hydroxyl; alkyl; alkenyl; alkynyl; cycloalkyl; alkoxy; S(O)ₐR^{bb}; or C(O)R^{cc};
each R^{e} is independently: halogen; cyano; nitro; hydroxyl; alkyl optionally substituted by one or more R^{a} wherein Ra is as defined above; OR^{aa}; S(O)ₐR^{bb}; C(O)R^{cc}; or NR^{dd}R^{ee};
each R^{f} is independently: halogen; cyano; nitro; hydroxyl; alkyl optionally substituted by one or more R^{a} wherein R^{a} is as defined above; OR^{aa}; S(O)ₐR^{bb}; C(O)R^{cc}; or NR^{dd}R^{ee};
each R^{aa} is independently: alkyl, haloalkyl, alkoxyalkyl, cycloalkylalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, dialkylaminocarbonyl, alkylimino, or dialkylimino;
each R^{bb} is independently: alkyl, haloalkyl, alkoxyalkyl, alkenyl, haloalkenyl, alkynyl, haloalkynyl, cycloalkyl, or alkylcarbonylamino;
a is an integer selected from 0, 1, and 2;
R^{cc} is hydrogen, hydroxyl, alkyl, haloalkyl, alkoxyalkyl, alkenyl, haloalkenyl, alkynyl, haloalkynyl, cycloalkyl, alkoxy, haloalkoxy, alkoxyalkoxy, arylalkoxy, cycloalkoxy, amino, alkylamino, dialkylamino, or alkylsulphonylamino;
R^{dd} is hydrogen, alkyl, haloalkyl, alkoxyalkyl, alkenyl, haloalkenyl, alkynyl, haloalkynyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl;
R^{ee} is hydrogen, alkyl, haloalkyl, alkoxyalkyl, alkenyl, haloalkenyl, alkynyl, haloalkynyl, or cycloalkyl;
or R^{dd} and R^{ee}, together with the N atom to which they are joined form a 4-6 membered, optionally containing one further heteroatom selected from O, N and S, said ring being optionally substituted by 1-4 groups selected from halogen, alkyl, alkoxy, alkylcarbonyl, alkoxycarbonyl, and oxo;
R^{ff}, R^{gg} and R^{hh} are each independently alkyl or haloalkyl;
Rⁱⁱ is alkyl, haloalkyl, alkoxy, or haloalkoxy;
R^{jj} is alkoxy or haloalkoxy;
R^{kk} and R^{LL} are each independently hydrogen or alkyl; or R^{kk} and R^{LL} together with the O atoms and B atom to which they are joined form a 5- or 6-membered heterocyclic ring optionally substituted 1-4 alkyl groups;
R^{mm} is hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl;
Rⁿⁿ is hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl;
R^{oo} is hydrogen, alkyl, haloalkyl, alkoxyalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl;
R^{pp} is hydrogen, alkyl, haloalkyl, alkoxyalkyl, or cycloalkyl;
R^{qq} is hydrogen, alkyl, haloalkyl, alkylcarbonyl, haloalkylcarbonyl, alkoxycarbonyl, aminocarbonyl, alkylaminocarbonyl, or dialkylaminocarbonyl; or R^{pp} and R^{qq} together with the nitrogen to which they are attached form a 5- or 6-membered optionally substituted ring, said ring optionally containing a further heteroatom selected from oxygen, sulphur and nitrogen, said substitution(s) being selected from: halogen and alkyl;
R⁵ is hydrogen, alkyl optionally substituted by one or more R^{p}, cycloalkyl optionally substituted by one or more R^{q}, phenyl optionally substituted by one or more R^{r}, heteroaryl optionally substituted by one or more R^{s}, alkenyl optionally substituted by one or more R^{t}, or alkynyl optionally substituted by one or more groups selected from: R^{u}, OR^{rr}, SO₂R^{ss}, C(O)R^{uu}, NR^{vv}R^{ww}, N═CR^{g}R^{h}, cyano, trialkylsilyl, and dialkyl phosphonyl;
each R^{g} is independently: hydrogen, alkyl optionally substituted by one or more R^{xx}, cycloalkyl optionally substituted by one or more R^{yy}, phenyl optionally substituted by one or more R^{zz}, or heteroaryl optionally substituted by one or more R^{ab}, NR^{ac}R^{ad}, OR^{ae}, and/or SR^{af};
each R^{h} is independently: alkyl optionally substituted by one or more R^{xx}, cycloalkyl optionally substituted by one or more R^{yy}, phenyl optionally substituted by one or more R^{zz}, or heteroaryl optionally substituted by one or more R^{ab}, NR^{ac}R^{ad}, OR^{ae}, and/or SR^{af}; or R^{g} and R^{h} together with the carbon atom to which they are joined to form a ring that is optionally substituted by 1-4 groups R^{ag};
each R^{p} is independently: halogen; cyano; nitro; hydroxyl; cycloalkyl; phenyl optionally substituted by one or more R^{aj}; heteroaryl optionally substituted by R^{ak}; alkoxy; alkoxyalkoxy; S(O)_{b}R^{am}; C(O)R^{an}; or NR^{ao}R^{ap};
each R^{g} is independently: halogen; cyano; nitro; hydroxyl; alkyl; haloalkyl; alkenyl; alkynyl; cycloalkyl; phenyl optionally substituted by one or more R^{aj}; heteroaryl optionally substituted by R^{ak}; alkoxy; S(O)_{b}R^{am}; or C(O)R^{an};
each R^{r} is independently halogen; cyano; nitro; hydroxyl; alkyl optionally substituted by one or more R^{aq}; alkenyl optionally substituted by one or more R^{ar}; alkynyl optionally substituted by one or more R^{as}; cycloalkyl optionally substituted by one or more R^{at}; OR^{al}; S(O)_{b}R^{am}; or C(O)R^{an};
each R^{s} is independently: halogen; cyano; nitro; hydroxyl; alkyl optionally substituted by one or more R^{aq}; alkenyl optionally substituted by one or more R^{ar}; alkynyl optionally substituted by one or more R^{as}; cycloalkyl optionally substituted by one or more R^{at}; OR^{al}; S(O)_{b}R^{am}; or C(O)R^{an};
each R^{t} is independently: halogen, cyano, cycloalkyl, phenyl, or alkoxycarbonyl;
each R^{u} is independently: halogen, cyano, cycloalkyl, phenyl, alkoxy, or alkoxycarbonyl;
each R^{rr} is independently: hydrogen; alkyl; haloalkyl; cycloalkyl; alkylcarbonyl; haloalkylcarbonyl; alkoxycarbonyl; aminocarbonyl; alkylaminocarbonyl; dialkylaminocarbonyl; or phenyl optionally substituted by one or more groups R^{zz};
each R^{ss} is independently: alkyl; haloalkyl; alkenyl; haloalkenyl; alkynyl; haloalkynyl; cycloalkyl; or phenyl optionally substituted by one or more groups R^{zz};
each R^{uu} is independently: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; NR^{ac}R^{ad}; or OR^{ae};
each R^{vv} is: hydrogen; alkyl optionally substituted by one or more R^{xx}; cycloalkyl optionally substituted by one or more R^{yy}; phenyl optionally substituted by one or more R^{zz}; heteroaryl optionally substituted by one or more R^{ab}; SO₂R^{ss}; or C(O)R^{uu};
each R^{ww} is: hydrogen, alkyl optionally substituted by one or more R^{xx};
cycloalkyl optionally substituted by one or more R^{yy}; phenyl optionally substituted by one or more R^{zz}; or heteroaryl optionally substituted by one or more R^{ab};
or R^{vv} and R^{ww} together with the nitrogen atom to which they are attached, form a 5-6 membered ring, which ring optionally contains an additional heteroatom selected from O, N and S, and is optionally substituted by 1-2 alkyl or alkylcarbonyl groups;
each R^{xx} is independently: halogen; cyano; alkoxy; cycloalkyl; phenyl optionally substituted by one or more R^{zz}; heteroaryl optionally substituted by one or more R^{ab}; or alkoxycarbonyl;
each R^{yy} is independently: halogen; cyano; alkyl; cycloalkyl; phenyl optionally substituted by one or more R^{zz}; heteroaryl optionally substituted by one or more R^{ab}; or alkoxycarbonyl;
each R^{zz} is independently: halogen; cyano; nitro; hydroxyl; alkyl optionally substituted by one or more R^{aq}; alkenyl optionally substituted by one or more R^{ar}; alkynyl optionally substituted by one or more R^{as}; OR^{al}; S(O)_{b}R^{am}; C(O)R^{an};
each R^{ab} is independently: halogen; cyano; hydroxyl; alkyl optionally substituted by one or more R^{aq}; alkenyl optionally substituted by one or more R^{ar}; alkynyl optionally substituted by one or more R^{as}; OR^{al}; S(O)_{b}R^{am}; or C(O)R^{an};
each R^{ac} is independently: hydrogen; alkyl; haloalkyl; alkoxyalkyl; alkylcarbonyl; alkoxycarbonyl; or phenyl optionally substituted with one or more R^{zz};
each R^{ad} is independently: hydrogen; alkyl; haloalkyl; alkoxyalkyl; or phenyl optionally substituted with one or more R^{zz};
or R^{ac} and R^{ad} together with the N-atom to which they are joined form a 4-6 membered heterocyclic ring;
each R^{ae} is independently: alkyl; phenylalkyl optionally substituted by one or more R^{zz}; or phenyl optionally substituted by one or more R^{zz};
each R^{af} is independently alkyl or phenyl;
each R^{ag} is independently alkyl or haloalkyl;
each R^{aj} is independently: halogen; cyano; nitro; alkyl; haloalkyl; alkoxyalkyl; alkenyl; alkynyl; alkoxy; haloalkoxy; alkylthio; haloalkylthio; alkylsulphonyl; haloalkylsulphonyl; alkoxycarbonyl; aminocarbonyl; alkylaminocarbonyl; dialkylaminocarbonyl; amino; alkylamino; dialkylamino; alkylcarbonylamino; alkylcarbonyl(alkyl)amino; or aminocarbonylamino;
each R^{ak} is independently: halogen; cyano; hydroxyl; alkyl; haloalkyl; alkoxyalkyl; alkenyl; alkynyl; alkoxy; haloalkoxy; alkylthio; haloalkylthio; alkylsulphonyl; haloalkylsulphonyl; alkoxycarbonyl; aminocarbonyl; alkylaminocarbonyl; dialkylaminocarbonyl; amino; alkylamino; dialkylamino; alkylcarbonylamino; alkylcarbonyl(alkyl)amino;or aminocarbonylamino;
each R^{al} is independently: alkyl, haloalkyl, alkoxyalkyl, or alkylsulphonyl;
each R^{am} is independently: alkyl, haloalkyl, alkenyl, alkynyl, phenyl, or alkylcarbonylamino;
b is an integer selected from 0, 1 and 2;
each R^{an} is independently: hydrogen, alkyl, haloalkyl, alkoxy, haloalkoxy, amino, alkylamino, dialkylamino, or alkylsulphonylamino;
each R^{ao} is independently hydrogen, alkyl, alkoxyalkyl, phenylalkyl, formyl, alkylcarbonyl, alkoxycarbonyl, aminocarbonyl, alkylaminocarbonyl, or dialkylaminocarbonyl;
each R^{ap} is independently: hydrogen, alkyl, alkoxyalkyl, or phenylalkyl;
or R^{ao} and R^{ap} together with the nitrogen atom to which they are attached form a 5-6 membered ring which optionally contains an additional heteroatom selected from O, N, and S, and is optionally substituted by 1-2 alkyl or alkylcarbonyl groups;
each R^{aq} is independently: halogen, alkoxy, cyano, alkoxycarbonyl, or alkylsulphonyl;
each R^{ar} is independently: halogen, cyano, nitro, alkoxy, alkylcarbonyl, alkoxycarbonyl, aminocarbonyl, alkylsulphonyl, or dialkyl phosphonyl;
each R^{as} is independently: halogen, cyano, trialkylsilyl, alkylcarbonyl, alkoxycarbonyl, aminocarbonyl, or alkylsulphonyl;
each R^{at} is independently: halogen, alkyl, or alkoxycarbonyl;
W is a direct bond, C₁₋₃ alkylene chain optionally substituted by 1-4 groups Rⁱ, C₁₋₃ alkenylene chain optionally substituted by 1-3 groups R^{j}, or C₁₋₃ alkynylene chain optionally substituted by 1-2 groups R^{k};
each Rⁱ is independently: halogen; cyano; nitro; alkyl; haloalkyl; hydroxyalkyl; alkoxyalkyl; carboxyalkyl; alkoxycarbonylalkyl; alkylcarbonyloxyalkyl; cycloalkyl; alkenyl; hydroxyl; alkoxy; haloalkoxy; alkylthio; haloalkylthio; alkylcarbonyl; alkoxycarbonyl; carboxy; alkylcarbonyloxy; aminocarbonyl; alkylaminocarbonyl; dialkylaminocarbonyl; or dialkylphosphonyl;
each R^{j} is independently: halogen; cyano; nitro; alkyl; haloalkyl; alkoxyalkyl; alkylcarbonyloxyalkyl; cycloalkyl; alkoxy; haloalkoxy; alkylthio; haloalkylthio; alkylcarbonyl; alkoxycarbonyl; aminocarbonyl, alkylaminocarbonyl; or dialkylaminocarbonyl;
each R^{k} is independently: halogen; cyano; nitro; alkyl; haloalkyl; alkoxyalkyl; alkoxy; haloalkoxy; alkylthio; haloalkylthio; alkylcarbonyl; alkoxycarbonyl; aminocarbonyl; alkylaminocarbonyl; or dialkylaminocarbonyl;
Q is: phenyl optionally substituted by 1-4 groups R⁶;
a 5- or 6-membered heteroaromatic ring containing 1-4 heteroatoms, wherein at least one of said heteroatoms is N and any further heteroatom is independently selected from N, S and O provided that there is no more than one O and/or S atom in said heteroaromatic ring, and wherein said heteroaromatic ring is optionally substituted by 1-4 groups R⁶; or, a 3-8 membered non-aromatic ring containing 0-3 heteroatoms selected independently from O, S and N, and wherein said 3-8 membered non-aromatic ring is optionally substituted by 1-8 groups R⁷;
each R⁶ is independently: halogen; cyano; nitro; azido; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; OR^{da}; S(O)_{d}R^{db}; C(O)R^{dc}; or NR^{dk}R^{de}; or 2 adjacent groups R⁶ together with the atoms to which they are joined to form a 5-7 membered ring, wherein said ring optionally contains 1 or 2 heteroatoms selected independently from O, S and N, and said ring is optionally substituted with 1-4 groups R⁸;
each R⁷ is independently: halogen; cyano; nitro; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; phenyl optionally substituted by one or more R^{v}; heteroaryl optionally substituted by one or more R^{v}; OR^{da}; S(O)_{d}R^{db}; C(O)R^{dc}; or NR^{dk}R^{de}; or any two geminal groups R⁷ together form a group selected from: oxo, ═CR^{dm}R^{dn}, ═NOR^{do}, and ═NNR^{dp}R^{dq}; or two groups R⁷ are together with the atoms to which they are joined and any intervening atoms form a 3-7 membered ring system, wherein said ring system optionally contains 1-2 heteroatoms independently selected from O, S and N and is optionally substituted by 1-4 groups R⁹;
each R⁸ is independently: halogen; cyano; nitro; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; OR^{da}; S(O)_{d}R^{db}; C(O)R^{dc}; or NR^{dk}R^{de}; or any two geminal groups R⁸ together form a group selected from: oxo, ═CR^{dm}R^{dn}, ═NOR^{do}, and ═NNR^{dp}R^{dq};
each R⁹ is independently: halogen; cyano; nitro; hydroxyl; alkyl optionally substituted by one or more R^{L}; alkenyl optionally substituted by one or more R^{m}; alkynyl optionally substituted by one or more Rⁿ; cycloalkyl optionally substituted by one or more R^{o}; OR^{da}; S(O)_{d}R^{db}; C(O)R^{dc}; or NR^{dk}R^{de}; or any two geminal groups R⁹ together form a group selected from: oxo, ═CR^{dm}R^{dn}, ═NOR^{do}, and ═NNR^{dp}R^{dq};
each R^{L} is independently: halogen; cyano; nitro; hydroxyl; cycloalkyl; OR^{da}; S(O)_{d}R^{db}; C(O)R^{dc}; or NR^{dk}R^{de};
each R^{m} is independently: halogen; cyano; nitro; hydroxyl; alkoxy; S(O)₂R^{db}; C(O)R^{dc}; or P(O)R^{di}R^{dj};
each Rⁿ is independently: halogen; cyano; alkoxy; S(O)₂R^{db}; C(O)R^{dc}; or SiR^{df}R^{dg}R^{dh};
each R^{o} is independently: halogen; cyano; nitro; hydroxyl; alkyl; alkenyl; alkynyl; cycloalkyl; alkoxy; S(O)_{d}R^{db}; or C(O)R^{dc};
each R^{v} is independently: halogen; cyano; nitro; hydroxyl; alkyl optionally substituted by one or more R^{L}; OR^{da}; S(O)_{d}R^{db}; C(O)R^{dc}; or NR^{dk}R^{de};
each R^{da} is independently: alkyl; haloalkyl; alkoxyalkyl; cycloalkylalkyl; alkenyl; haloalkenyl; alkynyl; haloalkynyl; cycloalkyl; alkylcarbonyl; haloalkylcarbonyl; alkoxycarbonyl; haloalkoxycarbonyl; alkylsulphonyl; haloalkylsulphonyl; aminocarbonyl; alkylaminocarbonyl; dialkylaminocarbonyl; alkylimino; or dialkylimino;
each R^{db} is independently: alkyl; haloalkyl; alkoxyalkyl; alkenyl; haloalkenyl; alkynyl; haloalkynyl; cycloalkyl; or alkylcarbonylamino;
d is an integer from 0 to 2;
each R^{dc} is independently: hydrogen; hydroxyl; alkyl; haloalkyl; alkoxyalkyl; alkenyl; haloalkenyl; alkynyl; haloalkynyl; cycloalkyl; alkoxy; haloalkoxy; alkoxyalkoxy; arylalkoxy; cycloalkoxy; amino; alkylamino; dialkylamino; or alkylsulphonylamino;
each R^{dk} is independently: hydrogen; alkyl; haloalkyl; alkoxyalkyl; alkenyl; haloalkenyl; alkynyl; haloalkynyl; cycloalkyl; alkylcarbonyl; haloalkylcarbonyl; alkoxycarbonyl; haloalkoxycarbonyl; alkylsulphonyl; haloalkylsulphonyl; aminocarbonyl; alkylaminocarbonyl; dialkylaminocarbonyl;
each R^{de} is independently: hydrogen; alkyl; haloalkyl; alkoxyalkyl; alkenyl; haloalkenyl; alkynyl; haloalkynyl; cycloalkyl; or R^{dk} and R^{de}, together with the N atom to which they are joined form a 4-6 membered saturated, unsaturated or aromatic ring, optionally containing one further heteroatom selected from O, N and S, said ring being optionally substituted by 1-4 groups selected from halogen, alkyl, alkoxy, alkylcarbonyl, alkoxycarbonyl, and oxo;
each R^{df}, R^{dg}, and R^{dh} is independently: alkyl or haloalkyl;
each R^{di} is independently: alkyl, haloalkyl, alkoxy, or haloalkoxy;
each R^{dj} is independently: alkoxy or haloalkoxy;
each R^{dm} is independently: hydrogen, halogen, cyano, nitro, alkyl, haloalkyl, alkoxyalkyl, cycloalkyl, alkylcarbonyl, haloalkylcarbonyl, alkoxycarbonyl, haloalkoxycarbonyl, alkylsulphonyl, haloalkylsulphonyl, aminocarbonyl, or alkylaminocarbonyl, dialkylaminocarbonyl;
each R^{dn} is independently: hydrogen, halogen, cyano, nitro, alkyl, haloalkyl, alkylcarbonyl, haloalkylcarbonyl, alkoxycarbonyl, haloalkoxycarbonyl, alkylsulphonyl, haloalkylsulphonyl, aminocarbonyl, alkylaminocarbonyl, or dialkylaminocarbonyl;
each R^{do} is independently: hydrogen, alkyl, haloalkyl, alkoxyalkyl, cycloalkyl, alkylcarbonyl, haloalkylcarbonyl, alkoxycarbonyl, haloalkoxycarbonyl, aminocarbonyl, alkylaminocarbonyl, or dialkylaminocarbonyl;
each R^{dp} is independently: hydrogen, alkyl, haloalkyl, alkoxyalkyl, or cycloalkyl;
each R^{dq} is independently: hydrogen, alkyl, haloalkyl, alkylcarbonyl, haloalkylcarbonyl, alkoxycarbonyl, aminocarbonyl, alkylaminocarbonyl, or dialkylaminocarbonyl; or R^{dp} and R^{dq} together with the nitrogen to which they are attached form a 5- or 6-membered ring, optionally containing an additional heteroatom selected from O, N and S, and being optionally substituted with one or more groups selected from halogen and alkyl;
Y is: halogen; cyano; nitro; hydroxyl; C₁₋₆ alkyl optionally substituted by 1-3 groups R^{ba}; C₁₋₆ haloalkyl optionally substituted by 1-3 groups R^{ba}; C₃₋₆ cycloalkyl optionally substituted by 1-3 groups R^{bc}; C₂₋₆ alkenyl optionally substituted by 1-3 groups R^{bd}; C₂₋₆ alkynyl optionally substituted by 1-3 groups R^{be}; phenyl optionally substituted by 1-4 groups R^{bf}; heteroaryl optionally substituted by 1-3 groups R^{bg}; OR^{bh}; NR^{bi}R^{bj}; S(O)_{c}R^{bk}; di(C₁₋₄)alkyl phosphonyl; or tri(C₁₋₄)alkylsilyl;
each R^{ba} is independently: cyano; nitro; hydroxyl; C₁₋₄ alkoxy; C₁₋₄ alkylthio; C₁₋₄ alkylcarbonyl; or C₁₋₄ alkoxycarbonyl;
each R^{bc} is independently: halogen; cyano; C₁₋₄ alkyl; C₁₋₄ alkoxy; or C₁₋₄ alkoxycarbonyl;
each R^{bd} is independently: halogen; cyano; C₁₋₄ alkylcarbonyl; or C₁₋₄ alkoxycarbonyl;
each R^{be} is independently: halogen; cyano; hydroxyl; C₁₋₄ alkoxycarbonyl; or tri(C₁₋₄)alkylsilyl;
each R^{bf} is independently: halogen; cyano; C₁₋₄ alkyl; C₁₋₄ haloalkyl; C₁₋₄ alkoxy(C₁₋₄)alkyl; C₁₋₄ alkoxy; C₁₋₄ haloalkoxy; C₁₋₄ alkylsulphonyl; or C₁₋₄ alkoxycarbonyl;
each R^{bg} is independently: halogen; cyano; C₁₋₄ alkyl; C₁₋₄ haloalkyl; C₁₋₄ alkoxy(C₁₋₄)alkyl; C₁₋₄ alkoxy; C₁₋₄ haloalkoxy; C₁₋₄ alkylsulphonyl; or C₁₋₄ alkoxycarbonyl;
each R^{bh} is independently: C₁₋₄ alkyl; C₁₋₄ haloalkyl; or C₁₋₄ alkylsulphonyl;
each R^{bi} is independently: hydrogen; C₁₋₄ alkyl; C₁₋₄ alkylcarbonyl; C₁₋₄ alkoxycarbonyl; aminocarbonyl; C₁₋₄ alkylaminocarbonyl; or di(C₁₋₄)alkylaminocarbonyl;
each R^{bj} is independently hydrogen or C₁₋₄ alkyl;
each R^{bk} is independently C₁₋₄ alkyl or C₁₋₄ haloalkyl;
c is an integer from 0 to 2;
Z is (O)ₘ—(U)ₙ—V;
m and n are as defined in claim 1;
U is CR^{w}R^{x};
R^{w} is hydrogen, halogen, hydroxyl, C₁₋₄ alkyl, C₁₋₄ haloalkyl, C₁₋₃ alkoxy(C_{1 1-3})alkyl, C₁₋₆ alkoxycarbonyl, C₁₋₄ alkoxy, C₁₋₄ alkylcarbonyloxy;
R^{x} is hydrogen, halogen, C₁₋₄ alkyl, C₁₋₄ haloalkyl, C₁₋₃ alkoxy(C₁₋₃)alkyl, C₁₋₄ alkoxy; or any geminal R^{w} and R^{x} together form a group selected from oxo, or ═NOR^{ca}, wherein R^{ca} is hydrogen or C₁₋₄ alkyl; or any geminal, vicinal or non-adjacent R^{w} and/or R^{x} together with the C atom(s) to which they are joined and any intervening atom form a 3-6 membered ring;
V is C(O)R^{cb}, C(S)R^{cd}, C(═NR^{ce})R^{cf}, CHIR^{cg}R^{ch}, CH(S[O]ₚR^{ci})(S[O]_{q}R^{cj}), CR^{ck}R^{ci}R^{cm}, or CH₂OR^{cn};
R^{cb} is hydrogen, hydroxyl, C₁₋₁₀ alkylthio, amino, C₁₋₆ alkylamino, di(C₁₋₄)alkylamino, or OR^{co});
R^{cd} alkoxy, C₁₋₁₀ alkylthio, amino, C₁₋₆ alkylamino, or di(C₁₋₄)alkylamino;
R^{ce} is hydrogen, C₁₋₆ alkyl, amino, C₁₋₆ alkylamino, di(C₁₋₄)alkylamino, C₁₋₆ alkoxy, or C₃₋₆ cycloalkoxy;
R^{cf} is hydrogen, C₁₋₆ alkoxy, C₁₋₆ alkylthio, amino, C₁₋₆ alkylamino, or di(C₁₋₄)alkylamino;
R^{cg} and R^{ch} are each independently C₁₋₄ alkoxy; or R^{cg} and R^{ch} together with the carbon to which they are joined form a dioxolane or dioxane ring, which is optionally substituted by 1-2C₁₋₂ alkyl groups;
R^{di} and R^{cj} are each independently C₁₋₄ alkyl; or R^{ci} and R^{cj} together with the carbon and sulphur atoms to which they are joined form a 5-6 membered ring, which is optionally substituted by 1-2C₁₋₂ alkyl groups;
p and q are as defined in claim 1,
R^{ck}, R^{cl}, and R^{cm} are each independently C₁₋₄ alkoxy;
R^{cn} is hydrogen or C₁₋₄ alkylcarbonyl;
R^{co} is C₁₋₂₀ alkyl optionally substituted by 1-3 groups R^{cq}, C₁₋₂₀ haloalkyl optionally substituted by 1-3 groups R^{cq}, or C₃₋₆ cycloalkyl;
each R^{cq} is independently: C₃₋₆ cycloalkyl, C₁₋₆ alkoxy, phenyl optionally substituted with 1-4 groups R^{cr}, or heteroaryl optionally substituted with 1-3 groups R^{cs};
and,
each R^{cr} and R^{cs} are independently: halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ alkoxycarbonyl, or amino.

4. A compound according to claim 3, wherein:
A is fluorine; chlorine; bromine; iodine;
C₁₋₆ alkyl optionally substituted by 1-3 groups R^{a}; C₁₋₆ haloalkyl optionally substituted by 1-3 groups R^{a}; C₂₋₆ alkenyl optionally substituted by 1-3 groups R^{b}; cycloalkyl optionally substituted by 1-4 groups R²; a ring system selected from: phenyl, thiophene, furan, pyrrole, isoxazole, isothiazole, oxazole, thiazole, imidazole, pyrazole, 1,2,3-oxadiazole,1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,3,4-oxatriazole, 1,2,3,4-thiatriazole, tetrazole, pyridine, pyridazine, pyrimidine, pyrazine, 1,2,3-triazine, 1,2,4-triazine, 1,3,5-triazine, and 1,2,4,5-tetrazine, wherein said ring system is optionally substituted by 1-3 groups R¹; a ring system selected from: aziridine, azetidine, pyrrolidine, piperidine, piperazine, azepane, oxetane, tetrahydrofuran, tetrahydropyran, morpholine, thiomorpholine, dihydrofuran, dihydropyran, dioxolane, 1,3-dioxane, 1,4-dioxane, 1,3-dioxepane, dithiolane, 1,3-dithiane, 1,4-dithiane, wherein said ring system is optionally substituted by 1-4 groups R²;
each R¹ is independently: halogen; cyano; nitro; hydroxyl; C₁₋₆ alkyl optionally substituted by 1-4 groups R^{a}; C₁₋₆ haloalkyl optionally substituted by 1-4 groups R^{a}; OR^{aa}; S(O)ₐR^{bb}; C(O)R^{cc}; or NR^{dd}R^{ee}; or 2 adjacent groups R¹ together with the atoms to which they are joined form a 5-or 6-membered ring optionally containing 1 or 2 heteroatoms selected from O, N and S, said ring being optionally substituted with 1-4 groups R³;
each R² is independently: halogen; cyano; nitro; hydroxyl; C₁₋₆ alkyl optionally substituted by 1-4 groups R^{a}; C₁₋₆ haloalkyl optionally substituted by 1-4 groups R^{a}; C₂₋₆ alkenyl optionally substituted by 1-3 groups R^{b}; OR^{aa}; S(O)ₐR^{bb}; C(O)R^{cc}; or NR^{dd}R^{ee}; or any two geminal groups R² together form a group selected from oxo, ═CR^{mm}Rⁿⁿ, ═NOR^{oo}, ═NNR^{pp}R^{qq}; or two groups R² are together with the atoms to which they are joined and any intervening atoms form a 3-7 membered ring system, wherein said ring system optionally contains 1-2 heteroatoms independently selected from O, S and N and is optionally substituted by 1-4 groups R⁴;
each R³ is independently: halogen; cyano; nitro; hydroxyl; C₁₋₆ alkyl optionally substituted by 1-4 groups R^{a}; C₁₋₆ haloalkyl optionally substituted by 1-4 groups R^{a}; OR^{aa}; S(O)ₐR^{bb}; C(O)R^{cc}; or NR^{dd}R^{ee}; or any two geminal groups R³ together form a group selected from oxo, ═CR^{mm}Rⁿⁿ, ═NOR^{oo}, or ═NNR^{pp}R^{qq};
each R⁴ is independently: halogen; cyano; nitro; hydroxyl; C₁₋₆ alkyl optionally substituted by 1-4 groups R^{a}; C₁₋₆ haloalkyl optionally substituted by 1-4 groups R^{a}; C₂₋₆ alkenyl optionally substituted by 1-3 groups R^{b}; OR^{aa}; S(O)ₐR^{bb}; C(O)R^{cc}; or NR^{dd}R^{ee}; or any two geminal groups R⁴ together form a group selected from oxo; ═CR^{mm}Rⁿⁿ; ═NOR^{oo}; ═NNR^{pp}R^{qq};
each R^{a} is independently: cyano, hydroxyl, C₃₋₆ cycloalkyl, OR^{aa}, S(O)ₐR^{bb}, C(O)R^{cc}, or NR^{dd}R^{ee};
each R^{b} is independently: halogen, cyano, nitro, C₁₋₆ alkoxy, S(O)₂R^{bb}, or C(O)R^{cc};
each R^{aa} is independently: C₁₋₆ alkyl, C₁₋₆ haloalkyl, C₁₋₄ alkoxy(C₁₋₄)alkyl, or C₁₋₆ alkylcarbonyl;
each R_{bb} is independently: C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino;
a is as defined in claim 3;
each R_{cc} is independently: hydrogen, hydroxyl, C₁₋₆ alkyl, C₁₋₆ haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino, or C₁₋₆ alkylsulphonylamino;
each R^{dd} is independently: hydrogen, C₁₋₆ alkyl, C₁₋₆ alkylcarbonyl, C₁₋₆ alkoxycarbonyl, C₁₋₆ alkylsulphonyl, C₁₋₆ haloalkylsulphonyl, aminocarbonyl, C₁₋₆ alkylaminocarbonyl, or di(C₁₋₄)alkylaminocarbonyl;
each R^{ee} is independently: hydrogen, C₁₋₆ alkyl, C₃₋₆ cycloalkyl; or R^{dd} and R^{ee}, together with the N atom to which they are joined form a 5- or 6-membered saturated ring optionally containing one further heteroatom selected from O, N and S, said ring being optionally substituted by 1 or 2C₁₋₆ alkyl or oxo groups;
each R^{mm} is independently: hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl;
each Rⁿⁿ is independently: hydrogen, halogen, cyano, nitro, C₁₋₆ alkyl, C₁₋₆ alkylcarbonyl, C₁₋₆ alkoxycarbonyl, C₁₋₆ alkylsulphonyl, or aminocarbonyl;
each R^{oo} is independently hydrogen, C₁₋₆ alkyl, or C₃₋₆ cycloalkyl;
each R^{pp} is independently hydrogen, C₁₋₆ alkyl, or C₃₋₆ cycloalkyl;
each R^{qq} is independently hydrogen or C₁₋₆ alkyl;
R⁵ is hydrogen; C₁₋₆ alkyl optionally substituted by 1-4 groups R^{p}; C₁₋₆ haloalkyl optionally substituted by 1-4 groups R^{p}; C₃₋₈ cycloalkyl optionally substituted by 1-4 groups R^{q}; phenyl optionally substituted by 1-3 groups R^{r}; heteroaryl optionally substituted by 1-3 groups R^{s}; SO₂R^{ss}; or C(O)R^{uu};
each R^{p} is independently: cyano; C₃₋₆ cycloalkyl; phenyl optionally substituted by 1-3 groups R^{aj}; heteroaryl optionally substituted by 1-3 groups R^{ak}, C₁₋₄ alkoxy; or C₁₋₄ alkoxy(C₁₋₄)alkoxy;
each R^{q} is independently halogen, cyano, C₁₋₄ alkyl, C₁₋₄ haloalkyl, C₃₋₆ cycloalkyl, phenyl optionally substituted by 1-3 groups R^{aj}, or C(O)R^{an};
each R^{r} is independently halogen, cyano, nitro, C₁₋₄ alkyl, C₁₋₄ haloalkyl, C₂₋₈ alkoxyalkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl;
each R^{s} is independently halogen, cyano, C₁₋₄ alkyl, C₁₋₄ haloalkyl, C₂₋₈ alkoxyalkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl;
each R^{ss} is independently C₁₋₄ alkyl, C₁₋₄ haloalkyl, or phenyl optionally substituted by 1-3 groups R^{zz};
each R^{uu} is independently: hydrogen; C₁₋₄ alkyl optionally substituted by 1-4 groups R^{xx}; C₁₋₄ haloalkyl optionally substituted by 1-4 groups R^{xx}; C₃₋₆ cycloalkyl optionally substituted by 1-4 groups R^{yy}; phenyl optionally substituted by 1-3 groups R^{zz}; heteroaryl optionally substituted by 1-3 groups R^{ab}; NR^{ac}R^{ad}; or OR^{ae};
each R^{xx} is independently: cyano, C₁₋₄ alkoxy, C₃₋₆ cycloalkyl, phenyl optionally substituted by 1-3 groups R^{zz}, heteroaryl optionally substituted by 1-3 groups R^{ab}, or C₁₋₄ alkoxycarbonyl;
each R^{yy} is independently: halogen, cyano, C₁₋₄ alkyl, or phenyl optionally substituted by 1-3 groups R^{zz};
each R^{zz} is independently: halogen, cyano, nitro, C₁₋₄ alkyl, C₁₋₄ haloalkyl, C₁₋₄ alkoxy(C₁₋₄)alkyl, C₁₋₄ alkoxy, C₁₋₄ haloalkoxy, C₁₋₄ alkylsulphonyl, C₁₋₄ haloalkylsulphonyl, or C(O)R^{an};
each R^{ab} is independently: halogen, cyano, C₁₋₄ alkyl, C₁₋₄ haloalkyl, C₁₋₄ alkoxy(C₁₋₄)alkyl, C₁₋₄ alkoxy, C₁₋₄ haloalkoxy, C₁₋₄ alkylsulphonyl, C₁₋₄ haloalkylsulphonyl, or C(O)R^{an};
each R^{ac} is independently hydrogen, C₁₋₄ alkyl, C₁₋₄ haloalkyl, or C₁₋₄ alkoxy(C₁₋₄)alkyl;
each R^{ad} is independently hydrogen, C₁₋₄ alkyl, C₁₋₄ haloalkyl, or C₁₋₄ alkoxy(C₁₋₄)alkyl; or R^{ac} and R^{ad} together with the N atom to which they are joined form a 5- or 6-membered heterocyclic ring;
each R^{ae} is independently C₁₋₄ alkyl, phenylC₁₋₄alkyl optionally substituted by 1-3 groups R^{zz}, or phenyl optionally substituted by 1-3 groups R^{zz};
each R^{aj} is independently halogen, cyano, nitro, C₁₋₄ alkyl, C₁₋₄ haloalkyl, C₂₋₈ alkoxyalkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl;
each R^{ak} is independently halogen, cyano, C₁₋₄ alkyl, C₁₋₄ haloalkyl, C₁₋₄ alkoxy(C₁₋₄)alkyl, C₁₋₄ alkoxy, C₁₋₄ haloalkoxy, C₁₋₄ alkylsulphonyl, C₁₋₄ haloalkylsulphonyl, or C₁₋₄ alkoxycarbonyl;
each R^{an} is independently C₁₋₄ alkyl, C₁₋₄ haloalkyl, C₁₋₄ alkoxy, C₁₋₄ haloalkoxy, amino, C₁₋₄ alkylamino, or di(C₁₋₄)alkylamino;
W is a direct bond, or a C₁₋₂ alkylene chain optionally substituted by 1-2 groups Rⁱ; wherein each Rⁱ is independently selected from: halogen, cyano, C₁₋₄ alkyl, C₁₋₄ haloalkyl, C₁₋₃ hydroxyalkyl, C₁₋₂ alkoxy(C₁₋₂)alkyl, carboxy(C₁₋₃)alkyl, C₁₋₃ alkoxycarbonyl(C₁₋₃)alkyl, C₁₋₄ alkoxycarbonyl, carboxy, aminocarbonyl, C₁₋₄ alkylaminocarbonyl, or di(C₁₋₃)alkylaminocarbonyl;
Q is a ring system selected from: phenyl, thiophene, furan, pyrrole, isoxazole, isothiazole, oxazole, thiazole, imidazole, pyrazole, 1,2,3-oxadiazole,1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,2,3-triazole, 1,2,4-triazole, 1,2,3,4-oxatriazole, 1,2,3,4-thiatriazole, tetrazole, pyridine, pyridazine, pyrimidine, pyrazine, 1,2,3-triazine, 1,2,4-triazine, 1,3,5-triazine, 1,2,4,5-tetrazine, wherein said ring system is optionally substituted by 1-3 groups R⁶; optionally substituted C₃₋₈ cycloalkyl or optionally substituted cycloalkenyl, wherein said substituents are selected from 1-4 groups R⁷; or a ring system selected from: aziridine, azetidine, pyrrolidine, piperidine, piperazine, azepane, oxetane, tetrahydrofuran, tetrahydropyran, morpholine, thiomorpholine, dihydrofuran, dihydropyran, dioxolane, 1,3-dioxane, 1,4-dioxane, 1,3-dioxepane, dithiolane, 1,3-dithiane, 1,4-dithiane, wherein said ring system is optionally substituted by 1-4 groups R⁷;
each R⁶ is independently: halogen; cyano; nitro; hydroxyl; C₁₋₆ alkyl optionally substituted by 1-4 groups R^{L}; C₁₋₆ haloalkyl optionally substituted by 1-4 groups R^{L}; OR^{da}; S(O)_{d}R^{db}; C(O)R^{dc}; NR^{dk}R^{de}; or 2 adjacent groups R⁶ together with the atoms to which they are joined form a 5- or 6-membered ring optionally containing 1 or 2 heteroatoms selected from O, S and N, and said ring is optionally substituted by 1-4 groups R⁸;
each R⁷ is independently: halogen; cyano; nitro; hydroxyl; C₁₋₆ alkyl optionally substituted by 1-4 groups R^{L}; C₁₋₆ haloalkyl optionally substituted by 1-4 groups R^{L}; C₂₋₆ alkenyl optionally substituted by 1-3 groups R^{m}; phenyl optionally substituted by 1-4 groups R^{v}; heteroaryl optionally substituted by 1-3 groups R^{v}; OR^{da}; S(O)_{d}R^{db}; C(O)R^{dc}; or NR^{dk}R^{de}; or any two geminal groups R⁷ together form a group selected from oxo, ═CR^{dm}R^{dn}, ═NOR^{do}, or ═NNR^{dp}R^{dq}; or two groups R⁷ together with the atoms to which they are joined and any intervening atoms form a 3-7 membered ring system, wherein said ring system optionally contains an heteroatom selected from O, N and S, and is optionally substituted by 1-4 groups R⁹;
each R⁸ is independently: halogen, cyano, nitro, hydroxyl, C₁₋₆ alkyl optionally substituted by 1-4 groups R^{L}, C₁₋₆ haloalkyl optionally substituted by 1-4 groups R^{L}, OR^{da}, S(O)_{d}R^{db}, C(O)R^{dc}, or NR^{dk}R^{de}; or any two geminal groups R⁸ together form a group selected from oxo, ═CR^{dm}R^{dn}, ═NOR^{do}, and ═NNR^{dp}R^{dq};
each R⁹ is independently: halogen; cyano; nitro; hydroxyl; C₁₋₆ alkyl optionally substituted by 1-4 groups R^{L}; C₁₋₆ haloalkyl optionally substituted by 1-4 groups R^{L}; C₂₋₆ alkenyl optionally substituted by 1-3 groups R^{m}; OR^{da}; S(O)_{d}R^{db}; C(O)R^{dc}; or NR^{dk}R^{de}; or any two geminal groups R⁹ together form a group selected from oxo, ═CR^{dm}R^{dn}; ═NOR^{do}, and ═NNR^{dp}R^{dq};
each R^{L} is independently cyano, hydroxyl, C₃₋₆ cycloalkyl, OR^{da}, S(O)_{d}R^{db}, C(O)R^{dc}, or NR^{dk}R^{de};
each R^{m} is independently halogen, cyano, nitro, C₁₋₆ alkoxy, S(O)₂R^{db}, or C(O)R^{dc};
each R^{v} is independently halogen, cyano, nitro, hydroxyl, alkyl optionally substituted by one or more R^{L}, OR^{da}, S(O)_{d}R^{db}, C(O)R^{dc}, or NR^{dk}R^{de};
each R^{da} is independently C₁₋₆ alkyl, C₁₋₆ haloalkyl, C₁₋₄ alkoxy(C₁₋₄)alkyl, or C₁₋₆ alkylcarbonyl;
each R^{db} is independently C₁₋₆ alkyl, C₁₋₆ haloalkyl, C₂₋₆ alkenyl, C₂₋₆ alkynyl, C₃₋₆ cycloalkyl, or C₁₋₆ alkylcarbonylamino;
d is an integer selected from 0, 1, and 2;
each R^{dc} is independently hydrogen, hydroxyl, C₁₋₆ alkyl, C₁₋₆ haloalkyl, C₃₋₆ cycloalkyl, C₁₋₆ alkoxy, phenyl(C₁₋₆)alkoxy, C₃₋₆ cycloalkoxy, amino, C₁₋₆ alkylamino, di(C₁₋₄)alkylamino, or C₁₋₆ alkylsulphonylamino;
each R^{dk} is independently hydrogen, C₁₋₆ alkyl, C₁₋₆ alkylcarbonyl, C₁₋₆ alkoxycarbonyl, C₁₋₆ alkylsulphonyl, C₁₋₆ haloalkylsulphonyl, aminocarbonyl, C₁₋₆ alkylaminocarbonyl, or di(C₁₋₄)alkylaminocarbonyl;
each R^{de} is independently hydrogen, C₁₋₆ alkyl, or C₃₋₆ cycloalkyl; or R^{dk} and R^{de}, together with the nitrogen atom to which they are joined form a 5- or 6-membered saturated ring optionally containing one further heteroatom selected from O, N, and S, said ring being optionally substituted by 1-2C₁₋₆ alkyl or oxo groups;
each R^{dm} is independently hydrogen, halogen, cyano, nitro, C₁₋₆ alkyl, C₃₋₆ cycloalkyl, C₁₋₆ alkylcarbonyl, C₁₋₆ alkoxycarbonyl, C₁₋₆ alkylsulphonyl, or aminocarbonyl;
each R^{dn} is independently hydrogen, halogen, cyano, nitro, C₁₋₆ alkyl, C₁₋₆ alkylcarbonyl, C₁₋₆ alkoxycarbonyl, C₁₋₆ alkylsulphonyl, or aminocarbonyl;
each R^{do} is independently hydrogen, C₁₋₆ alkyl, or C₃₋₆ cycloalkyl;
each R^{dp} is independently hydrogen, C₁₋₆ alkyl, or C₃₋₆ cycloalkyl;
each R^{dq} is independently hydrogen or C₁₋₆ alkyl;
Y is halogen, C₁₋₃ alkyl, C₁₋₃ haloalkyl, C₁₋₂ alkoxy(C₁₋₂)alkyl, cyclopropyl optionally substituted by 1-2 groups R^{bc}, C₂₋₄ alkenyl, C₂₋₄ haloalkenyl, or C₂₋₄ alkynyl optionally substituted by 1-2 groups R^{be}; wherein each R^{bc} is independently halogen or C₁₋₂ alkyl, and each R^{be} is independently halogen or tri(C₁₋₃)alkylsilyl;
Z is (O)ₘ—(CR^{w}R^{x})ₙ—C(O)R^{cb}, wherein:
m is as defined in claim 1, n is an integer of 0 or 1, and n≧m;
R^{w} is hydrogen or C₁₋₄ alkyl and R^{x} is hydrogen;
R^{cb} is hydroxyl, C₁₋₁₀ alkylthio, or OR^{co};
R^{co} is C₁₋₂₀ alkyl optionally substituted by 1-2 groups R^{cq} or C₁₋₂₀ haloalkyl optionally substituted by 1-2 groups R^{cq};
each R^{cq} is independently phenyl optionally substituted with 1-3 groups R^{cr}, or heteroaryl optionally substituted with 1-2 groups R^{cs};
each R^{cr} is independently halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, or C₁₋₄ alkoxycarbonyl;
and,
each R^{cs} is independently halogen, cyano, C₁₋₄ alkyl, C₁₋₄ haloalkyl, C₁₋₃ alkoxy(C₁₋₃)alkyl, C₁₋₄ alkoxy, C₁₋₄ haloalkoxy, C₁₋₄ alkylsulphonyl, C₁₋₄ or alkoxycarbonyl.

5. The compound according to claim 4 wherein:
A is F, Cl, Br, I
a ring system selected from: phenyl, thiophene, furan, pyrrole, isoxazole, isothiazole, oxazole, thiazole, imidazole, pyrazole, 1,2,4-oxadiazole, 1,3,4-oxadiazole, 1,2,4-thiadiazole, 1,3,4-thiadiazole, 1,2,3-triazole, 1,2,4-triazole, pyridine, pyridazine, pyrimidine, pyrazine, 1,2,4-triazine, and 1,3,5-triazine, wherein said ring system is optionally substituted by 1-3 groups R¹; or, C₃₋₆ cycloalkyl, optionally substituted by 1-4 groups R²;
each R¹ is independently: halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio; or 2 adjacent groups R¹ together with the atoms to which they are joined form a 6 membered aromatic ring optionally substituted with 1-2 groups selected from: halogen, cyano, C₁₋₄ alkyl, C₁₋₄ haloalkyl, C₁₋₄ alkoxy, C₁₋₄ haloalkoxy, C₁₋₄ alkylthio, and C₁₋₄ haloalkylthio;
each R² is independently: halogen, cyano, C₁₋₄ alkyl, C₁₋₄ haloalkyl, C₁₋₄ alkoxy, C₁₋₄ haloalkoxy, C₁₋₄ alkoxycarbonyl, or C₁₋₄ alkylaminocarbonyl; or any two geminal groups R² together form a group selected from oxo, ═CR^{mm}Rⁿⁿ, ═NOR^{oo}; or two groups R² together with the atoms to which they are joined and any intervening atoms form a 5-6 membered ring system, optionally substituted by 1-2 groups selected from: halogen, cyano, C₁₋₄ alkyl, C₁₋₄ haloalkyl, C₁₋₄ alkoxy, and C₁₋₄ haloalkoxy;
each R^{mm} and Rⁿⁿ are independently: hydrogen, halogen, cyano, nitro, C₁₋₄ alkyl, or C₁₋₄ alkoxycarbonyl;
each R^{oo} is independently hydrogen, C₁₋₄ alkyl, or C₃₋₆ cycloalkyl;
R⁵ is hydrogen; C₁₋₄ alkyl optionally substituted by 1-2 groups R^{p}; C₁₋₄ haloalkyl optionally substituted by 1-2 groups R^{p}; C₃₋₆ cycloalkyl optionally substituted by 1-2 groups R^{q}; SO₂R^{ss}; or C(O)R^{uu};
each R^{p} is independently phenyl optionally substituted by 1-3 groups R^{aj}, heteroaryl optionally substituted by 1-3 groups R^{ak}, or C₁₋₄ alkoxy;
each R^{q} is independently halogen or C₁₋₄ alkyl;
each R^{ss} is independently C₁₋₄ alkyl or phenyl optionally substituted by 1-3 groups R^{zz};
each R^{uu} is independently C₁₋₄ alkyl, phenyl optionally substituted by 1-3 groups R^{zz}, alkoxy, or NR^{ac}R^{ad};
each R^{zz} is independently halogen, C₁₋₄ alkyl, C₁₋₄ alkoxy, or C₁₋₄ alkylsulphonyl;
each R^{ac} and R^{ad} are independently hydrogen, or C₁₋₄ alkyl;
each R^{aj} and R^{ak} are independently halogen, C₁₋₄ alkyl, C₁₋₄ haloalkyl, C₁₋₄ alkoxy, C₁₋₄ haloalkoxy, C₁₋₄ alkylsulphonyl;
W is a methylene group optionally substituted by 1-2 groups Rⁱ, wherein each Rⁱ is independently: cyano, C₁₋₂ alkyl, C₁₋₂ haloalkyl, C₁₋₂ hydroxyalkyl, C₁₋₂ alkoxy(C₁₋₂)alkyl, C₁₋₃ alkoxycarbonyl, or carboxy;
Q is a ring system selected from: phenyl, thiophene, furan, pyrrole, isoxazole, isothiazole, oxazole, thiazole, imidazole, pyrazole, 1,2,4-oxadiazole, 1,3,4-oxadiazole, 1,2,4-thiadiazole, 1,3,4-thiadiazole, 1,2,3-triazole, 1,2,4-triazole, pyridine, pyridazine, pyrimidine, pyrazine, 1,2,4-triazine, and 1,3,5-triazine, wherein said ring system is optionally substituted by 1-3 groups R⁶; C₃₋₆ cycloalkyl optionally substituted by 1-4 groups R⁷; a ring system selected from: oxetane, tetrahydrofuran, tetrahydropyran, dihydrofuran, dihydropyran, dioxolane, 1,3-dioxane, dithiolane, and 1,3-dithiane, wherein said ring system is optionally substituted by 1-4 groups R⁷;
each R⁶ is independently: halogen, cyano, C₁₋₄ alkyl, C₁₋₄ haloalkyl, C₁₋₄ alkoxy, C₁₋₄ haloalkoxy, C₁₋₄ alkylthio, or C₁₋₄ haloalkylthio; or 2 adjacent groups R⁶ together with the atoms to which they are joined form a 6-membered aromatic ring optionally substituted with 1-2 groups selected independently from: halogen, cyano, C₁₋₄ alkyl, C₁₋₄ haloalkyl, C₁₋₄ alkoxy, C₁₋₄ haloalkoxy, C₁₋₄ alkylthio, and C₁₋₄ haloalkylthio;
each R⁷ is independently: halogen, cyano, C₁₋₄ alkyl, C₁₋₄ haloalkyl, C₁₋₄ alkoxy, C₁₋₄ haloalkoxy, C₁₋₄ alkoxycarbonyl, C₁₋₄ alkylaminocarbonyl, phenyl optionally substituted by 1-3 groups R^{v}, or heteroaryl optionally substituted by 1-3 groups R^{v}; or any two geminal groups R⁷ together form a group selected from oxo, ═CR^{dm}R^{dn}, ═NOR^{do}; or two groups R⁷ together with the atoms to which they are joined and any intervening atoms form a 5-6 membered ring system optionally substituted by 1 or 2 groups independently selected from: halogen, cyano, C₁₋₄ alkyl, C₁₋₄ haloalkyl, C₁₋₄ alkoxy, and C₁₋₄ haloalkoxy;
each R^{v} is independently chosen from: halogen, cyano, C₁₋₄ alkyl, C₁₋₄ haloalkyl, C₁₋₄ alkoxy, C₁₋₄ haloalkoxy, C₁₋₄ alkylthio, C₁₋₄ haloalkylthio;
each R^{dm} and R^{dn} are independently hydrogen, halogen, cyano, nitro, C₁₋₄ alkyl, or C₁₋₄ alkoxycarbonyl;
each R^{do} is independently hydrogen, C₁₋₄ alkyl, or C₃₋₆ cycloalkyl,
Y is halogen, C₁₋₂ alkyl, C₁₋₂ haloalkyl, or C₁₋₂ alkoxy(C₁₋₂)alkyl;
and,
Z is C(O)R^{cb} wherein R^{cb} is hydroxyl, C₁₋₁₀ alkoxy, or phenyl(C₁₋₂)alkoxy.

6. The compound according to claim 2 wherein:
A is F, Cl, Br, I, a phenyl ring optionally substituted by 1-3 groups R¹, or cyclopropyl optionally substituted by 1-2 groups R²;
each R¹ is independently: halogen, cyano, C₁₋₂ alkyl, C₁₋₂ haloalkyl, C₁₋₂ alkoxy, or C₁₋₂ haloalkoxy;
each R² is independently: halogen, cyano, C₁₋₂ alkyl, C₁₋₂ haloalkyl, C₁₋₂ alkoxy, C₁₋₂ haloalkoxy, or C₁₋₃ alkoxycarbonyl;
R⁵ is hydrogen;
Q is a ring selected from: phenyl, furan, thiophene, oxazole, and thiazole, wherein said ring is optionally substituted by 1-2 groups R⁶; or C₃₋₆ cycloalkyl optionally substituted by 1-2 groups R⁷;
each R⁶ is independently: halogen, cyano, C₁₋₂ alkyl, C₁₋₂ haloalkyl, C₁₋₂ alkoxy, or C₁₋₂ haloalkoxy;
or 2 adjacent groups R⁶ together with the atoms to which they are joined to form a 6 membered aromatic ring optionally substituted with 1-2 groups independently selected from: halogen, cyano, C₁₋₂ alkyl, C₁₋₂ haloalkyl, C₁₋₂ alkoxy, and C₁₋₂ haloalkoxy;
and,
each R⁷ is independently: halogen, cyano, C₁₋₂ alkyl, C₁₋₂ haloalkyl, C₁₋₂ alkoxy, C₁₋₂ haloalkoxy, or C₁₋₃ alkoxycarbonyl.

7. An herbicidal composition comprising a compound as defined in claim 1 and at least one agriculturally acceptable formulation adjuvant or diluent.

8. The herbicidal composition according to claim 7, further comprising a crop safener.

9. A compound as defined in claim 2, in admixture with at least one active ingredient selected from the group consisting of: an insecticide, an acaricide, a nematocide, a molluscicide, an herbicide, a fungicide, and a plant growth regulator.

10. (canceled)

11. A method of controlling weeds in crops of useful plants which comprises applying to said weeds or to the locus of said weeds, or to said crop of useful plants, a compound as defined in claim 1.
